SEQUENCE LISTING (362 sequences)

SEQ ID NO: 1 - DNA, 375 bp, synthetic construct
  source 1..375: mol_type = other DNA
ggcgggtcagggggatctggcgagtctctgtttaagggccccagagattacaaccctatcagcagcaccatttgccacctgaccaacgagagcgatggacatacaaccagcctgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagacctag

SEQ ID NO: 2 - DNA, 598 bp, synthetic construct
  source 1..598: mol_type = other DNA
tctagagtcgacccagatccgcatccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataat

SEQ ID NO: 3 - DNA, 732 bp, synthetic construct
  source 1..732: mol_type = other DNA
atggccacaaccatgggatcagtgtccaagggcgaagaactgattaaggagaacatgcacatgaaactctatatggaaggcacagtggacaaccaccacttcaaatgcactagcgagggcgagggcaagccctacgagggcacccagaccatgcggatcaaagtggtggaaggcggacctctaccttttgccttcgacatcctggccaccagctttctgtacggctccaagacctttatcaaccatacccagggcatccccgacttcttcaagcagtctttccctgagggctttacatgggagagggtgaccacctatgaagatggaggcgtgctgacagccacacaggacaccagcctgcaagacggttgtctgatctacaacgtgaagatccggggcgtgaacttcacatctaatggccctgtgatgcagaaaaagacactgggatgggaggccttcaccgagacactgtaccctgccgacggaggtcttgaaggcagaaacgacatggccctgaaactggtgggaggaagccacctcatcgccaacgctaagacaacataccggagcaagaagcctgccaagaacctgaaaatgcccggcgtgtactacgtggactacagactggaaagaatcaaggaagctaataacgaaacctacgtggaacagcacgaggtggccgtcgccagatactgcgacctgcctagcaagctgggccacaagctgaactag

SEQ ID NO: 4 - DNA, 378 bp, synthetic construct
  source 1..378: mol_type = other DNA
ggcgggtcagggggatctaagagcatgagcagcatggtctccgacacaagctgtaccttcccatctagcgacggcattttctggaagcactggatccagaccaaggacggccagtgcggctctcctctggtgagcaccagagacggcttcatcgtgggcatccactctgccagcaactttacaaacacaaacaattacttcaccagcgtgccaaagaacttcatggaactgctgaccaaccaggaggctcagcagtgggtgtccggctggcggctgaacgccgatagcgtgctgtggggcggccacaaagtgttcatggtgaagccagaggagcctttccaacctgtgaaggaggctacccagctgatgaacagctag

SEQ ID NO: 5 - protein, 124 aa, synthetic construct
GGSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQT

SEQ ID NO: 6 - protein, 243 aa, synthetic construct
MATTMGSVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 7 - protein, 125 aa, synthetic construct
GGSGGSKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNS

SEQ ID NO: 8 - DNA, 297 bp, synthetic construct
  source 1..297: mol_type = other DNA
atgatggagcctccaagaggacctcctgctaatggagccgagccctctagagccgtgggcaccgtgaaggtgtacctgcctaacaagcagagaaccgtcgtgaccgtgcgggacggcatgagcgtgtacgacagcctggataaggccctgaaggtgcggggcctgaaccaggactgctgtgtggtgtacagactgatcaagggcgctaaaaccgttacagcctgggacaccgccatcgcccctctcgacggcgaggagctgatcgtggaagtgctggaagatgtgcccctgacaatg

SEQ ID NO: 9 - DNA, 504 bp, synthetic construct
  source 1..504: mol_type = other DNA
atgatggaaccccccagaggacctcctgctaatggcgccgagcctagcagagccgtgggcaccgttaaggtgtacctgcctaacaagcagagaaccgtcgtgactgtgcgggacggaatgtctgtgtacgacagcctggataaggccctgaaggtgcggggcctgaaccaggactgctgcgtggtgtatagactgatcaagggcgctaaaacagtcacagcctgggacaccgccatcgcccctctggacggcgaggagctgatcgtggaggtgctggaagatgtgcccctcaccatgcacaacttcgtgcggaagaccttcttcagcctggccttttgcgacttctgcctgaaattcctgttccacggcttccgctgtcagacctgtggctacaagtttcatcagcactgcagcagcaaggtgccaacagtgtgcgtggacatgtccaccaacagacagcaaccttctagattctaccacagcgtgcaggatctgtccggcggc

SEQ ID NO: 10 - DNA, 243 bp, synthetic construct
  source 1..243: mol_type = other DNA
atgggacctaagtctccacagaaacctatcgtgcgggtgttcctgcctaacaagcagagaacagtcgtgcctgctagatgtggcgtgaccgtgcgggacagcctgaagaaagccctgatgatgagaggcctgatccccgagtgctgcgccgtgtacagaatccaggacggcgccaagaagcccatcggctgggataccgacatcagctggctgaccggcgaggaactgcacgtggaagtgctg

SEQ ID NO: 11 - DNA, 243 bp, synthetic construct
  source 1..243: mol_type = other DNA
atgtccaagaccagcaataccatcagagtgttcctgcctaacaagcagagaaccgtggtgaacgtgcggaacggcatgagcctgcacgactgtctgatgaaagccctgaaggtgcggggactgcagcccgagtgctgcgccgtgtttagactgctgcacgaacacaagggcgccaaggccagactcgactggaacacagatgccgcttctctgatcggcgaggaactgcaggtcgacttcctg

SEQ ID NO: 12 - DNA, 243 bp, synthetic construct
  source 1..243: mol_type = other DNA
atgtccaagaccagcaacacaatcgccgtgttcctccccaacaagcagagaaccgtggtgaacgtgcggaacggcatgagcctgcacgattgtctgatgaaagccctgaaggtgcggggactgcagcctgaatgctgcgccgtctttagactgctgcacgagcacaagggcaaaaaggccagactggactggaataccgacgccgcttctctgatcggcgaggaactgcaggtggacttcctg

SEQ ID NO: 13 - DNA, 243 bp, synthetic construct
  source 1..243: mol_type = other DNA
atgtccaagaccagcaacaccatcgccgtgttcctgcctaacaagcagagaaccgtggtgaacgtgcggaatggaatgagcctgcacgactgtctgatgaaagccctgaaggtgcggggcctgcagcccgagtgctgcgccgtctttagactgctgcacgaacacaagggcgccaaggctagactcgactggaacacagatgccgcttctctgatcggcgaggaactgcaggtggacttcctg

SEQ ID NO: 14 - DNA, 399 bp, synthetic construct
  source 1..399: mol_type = other DNA
atgtccaagacaagcaacaccatcagagtgtttctgcctaacaagcagagaaccgtggtgaacgtgcggaatggcatgagcctgcacgactgcctgatgaaagccctgaaagtgcggggcctgcagcctgagtgctgcgccgtgttccggctgctgcatgaacacaagggcgccaaggctagactggactggaatacagatgccgcttctctgatcggagaggaactgcaagtggacttcctggatcacgtgccactgaccacccacaacttcgccagaaagaccttcctgaagctggccttctgcgacatctgccagaagttcctcctgaacggcttcagatgtcagacctgtggctacaagtttcacgagcactgcagcaccaaggtccccacaatg

SEQ ID NO: 15 - DNA, 282 bp, synthetic construct
  source 1..282: mol_type = other DNA
atgaacaactgcatcttcatcgtgatccacagaagcaccaccagccaaacaatcaaagtgtccccagatgacacccctggcgccatcctgcagtcttttttcaccaagatggccaagaagaaaagcctgatggacatccccgagagccagagcgagcaggacttcgtgctgagagtgtgcggcagagatgaatacctggtcggagagacacctattaagaatttccagtgggtgcggcactgtctgaagaacggcgaggaaatccacgtggtgctggacacc

SEQ ID NO: 16 - DNA, 363 bp, synthetic construct
  source 1..363: mol_type = other DNA
atgagctctccagagctgcctaagcacatctacaacaagctggataagggccagatcatcgtggtgatctgggtcattgtgtcccctaacaacgacaagcagaaatatacactgaagatcaaccacgactgcgtgcccgagcaagtgatcgccgaggctatcagaaagaagaccagaagcatgctgctgagcagcgagcagctgaagctgtgtgtgctggaataccagggaaaatacatcctgaaagtgtgcggctgcgacgagtacttcctggaaaagtaccccctgtctcagtacaagtacatccggagctgcatcatgctcggcagaatgcctaatctgatgctgatggccaaggaaagc

SEQ ID NO: 17 - DNA, 327 bp, synthetic construct
  source 1..327: mol_type = other DNA
atgtccgcccagacctggggccctggaacactgcggctgccaaaccgggccctgctggtcaatgtgaagttcgagggcagcgaggaaagcttcacctttcaggtgtccacaaaggacgtgcccctggctctgatggcctgcgccctcagaaagaaagccaccgtgttcagacagcctctggtggaacagcccgaggattacaccctgcaggtgaacggcagacacgagtacctgtacggctcttatcctctgtgccagttccaatacatctgcagctgtctgcacagcggcctgacacctcacctgaccatggtgcatagctctagc

SEQ ID NO: 18 - DNA, 336 bp, synthetic construct
  source 1..336: mol_type = other DNA
atgggaggcagcgagagcttcttcgtgcaggtgcacgatgtgtctccagagcaacctagaacagtgatcaaggcccctcgggtgtccaccgcccaggacgtgatccagcagacactgtgtaaagccaagtactcctacagcatcctgagcaaccccaatcccagcgactacgtgctcctggaagaggtggtgaaggacaccacaaacaagaaaaccaccacccctaagagcagccagagagtgctgctggatcaggagtgcgtctttcaggcccagagcaagtggaagggcgccggcaagttcatcctgaaactgaaggaacaggttcaagcttct

SEQ ID NO: 19 - DNA, 819 bp, synthetic construct
  source 1..819: mol_type = other DNA
atggtggaatggcacatcagccggcctggccacatcgagacctttgacctgctgaccctgcaccctatcgagatcgctagacagctgacactgctggaaagcgacctgtaccgggccgtgcagccttccgagctggtgggcagcgtgtggaccaaggaagataaggaaatcaattctccaaacctgctgaaaatgatcagacacaccacaaatctgaccctgtggttcgaaaagtgcatcgtggaaaccgagaatctggaggagagagtggctgtggtctccagaatcatcgagatcctgcaagtgttccaggagctcaacaacttcaacggcgtgctggaagtggtttctgccatgaacagcagccctgtgtacagactggaccatacattcgagcagatcccttcccggcagaaaaagatcctggaggaggcccacgagctgagcgaggatcactacaagaagtatctggccaagctgcggagcattaaccccccctgtgtgcccttcttcggaatctacctgaccaacatccttaaaacagaggaaggcaaccctgaggtgctgaagagacacggaaaggagctgatcaatttcagcaagaggcggaaggtggccgagatcaccggcgagattcagcaataccagaaccagccttactgcctgagagtcgagtctgacatcaagagatttttcgaaaacctgaaccccatgggcaacagcatggaaaaggaattcaccgactacctgtttaacaagagcctggaaatcgagcctcgcaaccccaagcctctcccaagattccctaaaaagtacagc

SEQ ID NO: 20 - DNA, 282 bp, synthetic construct
  source 1..282: mol_type = other DNA
atggctacacattgttttcagcacctgctgagagtcgcctaccaggaccctagctccggctgcaccagcaagaccctcgccgtgccccccgaagcctctatcgccaccctgaaccagctgtgcgccacaaaattcagagtgacccaacctaacaccttcggcctgttcctgtacaaggaacagggatatcaccggctgcctccaggcgccctggcccacagactgcctaccaccggctacctggtgtaccggagagccgagtggcctgagacacagggcgct

SEQ ID NO: 21 - DNA, 1287 bp, synthetic construct
  source 1..1287: mol_type = other DNA
atgggcgtgatccccaccctggtcaagctgctgggcatccactgccagaacgccgccttgacagagatgtgcctggtggccttcggcaacctggccgagctggaatctagcaaggagcagttcgccagcacaaacatcgccgaagagctggtgaagctgttcaagaagcagatcgagcacgacaagagagagatgatcttcgaggtgctggcccctctggccgaaaacgacgccatcaagctgcagctggtggaagcaggcctggtcgaatgcctgctggaaatcgtgcaacagaaagtggacagcgacaaggaagatgacatcaccgaactgaagacaggctctgatctgatggtgctgctgctgttaggcgacgagagcatgcagaagctgtttgagggcggcaagggcagcgtgttccagcgggtgctgagctggatccccagcaacaaccaccaattgcagctggccggcgccctggccatcgccaacttcgccagaaatgacgccaattgcatccacatggtggataacggcatcgtggaaaagctgatggacctgctggatagacacgtggaggacggaaatgtgaccgtgcaacatgccgccctgagcgccctcagaaacctggccattcctgtgattaacaaggccaagatgctgtccgccggcgtgaccgaggccgtgctgaagttcctgaaatctgagatgcctccagtgcagtttaagctgctggggaccctgagaatgctcatcgacgctcaggccgaggccgctgaacagctgggaaaaaacgtgaagctagttgaaagactggtcgagtggtgtgaagccaaggaccacgccggcgtgatgggcgagtctaatagactgctgtctgccctgatccggcacagcaagagcaaagatgtgatcaagaccatcgtgcagagcggcggaatcaagcacctggtcaccatggccacaagcgagcacgtgatcatgcagaacgaggctcttgttgctctggccctgatcgccgctctggagctgggcaccgccgagaaggacctggaatccgctaaactggtgcagatcctgcacagactgctggctgatgagcggagcgcccctgagatcaaatacaacagcatggttctgatctgcgccctgatgggaagcgagtgtctccataaggaagtgcaggacctggcattcctggacgtggtgtccaaactgcggagccacgagaacaagtccgtggcccagcaggcttctctgaccgagcagaggctgacagtggaaagc

SEQ ID NO: 22 - DNA, 354 bp, synthetic construct
  source 1..354: mol_type = other DNA
atgtccagccccggcctgaccagcaaggaatctctctttgtgcggatcaacgccgctcacggcttcagcctgatccaggtggacaacaccaaggtgaccatgaaagagatcctgctgaaggccgttaagcggagaaagggatctcagaaagtgtccggccctcagtacagactggaaaagcagagcgagcctaatgtggctgtggatctggacagcacactggaaagccaaagcgcctgggagttctgcctggtcagagagaacagctctagagccgacggcgtgttcgaggaagatagccagatcgacatcgccacagtgcaggacatgctgagcagccaccactacaagtcc

SEQ ID NO: 23 - DNA, 489 bp, synthetic construct
  source 1..489: mol_type = other DNA
atgggcggccctacactgcaggagatcaagcaaaagatcgacagctataacacccgggaaaagaactgcctgggcatgaaactgtctgaagatggaacatacaccggcttcatcaaggtgcacctgaagctgcggagacctgtgaccgtgcctgccggcatcagaccacagagcatctacgacgccatcaaagaggtgaatctggctgctacaacagataagagaaccagcttctacctgcctctggacgccattaagcagctgcacatcagcagcaccaccaccgtgtccgaggtgatccagggcctgctgaaaaagttcatggtcgtggacaacccccagaagttcgccctgttcaagcggatccacaaggacggccaggtgctgtttcagaaactgagcatcgccgatagacccctgtacctgagactgctggccggacctgacaccgaggttctgtcttttgtgctcaaggaaaacgagacaggc

SEQ ID NO: 24 - DNA, 690 bp, synthetic construct
  source 1..690: mol_type = other DNA
atgtccgacgccgacaagctgagaaaggtgatctgcgagctgctcgagacagagagaacctacgtgaaggacctgaactgtctgatggaaagatacctgaaacctctgcagaaagagaccttcctgacacaggacgagctggatgtgctgtttggaaatctgaccgagatggtcgagttccaggtggaattcctgaagacactggaagatggcgtgcggctggtgcctgatctggaaaaactggaaaaggtggaccaatttaagaaagtgctgttcagcctgggcggcagcttcctgtattacgccgataggttcaagctgtacagcgccttctgcgcctctcacaccaaggtgccaaaggtccttgtgaaagccaagaccgatacagccttcaaggcctttctggacgcccaaaaccccaagcagcagcattctagcaccctggagagctacctgattaagcctatccagagaatcctgaagtaccccctcctgctgcgggaactgttcgccctgaccgacgctgagagcgaggaacactaccacctggacgtggccatcaagaccatgaacaaggttgcttcccacatcaacgagatgcagaagatccacgaggaattcggcgccgtgttcgaccagctgatcgctgagcagacaggcgagaagaaggaagtggccgacctgagcatg

SEQ ID NO: 25 - DNA, 333 bp, synthetic construct
  source 1..333: mol_type = other DNA
atgtctgtgtccgtgaccagcatcacatctacagtgctgcctcctgtgtacaaccagcagaacgaggacacctgcatcatcagaatcagcgtggaagataacaacggcaatatgtataagtccatcatgctgaccagccaggacaagacccctgccgtgatccagcgggccatgctgaagcacaacctggacagcgaccccgctgaagagtacgagctggtgcaggtgattagcgaggataaggaactggtcatccccgatagcgccaatgtgttctacgccatgaacagccaagtgaacttcgactttatcctgagaaagaaaaacagcatg

SEQ ID NO: 26 - DNA, 855 bp, synthetic construct
  source 1..855: mol_type = other DNA
atgtccgccgaattcacctgctttagtgaagatctggtggccgagcagctgacctacatggacgcccagctgttcaagaaagtggtcccacatcactgcctgggatgtatctggtccagacgggacaagaaagagaacaagcacctggcccctaccattagagctacaatcagccagttcaacaccctgacaaagtgcgtggtgtctacaatcctgggcggcaaggagctgaagacccaacaaagagccaagatcatcgagaagtggatcaacatcgcccacgagtgcagactgctgaagaacttctctagcctgagggctattgtgtccgctctgcagagcaacagcatctacagactcaaaaagacctgggccgccgtgcccagagatagaatgctgatgttcgaggaactgtctgatatctttagcgaccacaacaaccacctgaccagccgggaactgctgatgaaagaaggcacaagcaagttcgccaatctggacagcagcgttaaggaaaaccagaaacgcacccagcggagactgcagctgcaaaaggacatgggcgtgatgcagggcaccgtgccttacctgggcacattcctgaccgacctgaccatgctggacaccgccctgcaggactacatcgagggaggcctgatcaacttcgagaagcggcggagagagttcgaggtgatcgcccagatcaagctgctccagagcgcctgtaatagctactgcatgacccctgaccagaagtttatccagtggttccagagacagcagctgcttacagaagaagagtcttatgccctgagctgcgagatcgaggccgctgctgatgcctctaccacaagc

SEQ ID NO: 27 - DNA, 1212 bp, synthetic construct
  source 1..1212: mol_type = other DNA
atgtccgctgaattcacctgtttttctgaagacctggtggccgagcagctgacctacatggacgcccagctgttcaaaaaggtggtgccccaccactgtctgggctgcatctggagcagaagagataagaaggaaaacaagcacctggcccctaccatccgggctacaatcagccagttcaacaccctgaccaagtgcgtggtctctaccattctgggaggcaaggaactgaagacacagcaaagagccaagatcatcgaaaagtggattaacatcgcccacgagtgcagactgctgaagaacttcagcagcctgagggctatcgtgtccgccctgcagtccaacagcatctacagactgaagaaaacctgggccgccgtgccaagagatagaatgctgatgttcgaggaactgagcgatatcttcagcgaccacaacaatcacctgaccagccgggagctgctcatgaaagagggaaccagcaagtttgccaacctggactcttccgttaaggagaaccagaaaagaacccagcggcgcctccagctgcaaaaggacatgggcgtgatgcagggcacagtgccctacctgggcacctttctgaccgatctgacaatgctggataccgccctgcaagactacatcgagggcggcctgatcaacttcgagaagcggagacgggaattcgaggtgatcgcccagatcaagctgctgcagagcgcctgcaactcttattgtatgacacctgaccagaagttcatccagtggttccagagacagcagctgctgaccgaggaagagagctacgccctgtcatgcgagatcgaggccgctgctgatgccagcaccacaagcggcggatctggcggaagcggcggcagcagtgtgagcgtgacaagcatcacatctacagtgctgcctcctgtgtacaaccagcagaacgaggacacctgcatcatcagaatcagcgtggaagacaacaacggcaatatgtacaagagcatcatgctgaccagccaggacaagacccctgcagttatccagagagccatgctgaaacataatctggactccgatcctgccgaggaatacgagctggtccaggtgatttctgaggacaaggaactggtgatccccgactctgccaatgtgttctacgccatgaatagccaagtgaactttgacttcatcctgcggaagaaaaacagcatg

SEQ ID NO: 28 - DNA, 336 bp, synthetic construct
  source 1..336: mol_type = other DNA
atgggcagccccagaatccccctgccagctcagcagagcagcgaggccagagtgatccgggtgtccatcgacaacgaccacggcaacctgtacagaagcatcctgctcacatctcaggacaaggccccttctgtcgtgcggagggccctgcagaaacacaacgttccccaaccttgggcctgcgactaccagctgtttcaggtgctgcctggagatagagtgctgctgatccctgataatgccaacgtgttctacgccatgagccctgtggctcctcgggacttcatgctgagacgcaaggaaggcaccagaaacaccctgagcgtgtccccaagc

SEQ ID NO: 29 - DNA, 993 bp, synthetic construct
  source 1..993: mol_type = other DNA
atgatgcctgaggaagagtacagcgagttcaaggaactgatcctccagaaagagctgcatgtggtgtacgccctgtctcacgtgtgcggccaggatcggacactgctggccagcatcctgctgcggatctttctgcacgagaaactggaaagcctgctgctttgtaccctgaacgaccgggaaatcagcatggaagatgaggccacaacactgttccgggccaccaccctggcaagcaccctgatggaacagtatatgaaggccaccgccacacagttcgtgcaccacgccctgaaagacagcatccttaagatcatggaatccaagcagagctgcgagctgtcccctagcaagctggaaaagaacgaggacgtgaacaccaacctgacccacctgctgaacatcctgagcgagctggtggaaaagatcttcatggcctctgagatcctgcctcctacactgagatacatctacggctgtctgcagaagtctgttcagcataagtggcctaccaacaccaccatgaggaccagagtggtcagcggcttcgtgttcctgagactgatttgccccgccatcctgaaccctagaatgttcaacatcatctctgatagcccttccccaatcgccgccagaaccctgatcctggtcgccaagagcgtgcagaatctggctaatctggtggagttcggcgctaaggaaccctacatggaaggagtgaaccccttcatcaagtccaacaagcacagaatgatcatgtttctggacgagcttggaaatgtgcccgagctgccagacaccacagagcactctagaacagacctgagcagagatctggctgctctgcacgagatttgcgtggcccacagcgacgagctgcgcacactgagcaacgagagaggcgcccagcagcacgtgctgaagaagctgttggccatcacagagctcctgcaacaaaaacagaaccagtacaccaagaccaacgacgtgcgg

SEQ ID NO: 30 - DNA, 840 bp, synthetic construct
  source 1..840: mol_type = other DNA
atgggagagctgcctatcgccatggccctggctaatgtggtgccttgcagccagtgggacgagctggccagagtgctggttacactgttcgacagcagacacctgctgtatcagctgctgtggaacatgttcagcaaggaagtggaactcgcagatagcatgcagaccctgtttagaggcaatagcctggcttctaagatcatgaccttctgcttcaaggtgtacggcgccacatacctgcaaaagctgctcgaccccctgctgcggatcgtgatcacatctagcgactggcagcacgtgtccttcgaggtcgaccctaccagactggaacccagcgagagcctggaagagaaccagcggaacctgctgcagatgacagagaagttcttccacgccatcatcagcagcagctccgagttccctcctcagctgagaagcgtgtgccactgtctgtaccaggccacctgtcattctctgctgaacaaggccaccgtgaaagaaaagaaggaaaacaagaagtccgtggtcagccaaagatttccacagaacagcatcggcgctgtgggcagcgccatgttcctgcggttcatcaaccctgctattgtgtccccttacgaggccggcatcctggataagaaacctccacctagaatcgagagaggactgaagctgatgtctaaaatcctgcagtctatcgccaatcacgtgctgtttaccaaggaggaacacatgcgccccttcaacgacttcgtgaaaagcaacttcgatgccgccaggcggtttttcctggacatcgccagcgattgccccaccagcgacgccgtgaaccacagcctttct

SEQ ID NO: 31 - DNA, 1149 bp, synthetic construct
  source 1..1149: mol_type = other DNA
atggtcaccctgcaggagtggaacgaccctctggaccacgacctggaagcccagctgatctatagacatctgctgggcgtggaagccatgctgtgggagagacacagagagctgagcggaggcgcggaggccggcaccgtgcctaccagccccggcaaggtgcccgaggactctctggccagactgctgcgggtgctgcaggatctgagagaggcccacagctctggcggcagcggaggagttctgccttccagctactaccagcccctggtgcacttgctctgccacgaggtgaagctgggcatgcagggccctggccagctgatccccctgattgaggagacaacctccaccgagtgcagacaggacgtggcaaccaacctgctcaagctgtttctgggccagggcctggccaaggacttcctggatctgctgtttcagctcgagctgagcagaaccagtgaaaccaataccctgtttcggagcaacagcctggcttctaagagcatggaaagcttcctgaaagtggccggaatgcagtacctgcacggcgtgctgggacctatcatcaacaaggtgttcgaggaaaaaaagtacgtggaactggatcctagcaaggtcgaggtgaaagacgtgggctgcagcggtcttcatagacctcagacagaggctgaagtgctggagcagagcgcccagaccctgagggcccacctgggcgccctgctgtctgccctgtctagaagcgtgcgggcttgtcctgctgtggtgagagctacattcagacagctgttccggagagtgcgcgaaaggttcccaggcgctcaacacgagaacgtgccattcatcgccgtgacctccttcctgtgcctgcggttcttcagccccgccatcatgagccctaagctgttccacctgagagaacggcacgccgatgccagaacatctcggacactgctgctgctggccaaggccgtgcagaatgtgggcaacatggacacccctgccagcagagccaaagaggcctggatggaacccctgcagcctacagtgcggcagggcgttgcccaactgaaggatttcatcaccaagctagtcgacatcgaggaaaaggacgagctggacctgcaaagaacactgagcctgcaggcc

SEQ ID NO: 32 - DNA, 912 bp, synthetic construct
  source 1..912: mol_type = other DNA
atggttctgccttcttcttactaccagcctctggtgcacctgctgtgccacgaggtcaagctgggcatgcagggccccggccagctgatccctctgattgaggagacaacatccaccgagtgcagacaggacgtggctaccaatctgctgaaactgttcctgggccagggcctggctaaggatttcctggacctgctctttcagctcgagctgagcagaaccagcgagacaaacaccctgttcagaagcaacagcctggcatctaaaagcatggaaagctttctgaaggtggccggcatgcagtatctgcacggcgtgctgggccctatcatcaacaaggtgttcgaagaaaagaagtacgtggaactggatcctagcaaggtggaggtgaaggacgtgggctgtagcggcctgcatagaccacagaccgaggccgaggtgctggaacagagcgcccagaccctgagagcccacctgggagccctgctgtctgccctgtcccgcagcgtgcgggcctgccccgccgtggtgagagccaccttcagacagctgtttcggcgggtgagagaaagattccctggcgcccaacacgagaacgtgcccttcatcgccgtcacaagcttcctgtgcctgagattcttcagccccgctatcatgagcccaaagctgttccacctgcgggaaagacacgccgatgccagaacaagccggaccctgctgctgctagccaaggccgtgcagaacgtgggaaatatggacacccctgcttctagagccaaagaggcctggatggaacctctgcaacctacagtgcggcagggagtggcccaactgaaggacttcatcaccaagctcgtggacatcgaggaaaaggacgagctggatctgcagaggaccctgtccctgcaggct

SEQ ID NO: 33 - DNA, 801 bp, synthetic construct
  source 1..801: mol_type = other DNA
atggctagcaaccagcgggaagagtacctgctgctgagattgtttaagaccgccctgcaggaggaaatcaagtccaaggtggatcagatccaggagatcgtgacaggaaatcctaccgtgatcaagatggtggtgtccttcaacagaggcgccagaggacagaacgccctgaggcagattctggctcctgtggtgaaggaaatcatggacgacaagagcctgaacatcaaaaccgaccccgtggacatctacaagagctgggtcaatcaaatggaaagccaaacaggcgaggccagcaagctgccatacgacgtgacccctgagcaggccctggcacatgaggaagtgaaaacaagactggacagcagcatccggaacatgcgggccgtgacagataagttcctgagcgccatcgtttccagcgtggacaaaatcccctacggcatgagattcatcgccaaggtgctgaaggactctctgcacgagaagttccctgacgctggcgaagatgagctgctcaagatcatcggcaacctgctgtactacagatacatgaaccccgccatcgtcgcccctgatgccttcgatatcatcgacctgagcgccggaggccagctgaccaccgatcagcgcagaaacctgggcagcattgctaaaatgctgcaacacgccgcctctaacaagatgttcctgggcgacaatgcccacctgagcatcatcaacgagtacctgtctcagtcttatcagaagtttagacggttcttccagaccgcctgcgacgtgcctgagctgcagggcggc

SEQ ID NO: 34 - DNA, 945 bp, synthetic construct
  source 1..945: mol_type = other DNA
atggttctgcctagcgagcggtacaaggaactggctgagttcctcaccttccactacgccagactgtgcggcgccctggaacctgctctgccagcccaggccaaggaagagctggcagccgccatggtgagagtgctgcgagccaccggtagagcccaagccttggtgaccgacctgggcaccgccgagctggcccggtgcggcggcagagaggccctgctgtttagagagaacaccctggccacaaaggccatcgacgagtacatgaaactggtcgcccaggactacctgcaggagacactgggccaggtggtgcgcagactgtgcgcctctacagaggactgcgaggtggaccccagcaagtgccccgcttctgaactgcccgagcaccaggccagactgagaaacagctgtgaagaagtgttcgagaccatcatccacagctacgactggttccctgctgaactgggaatcgtgttcagcagctggcgggaagcctgcaaggagagaggctccgaggtgctgggacctagactggtgtgcgccagcctgttcctgaggctgctgtgtcctgctattctggctccttccctgtttggcctggccccagatcaccccgcccctggacctgcccggaccctcaccctgatcgccaaagtgatccagaacctggctaatagagccccattcggcgagaaggaagcctatatgggatttatgaacagcttcctcgaggaacatggccctgctatgcagtgcttcctggaccaggtcgccatggtggatgtggacgccgctccttctggctaccagggcagcggcgacctggccctgcaactggccgtgctgcacgcccagctgtgtacaatcttcgccgagctggatcagaccacgagagataccctggaacccctgcctacaatcctgcgggccatcgaggaaggc

SEQ ID NO: 35 - DNA, 249 bp, synthetic construct
  source 1..249: mol_type = other DNA
atggatccgagtaagacgtcaaacacaatcgccgtttttctccccaataaacagcggaccgtggtaaacgtccgaaacgggatgagtctccacgactgtctgatgaaagccttgaaggtgagggggcttcagcctgaatgttgcgctgtctttcgccttctccacgaacacaagggcgcaaaggcgaggcttgactggaatacagacgctgcaagtttgattggggaagaacttcaggtcgatttcctc

SEQ ID NO: 36 - DNA, 492 bp, synthetic construct
  source 1..492: mol_type = other DNA
atgggtacggaatataagctggtggtggtgggcgccggcggtgtgggcaagagtgcgctgaccatccagctgatccagaaccattttgtggacgaatacgaccccactatagaggattcctaccggaagcaggtcgtcattgatggggaaacgtgcctgttggacatcctggataccgccggccaggaggagtacagcgccatgcgggaccagtacatgcgcaccggggagggcgggtccgggggggatccgagtaagacgtcaaacacaatcgccgtttttctccccaataaacagcggaccgtggtaaacgtccgaaacgggatgagtctccacgactgtctgatgaaagccttgaaggtgagggggcttcagcctgaatgttgcgctgtctttcgccttctccacgaacacaagggcgcaaaggcgaggcttgactggaatacagacgctgcaagtttgattggggaagaacttcaggtcgatttcctc

SEQ ID NO: 37 - DNA, 525 bp, synthetic construct
  source 1..525: mol_type = other DNA
atgaatgtcgacgagccagttgagtgggaaaccccggacctgtctcaagcagagatcgaacagaagataaaggaatataatgcacaaatcaattccaatttgtttatgagtctcaacaaagacggctcatacacgggttttatcaaggtccaactcaaactggtaaggccggtgtccgttccaagtagtaagaagccgccttctctccaggatgctcgacgggggcccgggcggggaacctcagtacgcaggaggacctcattttatttgccaaaagacgcagtgaagcacttgcacgtattgtctcgaaccagggcacgcgaggtcatagaggctcttctgcgaaaattcttggtagtggacgatccgcgcaagttcgccctgttcgagcgagccgagcggcatggacaggtttatttgcggaagctccttgacgacgaacagccactcagactccgccttctcgcggggccctcagacaaagcattgagttttgtccttaaggagaatgattctggtgaggtc

SEQ ID NO: 38 - protein, 99 aa, synthetic construct
MMEPPRGPPANGAEPSRAVGTVKVYLPNKQRTVVTVRDGMSVYDSLDKALKVRGLNQDCCVVYRLIKGAKTVTAWDTAIAPLDGEELIVEVLEDVPLTM

SEQ ID NO: 39 - protein, 168 aa, synthetic construct
MMEPPRGPPANGAEPSRAVGTVKVYLPNKQRTVVTVRDGMSVYDSLDKALKVRGLNQDCCVVYRLIKGAKTVTAWDTAIAPLDGEELIVEVLEDVPLTMHNFVRKTFFSLAFCDFCLKFLFHGFRCQTCGYKFHQHCSSKVPTVCVDMSTNRQQPSRFYHSVQDLSGG

SEQ ID NO: 40 - protein, 81 aa, synthetic construct
MGPKSPQKPIVRVFLPNKQRTVVPARCGVTVRDSLKKALMMRGLIPECCAVYRIQDGAKKPIGWDTDISWLTGEELHVEVL

SEQ ID NO: 41 - protein, 81 aa, synthetic construct
MSKTSNTIRVFLPNKQRTVVNVRNGMSLHDCLMKALKVRGLQPECCAVFRLLHEHKGAKARLDWNTDAASLIGEELQVDFL

SEQ ID NO: 42 - protein, 81 aa, synthetic construct
MSKTSNTIAVFLPNKQRTVVNVRNGMSLHDCLMKALKVRGLQPECCAVFRLLHEHKGKKARLDWNTDAASLIGEELQVDFL

SEQ ID NO: 43 - protein, 81 aa, synthetic construct
MSKTSNTIAVFLPNKQRTVVNVRNGMSLHDCLMKALKVRGLQPECCAVFRLLHEHKGAKARLDWNTDAASLIGEELQVDFL

SEQ ID NO: 44 - protein, 133 aa, synthetic construct
MSKTSNTIRVFLPNKQRTVVNVRNGMSLHDCLMKALKVRGLQPECCAVFRLLHEHKGAKARLDWNTDAASLIGEELQVDFLDHVPLTTHNFARKTFLKLAFCDICQKFLLNGFRCQTCGYKFHEHCSTKVPTM

SEQ ID NO: 45 - protein, 94 aa, synthetic construct
MNNCIFIVIHRSTTSQTIKVSPDDTPGAILQSFFTKMAKKKSLMDIPESQSEQDFVLRVCGRDEYLVGETPIKNFQWVRHCLKNGEEIHVVLDT

SEQ ID NO: 46 - protein, 121 aa, synthetic construct
MSSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKES

SEQ ID NO: 47 - protein, 109 aa, synthetic construct
MSAQTWGPGTLRLPNRALLVNVKFEGSEESFTFQVSTKDVPLALMACALRKKATVFRQPLVEQPEDYTLQVNGRHEYLYGSYPLCQFQYICSCLHSGLTPHLTMVHSSS

SEQ ID NO: 48 - protein, 112 aa, synthetic construct
MGGSESFFVQVHDVSPEQPRTVIKAPRVSTAQDVIQQTLCKAKYSYSILSNPNPSDYVLLEEVVKDTTNKKTTTPKSSQRVLLDQECVFQAQSKWKGAGKFILKLKEQVQAS

SEQ ID NO: 49 - protein, 273 aa, synthetic construct
MVEWHISRPGHIETFDLLTLHPIEIARQLTLLESDLYRAVQPSELVGSVWTKEDKEINSPNLLKMIRHTTNLTLWFEKCIVETENLEERVAVVSRIIEILQVFQELNNFNGVLEVVSAMNSSPVYRLDHTFEQIPSRQKKILEEAHELSEDHYKKYLAKLRSINPPCVPFFGIYLTNILKTEEGNPEVLKRHGKELINFSKRRKVAEITGEIQQYQNQPYCLRVESDIKRFFENLNPMGNSMEKEFTDYLFNKSLEIEPRNPKPLPRFPKKYS

SEQ ID NO: 50 - protein, 94 aa, synthetic construct
MATHCFQHLLRVAYQDPSSGCTSKTLAVPPEASIATLNQLCATKFRVTQPNTFGLFLYKEQGYHRLPPGALAHRLPTTGYLVYRRAEWPETQGA

SEQ ID NO: 51 - protein, 429 aa, synthetic construct
MGVIPTLVKLLGIHCQNAALTEMCLVAFGNLAELESSKEQFASTNIAEELVKLFKKQIEHDKREMIFEVLAPLAENDAIKLQLVEAGLVECLLEIVQQKVDSDKEDDITELKTGSDLMVLLLLGDESMQKLFEGGKGSVFQRVLSWIPSNNHQLQLAGALAIANFARNDANCIHMVDNGIVEKLMDLLDRHVEDGNVTVQHAALSALRNLAIPVINKAKMLSAGVTEAVLKFLKSEMPPVQFKLLGTLRMLIDAQAEAAEQLGKNVKLVERLVEWCEAKDHAGVMGESNRLLSALIRHSKSKDVIKTIVQSGGIKHLVTMATSEHVIMQNEALVALALIAALELGTAEKDLESAKLVQILHRLLADERSAPEIKYNSMVLICALMGSECLHKEVQDLAFLDVVSKLRSHENKSVAQQASLTEQRLTVES

SEQ ID NO: 52 - protein, 118 aa, synthetic construct
MSSPGLTSKESLFVRINAAHGFSLIQVDNTKVTMKEILLKAVKRRKGSQKVSGPQYRLEKQSEPNVAVDLDSTLESQSAWEFCLVRENSSRADGVFEEDSQIDIATVQDMLSSHHYKS

SEQ ID NO: 53 - protein, 163 aa, synthetic construct
MGGPTLQEIKQKIDSYNTREKNCLGMKLSEDGTYTGFIKVHLKLRRPVTVPAGIRPQSIYDAIKEVNLAATTDKRTSFYLPLDAIKQLHISSTTTVSEVIQGLLKKFMVVDNPQKFALFKRIHKDGQVLFQKLSIADRPLYLRLLAGPDTEVLSFVLKENETG

SEQ ID NO: 54 - protein, 230 aa, synthetic construct
MSDADKLRKVICELLETERTYVKDLNCLMERYLKPLQKETFLTQDELDVLFGNLTEMVEFQVEFLKTLEDGVRLVPDLEKLEKVDQFKKVLFSLGGSFLYYADRFKLYSAFCASHTKVPKVLVKAKTDTAFKAFLDAQNPKQQHSSTLESYLIKPIQRILKYPLLLRELFALTDAESEEHYHLDVAIKTMNKVASHINEMQKIHEEFGAVFDQLIAEQTGEKKEVADLSM

SEQ ID NO: 55 - protein, 111 aa, synthetic construct
MSVSVTSITSTVLPPVYNQQNEDTCIIRISVEDNNGNMYKSIMLTSQDKTPAVIQRAMLKHNLDSDPAEEYELVQVISEDKELVIPDSANVFYAMNSQVNFDFILRKKNSM

SEQ ID NO: 56 - protein, 285 aa, synthetic construct
MSAEFTCFSEDLVAEQLTYMDAQLFKKVVPHHCLGCIWSRRDKKENKHLAPTIRATISQFNTLTKCVVSTILGGKELKTQQRAKIIEKWINIAHECRLLKNFSSLRAIVSALQSNSIYRLKKTWAAVPRDRMLMFEELSDIFSDHNNHLTSRELLMKEGTSKFANLDSSVKENQKRTQRRLQLQKDMGVMQGTVPYLGTFLTDLTMLDTALQDYIEGGLINFEKRRREFEVIAQIKLLQSACNSYCMTPDQKFIQWFQRQQLLTEEESYALSCEIEAAADASTTS

SEQ ID NO: 57 - protein, 404 aa, synthetic construct
MSAEFTCFSEDLVAEQLTYMDAQLFKKVVPHHCLGCIWSRRDKKENKHLAPTIRATISQFNTLTKCVVSTILGGKELKTQQRAKIIEKWINIAHECRLLKNFSSLRAIVSALQSNSIYRLKKTWAAVPRDRMLMFEELSDIFSDHNNHLTSRELLMKEGTSKFANLDSSVKENQKRTQRRLQLQKDMGVMQGTVPYLGTFLTDLTMLDTALQDYIEGGLINFEKRRREFEVIAQIKLLQSACNSYCMTPDQKFIQWFQRQQLLTEEESYALSCEIEAAADASTTSGGSGGSGGSSVSVTSITSTVLPPVYNQQNEDTCIIRISVEDNNGNMYKSIMLTSQDKTPAVIQRAMLKHNLDSDPAEEYELVQVISEDKELVIPDSANVFYAMNSQVNFDFILRKKNSM

SEQ ID NO: 58 - protein, 112 aa, synthetic construct
MGSPRIPLPAQQSSEARVIRVSIDNDHGNLYRSILLTSQDKAPSVVRRALQKHNVPQPWACDYQLFQVLPGDRVLLIPDNANVFYAMSPVAPRDFMLRRKEGTRNTLSVSPS

SEQ ID NO: 59 - protein, 331 aa, synthetic construct
MMPEEEYSEFKELILQKELHVVYALSHVCGQDRTLLASILLRIFLHEKLESLLLCTLNDREISMEDEATTLFRATTLASTLMEQYMKATATQFVHHALKDSILKIMESKQSCELSPSKLEKNEDVNTNLTHLLNILSELVEKIFMASEILPPTLRYIYGCLQKSVQHKWPTNTTMRTRVVSGFVFLRLICPAILNPRMFNIISDSPSPIAARTLILVAKSVQNLANLVEFGAKEPYMEGVNPFIKSNKHRMIMFLDELGNVPELPDTTEHSRTDLSRDLAALHEICVAHSDELRTLSNERGAQQHVLKKLLAITELLQQKQNQYTKTNDVR

SEQ ID NO: 60 - protein, 280 aa, synthetic construct
MGELPIAMALANVVPCSQWDELARVLVTLFDSRHLLYQLLWNMFSKEVELADSMQTLFRGNSLASKIMTFCFKVYGATYLQKLLDPLLRIVITSSDWQHVSFEVDPTRLEPSESLEENQRNLLQMTEKFFHAIISSSSEFPPQLRSVCHCLYQATCHSLLNKATVKEKKENKKSVVSQRFPQNSIGAVGSAMFLRFINPAIVSPYEAGILDKKPPPRIERGLKLMSKILQSIANHVLFTKEEHMRPFNDFVKSNFDAARRFFLDIASDCPTSDAVNHSLS

SEQ ID NO: 61 - protein, 383 aa, synthetic construct
MVTLQEWNDPLDHDLEAQLIYRHLLGVEAMLWERHRELSGGAEAGTVPTSPGKVPEDSLARLLRVLQDLREAHSSGGSGGVLPSSYYQPLVHLLCHEVKLGMQGPGQLIPLIEETTSTECRQDVATNLLKLFLGQGLAKDFLDLLFQLELSRTSETNTLFRSNSLASKSMESFLKVAGMQYLHGVLGPIINKVFEEKKYVELDPSKVEVKDVGCSGLHRPQTEAEVLEQSAQTLRAHLGALLSALSRSVRACPAVVRATFRQLFRRVRERFPGAQHENVPFIAVTSFLCLRFFSPAIMSPKLFHLRERHADARTSRTLLLLAKAVQNVGNMDTPASRAKEAWMEPLQPTVRQGVAQLKDFITKLVDIEEKDELDLQRTLSLQA

SEQ ID NO: 62 - protein, 304 aa, synthetic construct
MVLPSSYYQPLVHLLCHEVKLGMQGPGQLIPLIEETTSTECRQDVATNLLKLFLGQGLAKDFLDLLFQLELSRTSETNTLFRSNSLASKSMESFLKVAGMQYLHGVLGPIINKVFEEKKYVELDPSKVEVKDVGCSGLHRPQTEAEVLEQSAQTLRAHLGALLSALSRSVRACPAVVRATFRQLFRRVRERFPGAQHENVPFIAVTSFLCLRFFSPAIMSPKLFHLRERHADARTSRTLLLLAKAVQNVGNMDTPASRAKEAWMEPLQPTVRQGVAQLKDFITKLVDIEEKDELDLQRTLSLQA

SEQ ID NO: 63 - protein, 267 aa, synthetic construct
MASNQREEYLLLRLFKTALQEEIKSKVDQIQEIVTGNPTVIKMVVSFNRGARGQNALRQILAPVVKEIMDDKSLNIKTDPVDIYKSWVNQMESQTGEASKLPYDVTPEQALAHEEVKTRLDSSIRNMRAVTDKFLSAIVSSVDKIPYGMRFIAKVLKDSLHEKFPDAGEDELLKIIGNLLYYRYMNPAIVAPDAFDIIDLSAGGQLTTDQRRNLGSIAKMLQHAASNKMFLGDNAHLSIINEYLSQSYQKFRRFFQTACDVPELQGG

SEQ ID NO: 64 - protein, 315 aa, synthetic construct
MVLPSERYKELAEFLTFHYARLCGALEPALPAQAKEELAAAMVRVLRATGRAQALVTDLGTAELARCGGREALLFRENTLATKAIDEYMKLVAQDYLQETLGQVVRRLCASTEDCEVDPSKCPASELPEHQARLRNSCEEVFETIIHSYDWFPAELGIVFSSWREACKERGSEVLGPRLVCASLFLRLLCPAILAPSLFGLAPDHPAPGPARTLTLIAKVIQNLANRAPFGEKEAYMGFMNSFLEEHGPAMQCFLDQVAMVDVDAAPSGYQGSGDLALQLAVLHAQLCTIFAELDQTTRDTLEPLPTILRAIEEG

SEQ ID NO: 65 - protein, 83 aa, synthetic construct
MDPSKTSNTIAVFLPNKQRTVVNVRNGMSLHDCLMKALKVRGLQPECCAVFRLLHEHKGAKARLDWNTDAASLIGEELQVDFL

SEQ ID NO: 66 - protein, 164 aa, synthetic construct
MGTEYKLVVVGAGGVGKSALTIQLIQNHFVDEYDPTIEDSYRKQVVIDGETCLLDILDTAGQEEYSAMRDQYMRTGEGGSGGDPSKTSNTIAVFLPNKQRTVVNVRNGMSLHDCLMKALKVRGLQPECCAVFRLLHEHKGAKARLDWNTDAASLIGEELQVDFL

SEQ ID NO: 67 - protein, 175 aa, synthetic construct
MNVDEPVEWETPDLSQAEIEQKIKEYNAQINSNLFMSLNKDGSYTGFIKVQLKLVRPVSVPSSKKPPSLQDARRGPGRGTSVRRRTSFYLPKDAVKHLHVLSRTRAREVIEALLRKFLVVDDPRKFALFERAERHGQVYLRKLLDDEQPLRLRLLAGPSDKALSFVLKENDSGEV

SEQ ID NO: 68 - DNA, 156 bp, synthetic construct
  source 1..156: mol_type = other DNA
atggaagagcctaaggtgaagtgggacgaggaaaagaccgtgaaagagctgatcgagcggtacggcatccccgaggaagaggccaagaaggccgctaagaaaagatacgaggtggtcaagaaagtgatgctggaactgaagaaggaagcccagaag

SEQ ID NO: 69 - DNA, 225 bp, synthetic construct
  source 1..225: mol_type = other DNA
atggagctgaagctgatcatcaaagaggaaccaggcgagcctctgcctgaggaagaggtgaaggccctggaagaggcctatagatacgtgcagaaggcctacagcgaggagttcggcgacatcaagcctaagaccagaacaatctacatcagccagaaccccgagatccgggaaatcattgtctctagagccaaagctatcgtggaacgggccaaggctaaggaa

SEQ ID NO: 70 - DNA, 195 bp, synthetic construct
  source 1..195: mol_type = other DNA
atgatgaaaaagcctctgagattcccagatttcctgacccctgagcagaaagagctgctggccaagatccgggaaatcatggaaaagtacagacccgaggccaacgccgccagcgacgaggaaagacctaaggtgatcgccaaggtgtacgaggaagctaaggagctgttcgacaagctggaagagctgtttaag

SEQ ID NO: 71 - DNA, 261 bp, synthetic construct
  source 1..261: mol_type = other DNA
atggaggccgagaaagtgttcgccgctgtggaagaactgagaaagaagtaccgggacgaggaactgctgatcatcattgtggccagaacccctgaggaattcgagaaagctctgaagagagtgatcaagtacttcaagaagaaagccaaggtggtcgtggtgggcttcgagaagtacaaggaagagttcgagaagatcgccgagaagtataagcccgagctggaaaaaaagggcatcaagctggaactcgtgtttctgcct

SEQ ID NO: 72 - DNA, 180 bp, synthetic construct
  source 1..180: mol_type = other DNA
atgggagccctgcctaccctgagctacaccatcagctacaacaaggaaaccggcgagattatcgccgtggtgtctgatgaccccgagctgatcgagagagtgacaaaggaagccaaagagctggccaaggccatcggcgctaaaaaggtggtcgtgaacgtgaagcctacagtgatcctg

SEQ ID NO: 73 - DNA, 228 bp, synthetic construct
  source 1..228: mol_type = other DNA
atgtctgagatcgaggtgaccattacagctgacaccatcgaggaagccctgagaaagagcaaggtctacgccaaagccttcgccgacaacaagaacaccaaggtgacaatcatcctgcggcccaaggccctggccgaagatcctgccgagctggaagctgctaaagccgagatcaccgccctgttcaccgccaagggccacaccatcacaatcgtggaagagctgcct

SEQ ID NO: 74 - DNA, 252 bp, synthetic construct
  source 1..252: mol_type = other DNA
atggttgaggtggaggaactggaaggcgtgtccctgcctgaggtggtggaactcgccagacggctggaaaaggaagctctggagagccacaaacctggcgagatcgtggtcagagagattagagccaagctgaaggacggccgcgaggtgctgatcggagtggtggtggccagccccgagagagtgatcagcatctctggcagaaacgccgaggccgtgcggcggaggctggaaagaatcatcgagcagctg

SEQ ID NO: 75 - DNA, 201 bp, synthetic construct
  source 1..201: mol_type = other DNA
atggtcgagatcatcaccagaaagtacaagatcgtgaaaattaccaaggaagtgaacggcgaggaaatcgtgatcaccctgaaggccagactggaaagacctgaggaagtggaacggctggagaagcgggccgagagatacaaaaagctgtaccccggcgctgaggtgacagagtctgagagcgaggaggtgctggaagtg

SEQ ID NO: 76 - DNA, 180 bp, synthetic construct
  source 1..180: mol_type = other DNA
atgatggaagaggaagaggagctgagaaagcgggccgacagaaaattcgccatcaagaaagctatcgagaagcagaccggcttccctgtccacatcgtggcttctccagagctgtaccccatcctgaaggccaaggtggaagaggccggcgccaacctgacagtggaaagcgccgagcct

SEQ ID NO: 77 - DNA, 258 bp, synthetic construct
  source 1..258: mol_type = other DNA
atgatgaccaaggtggacatcacactgacattccccgacgaatacagagccaacgccgtgcggagagccgtcgccatcgtgaaaggactgagcaagattcctaacaccctggtgaccatcaccctgaagggcagcgccgccacatctaccgccgctgctaatgaaatcgccaccgagctgaaaaagatcggcgccgacaagatcacaatcaccacagatcctgctacaggcaccaccaccctgaccatcgagttcaag

SEQ ID NO: 78 - DNA, 204 bp, synthetic construct
  source 1..204: mol_type = other DNA
atgatgaaaatcaactacatccggatcacaggagatgtggaatacgtgtctagaagagccaagcagatcatcaagaccaagagccctgaggaagtggaaaagatcatcagagagctgcaggagaaagccggcaaggaagaaatcaccatcaacatcatcattgacgaggacaccggcgagacaaccatcgagctgatcgacgag

SEQ ID NO: 79 - DNA, 210 bp, synthetic construct
  source 1..210: mol_type = other DNA
atgatgatgaaaagaaagatcatcgtggacaccagacctaacacagtggaaaacatcctggaaatccggaaggagctgagaaagctgagcaaggagagcggcgagatcatccacgtgatcagaggatctgagaatgtgtccctggaactggtcatcgagaaggccaaagagctggctaaggaattcggcgccgagttcgagattcgggaa

SEQ ID NO: 80 - DNA, 183 bp, synthetic construct
  source 1..183: mol_type = other DNA
atggctagcaagctggaagttgtgatcaaggccacacctgagaacgccgaagaggccctgagaagagtgaagcggtacgccgaggtgatgaaaggcgtggacatcgtggtggaagtcaccgatgaggaagtggccgagatcctgaagaaagaggtgaccgagaagtacggcgtgaagatcctg

SEQ ID NO: 81 - protein, 52 aa, synthetic construct
MEEPKVKWDEEKTVKELIERYGIPEEEAKKAAKKRYEVVKKVMLELKKEAQK

SEQ ID NO: 82 - protein, 75 aa, synthetic construct
MELKLIIKEEPGEPLPEEEVKALEEAYRYVQKAYSEEFGDIKPKTRTIYISQNPEIREIIVSRAKAIVERAKAKE

SEQ ID NO: 83 - protein, 65 aa, synthetic construct
MMKKPLRFPDFLTPEQKELLAKIREIMEKYRPEANAASDEERPKVIAKVYEEAKELFDKLEELFK

SEQ ID NO: 84 - protein, 87 aa, synthetic construct
MEAEKVFAAVEELRKKYRDEELLIIIVARTPEEFEKALKRVIKYFKKKAKVVVVGFEKYKEEFEKIAEKYKPELEKKGIKLELVFLP

SEQ ID NO: 85 - protein, 60 aa, synthetic construct
MGALPTLSYTISYNKETGEIIAVVSDDPELIERVTKEAKELAKAIGAKKVVVNVKPTVIL

SEQ ID NO: 86 - protein, 76 aa, synthetic construct
MSEIEVTITADTIEEALRKSKVYAKAFADNKNTKVTIILRPKALAEDPAELEAAKAEITALFTAKGHTITIVEELP

SEQ ID NO: 87 - protein, 84 aa, synthetic construct
MVEVEELEGVSLPEVVELARRLEKEALESHKPGEIVVREIRAKLKDGREVLIGVVVASPERVISISGRNAEAVRRRLERIIEQL

SEQ ID NO: 88 - protein, 67 aa, synthetic construct
MVEIITRKYKIVKITKEVNGEEIVITLKARLERPEEVERLEKRAERYKKLYPGAEVTESESEEVLEV

SEQ ID NO: 89 - protein, 60 aa, synthetic construct
MMEEEEELRKRADRKFAIKKAIEKQTGFPVHIVASPELYPILKAKVEEAGANLTVESAEP

SEQ ID NO: 90 - protein, 86 aa, synthetic construct
MMTKVDITLTFPDEYRANAVRRAVAIVKGLSKIPNTLVTITLKGSAATSTAAANEIATELKKIGADKITITTDPATGTTTLTIEFK

SEQ ID NO: 91 - protein, 68 aa, synthetic construct
MMKINYIRITGDVEYVSRRAKQIIKTKSPEEVEKIIRELQEKAGKEEITINIIIDEDTGETTIELIDE

SEQ ID NO: 92 - protein, 70 aa, synthetic construct
MMMKRKIIVDTRPNTVENILEIRKELRKLSKESGEIIHVIRGSENVSLELVIEKAKELAKEFGAEFEIRE

SEQ ID NO: 93 - protein, 61 aa, synthetic construct
MASKLEVVIKATPENAEEALRRVKRYAEVMKGVDIVVEVTDEEVAEILKKEVTEKYGVKIL

SEQ ID NO: 94 - DNA, 318 bp, synthetic construct
  source 1..318: mol_type = other DNA
atggactacaaagacgatgacgacaagggatccgtttcttctgttccgaccaaactggaagttgttgctgcgaccccgactagcctgctgatcagctgggatgctcctgcagttaccgtcgattattacgttatcacgtacggtgaaaccggtggtaactccccggttcaaaagtttgaggtaccaggttccaagtctactgctaccatcagcggcctgaaaccgggtgtcgactataccatcactgtatacgcatggggttggcatggtcaggtttactactacatgggtagcccaatctcgattaactaccgtacc

SEQ ID NO: 95 - DNA, 291 bp, synthetic construct
  source 1..291: mol_type = other DNA
atggtcagcagcgtgcccaccaagctggaagtggtggccgctacacctaccagcctgctgatcagctgggatgcctcttctagctccgtgtcctactacagaatcacatacggcgagacaggcggcaacagccctgtgcaggagttcaccgttcctagctactacagcaccgccacaatctccggcctgaaacctggcgtggactacaccattaccgtgtacgcctactactggtacggatctccatactattattggaaggccagccccatcagcatcaactaccggacc

SEQ ID NO: 96 - DNA, 471 bp, synthetic construct
  source 1..471: mol_type = other DNA
atggatcttggaaagaaactcctggaagcagcccgtgctggacaagatgatgaagttcgtattctcatggcaaatggggcagatgtaaacgcatcagaccgatggggctggactccgcttcatctcgcggcgtggtggggtcatctcgaaattgttgaggtccttctcaaacatggagctgacgtgaacgcggctgacttgcatggccagactcctctccacctcgcagctatggttggacatctcgagatagtcgaggttctcttgaagtatggagcagacgtcaatgctaaagatactatgggcgccactccgctccatctcgcagcacaatctggtcatcttgagattgttgaagttctcctcaagaatggggctgatgtcaatgctcaagacaagtttgggaaaacagcttttgatatttcaattgataatggtaatgaagatttggcagaaatccttcaaaagtta

SEQ ID NO: 97 - DNA, 471 bp, synthetic construct
  source 1..471: mol_type = other DNA
atggatcttgggaagaaattgctcgaagctgcaagggctggacaggatgatgaagtgaggattctcatggcaaacggcgcagacgtcaatgcttccgaccggtggggatggacacctctccatcttgcagcctggtggggtcatttggaaattgtcgaggtattgttaaaacgcggggcggatgtcagtgctgctgatttgcatggacaatctccacttcatttagccgcgatggttgggcatctcgagattgttgaggtattgctcaaatatggggcggacgttaacgcgaaagatactatgggagcgactccacttcacctcgcagctaggtcaggacatttggaaatagtggaagagttgttgaagaatggtgcggacatgaatgcgcaagacaaatttggaaagacaacgtttgatatttcaactgataatggtaatgaagatctcgctgaaatacttcaaaagctt

SEQ ID NO: 98 - DNA, 279 bp, synthetic construct
  source 1..279: mol_type = other DNA
atggtgtctagcgtgcccaccaaactggaagtggtggccgccacacctaccagcctgctgatttcttgggatgccagcagcagctccgtgtcctactacagaatcacctacggcgagacaggcggcaacagccctgtgcaagagtttaccgtgcctggctactacagcaccgccaccatctctggactgaagcctggcgtggactacaccatcacagtgtacgctgtgtggcaaggcgtttggagatacgtgtcccctatcagcatcaactaccggacc

SEQ ID NO: 99 - protein, 106 aa, synthetic construct
MDYKDDDDKGSVSSVPTKLEVVAATPTSLLISWDAPAVTVDYYVITYGETGGNSPVQKFEVPGSKSTATISGLKPGVDYTITVYAWGWHGQVYYYMGSPISINYRT

SEQ ID NO: 100 - protein, 97 aa, synthetic construct
MVSSVPTKLEVVAATPTSLLISWDASSSSVSYYRITYGETGGNSPVQEFTVPSYYSTATISGLKPGVDYTITVYAYYWYGSPYYYWKASPISINYRT

SEQ ID NO: 101 - protein, 157 aa, synthetic construct
MDLGKKLLEAARAGQDDEVRILMANGADVNASDRWGWTPLHLAAWWGHLEIVEVLLKHGADVNAADLHGQTPLHLAAMVGHLEIVEVLLKYGADVNAKDTMGATPLHLAAQSGHLEIVEVLLKNGADVNAQDKFGKTAFDISIDNGNEDLAEILQKL

SEQ ID NO: 102 - protein, 157 aa, synthetic construct
MDLGKKLLEAARAGQDDEVRILMANGADVNASDRWGWTPLHLAAWWGHLEIVEVLLKRGADVSAADLHGQSPLHLAAMVGHLEIVEVLLKYGADVNAKDTMGATPLHLAARSGHLEIVEELLKNGADMNAQDKFGKTTFDISTDNGNEDLAEILQKL

SEQ ID NO: 103 - protein, 93 aa, synthetic construct
MVSSVPTKLEVVAATPTSLLISWDASSSSVSYYRITYGETGGNSPVQEFTVPGYYSTATISGLKPGVDYTITVYAVWQGVWRYVSPISINYRT

SEQ ID NO: 104 - DNA, 288 bp, synthetic construct
  source 1..288: mol_type = other DNA
atggtgtctagcgtgcccaccaaactggaagtggtggccgccacacctaccagcctgctgatttcttgggatgcccctgccgtgaccgtgttcttctacgtgatcacctacggcgagacaggccatggcgtgggagcctttcaggcctttaaggtgccaggcagcaagagcaccgccaccatctctggactgaaacccggcgtggactacaccatcaccgtgtacgccagaggctacagcaagcagggcccctacaagccctctccaatcagcatcaactaccggacc

SEQ ID NO: 105 - DNA, 288 bp, synthetic construct
  source 1..288: mol_type = other DNA
atggtgtctagcgtgcccaccaaactggaagtggtggccgccacacctaccagcctgctgatttcttgggatgcccctgccgtgaccgtgttcttctacatcattgcctacggcgagacaggccatggcgtgggagctttccaggcctttagagtgcctggcagcaagagcaccgccaccatctctggactgaaacccggcgtggactacaccatcaccgtgtacgccagaggctacagcaagcagggcccctacaagccctctccaatcagcatcaactaccggacc

SEQ ID NO: 106 - DNA, 279 bp, synthetic construct
  source 1..279: mol_type = other DNA
atggtgtctagcgtgcccaccaaactggaagtggtggccgccacacctacaagcctgctggtttcttgggatgcccctgccgtgaccgtggtgttctacgatatcacctacggcgagacaggcggcaacagccctgtgcaagagtttaccgtgtccggcagcaagagcaccgccacaatctctggactgaagcccggcgtggactacaccatcacagtgtacgccaagtacctgttttggagcggctacagcagccccatcagcatcaactacagaacc

SEQ ID NO: 107 - DNA, 1413 bp, synthetic construct
  source 1..1413: mol_type = other DNA
atgggggtatcatctgtaccgacaaagcttgaggtcgtcgctgctacgccaacttcattgttaatctcatgggacgctccagcagtcactgtcttcttttatgttataacatatggggaaactggacacggggtaggggcattccaagctttcaaagttcccgggagtaaatcaacagcgacaataagcggtcttaagccgggtgttgattatactattactgtatatgcgcgagggtattcaaaacaaggtccttataaaccttcacctatttctattaattataggactggcgggagtggtggaagcggcgagtctctgtttaagggccccagagattacaaccctatcagcagcaccatttgccacctgaccaacgagagcgatggacatacaaccagcctgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagaccggctctggcagcagcgaaaacctttatttccagtctggaggcggtggatccgagaatctctactttcagagtggagggtcagggagcgtgtctagcgtgcccaccaaactggaagtggtggccgccacacctaccagcctgctgatttcttgggatgcccctgccgtgaccgtgttcttctacgtgatcacctacggcgagacaggccatggcgtgggagcctttcaggcctttaaggtgccaggcagcaagagcaccgccaccatctctggactgaaacccggcgtggactacaccatcaccgtgtacgccagaggctacagcaagcagggcccctacaagccctctccaatcagcatcaactaccggaccggcgggtcagggggatctaagagcatgagcagcatggtctccgacacaagctgtaccttcccatctagcgacggcaccttctggaagcactggatccagaccaaggacggccagtgcggcaaccctctggtgagcaccagagacggcttcatcgtgggcatccactctgccagcaactttacaaacacaaacaattacttcgccagcgtgccaaagaacttcatggaactgctgaccaaccaggaggctcagcagtgggtgtccggctggcggctgaacgccgatagcgtgctgtggggcggccacaaagtgttcatggtgaagccagaggagcctttccaacctgtgaaggaggctacccagctgatgaacagctag

SEQ ID NO: 108 - DNA, 1395 bp, synthetic construct
  source 1..1395: mol_type = other DNA
atgggagtgtctagcgtgcccaccaaactggaagtggtggccgccacacctacaagcctgctggtttcttgggatgcccctgccgtgaccgtggtgttctacgatatcacctacggcgagacaggcggcaacagccctgtgcaagagtttaccgtgtccggcagcaagagcaccgccacaatctctggactgaagcccggcgtggactacaccatcacagtgtacgccaagtacctgttttggagcggctacagcagccccatcagcatcaactacagaaccggcgggagtggtggaagcggcgagtctctgtttaagggccccagagattacaaccctatcagcagcaccatttgccacctgaccaacgagagcgatggacatacaaccagcctgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagaccggctctggcagcagcgaaaacctttatttccagtctggaggcggtggatccgagaatctctactttcagagtggagggtcagggagcgtatccagcgtcccgactaagttagaggtcgttgcagctaccccaacctcactcttggtcagttgggacgctcccgcagttactgtcgtcttttatgacattacatatggtgaaactggtggtaattcaccggttcaggaattcactgtcagcggtagtaaatctactgctaccattagcgggttgaaaccaggagtcgattatacaataaccgtctatgctaaatatctcttctggtccgggtattccagtccaatatctattaattataggacgggcgggtcagggggatctaagagcatgagcagcatggtctccgacacaagctgtaccttcccatctagcgacggcaccttctggaagcactggatccagaccaaggacggccagtgcggcaaccctctggtgagcaccagagacggcttcatcgtgggcatccactctgccagcaactttacaaacacaaacaattacttcgccagcgtgccaaagaacttcatggaactgctgaccaaccaggaggctcagcagtgggtgtccggctggcggctgaacgccgatagcgtgctgtggggcggccacaaagtgttcatggtgaagccagaggagcctttccaacctgtgaaggaggctacccagctgatgaacagctag

SEQ ID NO: 109 - protein, 96 aa, synthetic construct
MVSSVPTKLEVVAATPTSLLISWDAPAVTVFFYVITYGETGHGVGAFQAFKVPGSKSTATISGLKPGVDYTITVYARGYSKQGPYKPSPISINYRT

SEQ ID NO: 110 - protein, 96 aa, synthetic construct
MVSSVPTKLEVVAATPTSLLISWDAPAVTVFFYIIAYGETGHGVGAFQAFRVPGSKSTATISGLKPGVDYTITVYARGYSKQGPYKPSPISINYRT

SEQ ID NO: 111 - protein, 93 aa, synthetic construct
MVSSVPTKLEVVAATPTSLLVSWDAPAVTVVFYDITYGETGGNSPVQEFTVSGSKSTATISGLKPGVDYTITVYAKYLFWSGYSSPISINYRT

SEQ ID NO: 112 - protein, 470 aa, synthetic construct
MGVSSVPTKLEVVAATPTSLLISWDAPAVTVFFYVITYGETGHGVGAFQAFKVPGSKSTATISGLKPGVDYTITVYARGYSKQGPYKPSPISINYRTGGSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTGSGSSENLYFQSGGGGSENLYFQSGGSGSVSSVPTKLEVVAATPTSLLISWDAPAVTVFFYVITYGETGHGVGAFQAFKVPGSKSTATISGLKPGVDYTITVYARGYSKQGPYKPSPISINYRTGGSGGSKSMSSMVSDTSCTFPSSDGTFWKHWIQTKDGQCGNPLVSTRDGFIVGIHSASNFTNTNNYFASVPKNFMELLTNQEAQQWVSGWRLNADSVLWGGHKVFMVKPEEPFQPVKEATQLMNS

SEQ ID NO: 113 - protein, 464 aa, synthetic construct
MGVSSVPTKLEVVAATPTSLLVSWDAPAVTVVFYDITYGETGGNSPVQEFTVSGSKSTATISGLKPGVDYTITVYAKYLFWSGYSSPISINYRTGGSGGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTGSGSSENLYFQSGGGGSENLYFQSGGSGSVSSVPTKLEVVAATPTSLLVSWDAPAVTVVFYDITYGETGGNSPVQEFTVSGSKSTATISGLKPGVDYTITVYAKYLFWSGYSSPISINYRTGGSGGSKSMSSMVSDTSCTFPSSDGTFWKHWIQTKDGQCGNPLVSTRDGFIVGIHSASNFTNTNNYFASVPKNFMELLTNQEAQQWVSGWRLNADSVLWGGHKVFMVKPEEPFQPVKEATQLMNS

SEQ ID NO: 114 - DNA, 417 bp, synthetic construct
  source 1..417: mol_type = other DNA
ggaagcgagggaagcggctctgaaggaagcggaagcgagggctctggttccgaaggaagcaagagcatgagcagcatggtctccgacacaagctgtaccttcccatctagcgacggcattttctggaagcactggatccagaccaaggacggccagtgcggctctcctctggtgagcaccagagacggcttcatcgtgggcatccactctgccagcaactttacaaacacaaacaattacttcaccagcgtgccaaagaacttcatggaactgctgaccaaccaggaggctcagcagtgggtgtccggctggcggctgaacgccgatagcgtgctgtggggcggccacaaagtgttcatggtgaagccagaggagcctttccaacctgtgaaggaggctacccagctgatgaacagc

SEQ ID NO: 115 - DNA, 399 bp, synthetic construct
  source 1..399: mol_type = other DNA
ggaagcgagggaagcggctctgaaggaagcggaagcgagggctctggcgagtctctgtttaagggccccagagattacaaccctatcagcagcaccatttgccacctgaccaacgagagcgatggacatacaaccagcctgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagacc

SEQ ID NO: 116 - protein, 139 aa, synthetic construct
GSEGSGSEGSGSEGSGSEGSKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNS

SEQ ID NO: 117 - protein, 133 aa, synthetic construct
GSEGSGSEGSGSEGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQT

SEQ ID NO: 118 - DNA, 285 bp, synthetic construct
  source 1..285: mol_type = other DNA
ggctacaccatcgagttcaccgtggtcaagacccctggcatgcctgatgagctgcacgagatcctgaacgagggcatgaaggaactgagcaaagaactgaccgagctggtcaacaagaacgccagcctggaagagatcgagaagctgctggaagagtacaaagagaaaaccatccagaagctgaaagagaacaacttcagcgaccacgacatcgagtgggccgagaaggtgttcgaggttgtgcggaagctggccaaagagacatgggagagaatcaagaacatg

SEQ ID NO: 119 - DNA, 297 bp, synthetic construct
  source 1..297: mol_type = other DNA
agcctggaagaggaagtggaacggatcttcagagagagcgccgagctgctgagacagatgatcgagaacatccaggacagagccctggtcaccgagatcgctagaagaatcgccgagagagtgatgcccctgcctgaggaagagagagaacggctgatcagacggctggaagagtgggtccgaagagaattccctggcgtgtggctggaaatcagctacgagcctaccatggacgccgaaggcagacctgccatgctggtcaccttcacctttacattccccgaggacttcaagctg

SEQ ID NO: 120 - DNA, 330 bp, synthetic construct
  source 1..330: mol_type = other DNA
atgagcagagaggaactgctggaacgggtgttcgagcggatcagcgagagataccccagattcagcgtggaagagatccggcgggaagtgcggagaggcgaaagactgtgggacgagctgatcaagcccagcacaccccgggacaaagaaaccgtggaagccgtgttcaagtacctgcggagccagagcgagatcgtgtatgtgcggggaatcgagcaagtgatgggactgcctgaaggcgacacccgggaatctgtcagatgggagatcgacggccagagatacgaggtgttcttcatggacgagatgatgaagcggctggccgaggaa

SEQ ID NO: 121 - DNA, 246 bp, synthetic construct
  source 1..246: mol_type = other DNA
agcagaatggaagaggtggtggaacggatccgggaagagatcgagagagccaccggcagagaagtccgctacgtgggctacagaatgatcgaagaggacggcagaaaggcccacgtgttcaccgcctactttccacctagctggtccagagaggaacgcgagacaggcatcagagtgatggaagaagtcgtgcgcgaggcctttcctgacaccagaggcgagatgtacagcaccaccgagatggtg

SEQ ID NO: 122 - DNA, 213 bp, synthetic construct
  source 1..213: mol_type = other DNA
ggagaagtgcgcgtgaccgtgatgaagacccctgaaggcatggaaatctaccgggtgttcgagcggtacagacggcggagacagagagtgacccacagagagatcctggaaattctggaacggtacagccggaagctgggcctgcctattagaagaggcgagagagtgatcgagctgggccacctggctagagtggaattcatcgaggtgccc

SEQ ID NO: 123 - DNA, 216 bp, synthetic construct
  source 1..216: mol_type = other DNA
ggcaaggtggtggttacacttcctggcggacatgaagtggtggccggcgaaggatccgtgacattcatcccacctggcaccagcgaggaagtgacagtgatggccagcatcgagagcgtgggaagagtgcgcgtggaattcaccagccggatgacccaagaggaactgaaagaggtggtcgcccggctgcagaaaacctggacagtgaagcagacc

SEQ ID NO: 124 - DNA, 255 bp, synthetic construct
  source 1..255: mol_type = other DNA
tctgccctgctgcgcgagatggaaagactgatgctggaacggctggtggaagagggctttgatgccgccgagctggaaacaagagtggcccctgaagccagacctgacgctcctgatcagcggtggatgagatacgagacaaccgtgaccatgcctgaccggcggattaccgaggccgacagagaagagatgcgcgagagaatccagcctgtttggtgggaagtcgccgaagagtacagctacagagcccacaca

SEQ ID NO: 125 - DNA, 285 bp, synthetic construct
  source 1..285: mol_type = other DNA
tctgccgagctggaagagatgaagagagagctgcagcggcggatcagagaagccggactggatgatgtgaccctgctggacgttcgctgggagcccgagaatcctgattacgtggtgctggacttcatgaccagaggctggcccttcgagcaagaggaagcccagagaagattcagagaggccttcgagcggcagagaagagaactgagaggcgccagcttcgtggacaagatggttcgagccctgcgggacatgctggaagccctgaaagccgagagagagagc

SEQ ID NO: 126 - DNA, 285 bp, synthetic construct
  source 1..285: mol_type = other DNA
tctgccgagctggaacggatgaaggaagaactgcagcggagagtcagagaggccggactggacgacgtggaaatcctggacgttcgctgggagcccgagaatcccgattacgtggtgctggacttcatgaccagaggctggcccttcgagcaagaggaagcccagagagagttcagacgggccttcgagagagagcgggaaacactgagaggcgccagcttcgtggacaagatggttcgagcactgcaggccatgctggaaagactgaaagccgccagagaagcc

SEQ ID NO: 127 - DNA, 312 bp, synthetic construct
  source 1..312: mol_type = other DNA
cctcctgttcctacacagtggcaagtgatcgagttcgacagccggaacaaagaagatgtgctgagcaccctgaagctggtgctgaagcggatgcggaaatacgtgtccgagcacaaggacgaccccgtgtcacccttcgaggaaatgcagaaggcccatcctgagcacacccctgagacacagcggcggaacattctgagaatggccagcaccatcctgtccgaagaggaactggaaatcgtgaaagagtacctcgaggacggcagctacgaggccctgctgaaactggtggaattcctggaaagcaagctg

SEQ ID NO: 128 - DNA, 321 bp, synthetic construct
  source 1..321: mol_type = other DNA
atggaaaagctgagcaaagaagagatgctgaaagaagtgcgcgactggctgctgaagcactaccccgagatcgaggaagatcccgagctgaaagagctgctggacgaactgctggaaaagatgaaggaactgttcgatcggagcagcagcgacatcaccgtgacaccctgggagaaagacggcatctggtacagaaccatcgagttcgaggtggaatacgaccccgagacagacgaccctgagaaggactttgccgagctggaagaactgatcaacaagctgaacagcctgctgcctcatcctatctacaagtggacccgg

SEQ ID NO: 129 - DNA, 216 bp, synthetic construct
  source 1..216: mol_type = other DNA
agcagcaaagagatcgaggaacggcagaagaagaaagccctggaaaaggtggaagagatcctgaagaagatgaagtccgagaagcacaaagagcggagcatcttcgaagtgacctacagcatgcccgacttcaagaacggcaagatgaaggaagtgaaagaagtctggacctacgaggaagccgagaagaagattgaggaagagaaaaagaaagag

SEQ ID NO: 130 - DNA, 216 bp, synthetic construct
  source 1..216: mol_type = other DNA
agcatggaagagatcaaagagcggcagagaggcaaggccctggaaaaagtgaaagaaatcctcgagaagatgaagtccgagaagcacaaagaacggtccatcttcgaagtgacctacagcatgcccgacttcgagaacggcaagatgatcgaagtcaaagaggtgctgacctacaaagaagccctggaagaaatcgagaagtgggagaagaagaag

SEQ ID NO: 131 - DNA, 291 bp, synthetic construct
  source 1..291: mol_type = other DNA
agcctggaagagttcatcgtggaagtgtacagcgagtacctgcggatctacgagaagttcaacggcgacctgagagaagtgggcagagccctgatggaattcctgggcgagctggtcaaacggggcagagtgaaagaattctacgagctggtggaatttctcgagagcaacggcatcttccccgagaaagagtacatccccggcaaccagatgcggtacaccttcaccagcagacagatccccaagatgatggaagtgctgaagaaggtctacgaggaagaactgaagaag

SEQ ID NO: 132 - DNA, 291 bp, synthetic construct
  source 1..291: mol_type = other DNA
agcctgagagatctgatcgtggaagtgtacagcaagtacctcgagatctacgagaagcacaacggcgacctgagagagacaggcagagcctggatggcctttctgggagaactggtggctcagggcagagtgaaagaattctacgagctggtggacttcctggaaagcaacggcatcttccccgagaaagagtacatccccggcaaccagatgcggtacaccttcaccagcagacagatccccaaggccatggaaatcctgaagaaagtgtacgaggaagaactggccaag

SEQ ID NO: 133 - DNA, 273 bp, synthetic construct
  source 1..273: mol_type = other DNA
atgcagatgagccccgaagagtacatcgacagcctgttcagcaagaccaagaacacccctgaagagatcatcgaggtggtcaaagaagtgcggaagctgttccccaacatggacatcgagctgagcaccaagagcggctacaaagagtatatccccgccagcacctccgaggaagagagagagaagcggctgagagagttcttcgagaagtaccccagagaggaaatcgagatcaccttcgtgatccccagagaagaggtggacgccttcaag

SEQ ID NO: 134 - DNA, 345 bp, synthetic construct
  source 1..345: mol_type = other DNA
atggtcatcgagatccccagcgagaagaagaaactgcggctggaagtggtcatgcggaggcccgatgatggaatcgccaccaccgtgaccatcgaggtggaaagcgaagaactggtgctggaaatccgggacgatagcgaggtgccacacatcgtgctgagattccccgagggcaaagtgaccctgacactgcctaacggcgaagtggaagtgctggaacccaagaacgccacactggtggtgtacatgagaggcaccgaccggatggttatcgacttcatcgagacaatggacggcgtgttccccaccgacgaagagatcgaagaatgggccagagaggacgac

SEQ ID NO: 135 - DNA, 273 bp, synthetic construct
  source 1..273: mol_type = other DNA
agcgagagagagaggctggaaagagccctggaactgctggaccggatgctggaaaagctggaagaggctctggaaggcaaagtgaaggactgggaagccttcttcagagagtggaacgagctgttcgccgagttcctgagactgaccggcgaggacagacagaccagaaccatcgagatcgagtacacccctgacgaggtgcccgaagaggacctgcggaagctgaaagaactggtggaaagaatgacccgggaagtgcgcgagagagccgaa

SEQ ID NO: 136 - DNA, 348 bp, synthetic construct
  source 1..348: mol_type = other DNA
agcgagagcatcaagaagctgatcgaagagaagctgaaagagatgtacgagctgcccggcgtgggagagagagccagagaacatctggaacgcgtgatggaactgatgctgagattcgccgagcggagagctgctggtggacctagaccttctccagccgaagtggacgagctgatggaacacctgagagaaatggtctgggccatgagagaggctggcgcctccaaagagacaatcgccaagggcctgaagctgattgaggaagtgaccagcaatcccgagatgggcttcgagatcagctacgagtttctgcctggcggctacatgcggatcaccttcacatggcat

SEQ ID NO: 137 - DNA, 357 bp, synthetic construct
  source 1..357: mol_type = other DNA
gccaccagatacagaagagtgaccttcgagttcgagcccagaggcgacagagccgaactggaagctgtgtgggcccagtaccaagaggtggtggaccggctgatccggtacttcgagcggagagtggaagaactcgaggaagagatccggcgggtcgagcgcgagaatgaggaagaaggcacaggcgactgggaaagagtggaacggctgagaagagagcggcgggaagccgaagaaatcgtgcggagactgagagagctgcggcgcgagctggaagaaagattccctgtggatcctctgggcaccctgatggaagccgccagaagaatcgacgagatcctgcctgaggaattccac

SEQ ID NO: 138 - DNA, 291 bp, synthetic construct
  source 1..291: mol_type = other DNA
cctcctaagcctagcggcttcaccaaagaacacgaggaaatgctggaaaagttccgcgagatcgtggaacggcggaacagcgagtacctgcctgagttcctgaagaagctggaagaactggaaaagatcctgaaagaacacatgacccctgaggacaccatcagcatgagacacgccctggacgaagagggcagacccgtgatggtcatcgagttcatctttcgggaccccgagctgcacgagaagatgaaggaactggtggacgagctgaatgagctgatgagcaagatg

SEQ ID NO: 139 - DNA, 264 bp, synthetic construct
  source 1..264: mol_type = other DNA
gaggtgtacgacctggacaccatgagcagagaggaactgagagagctgctggaacggatcgagcggaccgtggaatggatgctgagcagactgcctgacctgggcgaaagagtcagagagcttcggcggagagcctttgaaggcgtggaagaagtgcgcagagccctggaagaaggggccccactgcaagaagtgatcggcctgatgagcggcatgagccccatcaaccgggaaatccaagagctgtgggacgaagtgaacgcc

SEQ ID NO: 140 - DNA, 291 bp, synthetic construct
  source 1..291: mol_type = other DNA
gccacagtgacagtggtggacggcaaagaattctacgtgccccacggccggctgatcgtggaaaagaaaggcgacacctggtacatcaccctggaattcgagaagcccgacgagagccacgtgaaggctgtggtggaagccttcaagtacctggtggaacagggcgaagatccccggaacttcatcctggtggtcacctactctccccggaagggcgagaaaaccagccgggaagagatgcggagaatcaccgaggaaatgatgaagaagatcctggaagagatcgacaag

SEQ ID NO: 141 - protein, 95 aa, synthetic construct
GYTIEFTVVKTPGMPDELHEILNEGMKELSKELTELVNKNASLEEIEKLLEEYKEKTIQKLKENNFSDHDIEWAEKVFEVVRKLAKETWERIKNM

SEQ ID NO: 142 - protein, 99 aa, synthetic construct
SLEEEVERIFRESAELLRQMIENIQDRALVTEIARRIAERVMPLPEEERERLIRRLEEWVRREFPGVWLEISYEPTMDAEGRPAMLVTFTFTFPEDFKL

SEQ ID NO: 143 - protein, 110 aa, synthetic construct
MSREELLERVFERISERYPRFSVEEIRREVRRGERLWDELIKPSTPRDKETVEAVFKYLRSQSEIVYVRGIEQVMGLPEGDTRESVRWEIDGQRYEVFFMDEMMKRLAEE

SEQ ID NO: 144 - protein, 82 aa, synthetic construct
SRMEEVVERIREEIERATGREVRYVGYRMIEEDGRKAHVFTAYFPPSWSREERETGIRVMEEVVREAFPDTRGEMYSTTEMV

SEQ ID NO: 145 - protein, 71 aa, synthetic construct
GEVRVTVMKTPEGMEIYRVFERYRRRRQRVTHREILEILERYSRKLGLPIRRGERVIELGHLARVEFIEVP

SEQ ID NO: 146 - protein, 72 aa, synthetic construct
GKVVVTLPGGHEVVAGEGSVTFIPPGTSEEVTVMASIESVGRVRVEFTSRMTQEELKEVVARLQKTWTVKQT

SEQ ID NO: 147 - protein, 85 aa, synthetic construct
SALLREMERLMLERLVEEGFDAAELETRVAPEARPDAPDQRWMRYETTVTMPDRRITEADREEMRERIQPVWWEVAEEYSYRAHT

SEQ ID NO: 148 - protein, 95 aa, synthetic construct
SAELEEMKRELQRRIREAGLDDVTLLDVRWEPENPDYVVLDFMTRGWPFEQEEAQRRFREAFERQRRELRGASFVDKMVRALRDMLEALKAERES

SEQ ID NO: 149 - protein, 95 aa, synthetic construct
SAELERMKEELQRRVREAGLDDVEILDVRWEPENPDYVVLDFMTRGWPFEQEEAQREFRRAFERERETLRGASFVDKMVRALQAMLERLKAAREA

SEQ ID NO: 150 - protein, 104 aa, synthetic construct
PPVPTQWQVIEFDSRNKEDVLSTLKLVLKRMRKYVSEHKDDPVSPFEEMQKAHPEHTPETQRRNILRMASTILSEEELEIVKEYLEDGSYEALLKLVEFLESKL

SEQ ID NO: 151 - protein, 107 aa, synthetic construct
MEKLSKEEMLKEVRDWLLKHYPEIEEDPELKELLDELLEKMKELFDRSSSDITVTPWEKDGIWYRTIEFEVEYDPETDDPEKDFAELEELINKLNSLLPHPIYKWTR

SEQ ID NO: 152 - protein, 72 aa, synthetic construct
SSKEIEERQKKKALEKVEEILKKMKSEKHKERSIFEVTYSMPDFKNGKMKEVKEVWTYEEAEKKIEEEKKKE

SEQ ID NO: 153 - protein, 72 aa, synthetic construct
SMEEIKERQRGKALEKVKEILEKMKSEKHKERSIFEVTYSMPDFENGKMIEVKEVLTYKEALEEIEKWEKKK

SEQ ID NO: 154 - protein, 97 aa, synthetic construct
SLEEFIVEVYSEYLRIYEKFNGDLREVGRALMEFLGELVKRGRVKEFYELVEFLESNGIFPEKEYIPGNQMRYTFTSRQIPKMMEVLKKVYEEELKK

SEQ ID NO: 155 - protein, 97 aa, synthetic construct
SLRDLIVEVYSKYLEIYEKHNGDLRETGRAWMAFLGELVAQGRVKEFYELVDFLESNGIFPEKEYIPGNQMRYTFTSRQIPKAMEILKKVYEEELAK

SEQ ID NO: 156 - protein, 91 aa, synthetic construct
MQMSPEEYIDSLFSKTKNTPEEIIEVVKEVRKLFPNMDIELSTKSGYKEYIPASTSEEEREKRLREFFEKYPREEIEITFVIPREEVDAFK

SEQ ID NO: 157 - protein, 115 aa, synthetic construct
MVIEIPSEKKKLRLEVVMRRPDDGIATTVTIEVESEELVLEIRDDSEVPHIVLRFPEGKVTLTLPNGEVEVLEPKNATLVVYMRGTDRMVIDFIETMDGVFPTDEEIEEWAREDD

SEQ ID NO: 158 - protein, 91 aa, synthetic construct
SERERLERALELLDRMLEKLEEALEGKVKDWEAFFREWNELFAEFLRLTGEDRQTRTIEIEYTPDEVPEEDLRKLKELVERMTREVRERAE

SEQ ID NO: 159 - protein, 116 aa, synthetic construct
SESIKKLIEEKLKEMYELPGVGERAREHLERVMELMLRFAERRAAGGPRPSPAEVDELMEHLREMVWAMREAGASKETIAKGLKLIEEVTSNPEMGFEISYEFLPGGYMRITFTWH

SEQ ID NO: 160 - protein, 119 aa, synthetic construct
ATRYRRVTFEFEPRGDRAELEAVWAQYQEVVDRLIRYFERRVEELEEEIRRVERENEEEGTGDWERVERLRRERREAEEIVRRLRELRRELEERFPVDPLGTLMEAARRIDEILPEEFH

SEQ ID NO: 161 - protein, 97 aa, synthetic construct
PPKPSGFTKEHEEMLEKFREIVERRNSEYLPEFLKKLEELEKILKEHMTPEDTISMRHALDEEGRPVMVIEFIFRDPELHEKMKELVDELNELMSKM

SEQ ID NO: 162 - protein, 88 aa, synthetic construct
EVYDLDTMSREELRELLERIERTVEWMLSRLPDLGERVRELRRRAFEGVEEVRRALEEGAPLQEVIGLMSGMSPINREIQELWDEVNA

SEQ ID NO: 163 - protein, 97 aa, synthetic construct
ATVTVVDGKEFYVPHGRLIVEKKGDTWYITLEFEKPDESHVKAVVEAFKYLVEQGEDPRNFILVVTYSPRKGEKTSREEMRRITEEMMKKILEEIDK

SEQ ID NO: 164 - DNA, 378 bp, synthetic construct
  source 1..378: mol_type = other DNA
gcccaggtgcagctccaggagtccgggggcgggttggtgcaggctggagggtcactgaggttgtcttgcgccgcgagtgaacggacgtttagcacctacgccatgggatggttcagacaggctccaggccgagagcgcgaattcctggcccaaatcaattggtcaggcacaaccacatattacgcagagtctgtcaaggacagaactactataagccgggataacgcaaaaaataccgtgtatctcgaaatgaataacctgaacgccgatgacaccggtatctatttttgtgctgcacatcctcagaggggatggggttctactcttggatggacgtactggggccaggggacacaagtaactgttagttccggcggc

SEQ ID NO: 165 - DNA, 774 bp, synthetic construct
  source 1..774: mol_type = other DNA
ggggcccaggtcaaactgcagcagtctggggctgaactggcaaaacctggggcctcagtgaagatgtcctgcaagacttctggctacacctttactagctactggatgaactgggtaaaacagaggcctggacagggtctggaatggattggatacattaatcctaccactggttatactaagtacaatcagaagttcaaggacaaggccacattgactgcagacaaatcctccagcacggcctacatgcaactgagcagcctgaccaatgtggactctgcagtctattattgtacaactggttactcttattttgactactggggccaagggaccacggtcaccgtctcctcaggtggaggcggttcaggcggaggtggctctggcggtggcggatcggacatcgagctcactcagtctccagcaatcatgtctgcatctccaggggagaaggtcaccataacctgcagtgccagctcaagtgtaaattacatgcactggttccagcagaagccaggcacttctcccaaactctggatttctagcacatccaacctggcttctggagtccctgctcgcttcagtggcagtggatctgggacctcttactctctcacaatcagccggatggaggctgaagatgctgccacttattactgccagcaaaggagtagttacccatacacgttcggagggggcaccaagctgcaaatcaaacgggcggccgcaggtgcgccggtgccgtatccggatccgctggaaccgcgtgccgca

SEQ ID NO: 166 - DNA, 255 bp, synthetic construct
  source 1..255: mol_type = other DNA
attccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatc

SEQ ID NO: 167 - DNA, 222 bp, synthetic construct
  source 1..222: mol_type = other DNA
ctgagtatccagcgctgcatccagtctctggtccatgcttgccagtgtcggaatgccaattgctcactgccatcctgccagaagatgaagcgggttgtgcagcataccaagggttgcaaacggaaaaccaatggcgggtgccccatctgcaagcagctcattgccctctgctgctaccatgccaagcactgccaggagaacaaatgcccggtgccgttctgc

SEQ ID NO: 168 - DNA, 684 bp, synthetic construct
  source 1..684: mol_type = other DNA
tccacgaacaagcggaccaacttgaagaagcccaactcggagcggacggggcacgggctgagagtccggtttaaccccctggcactgctcctagacgcgtctctggaaggagagttcgatctggtgcagaggatcatctatgaggtggaagatcccagcaagcccaacgacgaagggatcaccccactgcacaacgccgtctgcgccggccaccatcacatcgtgaagttcctgctggattttggtgtcaacgtgaatgctgctgatagtgatggatggacgccgctgcactgcgctgcctcttgtaacagcgttcacctctgcaaacagctggtggagagtggtgccgccatttttgcctcaaccataagcgacattgaaactgctgcagacaagtgtgaggagatggaggaaggctacatccagtgctcccagtttctatatggggtgcaggaaaagctgggtgtgatgaacaaaggtgtggcgtatgctctgtgggactacgaggcccagaacagtgacgagctgtccttccacgaaggggacgccctcaccatcctgaggcgcaaggacgaaagcgagactgagtggtggtgggctcgccttggagaccgggagggctatgtgcccaaaaacctgctggggctgtatccacggatcaaaccccgacagcgaacactcgcc

SEQ ID NO: 169 - DNA, 279 bp, synthetic construct
  source 1..279: mol_type = other DNA
gatatccaggtgcaggtgaacatcgatgacaatggcaagaacttcgactacacctacacagtgaccaccgagtccgagctgcagaaggtgctgaacgagctgaaggattacatcaagaagcagggcgccaagcgggtgcgcatcagcatcacagctcggactaagaaggaggctgagaagttcgccgccatcctcatcaaggtgttcgccgagctgggatacaacgacatcaacgtgaccttcgacggtgacaccgtcaccgtcgagggtcagctggag

SEQ ID NO: 170 - protein, 126 aa, synthetic construct
AQVQLQESGGGLVQAGGSLRLSCAASERTFSTYAMGWFRQAPGREREFLAQINWSGTTTYYAESVKDRTTISRDNAKNTVYLEMNNLNADDTGIYFCAAHPQRGWGSTLGWTYWGQGTQVTVSSGG

SEQ ID NO: 171 - protein, 258 aa, synthetic construct
GAQVKLQQSGAELAKPGASVKMSCKTSGYTFTSYWMNWVKQRPGQGLEWIGYINPTTGYTKYNQKFKDKATLTADKSSSTAYMQLSSLTNVDSAVYYCTTGYSYFDYWGQGTTVTVSSGGGGSGGGGSGGGGSDIELTQSPAIMSASPGEKVTITCSASSSVNYMHWFQQKPGTSPKLWISSTSNLASGVPARFSGSGSGTSYSLTISRMEAEDAATYYCQQRSSYPYTFGGGTKLQIKRAAAGAPVPYPDPLEPRAA

SEQ ID NO: 172 - protein, 85 aa, synthetic construct
IPASEQETLVRPKPLLLKLLKSVGAQKDTYTMKEVLFYLGQYIMTKRLYDEKQQHIVYCSNDLLGDLFGVPSFSVKEHRKIYTMI

SEQ ID NO: 173 - protein, 74 aa, synthetic construct
LSIQRCIQSLVHACQCRNANCSLPSCQKMKRVVQHTKGCKRKTNGGCPICKQLIALCCYHAKHCQENKCPVPFC

SEQ ID NO: 174 - protein, 228 aa, synthetic construct
STNKRTNLKKPNSERTGHGLRVRFNPLALLLDASLEGEFDLVQRIIYEVEDPSKPNDEGITPLHNAVCAGHHHIVKFLLDFGVNVNAADSDGWTPLHCAASCNSVHLCKQLVESGAAIFASTISDIETAADKCEEMEEGYIQCSQFLYGVQEKLGVMNKGVAYALWDYEAQNSDELSFHEGDALTILRRKDESETEWWWARLGDREGYVPKNLLGLYPRIKPRQRTLA

SEQ ID NO: 175 - protein, 93 aa, synthetic construct
DIQVQVNIDDNGKNFDYTYTVTTESELQKVLNELKDYIKKQGAKRVRISITARTKKEAEKFAAILIKVFAELGYNDINVTFDGDTVTVEGQLE

SEQ ID NO: 176 - DNA, 1188 bp, synthetic construct
  source 1..1188: mol_type = other DNA
gagggcagcggcagcgagggctccggcagcgagggcagcggctccgaaggcagctcaaaagcactcctgaagggagtccgcgactttaatcctatatcagcgtgtgtatgtttgcttgaaaactcctctgatgggcattcagaaaggctttttggtattgggtttggtccctacataatagcaaaccaacacctgtttcgacgcaataacggcgagcttacgattaagacgatgcacggggaatttaaagtgaaaaacagcacacaattgcaaatgaaacccgtcgaagggagagacatcattgtaataaagatggcaaaggatttcccaccctttccgcagaaattgaaattccggcaacctactataaaggatcgagtttgtatggtttctacgaactttcagcaaggcagcggcgaaggtcgggggagtttgcttacctgtggcgatgtcgaggagaatccagggcccggatccgtgtccaagggcgaagaactgattaaggagaacatgcacatgaaactctatatggaaggcacagtggacaaccaccacttcaaatgcactagcgagggcgagggcaagccctacgagggcacccagaccatgcggatcaaagtggtggaaggcggacctctaccttttgccttcgacatcctggccaccagctttctgtacggctccaagacctttatcaaccatacccagggcatccccgacttcttcaagcagtctttccctgagggctttacatgggagagggtgaccacctatgaagatggaggcgtgctgacagccacacaggacaccagcctgcaagacggttgtctgatctacaacgtgaagatccggggcgtgaacttcacatctaatggccctgtgatgcagaaaaagacactgggatgggaggccttcaccgagacactgtaccctgccgacggaggtcttgaaggcagaaacgacatggccctgaaactggtgggaggaagccacctcatcgccaacgctaagacaacataccggagcaagaagcctgccaagaacctgaaaatgcccggcgtgtactacgtggactacagactggaaagaatcaaggaagctaataacgaaacctacgtggaacagcacgaggtggccgtcgccagatactgcgacctgcctagcaagctgggccacaagctgaactag

SEQ ID NO: 177 - DNA, 414 bp, synthetic construct
  source 1..414: mol_type = other DNA
gagggcagcggcagcgagggctccggcagcgagggcagcggctccgaaggcagcaaatcagtctcctccctcgtgtccgaatcctctcacatcgttcacaaggaagataccagtttctggcagcattggattaccacaaaagacgggcagtgcggctcaccgctggtgtctgacattgacgggaacattctgggaatccactccctgacacacaccactaacggctcaaactatttcgttgagttcccagagaagttcgtagctacatatctggacgctgcggacggctggtgcaagaactggaaattcaacgcggacaagatttcatggggtagtttcacgcttgtcgaagacgcgcccgaggacgactttatggcgaaaaaaaccgtggctgctattatggatagcggctag

SEQ ID NO: 178 - protein, 395 aa, synthetic construct
EGSGSEGSGSEGSGSEGSSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQGSGEGRGSLLTCGDVEENPGPGSVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 179 - protein, 137 aa, synthetic construct
EGSGSEGSGSEGSGSEGSKSVSSLVSESSHIVHKEDTSFWQHWITTKDGQCGSPLVSDIDGNILGIHSLTHTTNGSNYFVEFPEKFVATYLDAADGWCKNWKFNADKISWGSFTLVEDAPEDDFMAKKTVAAIMDSG

SEQ ID NO: 180 - DNA, 189 bp, synthetic construct
  source 1..189: mol_type = other DNA
atgccgcagctggactccggcgggggcggcgcgggcggcggcgacgacctcggcgcgccggacgagctgctggccttccaggatgaaggcgaggagcaggacgacaagagccgcgacagcgccgccggtcccgagcgcgacctggccgagctcaagtcgtcgctcgtgaacgagtccgagggcggatcc

SEQ ID NO: 181 - DNA, 189 bp, synthetic construct
  source 1..189: mol_type = other DNA
atgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctggatcc

SEQ ID NO: 182 - DNA, 201 bp, synthetic construct
  source 1..201: mol_type = other DNA
atgtgtcacaagctgcctcccgcccccgcttggcaccacttcccgccccgctgtgtggacatgggctgtgccgggctccgggatgcacacgaggagaaccctgagagcatcctggacgagcacgtacagcgtgtgctgaggacacctggccgccagtcgcctgggcctggccatcgctccccggacagtgggcacggatcc

SEQ ID NO: 183 - DNA, 147 bp, synthetic construct
  source 1..147: mol_type = other DNA
atgaatcccgatggcctatctcaggagcagctggagcaccgggagcgctccttacaaactctcagagatatccagcgcatgctttttcctgatgagaaagaattcacaggagcacaaagtgggggaccgcagcagaatcctggatcc

SEQ ID NO: 184 - DNA, 180 bp, synthetic construct
  source 1..180: mol_type = other DNA
atggtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggatcc

SEQ ID NO: 185 - DNA, 249 bp, synthetic construct
  source 1..249: mol_type = other DNA
atgaaccgcgagggagctcccgggaagagtccggaggagatgtacattcagcagaaggtccgagtgctgctcatgctgcggaagatgggatcaaacctgacagccagcgaggaggagttcctgcgcacctatgcaggggtggtcaacagccagctcagccagctgcctccgcactccatcgaccagggtgcagaggacgtggtgatggcgttttccaggtcggagacggaagaccggaggcagggatcc

SEQ ID NO: 186 - DNA, 630 bp, synthetic construct
  source 1..630: mol_type = other DNA
atgaaggcccatgttttggctgcatctgttgaacaagcaactgagaatttcttggagaagggggataaaattgcgaaggagagccagtttctcaaggaggagcttgtggctgctgtagaagatgttcgaaaacaaggtgatttgatgaaggctgctgcaggagagttcgcagatgatccctgctcttctgtgaagcgaggcaacatggttcgggcagctcgagctttgctctctgctgttacccggttgctgattttggctgacatggcagatgtctacaaattacttgttcagctgaaagttgtggaagatggtatcttgaagttgaggaatgctggcaatgaacaagacttaggaatccagtataaagccctaaaacctgaagtggataagctgaacattatggcagccaaaagacaacaggaattgaaagatgttggccatcgtgatcagatggctgcagctagaggaatcctgcagaagaacgttccgatcctctatactgcatcccaggcatgcctacagcaccctgatgtcgcagcctataaggccaacagggacctgatatacaagcagctgcagcaggcggtcacaggcatttccaatgcagcccaggccactgccggatcc

SEQ ID NO: 187 - protein, 63 aa, synthetic construct
MPQLDSGGGGAGGGDDLGAPDELLAFQDEGEEQDDKSRDSAAGPERDLAELKSSLVNESEGGS

SEQ ID NO: 188 - protein, 63 aa, synthetic construct
MPQLSGGGGGGGGDPELCATDEMIPFKDEGDPQKEKIFAEISHPEEEGDLADIKSSLVNESGS

SEQ ID NO: 189 - protein, 67 aa, synthetic construct
MCHKLPPAPAWHHFPPRCVDMGCAGLRDAHEENPESILDEHVQRVLRTPGRQSPGPGHRSPDSGHGS

SEQ ID NO: 190 - protein, 49 aa, synthetic construct
MNPDGLSQEQLEHRERSLQTLRDIQRMLFPDEKEFTGAQSGGPQQNPGS

SEQ ID NO: 191 - protein, 60 aa, synthetic construct
MVTRNDVAPTLMSVPRYLPRPANPDEIGNFIDENLKAADTDPTAPPYDSLLVFDYEGSGS

SEQ ID NO: 192 - protein, 83 aa, synthetic construct
MNREGAPGKSPEEMYIQQKVRVLLMLRKMGSNLTASEEEFLRTYAGVVNSQLSQLPPHSIDQGAEDVVMAFSRSETEDRRQGS

SEQ ID NO: 193 - protein, 210 aa, synthetic construct
MKAHVLAASVEQATENFLEKGDKIAKESQFLKEELVAAVEDVRKQGDLMKAAAGEFADDPCSSVKRGNMVRAARALLSAVTRLLILADMADVYKLLVQLKVVEDGILKLRNAGNEQDLGIQYKALKPEVDKLNIMAAKRQQELKDVGHRDQMAAARGILQKNVPILYTASQACLQHPDVAAYKANRDLIYKQLQQAVTGISNAAQATAGS

SEQ ID NO: 194 - DNA, 435 bp, synthetic construct
  source 1..435: mol_type = other DNA
atggctcatgtgcagctgcaggagtctgggggaggcttggtgcaggctgggggttctctgacgctctcctgtgcagcctctggaaacaccttcaatatcgtcgccatgggctggtaccgccagggtccagggaagcagcgcgagttggtcgcaactattactatgaatagtggtatcacaaactatcaagactccgtgaagggccggttcaccatctccagagacaaagccaagaacacaagtgatctgcgaatgagcgacctgaaacctgaggacacggccgtgtattactgtaacgctctatcgggacttcccccctatgcgactacttactggggccaggggacccaggtcaccgtctcctcaagaagcttcgaattctgcagtcgacggtaccgcgggcccgggatccaccggccggtcgccaccggatcc

SEQ ID NO: 195 - DNA, 429 bp, synthetic construct
  source 1..429: mol_type = other DNA
atggctcaggtgcagctggtggagtctgggggaggattggtgcaggctgggggctcaatgagactctcctgcgcaacctctgtgaacaccttcagtggttatactgtggcctggttccgccgggctccagggaaggagcgtgagtttgtaggagctcttaactggagtagtggtagtacatggtatgcagactccgtgaagggccgattcaccatttccagagacagcgccaagaactcggtttttctgcaaatgaacagcctgaaacctgaggactcggccgtctattattgtaatgccattgatagcgactatggagatgactactggggccaggggacccaggtcaccgtctccagcagaagcttcgaattctgcagtcgacggtaccgcgggcccgggatccaccggccggtcgccaccggatcc

SEQ ID NO: 196 - DNA, 435 bp, synthetic construct
  source 1..435: mol_type = other DNA
atggctgatgtgcagctggtggagtctgggggaggattggtgcaggctgggggttctttgagactctcctgttcagcctctggatttaccttcagaaactatgtcatggcatggttccgccaggctccgggaaaggagcgtgaatttgtagcagctgttagcatgagcggaagtacatggtatggagactccgtgaagggccgattcaccatttccagagagaatgccaagaattcggtgtatctgcaaatgaacaacctgaaacctgaggacacggccgtctattactgtactgcagtcaacagcgacccatggggaaatgaacatgactaccggggccaggggacccaggtcaccgtctccagcagaagcttcgaattctgcagtcgacggtaccgcgggcccgggatccaccggccggtcgccaccggatcc

SEQ ID NO: 197 - DNA, 420 bp, synthetic construct
  source 1..420: mol_type = other DNA
atggctcaggtgcagctggtggagtctgggggaggcttggtgcagcctggggggtctctgagactctcctgtgcagcctctggagtcatcttcagtatctatgccatgggctggtaccgccaggctccagggaagcagcgcgagttgatcgcagcgattactagtggtggtagcacaaactatgcagactccgtgaagggccgattcaccatctccagagacaacgccaagaacacggtgtatctgcgaatgaacagcctgaaacctgaggacacggccgtctattactgtaatgcgcgccggtattctcgttccgactggggccaggggacccaggtcaccgtctcctcaagaagcttcgaattctgcagtcgacggtaccgcgggcccgggatccaccggccggtcgccaccggatcc

SEQ ID NO: 198 - DNA, 1158 bp, synthetic construct
  source 1..1158: mol_type = other DNA
atgcctcaactggattctggcggaggcggagctggtggcggagatgatcttggagcacctgatgagctgctggcattccaggacgagggcgaagaacaggacgacaagagcagagattctgccgccggacctgagagagatctggccgagctgaagtccagcctggtcaatgaatctgaaggcgccgctggcggcgctggaattcctggtgttcctggcgctggtgctggtgcaagaggcgaagctgaagccctgggaagagaacacgctgcccagagactgttccccgacaagctgcctgagcctctggaagatggactgaaggcccctgagtgtaccagcggcatgtacaaagaaaccgtgtacagcgccttcaacctgctgatgcactaccctccacctagcggagcaggacagcatcctcaacctcagcctccactgcacaaggccaatcagccacctcatggcgtgccacagctgagcctgtacgagcacttcaacagccctcatcctactccagctccagccgacatcagccagaaacaggtgcacagacctctgcagacccctgacctgagcggcttttacagcctgacaagcggcagcatgggacagctgcctcataccgtgtcctggttcacacaccccagcctgatgcttggaagcggagtgcctggacaccctgccgctattcctcatcctgccatcgtgcctccaagcggcaagcaagagctgcagcccttcgaccggaacctgaaaacacaggccgagagcaaggccgagaaagaggccaagaagcccaccatcaagaagcctctgaacgccttcatgctgtacatgaaagaaatgcgggccaaagtgatcgccgagtgcaccctgaaagagtccgccgccatcaaccagatcctgggcagaagatggcacgccctgtccagagaggaacaggccaagtactacgagctggcccggaaagaacggcagctgcacatgcaactgtaccctggctggagcgccagagacaactacggcaagaagaagcggcggagcagagagaagcaccaagagtctacaaccggcggcaagagaaacgccttcggcacatatcccgagaaagccgctgctcccgctcctttcctgcctatgactgttctgggatcc

SEQ ID NO: 199 - protein, 145 aa, synthetic construct
MAHVQLQESGGGLVQAGGSLTLSCAASGNTFNIVAMGWYRQGPGKQRELVATITMNSGITNYQDSVKGRFTISRDKAKNTSDLRMSDLKPEDTAVYYCNALSGLPPYATTYWGQGTQVTVSSRSFEFCSRRYRGPGIHRPVATGS

SEQ ID NO: 200 - protein, 143 aa, synthetic construct
MAQVQLVESGGGLVQAGGSMRLSCATSVNTFSGYTVAWFRRAPGKEREFVGALNWSSGSTWYADSVKGRFTISRDSAKNSVFLQMNSLKPEDSAVYYCNAIDSDYGDDYWGQGTQVTVSSRSFEFCSRRYRGPGIHRPVATGS

SEQ ID NO: 201 - protein, 145 aa, synthetic construct
MADVQLVESGGGLVQAGGSLRLSCSASGFTFRNYVMAWFRQAPGKEREFVAAVSMSGSTWYGDSVKGRFTISRENAKNSVYLQMNNLKPEDTAVYYCTAVNSDPWGNEHDYRGQGTQVTVSSRSFEFCSRRYRGPGIHRPVATGS

SEQ ID NO: 202 - protein, 140 aa, synthetic construct
MAQVQLVESGGGLVQPGGSLRLSCAASGVIFSIYAMGWYRQAPGKQRELIAAITSGGSTNYADSVKGRFTISRDNAKNTVYLRMNSLKPEDTAVYYCNARRYSRSDWGQGTQVTVSSRSFEFCSRRYRGPGIHRPVATGS

SEQ ID NO: 203 - protein, 386 aa, synthetic construct
MPQLDSGGGGAGGGDDLGAPDELLAFQDEGEEQDDKSRDSAAGPERDLAELKSSLVNESEGAAGGAGIPGVPGAGAGARGEAEALGREHAAQRLFPDKLPEPLEDGLKAPECTSGMYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIKKPLNAFMLYMKEMRAKVIAECTLKESAAINQILGRRWHALSREEQAKYYELARKERQLHMQLYPGWSARDNYGKKKRRSREKHQESTTGGKRNAFGTYPEKAAAPAPFLPMTVLGS

SEQ ID NO: 204 - DNA, 1461 bp, synthetic construct
  source 1..1461: mol_type = other DNA
atgggctccggcgagtctctgtttaagggccccagagattacaaccctatcagcagcaccatttgccacctgaccaacgagagcgatggacatacaaccagcctgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagaccaagagcatgagcagcatggtctccgacacaagctgtaccttcccatctagcgacggcattttctggaagcactggatccagaccaaggacggccagtgcggctctcctctggtgagcaccagagacggcttcatcgtgggcatccactctgccagcaactttacaaacacaaacaattacttcaccagcgtgccaaagaacttcatggaactgctgaccaaccaggaggctcagcagtgggtgtccggctggcggctgaacgccgatagcgtgctgtggggcggccacaaagtgttcatggtgaagccagaggagcctttccaacctgtgaaggaggctacccagctgatgaacagcggctctgagggcagcggcagcgagggatcagtgtccaagggcgaagaactgattaaggagaacatgcacatgaaactctatatggaaggcacagtggacaaccaccacttcaaatgcactagcgagggcgagggcaagccctacgagggcacccagaccatgcggatcaaagtggtggaaggcggacctctaccttttgccttcgacatcctggccaccagctttctgtacggctccaagacctttatcaaccatacccagggcatccccgacttcttcaagcagtctttccctgagggctttacatgggagagggtgaccacctatgaagatggaggcgtgctgacagccacacaggacaccagcctgcaagacggttgtctgatctacaacgtgaagatccggggcgtgaacttcacatctaatggccctgtgatgcagaaaaagacactgggatgggaggccttcaccgagacactgtaccctgccgacggaggtcttgaaggcagaaacgacatggccctgaaactggtgggaggaagccacctcatcgccaacgctaagacaacataccggagcaagaagcctgccaagaacctgaaaatgcccggcgtgtactacgtggactacagactggaaagaatcaaggaagctaataacgaaacctacgtggaacagcacgaggtggccgtcgccagatactgcgacctgcctagcaagctgggccacaagctgaactag

SEQ ID NO: 205 - DNA, 726 bp, synthetic construct
  source 1..726: mol_type = other DNA
atgggatcctcaaaagcactcctgaagggagtccgcgactttaatcctatatcagcgtgtgtatgtttgcttgaaaactcctctgatgggcattcagaaaggctttttggtattgggtttggtccctacataatagcaaaccaacacctgtttcgacgcaataacggcgagcttacgattaagacgatgcacggggaatttaaagtgaaaaacagcacacaattgcaaatgaaacccgtcgaagggagagacatcattgtaataaagatggcaaaggatttcccaccctttccgcagaaattgaaattccggcaacctactataaaggatcgagtttgtatggtttctacgaactttcagcaaaaatcagtctcctccctcgtgtccgaatcctctcacatcgttcacaaggaagataccagtttctggcagcattggattaccacaaaagacgggcagtgcggctcaccgctggtgtctatcattgacgggaacattctgggaatccactccctgacacacaccactaacggctcaaactatttcgttgagttcccagagaagttcgtagctacatatctggacgctgcggacggctggtgcaagaactggaaattcaacgcggacaagatttcatggggtagtttcacgcttgtcgaagacgcgcccgaggacgactttatggcgaaaaaaaccgtggctgctattatggatagcggcagctag

SEQ ID NO: 206 - DNA, 1527 bp, synthetic construct
  source 1..1527: mol_type = other DNA
atggggtccagccccgaggatgagattcaggccctggaagaggaaaacgcccagctggaacaagagaacgccgctctggaagaagagattgcccagctcgagtacggcggtggctcagggagcggctctgtggtgcttgtagggcgattgctgctctctggttctgggtccgccccaataacggcctacgcccaacagacacgcggtctgctggggtgcattattacctcacttacgggcagagacaaaaaccaggctgagggagaagttcagattgtttcaacggcggctcagacctttcttgccacctgtataaacggagtatgctggacagtctaccatggtgcagggacccgcacaatagcgagtcccaaagggcccgtcatacagatgtatactaacgtcgataaggacctcgtgggttggcccgctcctcaaggcacgcgatccctcacaccttgtgcgtgcggcagttcagacctctatctggtgactcgccatgccgacgtaatccctgttcgccggaggggtgatagtagagggagcttgctcagtcctaggccgatttcctatctcaagggctcaagcggcggcccattgctctgcccggccggccacgcggtaggaattttccgggccgcagtttgcacacgcggcgtcgcaaaagcagtagactttataccagtagagaaccttgaaacaacgatgaggtcccctgtatttacagataacagttcccctccggctgtcagcggcagcggcgaaggtcgggggagtttgcttacctgtggcgatgtcgaggagaatccagggcccggatccgtgagcaagggcgaagaactgatcaaagaaaacatgcacatgaagctgtacatggaaggcaccgtgaacaaccaccacttcaagtgcaccagcgaaggcgagggcaagccttatgagggcacccagaccatgcggatcaaggtggtggaaggtggccctctgcctttcgcctttgatatcctggccaccagctttatgtacggcagcaagaccttcatcaatcacacccagggcatccccgatttcttcaagcagagcttccccgagggcttcacctgggagagaagcaccacatatgaggacggcggcgtgctgacagccacacaggatacttctctgcaggacggctgcctgatctacaacgtgaagatccggggcgtgaacttccccagcaatggccccgtgatgcagaagaaaaccctcggctgggaagccagcaccgagatgctgtatcctgccgatggtggcctggaaggcagagactatatggccctgaagctcgttggcggcggacacctgatctgcaatgccaagaccacctacagaagcaagaagcccgccaagaacctgaagatgcccggcgtgtactacgtggacagacggctggaaagaatcaaagaggccgacaaagaaaccagcgtggaacagcacgaagtggccgtggccagatactgcgatctgccttctaagctgggccacaagtag

SEQ ID NO: 207 - DNA, 708 bp, synthetic construct
  source 1..708: mol_type = other DNA
ggggagagtttgtttaaggggccaagggactataacccaatatcatccactatttgccacctcactaacgagagcgatggacatacaacctctctctacgggataggcttcggtcctttcatcattaccaataagcatctgtttcgccgaaacaacggtactcttctggttcaatctcttcatggtgtcttcaaggtgaaaaacaccactacgcttcagcaacacctgattgatggtagggatatgataattatcagaatgccgaaagatttcccaccttttccacagaagctgaaattcagggaaccgcagagagaggagaggatttgtttggtaacgaccaacttccagacgaagagtatgagttctatggtgtccgacactagctgcacgttcccctcaagtgatgggacattctggaaacactggatacagactaaagacggacagtgtggaaacccattggtttccacccgagatggttttatcgtgggtatccatagcgcctctaatttcacaaacacgaacaactatttcgcttcagtgcccaaaaactttatggagctgctcacaaaccaagaagcgcagcagtgggtaagcggttggagacttaacgctgactcagttctctggggggggcacaaagtattcatggtaaagccagaggagccattccaaccagtcaaagaagccacacaacttatgaac

SEQ ID NO: 208 - DNA, 708 bp, synthetic construct
  source 1..708: mol_type = other DNA
ggcgagtctctgtttaagggccccagagattacaaccctatcagcagcaccatttgccacctgaccaacgagagcgatggacatacaaccagcctgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagaccaagagcatgagcagcatggtctccgacacaagctgtaccttcccatctagcgacggcaccttctggaagcactggatccagaccaaggacggccagtgcggcaaccctctggtgagcaccagagacggcttcatcgtgggcatccactctgccagcaactttacaaacacaaacaattacttcgccagcgtgccaaagaacttcatggaactgctgaccaaccaggaggctcagcagtgggtgtccggctggcggctgaacgccgatagcgtgctgtggggcggccacaaagtgttcatggtgaagccagaggagcctttccaacctgtgaaggaggctacccagctgatgaac

SEQ ID NO: 209 - DNA, 708 bp, synthetic construct
  source 1..708: mol_type = other DNA
ggcgagatcctgtttaagggccagagagattacaaccctatcagcagcaccatttgccacctgaccaacgagagcgatggacatacaaccacactgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagaccaagagcatgagcagcatggtctccgacacaagctgtaccttcccatctagcgacggcattttctggaagcactggatccagaccaaggacggccagtgcggctctcctctggtgagcaccagagacggcttcatcgtgggcatccactctgccagcaactttgctaacacaaacaattacttcaccagcgtgccaaagaacttcatggaactgctgaccaaccaggaggctcagcagtgggtgtccggctggcggctgaacgccgatagcgtgctgtggggcggccacaaagtgttcatgcggaagccagaggagcctttccaacctgtgaaggaggtgacccagctgatgaac

SEQ ID NO: 210 - protein, 486 aa, synthetic construct
MGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNSGSEGSGSEGSVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 211 - protein, 239 aa, synthetic construct
GSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNS

SEQ ID NO: 212 - protein, 241 aa, synthetic construct
MGSSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQKSVSSLVSESSHIVHKEDTSFWQHWITTKDGQCGSPLVSIIDGNILGIHSLTHTTNGSNYFVEFPEKFVATYLDAADGWCKNWKFNADKISWGSFTLVEDAPEDDFMAKKTVAAIMDSGS

SEQ ID NO: 213 - protein, 236 aa, synthetic construct
SKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQKSVSSLVSESSHIVHKEDTSFWQHWITTKDGQCGSPLVSIIDGNILGIHSLTHTTNGSNYFVEFPEKFVATYLDAADGWCKNWKFNADKISWGSFTLVEDAPEDDFMAKKTVAAIMDS

SEQ ID NO: 214 - protein, 508 aa, synthetic construct
MGSSPEDEIQALEEENAQLEQENAALEEEIAQLEYGGGSGSGSVVLVGRLLLSGSGSAPITAYAQQTRGLLGCIITSLTGRDKNQAEGEVQIVSTAAQTFLATCINGVCWTVYHGAGTRTIASPKGPVIQMYTNVDKDLVGWPAPQGTRSLTPCACGSSDLYLVTRHADVIPVRRRGDSRGSLLSPRPISYLKGSSGGPLLCPAGHAVGIFRAAVCTRGVAKAVDFIPVENLETTMRSPVFTDNSSPPAVSGSGEGRGSLLTCGDVEENPGPGSVSKGEELIKENMHMKLYMEGTVNNHHFKCTSEGEGKPYEGTQTMRIKVVEGGPLPFAFDILATSFMYGSKTFINHTQGIPDFFKQSFPEGFTWERSTTYEDGGVLTATQDTSLQDGCLIYNVKIRGVNFPSNGPVMQKKTLGWEASTEMLYPADGGLEGRDYMALKLVGGGHLICNAKTTYRSKKPAKNLKMPGVYYVDRRLERIKEADKETSVEQHEVAVARYCDLPSKLGHK

SEQ ID NO: 215 - protein, 193 aa, synthetic construct
APITAYAQQTRGLLGCIITSLTGRDKNQAEGEVQIVSTAAQTFLATCINGVCWTVYHGAGTRTIASPKGPVIQMYTNVDKDLVGWPAPQGTRSLTPCACGSSDLYLVTRHADVIPVRRRGDSRGSLLSPRPISYLKGSSGGPLLCPAGHAVGIFRAAVCTRGVAKAVDFIPVENLETTMRSPVFTDNSSPPAV

SEQ ID NO: 216 - protein, 236 aa, synthetic construct
GESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGTFWKHWIQTKDGQCGNPLVSTRDGFIVGIHSASNFTNTNNYFASVPKNFMELLTNQEAQQWVSGWRLNADSVLWGGHKVFMVKPEEPFQPVKEATQLMN

SEQ ID NO: 217 - protein, 236 aa, synthetic construct
GEILFKGQRDYNPISSTICHLTNESDGHTTTLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFANTNNYFTSVPKNFMELLTNQEAQQWVSGWRLNADSVLWGGHKVFMRKPEEPFQPVKEVTQLMN

SEQ ID NO: 218 - DNA, 966 bp, synthetic construct
  source 1..966: mol_type = other DNA
atggaaaacactgagaacagcgtcgatagcaagtccattaagaacctcgaacctaaaatcatacatggcagtgagagcatggatagcggcataagccttgacaactcctataaaatggactacccggagatgggtctgtgcataataatcaacaacaagaacttccacaaatccacggggatgacaagtaggtctggaaccgatgtcgatgcggcaaacttgcgcgagacctttagaaacctcaagtacgaggtacgcaataaaaacgatctcactcgcgaggaaatagtagagctgatgcgagatgtcagtaaggaagatcattccaagagatcctccttcgtctgcgtcttgttgagtcacggagaggaagggatcatattcggtacgaacggcccagtggatttgaaaaagataaccaatttctttagaggtgaccggtgccgctctctcacaggaaagccaaaactctttattatccaagcatgtaggggtacggaactggattgtggaatcgaaactgagaacttgtacttccagtccggggtagatgacgacatggcctgccataaaatccctgtcgaggccgattttctctacgcctactctactgcgcctgggtattacagctggagaaacagcaaggatggttcctggtttatacaaagcctgtgtgctatgctcaaacagtatgcggataagctggagttcatgcatatccttaccagagtaaatcgcaaagtggccacagagtttgagtcattttcttttgacgcaacatttcacgccaagaagcagattccttgtatagtatctatgctgacgaaagagctgtacttctatcacagcaccggcggatctgaaaccgtgcggtttcaatccagcggcggtggaggacggagcaaactgaatccacccgatgagtctggacccggttgtatgtcatgtaaatgtgtgttgagctag

SEQ ID NO: 219 - DNA, 981 bp, synthetic construct
  source 1..981: mol_type = other DNA
atggaaaacactgagaacagcgtcgatagcaagtccattaagaacctcgaacctaaaatcatacatggcagtgagagcatggatagcggcataagccttgacaactcctataaaatggactacccggagatgggtctgtgcataataatcaacaacaagaacttccacaaatccacggggatgacaagtaggtctggaaccgatgtcgatgcggcaaacttgcgcgagacctttagaaacctcaagtacgaggtacgcaataaaaacgatctcactcgcgaggaaatagtagagctgatgcgagatgtcagtaaggaagatcattccaagagatcctccttcgtctgcgtcttgttgagtcacggagaggaagggatcatattcggtacgaacggcccagtggatttgaaaaagataaccaatttctttagaggtgaccggtgccgctctctcacaggaaagccaaaactctttattatccaagcatgtaggggtacggaactggattgtggaatcgaaactgagaacttgtacttccagtccggggtagatgacgacatggcctgccataaaatccctgtcgaggccgattttctctacgcctactctactgcgcctgggtattacagctggagaaacagcaaggatggttcctggtttatacaaagcctgtgtgctatgctcaaacagtatgcggataagctggagttcatgcatatccttaccagagtaaatcgcaaagtggccacagagtttgagtcattttcttttgacgcaacatttcacgccaagaagcagattccttgtatagtatctatgctgacgaaagagctgtacttctatcacagcaccggatccgagggcagcggcagcgagggctccggcagcgagggcagcggctccgaaggcagccggagcaaactgaatccacccgatgagtctggacccggttgtatgtcatgtaaatgtgtgttgagctag

SEQ ID NO: 220 - protein, 321 aa, synthetic construct
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETENLYFQSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHSTGGSETVRFQSSGGGGRSKLNPPDESGPGCMSCKCVLS

SEQ ID NO: 221 - protein, 326 aa, synthetic construct
MENTENSVDSKSIKNLEPKIIHGSESMDSGISLDNSYKMDYPEMGLCIIINNKNFHKSTGMTSRSGTDVDAANLRETFRNLKYEVRNKNDLTREEIVELMRDVSKEDHSKRSSFVCVLLSHGEEGIIFGTNGPVDLKKITNFFRGDRCRSLTGKPKLFIIQACRGTELDCGIETENLYFQSGVDDDMACHKIPVEADFLYAYSTAPGYYSWRNSKDGSWFIQSLCAMLKQYADKLEFMHILTRVNRKVATEFESFSFDATFHAKKQIPCIVSMLTKELYFYHSTGSEGSGSEGSGSEGSGSEGSRSKLNPPDESGPGCMSCKCVLS

SEQ ID NO: 222 - DNA, 738 bp, synthetic construct
  source 1..738: mol_type = other DNA
atgacaatgttcgagaacgtgacaagagccctggccagacagctgaaccccagaggcgatctgacacctctggacagcctgattgacttcaagcggttccaccctttttgcctggtgctgcggaagagaaagagcacgctgttctggggcgctagatacgtgcggaccgactacaccctcctggatgtgctggagcctgggagctcccctagcgaccccaccgacacaggcaatttcggctttaagaacatgctggacaccagagtggaaggcgacgtggacgttcctaagaccgtgaaggtaaagggtacagccggcctgagccagaatagcaccctggaagtgcagaccctgagcgtggcccctaaggccctggagaccgtgcaggagagaaaactggctgctgatcaccccttcctgaaagagatgcaggaccagggcgaaaacctgtatgtggtgatggaagttgtggaaaccgtgcaggaggtcactctggaaagagccggaaaggccgaagcctgcttcagcctgcccttcttcgcccctctgggcctccaaggcagcatcaaccacaaggaagccgtgaccatcccaaagggctgtgtgctggccttccgggtgcggcagctgatggtgaaaggcaaagacgagtgggatatcccccacatctgcaacgacaacatgcagacattcccccccggcgagaagtctggcgaggaaaaagtgatcctgatccag

SEQ ID NO: 223 - DNA, 732 bp, synthetic construct
  source 1..732: mol_type = other DNA
atgttcagcgtgttcgaggagatcacccggatcgtggtcaaagaaatggacgctggcggagacatgatcgccgtgcggagcctggttgacgccgacagattcagatgttttcacctggtcggagagaaaagaaccttcttcggctgcagacactacacaaccggcctgacactgatggatatcctggacaccgacggcgataagtggctggatgagctggattccggcctgcaaggccagaaagccgagttccagatcctggacaacgtggacagcaccggcgagctgatcgtgcggctgcccaaggaaatcaccatcagcggctccttccaaggcttccaccaccagaagatcaagatttcagagaatcggatcagccaacagtacctggccaccctggaaaacagaaagctgaagagggaactgcctttcagctttagaagcatcaacacaagagagaacctgtacctggtgacagagacactggagaccgtgaaggaagagacactgaagagtgatagacagtataagttctggagccagatcagccagggccacctgtcctacaagcacaagggccagcgcgaagtgaccattcctccaaatagagtgctgagctacagagtgaagcagctggttttcccaaacaaggaaaccatgaacatccacttccggggcaagaccaagagctttcctgaggaaaaggacggcgccagcagctgcctgggaaag

SEQ ID NO: 224 - DNA, 720 bp, synthetic construct
  source 1..720: mol_type = other DNA
atgcctagcatgctggagcggatcagcaagaacctggtgaaggaaatcggaagcaaggacctgacccctgtgaaatacctgctcagcgctacaaagctgcggcagttcgtgatcctgcggaagaagaaagatagcagaagcagtttttgggagcagtctgactacgtgcccgtggaattctccctgaacgacatcctggagccttcctctagcgtgctggaaaccgttgtgaccggcccattccacttcagcgacatcatgatccagaaacacaaggccgacatgggcgttaatgtgggcatcgaggtgagcgtgagcggcgaggcctctgtcgaccacggctgctctctggagttccagatcgtgaccatcccctctccaaacctggaagatttccagaagagaaagctgctggatcccgagcctagcttcctgaaagagtgcagacggcggggcgacaacctgtacgtggtgacagaagccgtggaacttatcaacaacaccgtgctgtacgacagcagcagcgtgaacatcctgggcaagatcgccctgtggatcacatacggcaagggccagggccaaggagaatctctgagagtgaagaagaaagccctgaccctgcagaaaggcatggtgatggcctacaagagaaagcagctggtgatcaaggagaaggctatcctgatctctgatgacgatgagcagagaacattccaggat

SEQ ID NO: 225 - DNA, 825 bp, synthetic construct
  source 1..825: mol_type = other DNA
atgggctctgccttcgagagagtggtcagaagggtggtgcaggagctcgaccacggcggcgaattcatccccgtgacaagcctgcaaagcagcacaggcttccagccctactgcctggtggttagaaagccctcttcttcttggttctggaagcctagatacaagtgcgtgaacctgagcatcaaggacatcctggaacctgatgccgctgaacctgatgtgcagagaggccggagcttccacttctacgacgccatggacggccagatccagggcagcgtggaactcgccgccccaggccaagccaagatcgccggcggagctgctgtgtccgatagcagctctaccagcatgaacgtgtattctctgtccgtggaccccaacacctggcagaccctgctgcacgagagacacctgcggcagccagagcacaaggtgctgcagcagctgagatctagaggcgacaacgtgtacgtggtgaccgaggtgctccagacccagaaagaggtggaagtcacaagaacccacaagcgggaaggctccggaagattcagcctgcccggcgccacatgtctgcagggcgagggccagggccacctgagccagaagaaaaccgtgaccattcctagcggcagcaccctggcctttcgggtggcccagctggtcatcgacagcgacctggacgtgctgctgttccccgataagaagcagcggacctttcagcctcctgccaccggccacaaaagatccacctccgagggagcctggcctcagctgcctagcggcctgagcatgatgcggtgcctgcacaacttcctgactgat

SEQ ID NO: 226 - DNA, 810 bp, synthetic construct
  source 1..810: mol_type = other DNA
atgttcgccaaggccacaagaaatttcttgcgggaagtggacgctgatggcgacctgatcgccgtgtctaatctgaacgacagcgataagctgcaactgctgagcctggtcaccaaaaagaagcggttctggtgctggcagcggcctaagtaccagtttctgagcctgacactgggagatgtgctgattgaggaccaattccccagccccgtggtggtcgagagcgactttgtgaaatacgagggcaaattcgccaaccacgtgtccggcaccctggagacagccctgggcaaggtgaagctgaacctgggcggctctagcagagtggaatctcagagcagcttcggcaccctgcgcaagcaggaggtcgacctgcagcagctgatcagagattctgctgaacggaccatcaacctgagaaaccctgtgctgcaacaggtgctggaaggcagaaacgaggtgctgtgcgtgctgacccagaagatcacaacaatgcagaaatgtgtgatcagcgagcatatgcaggtggaggaaaagtgcggcggaatcgtgggcatccagaccaagaccgtgcaggttagcgccactgaggatggcaacgtgaccaaggacagcaatgtggtgctggaaatccctgctgctacaaccatcgcctacggcgtgatcgagctgtacgtgaagctggatggccagttcgagttctgcctgctgagaggcaagcagggaggcttcgagaacaagaaaagaatcgactccgtgtacctggaccccctggtctttagagaattcgccttcatcgatatgcctgac

SEQ ID NO: 227 - protein, 246 aa, synthetic construct
MTMFENVTRALARQLNPRGDLTPLDSLIDFKRFHPFCLVLRKRKSTLFWGARYVRTDYTLLDVLEPGSSPSDPTDTGNFGFKNMLDTRVEGDVDVPKTVKVKGTAGLSQNSTLEVQTLSVAPKALETVQERKLAADHPFLKEMQDQGENLYVVMEVVETVQEVTLERAGKAEACFSLPFFAPLGLQGSINHKEAVTIPKGCVLAFRVRQLMVKGKDEWDIPHICNDNMQTFPPGEKSGEEKVILIQ

SEQ ID NO: 228 - protein, 244 aa, synthetic construct
MFSVFEEITRIVVKEMDAGGDMIAVRSLVDADRFRCFHLVGEKRTFFGCRHYTTGLTLMDILDTDGDKWLDELDSGLQGQKAEFQILDNVDSTGELIVRLPKEITISGSFQGFHHQKIKISENRISQQYLATLENRKLKRELPFSFRSINTRENLYLVTETLETVKEETLKSDRQYKFWSQISQGHLSYKHKGQREVTIPPNRVLSYRVKQLVFPNKETMNIHFRGKTKSFPEEKDGASSCLGK

SEQ ID NO: 229 - protein, 240 aa, synthetic construct
MPSMLERISKNLVKEIGSKDLTPVKYLLSATKLRQFVILRKKKDSRSSFWEQSDYVPVEFSLNDILEPSSSVLETVVTGPFHFSDIMIQKHKADMGVNVGIEVSVSGEASVDHGCSLEFQIVTIPSPNLEDFQKRKLLDPEPSFLKECRRRGDNLYVVTEAVELINNTVLYDSSSVNILGKIALWITYGKGQGQGESLRVKKKALTLQKGMVMAYKRKQLVIKEKAILISDDDEQRTFQD

SEQ ID NO: 230 - protein, 275 aa, synthetic construct
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTD

SEQ ID NO: 231 - protein, 270 aa, synthetic construct
MFAKATRNFLREVDADGDLIAVSNLNDSDKLQLLSLVTKKKRFWCWQRPKYQFLSLTLGDVLIEDQFPSPVVVESDFVKYEGKFANHVSGTLETALGKVKLNLGGSSRVESQSSFGTLRKQEVDLQQLIRDSAERTINLRNPVLQQVLEGRNEVLCVLTQKITTMQKCVISEHMQVEEKCGGIVGIQTKTVQVSATEDGNVTKDSNVVLEIPAATTIAYGVIELYVKLDGQFEFCLLRGKQGGFENKKRIDSVYLDPLVFREFAFIDMPD

SEQ ID NO: 232 - DNA, 1359 bp, synthetic construct
  source 1..1359: mol_type = other DNA
atgacaatgttcgagaacgtgacaagagccctggccagacagctgaaccccagaggcgatctgacacctctggacagcctgattgacttcaagcggttccaccctttttgcctggtgctgcggaagagaaagagcacgctgttctggggcgctagatacgtgcggaccgactacaccctcctggatgtgctggagcctgggagctcccctagcgaccccaccgacacaggcaatttcggctttaagaacatgctggacaccagagtggaaggcgacgtggacgttcctaagaccgtgaaggtaaagggtacagccggcctgagccagaatagcaccctggaagtgcagaccctgagcgtggcccctaaggccctggagaccgtgcaggagagaaaactggctgctgatcaccccttcctgaaagagatgcaggaccagggcgaaaacctgtatgtggtgatggaagttgtggaaaccgtgcaggaggtcactctggaaagagccggaaaggccgaagcctgcttcagcctgcccttcttcgcccctctgggcctccaaggcagcatcaaccacaaggaagccgtgaccatcccaaagggctgtgtgctggccttccgggtgcggcagctgatggtgaaaggcaaagacgagtgggatatcccccacatctgcaacgacaacatgcagacattcccccccggcgagaagtctggcgaggaaaaagtgatcctgatccaggaaaacctgtacttccagtctgcctctgatgtgggcgacgtgcacgagggatttaggaccctgaaggaagaggtgcaaagagagacacagcaagtggagaaactgtctagagtgggccagagcagccttctgtctagcctgtccaagctgctgggcaagaagaaggagctgcaagacctggagctggccctggagggcgcactggataagggccatgaggtgaccttggaggccctgccaaaggacgtcctgctgagcaaggaagccgtgggcgccatcctgtacttcgtgggcgctctgaccgagctgagcgaggcccagcagaagctgctggtgaagtccatggaaaagaaaatcctgcctgtgcagctgaagctggtggagagcaccatggaacagaactttctgctggacaaggagggcgtgttccctctgcagcctgagctcctgagctctctgggagatgaggaactgaccctgacagaggccctggtcggactgtccggcctggaagtgcagcggagcggccctcagtacatgtgggatcctgatacactccctagactgtgcgccctgtacgccggcctcagcctgctgcagcagctgaccaaggcttcttaa

SEQ ID NO: 233 - DNA, 1272 bp, synthetic construct
  source 1..1272: mol_type = other DNA
atgttcagcgtgttcgaggagatcacccggatcgtggtcaaagaaatggacgctggcggagacatgatcgccgtgcggagcctggttgacgccgacagattcagatgttttcacctggtcggagagaaaagaaccttcttcggctgcagacactacacaaccggcctgacactgatggatatcctggacaccgacggcgataagtggctggatgagctggattccggcctgcaaggccagaaagccgagttccagatcctggacaacgtggacagcaccggcgagctgatcgtgcggctgcccaaggaaatcaccatcagcggctccttccaaggcttccaccaccagaagatcaagatttcagagaatcggatcagccaacagtacctggccaccctggaaaacagaaagctgaagagggaactgcctttcagctttagaagcatcaacacaagagagaacctgtacctggtgacagagacactggagaccgtgaaggaagagacactgaagagtgatagacagtataagttctggagccagatcagccagggccacctgtcctacaagcacaagggccagcgcgaagtgaccattcctccaaatagagtgctgagctacagagtgaagcagctggttttcccaaacaaggaaaccatgaacatccacttccggggcaagaccaagagctttcctgaggaaaaggacggcgccagcagctgcctgggaaaggaaaacctgtacttccagtctagcctgggtagcgaggattctcgcaatatgaaagagaagctggaggacatggaatctgtgctgaaggacctgaccgaggaaaagcggaaggatgtgctcaacagcctggccaagtgcctgggcaaagaggacatcagacaggacctggagcagcgggtgtccgaggtgctgatctctggcgagctgcatatggaggatcctgataagcctctgctgagctctctgtttaacgccgccggcgtgctggtcgaggccagagccaaggccatcctggacttcctggacgctcttctggagctgtctgaagaacagcagttcgtggccgaggcactggaaaagggaacactccctctgctgaaagaccaggtgaagagcgtgatggaacagaactgggatgagctggcttctagcccccccgacatggactacgaccccgaggccagaatcctgtgcgccctgtacgtggtggtgtccatcctgctggaactggctgaaggccctacctctgtgagcagctaa

SEQ ID NO: 234 - DNA, 1548 bp, synthetic construct
  source 1..1548: mol_type = other DNA
atgcctagcatgctggagcggatcagcaagaacctggtgaaggaaatcggaagcaaggacctgacccctgtgaaatacctgctcagcgctacaaagctgcggcagttcgtgatcctgcggaagaagaaagatagcagaagcagtttttgggagcagtctgactacgtgcccgtggaattctccctgaacgacatcctggagccttcctctagcgtgctggaaaccgttgtgaccggcccattccacttcagcgacatcatgatccagaaacacaaggccgacatgggcgttaatgtgggcatcgaggtgagcgtgagcggcgaggcctctgtcgaccacggctgctctctggagttccagatcgtgaccatcccctctccaaacctggaagatttccagaagagaaagctgctggatcccgagcctagcttcctgaaagagtgcagacggcggggcgacaacctgtacgtggtgacagaagccgtggaacttatcaacaacaccgtgctgtacgacagcagcagcgtgaacatcctgggcaagatcgccctgtggatcacatacggcaagggccagggccaaggagaatctctgagagtgaagaagaaagccctgaccctgcagaaaggcatggtgatggcctacaagagaaagcagctggtgatcaaggagaaggctatcctgatctctgatgacgatgagcagagaacattccaggatgaaaacctgtacttccagtctgagtacgagattagcgagatggtgggctactgcgccgctagaagcgagggcctactgcccagcttccacaccatcagccccaccctgtttaatgccagcagcaacgatatgaaactgaagcctgaactgttcctgacacagcagtttctatccggacatctgcctaagtacgagcaagtgcacatcctgcctgtgggcagaattgaggaacctttttggcagaacttcaagcacctgcaggaggaggtgttccaaaagatcaagacccttgctcagctgtctaaggacgtccaggatgtgatgttctacagcatcctggccatgctgcgcgacagaggcgccctgcaggacctgatgaacatgctggaactggacagcagcggccacctggacggccctggaggcgctatcctgaagaaactgcaacaggacagcaaccacgcctggttcaaccctaaggatcctatcctctacctgctggaagccatcatggtcctgtcagacttccagcacgacctgctggcatgcagcatggaaaagaggattctgctgcagcaacaggagctcgttagaagcatcctggagcctaatttcagatacccctggtccatccctttcaccctgaagcccgagctgctcgcccctctgcagagcgagggcctggccattacatatggcctgctggaagagtgtggcctgagaatggaactggataaccccagatccacctgggatgtggaagccaagatgcctctgtccgccctgtatggcaccctgtctctgctgcagcagctggccgaggcctaa

SEQ ID NO: 235 - DNA, 1476 bp, synthetic construct
  source 1..1476: mol_type = other DNA
atgggctctgccttcgagagagtggtcagaagggtggtgcaggagctcgaccacggcggcgaattcatccccgtgacaagcctgcaaagcagcacaggcttccagccctactgcctggtggttagaaagccctcttcttcttggttctggaagcctagatacaagtgcgtgaacctgagcatcaaggacatcctggaacctgatgccgctgaacctgatgtgcagagaggccggagcttccacttctacgacgccatggacggccagatccagggcagcgtggaactcgccgccccaggccaagccaagatcgccggcggagctgctgtgtccgatagcagctctaccagcatgaacgtgtattctctgtccgtggaccccaacacctggcagaccctgctgcacgagagacacctgcggcagccagagcacaaggtgctgcagcagctgagatctagaggcgacaacgtgtacgtggtgaccgaggtgctccagacccagaaagaggtggaagtcacaagaacccacaagcgggaaggctccggaagattcagcctgcccggcgccacatgtctgcagggcgagggccagggccacctgagccagaagaaaaccgtgaccattcctagcggcagcaccctggcctttcgggtggcccagctggtcatcgacagcgacctggacgtgctgctgttccccgataagaagcagcggacctttcagcctcctgccaccggccacaaaagatccacctccgagggagcctggcctcagctgcctagcggcctgagcatgatgcggtgcctgcacaacttcctgactgatgaaaacctgtacttccagtctggagtgcccgccgagggcgcttttaccgaggacttccagggcctgcgagccgaagtggagacaatcagcaaggaactggaactgctggatagagagctgtgccaactgctgctggaaggcctggaaggcgtgctgcgcgaccagctggctctgagagccctggaagaggccctggaacaaggccagagcctgggccctgttgagcccctggacggccctgccggcgccgtgctggaatgtctggtgctgagcagcggcatgctggtgcctgagctggctatccccgtggtgtacctgctaggcgccctgaccatgctgtctgagacacagcataagctgctcgcagaagccctggagagccagacactgctggggcctctggagctggtgggcagcctgctggagcagagcgccccttggcaggagcggtctacaatgagtctgcctccaggtctccttggaaatagctggggcgagggcgctcctgcttgggtgctgctggatgagtgcggcctggaactgggcgaggacacccctcacgtgtgttgggagccacaggcccagggcagaatgtgcgccctgtacgccagcctggccttgctgagcggcctgagccaggagcctcattaa

SEQ ID NO: 236 - DNA, 1512 bp, synthetic construct
  source 1..1512: mol_type = other DNA
atgttcgccaaggccacaagaaatttcttgcgggaagtggacgctgatggcgacctgatcgccgtgtctaatctgaacgacagcgataagctgcaactgctgagcctggtcaccaaaaagaagcggttctggtgctggcagcggcctaagtaccagtttctgagcctgacactgggagatgtgctgattgaggaccaattccccagccccgtggtggtcgagagcgactttgtgaaatacgagggcaaattcgccaaccacgtgtccggcaccctggagacagccctgggcaaggtgaagctgaacctgggcggctctagcagagtggaatctcagagcagcttcggcaccctgcgcaagcaggaggtcgacctgcagcagctgatcagagattctgctgaacggaccatcaacctgagaaaccctgtgctgcaacaggtgctggaaggcagaaacgaggtgctgtgcgtgctgacccagaagatcacaacaatgcagaaatgtgtgatcagcgagcatatgcaggtggaggaaaagtgcggcggaatcgtgggcatccagaccaagaccgtgcaggttagcgccactgaggatggcaacgtgaccaaggacagcaatgtggtgctggaaatccctgctgctacaaccatcgcctacggcgtgatcgagctgtacgtgaagctggatggccagttcgagttctgcctgctgagaggcaagcagggaggcttcgagaacaagaaaagaatcgactccgtgtacctggaccccctggtctttagagaattcgccttcatcgatatgcctgacgaaaacctgtacttccagtctgccgcccacggcatctccagccaagacggtcctctgagcgttctgaagcaagccacactgctgttggagagaaacttccaccctttcgccgagctgcctgagccacagcagaccgccctgagcgatatcttccaggccgtcctgttcgacgacgagctgctgatggtgctcgaacctgtgtgcgacgacctggtgagcggactgagccctacagtggctgtgctaggagagctgaagcccagacagcagcaggacctggtggcctttctccagctggttggctgtagcctgcagggcggctgtcctggccccgaggacgccggcagcaagcaactgttcatgaccgcctatttcctggtgagcgccctggccgaaatgcccgacagcgctgccgctcttctgggcacctgctgcaaactgcagatcatcccaaccctgtgtcacctgctgcgggccctgtctgatgatggagtgtccgacctggaagatcctaccctgacgcctctgaaagacaccgagaggttcggcattgtgcagagactgtttgcatctgccgacatctctctggaacggctgaagagctccgtgaaggccgtgatcctgaaggacagcaaggtgttcccactgcttctctgcatcaccctgaacggcctgtgcgccctgggcagagagcacagttaa

SEQ ID NO: 237 - protein, 452 aa, synthetic construct
MTMFENVTRALARQLNPRGDLTPLDSLIDFKRFHPFCLVLRKRKSTLFWGARYVRTDYTLLDVLEPGSSPSDPTDTGNFGFKNMLDTRVEGDVDVPKTVKVKGTAGLSQNSTLEVQTLSVAPKALETVQERKLAADHPFLKEMQDQGENLYVVMEVVETVQEVTLERAGKAEACFSLPFFAPLGLQGSINHKEAVTIPKGCVLAFRVRQLMVKGKDEWDIPHICNDNMQTFPPGEKSGEEKVILIQENLYFQSASDVGDVHEGFRTLKEEVQRETQQVEKLSRVGQSSLLSSLSKLLGKKKELQDLELALEGALDKGHEVTLEALPKDVLLSKEAVGAILYFVGALTELSEAQQKLLVKSMEKKILPVQLKLVESTMEQNFLLDKEGVFPLQPELLSSLGDEELTLTEALVGLSGLEVQRSGPQYMWDPDTLPRLCALYAGLSLLQQLTKAS

SEQ ID NO: 238 - protein, 423 aa, synthetic construct
MFSVFEEITRIVVKEMDAGGDMIAVRSLVDADRFRCFHLVGEKRTFFGCRHYTTGLTLMDILDTDGDKWLDELDSGLQGQKAEFQILDNVDSTGELIVRLPKEITISGSFQGFHHQKIKISENRISQQYLATLENRKLKRELPFSFRSINTRENLYLVTETLETVKEETLKSDRQYKFWSQISQGHLSYKHKGQREVTIPPNRVLSYRVKQLVFPNKETMNIHFRGKTKSFPEEKDGASSCLGKENLYFQSSLGSEDSRNMKEKLEDMESVLKDLTEEKRKDVLNSLAKCLGKEDIRQDLEQRVSEVLISGELHMEDPDKPLLSSLFNAAGVLVEARAKAILDFLDALLELSEEQQFVAEALEKGTLPLLKDQVKSVMEQNWDELASSPPDMDYDPEARILCALYVVVSILLELAEGPTSVSS

SEQ ID NO: 239 - protein, 515 aa, synthetic construct
MPSMLERISKNLVKEIGSKDLTPVKYLLSATKLRQFVILRKKKDSRSSFWEQSDYVPVEFSLNDILEPSSSVLETVVTGPFHFSDIMIQKHKADMGVNVGIEVSVSGEASVDHGCSLEFQIVTIPSPNLEDFQKRKLLDPEPSFLKECRRRGDNLYVVTEAVELINNTVLYDSSSVNILGKIALWITYGKGQGQGESLRVKKKALTLQKGMVMAYKRKQLVIKEKAILISDDDEQRTFQDENLYFQSEYEISEMVGYCAARSEGLLPSFHTISPTLFNASSNDMKLKPELFLTQQFLSGHLPKYEQVHILPVGRIEEPFWQNFKHLQEEVFQKIKTLAQLSKDVQDVMFYSILAMLRDRGALQDLMNMLELDSSGHLDGPGGAILKKLQQDSNHAWFNPKDPILYLLEAIMVLSDFQHDLLACSMEKRILLQQQELVRSILEPNFRYPWSIPFTLKPELLAPLQSEGLAITYGLLEECGLRMELDNPRSTWDVEAKMPLSALYGTLSLLQQLAEA

SEQ ID NO: 240 - protein, 491 aa, synthetic construct
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDENLYFQSGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 241 - protein, 503 aa, synthetic construct
MFAKATRNFLREVDADGDLIAVSNLNDSDKLQLLSLVTKKKRFWCWQRPKYQFLSLTLGDVLIEDQFPSPVVVESDFVKYEGKFANHVSGTLETALGKVKLNLGGSSRVESQSSFGTLRKQEVDLQQLIRDSAERTINLRNPVLQQVLEGRNEVLCVLTQKITTMQKCVISEHMQVEEKCGGIVGIQTKTVQVSATEDGNVTKDSNVVLEIPAATTIAYGVIELYVKLDGQFEFCLLRGKQGGFENKKRIDSVYLDPLVFREFAFIDMPDENLYFQSAAHGISSQDGPLSVLKQATLLLERNFHPFAELPEPQQTALSDIFQAVLFDDELLMVLEPVCDDLVSGLSPTVAVLGELKPRQQQDLVAFLQLVGCSLQGGCPGPEDAGSKQLFMTAYFLVSALAEMPDSAAALLGTCCKLQIIPTLCHLLRALSDDGVSDLEDPTLTPLKDTERFGIVQRLFASADISLERLKSSVKAVILKDSKVFPLLLCITLNGLCALGREHS

SEQ ID NO: 242 - DNA, 3081 bp, synthetic construct
  source 1..3081: mol_type = other DNA
atgtcaaaagcactcctgaagggagtccgcgactttaatcctatatcagcgtgtgtatgtttgcttgaaaactcctctgatgggcattcagaaaggctttttggtattgggtttggtccctacataatagcaaaccaacacctgtttcgacgcaataacggcgagcttacgattaagacgatgcacggggaatttaaagtgaaaaacagcacacaattgcaaatgaaacccgtcgaagggagagacatcattgtaataaagatggcaaaggatttcccaccctttccgcagaaattgaaattccggcaacctactataaaggatcgagtttgtatggtttctacgaactttcagcaaggatccgagggcagcggcagcgagggctccggcagccctgaggacgagatccagcaactggaagaggaaatcgcccagctggagcagaaaaacgccgctctgaaggaaaagaaccaggccctgaagtacggcggcggatctggcggcggcgagaacttgtacttccagagtggatctagcggctccggctctcctgaggacgagctgcaaagcaacgaggaagaactgcagcagaacgagcagaagctgcagcagatcaagcagaaactgcagagcatcaagtacggcggctcggaaaacctttacttccaatcaggatctaaatcagtctcctccctcgtgtccgaatcctctcacatcgttcacaaggaagataccagtttctggcagcattggattaccacaaaagacgggcaggcgggctcaccgctggtgtctatcattgacgggaacattctgggaatccactccctgacacacaccactaacggctcaaactatttcgttgagttcccagagaagttcgtagctacatatctggacgctgcggacggctggtgcaagaactggaaattcaacgcggacaagatttcatggggtagtttcacgcttgtcgaagacgcgcccgaggacgactttatggcgaaaaaaaccgtggctgctattatggatggcagcggcgaaggtcgggggagtttgcttacctgtggcgatgtcgaggagaatccagggcccggatccagtaaagcccttctcaaaggtgtgcgagatttcaatcccatatccgcctgtgtctgcctcctggaaaatagttcagacgggcacagcgaaaggctctttggaatcggcttcgggccgtatattattgctaatcagaggttgttccggcgcaacaacggagagttgacaataaagaccatgcacggtgaatttaaagtgaagaatagtacccaactccagatgaaaccagtggaggggaggaacattattgtcataaaaatggctaaagatttccctccttttccacaaaaattgaaattcaggcaacctacaatcaaagatcgcgtctgcatggtttctacgaattttcagcaaggcagcgaaaacctttacttccaatcaggctcgtctcctgaggacaagaaccagcagctgaaagagaagaaccagagcctgaagcaggagatccaaagcctggaagaggaaatccagcagctggaatacggcgggtcttctggatctggcgagaacttgtacttccagagtggcggatctggcggcggcagccctgaggacaagatcgcccagctgaagcaaaagatccaggccctgaaacaggagaaccagcagctggaagaggaaaacgccgctctggagtactccggcagcgagggcagcggctccgaaggcagcaaatcagtctcctccctcgtgtccgaatcctctcacatcgttcacaaggaagataccagtttctggcagcattggattaccacaaaagacgggcagtgcggctcaccgctggtgtctatcattgacgggaacattctgggaatccactccctgacacacaccactaacggctcaaactatttcgttgagttcccagagaagttcgtagctacatatctggacgctgcggacggctggtgcaagaactggaaattcaacgcggacaagatttcatggggtagtttcacgcttgtcgaagacgcgcccgaggacgactttatggcgaaaaaaaccgtggctgctattatggatagcggcagcggcgccacaaacttctctctgctaaagcaagcaggtgatgttgaagaaaaccccgggcctggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtcgacgctggagatttcctag

SEQ ID NO: 243 - protein, 1026 aa, synthetic construct
MSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQGSEGSGSEGSGSPEDEIQQLEEEIAQLEQKNAALKEKNQALKYGGGSGGGENLYFQSGSSGSGSPEDELQSNEEELQQNEQKLQQIKQKLQSIKYGGSENLYFQSGSKSVSSLVSESSHIVHKEDTSFWQHWITTKDGQAGSPLVSIIDGNILGIHSLTHTTNGSNYFVEFPEKFVATYLDAADGWCKNWKFNADKISWGSFTLVEDAPEDDFMAKKTVAAIMDGSGEGRGSLLTCGDVEENPGPGSSKALLKGVRDFNPISACVCLLENSSDGHSERLFGIGFGPYIIANQRLFRRNNGELTIKTMHGEFKVKNSTQLQMKPVEGRNIIVIKMAKDFPPFPQKLKFRQPTIKDRVCMVSTNFQQGSENLYFQSGSSPEDKNQQLKEKNQSLKQEIQSLEEEIQQLEYGGSSGSGENLYFQSGGSGGGSPEDKIAQLKQKIQALKQENQQLEEENAALEYSGSEGSGSEGSKSVSSLVSESSHIVHKEDTSFWQHWITTKDGQCGSPLVSIIDGNILGIHSLTHTTNGSNYFVEFPEKFVATYLDAADGWCKNWKFNADKISWGSFTLVEDAPEDDFMAKKTVAAIMDSGSGATNFSLLKQAGDVEENPGPGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEIS

SEQ ID NO: 244 - protein, 363 aa, synthetic construct
MSKALLKGVRDFNPISACVCLLENSSDGHSERLFGIGFGPYIIANQHLFRRNNGELTIKTMHGEFKVKNSTQLQMKPVEGRDIIVIKMAKDFPPFPQKLKFRQPTIKDRVCMVSTNFQQGSEGSGSEGSGSPEDEIQQLEEEIAQLEQKNAALKEKNQALKYGGGSGGGENLYFQSGSSGSGSPEDELQSNEEELQQNEQKLQQIKQKLQSIKYGGSENLYFQSGSKSVSSLVSESSHIVHKEDTSFWQHWITTKDGQAGSPLVSIIDGNILGIHSLTHTTNGSNYFVEFPEKFVATYLDAADGWCKNWKFNADKISWGSFTLVEDAPEDDFMAKKTVAAIMDGSGEGRGSLLTCGDVEENPG

SEQ ID NO: 245 - protein, 366 aa, synthetic construct
PGSSKALLKGVRDFNPISACVCLLENSSDGHSERLFGIGFGPYIIANQRLFRRNNGELTIKTMHGEFKVKNSTQLQMKPVEGRNIIVIKMAKDFPPFPQKLKFRQPTIKDRVCMVSTNFQQGSENLYFQSGSSPEDKNQQLKEKNQSLKQEIQSLEEEIQQLEYGGSSGSGENLYFQSGGSGGGSPEDKIAQLKQKIQALKQENQQLEEENAALEYSGSEGSGSEGSKSVSSLVSESSHIVHKEDTSFWQHWITTKDGQCGSPLVSIIDGNILGIHSLTHTTNGSNYFVEFPEKFVATYLDAADGWCKNWKFNADKISWGSFTLVEDAPEDDFMAKKTVAAIMDSGSGATNFSLLKQAGDVEENPG

SEQ ID NO: 246 - protein, 297 aa, synthetic construct
PGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEIS

SEQ ID NO: 247 - protein, 7 aa, synthetic construct
ENLYFQS

SEQ ID NO: 248 - DNA, 21 bp, synthetic construct
  source 1..21: mol_type = other DNA
gaaaacctttatttccagtct

SEQ ID NO: 249 - DNA, 21 bp, synthetic construct
  source 1..21: mol_type = other DNA
gagaatctctactttcagagt

SEQ ID NO: 250 - DNA, 21 bp, synthetic construct
  source 1..21: mol_type = other DNA
gagaacttgtacttccagtcc

SEQ ID NO: 251 - DNA, 21 bp, synthetic construct
  source 1..21: mol_type = other DNA
gagaacttgtacttccagagt

SEQ ID NO: 252 - DNA, 21 bp, synthetic construct
  source 1..21: mol_type = other DNA
gaaaacctttacttccaatca

SEQ ID NO: 253 - protein, 7 aa, synthetic construct
ETVRFQS

SEQ ID NO: 254 - DNA, 21 bp, synthetic construct
  source 1..21: mol_type = other DNA
gaaaccgtgcggtttcaatcc

SEQ ID NO: 255 - protein, 11 aa, synthetic construct
DLEVVTSTWVL

SEQ ID NO: 256 - protein, 10 aa, synthetic construct
DEMEECASHL

SEQ ID NO: 257 - protein, 10 aa, synthetic construct
DCSTPCSGSW

SEQ ID NO: 258 - protein, 12 aa, synthetic construct
EDVVCCSMSYTW

SEQ ID NO: 259 - DNA, 1641 bp, synthetic construct
  source 1..1641: mol_type = other DNA
atggggtccaaaggagaagaactgtttaccggtgttgtgccaattttggttgaactcgatggtgatgtcaacggacataagttctcagtgagaggcgaaggagaaggtgacgccaccattggaaaattgactcttaaattcatctgtactactggtaaacttcctgtaccatggccgactctcgtaacaacgcttacgtacggagttcagtgcttttcgagatacccagaccatatgaaaagacatgacttttttaagtcggctatgcctgaaggttacgtgcaagaaagaacaatttcgttcaaagatgatggaaaatataaaactagagcagttgttaaatttgaaggagatactttggttaaccgcattgaactgaaaggaacagattttaaagaagatggtaatattcttggacacaaactcgaatacaattttaatagtcataacgtatacatcactgctgataagcaaaagaacggaattaaagcgaatttcacagtacgccataatgtagaagatggcagtgttcaacttgccgaccattaccaacaaaacacccctattggagacggtccggtacttcttcctgataatcactacctctcaacacaaacagtcctgagcaaagatccaaatgaaaaaggaggcagcggatccaccactaacattgcgccggctctggacggtgcgtttcatcgtatcacttcttcatcctccctgatgggcctgtgtgacgaaaccgcgactattatccgtgagattactggctacgaccgtgtgatggtagtacgtttcgatgaagagggtaatgccgaaattctgtccgaacgtcgtcgtgcggacctggaagcgttcctgggtaaccgctacccggcgtctactattccgcagatcgctcgtcgcctgtacgaacataaccgtgttcgcctgctggtagatgtgaactatactccggttccgctacagccgcgcatcagcccgctgaacggtcgtgatctggatatgtccctgtcttgcctgcgctctatgtccccggtccaccagaaatacatgcaggacatgggcgttggcgcgaccctggtttgctctctgatggtgtctggtcgtttgtggggtctgatcgcttgccaccactacgaaccgcgcttcgttccgttccacattcgcgctgctggcgaagcgctggcggaaacttgtgcgaaccgcatcgcgacgctggagagcgaaaatctctacttccagagtgaattctgtgaacgcgagcagatccacctggcgggctccattcagccgcacggtatcctgctggctgtgaaagagccagacaacgtggtgatccaggcttctatcaacgctgcggagttcctgaacaccaactctgttgttggccgtccgctgcgtgacctgggcggcgatctgcctttgcagatcctgccgcacctgaacggcccgctgcacctggctccggtgaccctgcgttgtaccgtgggttctccgccgcgccgtgtggactgtaccattcaccgtccgtctaacggcggcctgatcgtagaactggaaccagcaaccaagctcgagggaggcagccgggaccacatggtgctgcacgagtacgtgaacgccgccggcatcacatag

SEQ ID NO: 260 - DNA, 1743 bp, synthetic construct
  source 1..1743: mol_type = other DNA
atggggtccaaaggagaagaactgtttaccggtgttgtgccaattttggttgaactcgatggtgatgtcaacggacataagttctcagtgagaggcgaaggagaaggtgacgccaccattggaaaattgactcttaaattcatctgtactactggtaaacttcctgtaccatggccgactctcgtaacaacgcttacgtacggagttcagtgcttttcgagatacccagaccatatgaaaagacatgacttttttaagtcggctatgcctgaaggttacgtgcaagaaagaacaatttcgttcaaagatgatggaaaatataaaactagagcagttgttaaatttgaaggagatactttggttaaccgcattgaactgaaaggaacagattttaaagaagatggtaatattcttggacacaaactcgaatacaattttaatagtcataacgtatacatcactgctgataagcaaaagaacggaattaaagcgaatttcacagtacgccataatgtagaagatggcagtgttcaacttgccgaccattaccaacaaaacacccctattggagacggtccggtacttcttcctgataatcactacctctcaacacaaacagtcctgagcaaagatccaaatgaaaaaggaggcagcggatccaccactaacattgcgccggctctggacggtgcgtttcatcgtatcacttcttcatcctccctgatgggcctgtgtgacgaaaccgcgactattatccgtgagattactggctacgaccgtgtgatggtagtacgtttcgatgaagagggtaatgccgaaattctgtccgaacgtcgtcgtgcggacctggaagcgttcctgggtaaccgctacccggcgtctactattccgcagatcgctcgtcgcctgtacgaacataaccgtgttcgcctgctggtagatgtgaactatactccggttccgctacagccgcgcatcagcccgctgaacggtcgtgatctggatatgtccctgtcttgcctgcgctctatgtccccggtccaccagaaatacatgcaggacatgggcgttggcgcgaccctggtttgctctctgatggtgtctggtcgtttgtggggtctgatcgcttgccaccactacgaaccgcgcttcgttccgttccacattcgcgctgctggcgaagcgctggcggaaacttgtgcgaaccgcatcgcgacgctggagagcgaaaatctctacttccagagtgaattctgtgaacgcgagcagatccacctggcgggctccattcagccgcacggtatcctgctggctgtgaaagagccagacaacgtggtgatccaggcttctatcaacgctgcggagttcctgaacaccaactctgttgttggccgtccgctgcgtgacctgggcggcgatctgcctttgcagatcctgccgcacctgaacggcccgctgcacctggctccggtgaccctgcgttgtaccgtgggttctccgccgcgccgtgtggactgtaccattcaccgtccgtctaacggcggcctgatcgtagaactggaaccagcaaccaagctcgagggaggcagccgggaccacatggtgctgcacgagtacgtgaacgccgccggcatcacatctggcctgagaagcagagcccaggccagcatggaccccaagaaaaaacggaaggtggatcctaagaagaaaagaaaggtggaccctaagaagaagcggaagtag

SEQ ID NO: 261 - DNA, 1722 bp, synthetic construct
  source 1..1722: mol_type = other DNA
atggggtccaaaggagaagaactgtttaccggtgttgtgccaattttggttgaactcgatggtgatgtcaacggacataagttctcagtgagaggcgaaggagaaggtgacgccaccattggaaaattgactcttaaattcatctgtactactggtaaacttcctgtaccatggccgactctcgtaacaacgcttacgtacggagttcagtgcttttcgagatacccagaccatatgaaaagacatgacttttttaagtcggctatgcctgaaggttacgtgcaagaaagaacaatttcgttcaaagatgatggaaaatataaaactagagcagttgttaaatttgaaggagatactttggttaaccgcattgaactgaaaggaacagattttaaagaagatggtaatattcttggacacaaactcgaatacaattttaatagtcataacgtatacatcactgctgataagcaaaagaacggaattaaagcgaatttcacagtacgccataatgtagaagatggcagtgttcaacttgccgaccattaccaacaaaacacccctattggagacggtccggtacttcttcctgataatcactacctctcaacacaaacagtcctgagcaaagatccaaatgaaaaaggaggcagcggatccaccactaacattgcgccggctctggacggtgcgtttcatcgtatcacttcttcatcctccctgatgggcctgtgtgacgaaaccgcgactattatccgtgagattactggctacgaccgtgtgatggtagtacgtttcgatgaagagggtaatgccgaaattctgtccgaacgtcgtcgtgcggacctggaagcgttcctgggtaaccgctacccggcgtctactattccgcagatcgctcgtcgcctgtacgaacataaccgtgttcgcctgctggtagatgtgaactatactccggttccgctacagccgcgcatcagcccgctgaacggtcgtgatctggatatgtccctgtcttgcctgcgctctatgtccccggtccaccagaaatacatgcaggacatgggcgttggcgcgaccctggtttgctctctgatggtgtctggtcgtttgtggggtctgatcgcttgccaccactacgaaccgcgcttcgttccgttccacattcgcgctgctggcgaagcgctggcggaaacttgtgcgaaccgcatcgcgacgctggagagcgaaaatctctacttccagagtgaattctgtgaacgcgagcagatccacctggcgggctccattcagccgcacggtatcctgctggctgtgaaagagccagacaacgtggtgatccaggcttctatcaacgctgcggagttcctgaacaccaactctgttgttggccgtccgctgcgtgacctgggcggcgatctgcctttgcagatcctgccgcacctgaacggcccgctgcacctggctccggtgaccctgcgttgtaccgtgggttctccgccgcgccgtgtggactgtaccattcaccgtccgtctaacggcggcctgatcgtagaactggaaccagcaaccaagctcgagggaggcagccgggaccacatggtgctgcacgagtacgtgaacgccgccggcatcacaagcggcggtggaggacggagcaaactgaatccacccgatgagtctggacccggttgtatgtcatgtaaatgtgtgttgagctag

SEQ ID NO: 262 - DNA, 1641 bp, synthetic construct
  source 1..1641: mol_type = other DNA
atggggtccaaaggagaagaactgtttaccggtgttgtgccaattttggttgaactcgatggtgatgtcaacggacataagttctcagtgagaggcgaaggagaaggtgacgccaccattggaaaattgactcttaaattcatctgtactactggtaaacttcctgtaccatggccgactctcgtaacaacgcttacgtacggagttcagtgcttttcgagatacccagaccatatgaaaagacatgacttttttaagtcggctatgcctgaaggttacgtgcaagaaagaacaatttcgttcaaagatgatggaaaatataaaactagagcagttgttaaatttgaaggagatactttggttaaccgcattgaactgaaaggaacagattttaaagaagatggtaatattcttggacacaaactcgaatacaattttaatagtcataacgtatacatcactgctgataagcaaaagaacggaattaaagcgaatttcacagtacgccataatgtagaagatggcagtgttcaacttgccgaccattaccaacaaaacacccctattggagacggtccggtacttcttcctgataatcactacctctcaacacaaacagtcctgagcaaagatccaaatgaaaaaggaggcagcggatccaccactaacattgcgccggctctggacggtgcgtttcatcgtatcacttcttcatcctccctgatgggcctgtgtgacgaaaccgcgactattatccgtgagattactggctacgaccgtgtgatggtagtacgtttcgatgaagagggtaatgccgaaattctgtccgaacgtcgtcgtgcggacctggaagcgttcctgggtaaccgctacccggcgtctactattccgcagatcgctcgtcgcctgtacgaacataaccgtgttcgcctgctggtagatgtgaactatactccggttccgctacagccgcgcatcagcccgctgaacggtcgtgatctggatatgtccctgtcttgcctgcgctctatgtccccggtccaccagaaatacatgcaggacatgggcgttggcgcgaccctggtttgctctctgatggtgtctggtcgtttgtggggtctgatcgcttgccaccactacgaaccgcgcttcgttccgttccacattcgcgctgctggcgaagcgctggcggaaacttgtgcgaaccgcatcgcgacgctggagagcgaaactgtcagattccaatctgaattctgtgaacgcgagcagatccacctggcgggctccattcagccgcacggtatcctgctggctgtgaaagagccagacaacgtggtgatccaggcttctatcaacgctgcggagttcctgaacaccaactctgttgttggccgtccgctgcgtgacctgggcggcgatctgcctttgcagatcctgccgcacctgaacggcccgctgcacctggctccggtgaccctgcgttgtaccgtgggttctccgccgcgccgtgtggactgtaccattcaccgtccgtctaacggcggcctgatcgtagaactggaaccagcaaccaagctcgagggaggcagccgggaccacatggtgctgcacgagtacgtgaacgccgccggcatcacatag

SEQ ID NO: 263 - DNA, 1743 bp, synthetic construct
  source 1..1743: mol_type = other DNA
atggggtccaaaggagaagaactgtttaccggtgttgtgccaattttggttgaactcgatggtgatgtcaacggacataagttctcagtgagaggcgaaggagaaggtgacgccaccattggaaaattgactcttaaattcatctgtactactggtaaacttcctgtaccatggccgactctcgtaacaacgcttacgtacggagttcagtgcttttcgagatacccagaccatatgaaaagacatgacttttttaagtcggctatgcctgaaggttacgtgcaagaaagaacaatttcgttcaaagatgatggaaaatataaaactagagcagttgttaaatttgaaggagatactttggttaaccgcattgaactgaaaggaacagattttaaagaagatggtaatattcttggacacaaactcgaatacaattttaatagtcataacgtatacatcactgctgataagcaaaagaacggaattaaagcgaatttcacagtacgccataatgtagaagatggcagtgttcaacttgccgaccattaccaacaaaacacccctattggagacggtccggtacttcttcctgataatcactacctctcaacacaaacagtcctgagcaaagatccaaatgaaaaaggaggcagcggatccaccactaacattgcgccggctctggacggtgcgtttcatcgtatcacttcttcatcctccctgatgggcctgtgtgacgaaaccgcgactattatccgtgagattactggctacgaccgtgtgatggtagtacgtttcgatgaagagggtaatgccgaaattctgtccgaacgtcgtcgtgcggacctggaagcgttcctgggtaaccgctacccggcgtctactattccgcagatcgctcgtcgcctgtacgaacataaccgtgttcgcctgctggtagatgtgaactatactccggttccgctacagccgcgcatcagcccgctgaacggtcgtgatctggatatgtccctgtcttgcctgcgctctatgtccccggtccaccagaaatacatgcaggacatgggcgttggcgcgaccctggtttgctctctgatggtgtctggtcgtttgtggggtctgatcgcttgccaccactacgaaccgcgcttcgttccgttccacattcgcgctgctggcgaagcgctggcggaaacttgtgcgaaccgcatcgcgacgctggagagcgaaactgtcagattccaatctgaattctgtgaacgcgagcagatccacctggcgggctccattcagccgcacggtatcctgctggctgtgaaagagccagacaacgtggtgatccaggcttctatcaacgctgcggagttcctgaacaccaactctgttgttggccgtccgctgcgtgacctgggcggcgatctgcctttgcagatcctgccgcacctgaacggcccgctgcacctggctccggtgaccctgcgttgtaccgtgggttctccgccgcgccgtgtggactgtaccattcaccgtccgtctaacggcggcctgatcgtagaactggaaccagcaaccaagctcgagggaggcagccgggaccacatggtgctgcacgagtacgtgaacgccgccggcatcacatctggcctgagaagcagagcccaggccagcatggaccccaagaaaaaacggaaggtggatcctaagaagaaaagaaaggtggaccctaagaagaagcggaagtag

SEQ ID NO: 264 - DNA, 1722 bp, synthetic construct
  source 1..1722: mol_type = other DNA
atggggtccaaaggagaagaactgtttaccggtgttgtgccaattttggttgaactcgatggtgatgtcaacggacataagttctcagtgagaggcgaaggagaaggtgacgccaccattggaaaattgactcttaaattcatctgtactactggtaaacttcctgtaccatggccgactctcgtaacaacgcttacgtacggagttcagtgcttttcgagatacccagaccatatgaaaagacatgacttttttaagtcggctatgcctgaaggttacgtgcaagaaagaacaatttcgttcaaagatgatggaaaatataaaactagagcagttgttaaatttgaaggagatactttggttaaccgcattgaactgaaaggaacagattttaaagaagatggtaatattcttggacacaaactcgaatacaattttaatagtcataacgtatacatcactgctgataagcaaaagaacggaattaaagcgaatttcacagtacgccataatgtagaagatggcagtgttcaacttgccgaccattaccaacaaaacacccctattggagacggtccggtacttcttcctgataatcactacctctcaacacaaacagtcctgagcaaagatccaaatgaaaaaggaggcagcggatccaccactaacattgcgccggctctggacggtgcgtttcatcgtatcacttcttcatcctccctgatgggcctgtgtgacgaaaccgcgactattatccgtgagattactggctacgaccgtgtgatggtagtacgtttcgatgaagagggtaatgccgaaattctgtccgaacgtcgtcgtgcggacctggaagcgttcctgggtaaccgctacccggcgtctactattccgcagatcgctcgtcgcctgtacgaacataaccgtgttcgcctgctggtagatgtgaactatactccggttccgctacagccgcgcatcagcccgctgaacggtcgtgatctggatatgtccctgtcttgcctgcgctctatgtccccggtccaccagaaatacatgcaggacatgggcgttggcgcgaccctggtttgctctctgatggtgtctggtcgtttgtggggtctgatcgcttgccaccactacgaaccgcgcttcgttccgttccacattcgcgctgctggcgaagcgctggcggaaacttgtgcgaaccgcatcgcgacgctggagagcgaaactgtcagattccaatctgaattctgtgaacgcgagcagatccacctggcgggctccattcagccgcacggtatcctgctggctgtgaaagagccagacaacgtggtgatccaggcttctatcaacgctgcggagttcctgaacaccaactctgttgttggccgtccgctgcgtgacctgggcggcgatctgcctttgcagatcctgccgcacctgaacggcccgctgcacctggctccggtgaccctgcgttgtaccgtgggttctccgccgcgccgtgtggactgtaccattcaccgtccgtctaacggcggcctgatcgtagaactggaaccagcaaccaagctcgagggaggcagccgggaccacatggtgctgcacgagtacgtgaacgccgccggcatcacaagcggcggtggaggacggagcaaactgaatccacccgatgagtctggacccggttgtatgtcatgtaaatgtgtgttgagctag

SEQ ID NO: 265 - protein, 546 aa, synthetic construct
MGSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKGGSGSTTNIAPALDGAFHRITSSSSLMGLCDETATIIREITGYDRVMVVRFDEEGNAEILSERRRADLEAFLGNRYPASTIPQIARRLYEHNRVRLLVDVNYTPVPLQPRISPLNGRDLDMSLSCLRSMSPVHQKYMQDMGVGATLVCSLMVSGRLWGLIACHHYEPRFVPFHIRAAGEALAETCANRIATLESENLYFQSEFCEREQIHLAGSIQPHGILLAVKEPDNVVIQASINAAEFLNTNSVVGRPLRDLGGDLPLQILPHLNGPLHLAPVTLRCTVGSPPRRVDCTIHRPSNGGLIVELEPATKLEGGSRDHMVLHEYVNAAGIT

SEQ ID NO: 266 - protein, 580 aa, synthetic construct
MGSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKGGSGSTTNIAPALDGAFHRITSSSSLMGLCDETATIIREITGYDRVMVVRFDEEGNAEILSERRRADLEAFLGNRYPASTIPQIARRLYEHNRVRLLVDVNYTPVPLQPRISPLNGRDLDMSLSCLRSMSPVHQKYMQDMGVGATLVCSLMVSGRLWGLIACHHYEPRFVPFHIRAAGEALAETCANRIATLESENLYFQSEFCEREQIHLAGSIQPHGILLAVKEPDNVVIQASINAAEFLNTNSVVGRPLRDLGGDLPLQILPHLNGPLHLAPVTLRCTVGSPPRRVDCTIHRPSNGGLIVELEPATKLEGGSRDHMVLHEYVNAAGITSGLRSRAQASMDPKKKRKVDPKKKRKVDPKKKRK

SEQ ID NO: 267 - protein, 573 aa, synthetic construct
MGSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKGGSGSTTNIAPALDGAFHRITSSSSLMGLCDETATIIREITGYDRVMVVRFDEEGNAEILSERRRADLEAFLGNRYPASTIPQIARRLYEHNRVRLLVDVNYTPVPLQPRISPLNGRDLDMSLSCLRSMSPVHQKYMQDMGVGATLVCSLMVSGRLWGLIACHHYEPRFVPFHIRAAGEALAETCANRIATLESENLYFQSEFCEREQIHLAGSIQPHGILLAVKEPDNVVIQASINAAEFLNTNSVVGRPLRDLGGDLPLQILPHLNGPLHLAPVTLRCTVGSPPRRVDCTIHRPSNGGLIVELEPATKLEGGSRDHMVLHEYVNAAGITSGGGGRSKLNPPDESGPGCMSCKCVLS

SEQ ID NO: 268 - protein, 546 aa, synthetic construct
MGSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKGGSGSTTNIAPALDGAFHRITSSSSLMGLCDETATIIREITGYDRVMVVRFDEEGNAEILSERRRADLEAFLGNRYPASTIPQIARRLYEHNRVRLLVDVNYTPVPLQPRISPLNGRDLDMSLSCLRSMSPVHQKYMQDMGVGATLVCSLMVSGRLWGLIACHHYEPRFVPFHIRAAGEALAETCANRIATLESETVRFQSEFCEREQIHLAGSIQPHGILLAVKEPDNVVIQASINAAEFLNTNSVVGRPLRDLGGDLPLQILPHLNGPLHLAPVTLRCTVGSPPRRVDCTIHRPSNGGLIVELEPATKLEGGSRDHMVLHEYVNAAGIT

SEQ ID NO: 269 - protein, 580 aa, synthetic construct
MGSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKGGSGSTTNIAPALDGAFHRITSSSSLMGLCDETATIIREITGYDRVMVVRFDEEGNAEILSERRRADLEAFLGNRYPASTIPQIARRLYEHNRVRLLVDVNYTPVPLQPRISPLNGRDLDMSLSCLRSMSPVHQKYMQDMGVGATLVCSLMVSGRLWGLIACHHYEPRFVPFHIRAAGEALAETCANRIATLESETVRFQSEFCEREQIHLAGSIQPHGILLAVKEPDNVVIQASINAAEFLNTNSVVGRPLRDLGGDLPLQILPHLNGPLHLAPVTLRCTVGSPPRRVDCTIHRPSNGGLIVELEPATKLEGGSRDHMVLHEYVNAAGITSGLRSRAQASMDPKKKRKVDPKKKRKVDPKKKRK

SEQ ID NO: 270 - protein, 573 aa, synthetic construct
MGSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKGGSGSTTNIAPALDGAFHRITSSSSLMGLCDETATIIREITGYDRVMVVRFDEEGNAEILSERRRADLEAFLGNRYPASTIPQIARRLYEHNRVRLLVDVNYTPVPLQPRISPLNGRDLDMSLSCLRSMSPVHQKYMQDMGVGATLVCSLMVSGRLWGLIACHHYEPRFVPFHIRAAGEALAETCANRIATLESETVRFQSEFCEREQIHLAGSIQPHGILLAVKEPDNVVIQASINAAEFLNTNSVVGRPLRDLGGDLPLQILPHLNGPLHLAPVTLRCTVGSPPRRVDCTIHRPSNGGLIVELEPATKLEGGSRDHMVLHEYVNAAGITSGGGGRSKLNPPDESGPGCMSCKCVLS

SEQ ID NO: 271 - DNA, 1176 bp, synthetic construct
  source 1..1176: mol_type = other DNA
gaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 272 - DNA, 1176 bp, synthetic construct
  source 1..1176: mol_type = other DNA
gaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcactgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 273 - DNA, 1176 bp, synthetic construct
  source 1..1176: mol_type = other DNA
gaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctgcgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 274 - DNA, 1176 bp, synthetic construct
  source 1..1176: mol_type = other DNA
gaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccagaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagac

SEQ ID NO: 275 - DNA, 2343 bp, synthetic construct
  source 1..2343: mol_type = other DNA
atggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctg

SEQ ID NO: 276 - DNA, 2343 bp, synthetic construct
  source 1..2343: mol_type = other DNA
atggctactcaagctgatttgatggagttggacatggccatggaaccagacagaaaagcggctgttagtcactggcagcaacagtcttacctggactatggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctg

SEQ ID NO: 277 - DNA, 1338 bp, synthetic construct
  source 1..1338: mol_type = other DNA
atgacaatgttcgagaacgtgacaagagccctggccagacagctgaaccccagaggcgatctgacacctctggacagcctgattgacttcaagcggttccaccctttttgcctggtgctgcggaagagaaagagcacgctgttctggggcgctagatacgtgcggaccgactacaccctcctggatgtgctggagcctgggagctcccctagcgaccccaccgacacaggcaatttcggctttaagaacatgctggacaccagagtggaaggcgacgtggacgttcctaagaccgtgaaggtaaagggtacagccggcctgagccagaatagcaccctggaagtgcagaccctgagcgtggcccctaaggccctggagaccgtgcaggagagaaaactggctgctgatcaccccttcctgaaagagatgcaggaccagggcgaaaacctgtatgtggtgatggaagttgtggaaaccgtgcaggaggtcactctggaaagagccggaaaggccgaagcctgcttcagcctgcccttcttcgcccctctgggcctccaaggcagcatcaaccacaaggaagccgtgaccatcccaaagggctgtgtgctggccttccgggtgcggcagctgatggtgaaaggcaaagacgagtgggatatcccccacatctgcaacgacaacatgcagacattcccccccggcgagaagtctggcgaggaaaaagtgatcctgatccaggcctctgatgtgggcgacgtgcacgagggatttaggaccctgaaggaagaggtgcaaagagagacacagcaagtggagaaactgtctagagtgggccagagcagccttctgtctagcctgtccaagctgctgggcaagaagaaggagctgcaagacctggagctggccctggagggcgcactggataagggccatgaggtgaccttggaggccctgccaaaggacgtcctgctgagcaaggaagccgtgggcgccatcctgtacttcgtgggcgctctgaccgagctgagcgaggcccagcagaagctgctggtgaagtccatggaaaagaaaatcctgcctgtgcagctgaagctggtggagagcaccatggaacagaactttctgctggacaaggagggcgtgttccctctgcagcctgagctcctgagctctctgggagatgaggaactgaccctgacagaggccctggtcggactgtccggcctggaagtgcagcggagcggccctcagtacatgtgggatcctgatacactccctagactgtgcgccctgtacgccggcctcagcctgctgcagcagctgaccaaggcttcttaa

SEQ ID NO: 278 - DNA, 1251 bp, synthetic construct
  source 1..1251: mol_type = other DNA
atgttcagcgtgttcgaggagatcacccggatcgtggtcaaagaaatggacgctggcggagacatgatcgccgtgcggagcctggttgacgccgacagattcagatgttttcacctggtcggagagaaaagaaccttcttcggctgcagacactacacaaccggcctgacactgatggatatcctggacaccgacggcgataagtggctggatgagctggattccggcctgcaaggccagaaagccgagttccagatcctggacaacgtggacagcaccggcgagctgatcgtgcggctgcccaaggaaatcaccatcagcggctccttccaaggcttccaccaccagaagatcaagatttcagagaatcggatcagccaacagtacctggccaccctggaaaacagaaagctgaagagggaactgcctttcagctttagaagcatcaacacaagagagaacctgtacctggtgacagagacactggagaccgtgaaggaagagacactgaagagtgatagacagtataagttctggagccagatcagccagggccacctgtcctacaagcacaagggccagcgcgaagtgaccattcctccaaatagagtgctgagctacagagtgaagcagctggttttcccaaacaaggaaaccatgaacatccacttccggggcaagaccaagagctttcctgaggaaaaggacggcgccagcagctgcctgggaaagagcctgggtagcgaggattctcgcaatatgaaagagaagctggaggacatggaatctgtgctgaaggacctgaccgaggaaaagcggaaggatgtgctcaacagcctggccaagtgcctgggcaaagaggacatcagacaggacctggagcagcgggtgtccgaggtgctgatctctggcgagctgcatatggaggatcctgataagcctctgctgagctctctgtttaacgccgccggcgtgctggtcgaggccagagccaaggccatcctggacttcctggacgctcttctggagctgtctgaagaacagcagttcgtggccgaggcactggaaaagggaacactccctctgctgaaagaccaggtgaagagcgtgatggaacagaactgggatgagctggcttctagcccccccgacatggactacgaccccgaggccagaatcctgtgcgccctgtacgtggtggtgtccatcctgctggaactggctgaaggccctacctctgtgagcagctaa

SEQ ID NO: 279 - DNA, 1527 bp, synthetic construct
  source 1..1527: mol_type = other DNA
atgcctagcatgctggagcggatcagcaagaacctggtgaaggaaatcggaagcaaggacctgacccctgtgaaatacctgctcagcgctacaaagctgcggcagttcgtgatcctgcggaagaagaaagatagcagaagcagtttttgggagcagtctgactacgtgcccgtggaattctccctgaacgacatcctggagccttcctctagcgtgctggaaaccgttgtgaccggcccattccacttcagcgacatcatgatccagaaacacaaggccgacatgggcgttaatgtgggcatcgaggtgagcgtgagcggcgaggcctctgtcgaccacggctgctctctggagttccagatcgtgaccatcccctctccaaacctggaagatttccagaagagaaagctgctggatcccgagcctagcttcctgaaagagtgcagacggcggggcgacaacctgtacgtggtgacagaagccgtggaacttatcaacaacaccgtgctgtacgacagcagcagcgtgaacatcctgggcaagatcgccctgtggatcacatacggcaagggccagggccaaggagaatctctgagagtgaagaagaaagccctgaccctgcagaaaggcatggtgatggcctacaagagaaagcagctggtgatcaaggagaaggctatcctgatctctgatgacgatgagcagagaacattccaggatgagtacgagattagcgagatggtgggctactgcgccgctagaagcgagggcctactgcccagcttccacaccatcagccccaccctgtttaatgccagcagcaacgatatgaaactgaagcctgaactgttcctgacacagcagtttctatccggacatctgcctaagtacgagcaagtgcacatcctgcctgtgggcagaattgaggaacctttttggcagaacttcaagcacctgcaggaggaggtgttccaaaagatcaagacccttgctcagctgtctaaggacgtccaggatgtgatgttctacagcatcctggccatgctgcgcgacagaggcgccctgcaggacctgatgaacatgctggaactggacagcagcggccacctggacggccctggaggcgctatcctgaagaaactgcaacaggacagcaaccacgcctggttcaaccctaaggatcctatcctctacctgctggaagccatcatggtcctgtcagacttccagcacgacctgctggcatgcagcatggaaaagaggattctgctgcagcaacaggagctcgttagaagcatcctggagcctaatttcagatacccctggtccatccctttcaccctgaagcccgagctgctcgcccctctgcagagcgagggcctggccattacatatggcctgctggaagagtgtggcctgagaatggaactggataaccccagatccacctgggatgtggaagccaagatgcctctgtccgccctgtatggcaccctgtctctgctgcagcagctggccgaggcctaa

SEQ ID NO: 280 - DNA, 1455 bp, synthetic construct
  source 1..1455: mol_type = other DNA
atgggctctgccttcgagagagtggtcagaagggtggtgcaggagctcgaccacggcggcgaattcatccccgtgacaagcctgcaaagcagcacaggcttccagccctactgcctggtggttagaaagccctcttcttcttggttctggaagcctagatacaagtgcgtgaacctgagcatcaaggacatcctggaacctgatgccgctgaacctgatgtgcagagaggccggagcttccacttctacgacgccatggacggccagatccagggcagcgtggaactcgccgccccaggccaagccaagatcgccggcggagctgctgtgtccgatagcagctctaccagcatgaacgtgtattctctgtccgtggaccccaacacctggcagaccctgctgcacgagagacacctgcggcagccagagcacaaggtgctgcagcagctgagatctagaggcgacaacgtgtacgtggtgaccgaggtgctccagacccagaaagaggtggaagtcacaagaacccacaagcgggaaggctccggaagattcagcctgcccggcgccacatgtctgcagggcgagggccagggccacctgagccagaagaaaaccgtgaccattcctagcggcagcaccctggcctttcgggtggcccagctggtcatcgacagcgacctggacgtgctgctgttccccgataagaagcagcggacctttcagcctcctgccaccggccacaaaagatccacctccgagggagcctggcctcagctgcctagcggcctgagcatgatgcggtgcctgcacaacttcctgactgatggagtgcccgccgagggcgcttttaccgaggacttccagggcctgcgagccgaagtggagacaatcagcaaggaactggaactgctggatagagagctgtgccaactgctgctggaaggcctggaaggcgtgctgcgcgaccagctggctctgagagccctggaagaggccctggaacaaggccagagcctgggccctgttgagcccctggacggccctgccggcgccgtgctggaatgtctggtgctgagcagcggcatgctggtgcctgagctggctatccccgtggtgtacctgctaggcgccctgaccatgctgtctgagacacagcataagctgctcgcagaagccctggagagccagacactgctggggcctctggagctggtgggcagcctgctggagcagagcgccccttggcaggagcggtctacaatgagtctgcctccaggtctccttggaaatagctggggcgagggcgctcctgcttgggtgctgctggatgagtgcggcctggaactgggcgaggacacccctcacgtgtgttgggagccacaggcccagggcagaatgtgcgccctgtacgccagcctggccttgctgagcggcctgagccaggagcctcattaa

SEQ ID NO: 281 - DNA, 1491 bp, synthetic construct
  source 1..1491: mol_type = other DNA
atgttcgccaaggccacaagaaatttcttgcgggaagtggacgctgatggcgacctgatcgccgtgtctaatctgaacgacagcgataagctgcaactgctgagcctggtcaccaaaaagaagcggttctggtgctggcagcggcctaagtaccagtttctgagcctgacactgggagatgtgctgattgaggaccaattccccagccccgtggtggtcgagagcgactttgtgaaatacgagggcaaattcgccaaccacgtgtccggcaccctggagacagccctgggcaaggtgaagctgaacctgggcggctctagcagagtggaatctcagagcagcttcggcaccctgcgcaagcaggaggtcgacctgcagcagctgatcagagattctgctgaacggaccatcaacctgagaaaccctgtgctgcaacaggtgctggaaggcagaaacgaggtgctgtgcgtgctgacccagaagatcacaacaatgcagaaatgtgtgatcagcgagcatatgcaggtggaggaaaagtgcggcggaatcgtgggcatccagaccaagaccgtgcaggttagcgccactgaggatggcaacgtgaccaaggacagcaatgtggtgctggaaatccctgctgctacaaccatcgcctacggcgtgatcgagctgtacgtgaagctggatggccagttcgagttctgcctgctgagaggcaagcagggaggcttcgagaacaagaaaagaatcgactccgtgtacctggaccccctggtctttagagaattcgccttcatcgatatgcctgacgccgcccacggcatctccagccaagacggtcctctgagcgttctgaagcaagccacactgctgttggagagaaacttccaccctttcgccgagctgcctgagccacagcagaccgccctgagcgatatcttccaggccgtcctgttcgacgacgagctgctgatggtgctcgaacctgtgtgcgacgacctggtgagcggactgagccctacagtggctgtgctaggagagctgaagcccagacagcagcaggacctggtggcctttctccagctggttggctgtagcctgcagggcggctgtcctggccccgaggacgccggcagcaagcaactgttcatgaccgcctatttcctggtgagcgccctggccgaaatgcccgacagcgctgccgctcttctgggcacctgctgcaaactgcagatcatcccaaccctgtgtcacctgctgcgggccctgtctgatgatggagtgtccgacctggaagatcctaccctgacgcctctgaaagacaccgagaggttcggcattgtgcagagactgtttgcatctgccgacatctctctggaacggctgaagagctccgtgaaggccgtgatcctgaaggacagcaaggtgttcccactgcttctctgcatcaccctgaacggcctgtgcgccctgggcagagagcacagttaa

SEQ ID NO: 282 - skipped

SEQ ID NO: 283 - DNA, 2073 bp, synthetic construct
  source 1..2073: mol_type = other DNA
atggggaaaagacctgccgccacaaagaaagccggccaggccaagaagaagaaaggctctggcggaagcattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttcggcggaggcagtggcggtggcggatctggacggaacaccttcgctgcctctgtggtggtgccctacgagcctcctggaagcgagggaagcggctctgaaggaagcggaagcgagggctctggcgagtctctgtttaagggccccagagattacaaccctatcagcagcaccatttgccacctgaccaacgagagcgatggacatacaaccagcctgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagaccagcggaagcgagggcagcggatccgaaggcgtgtctaagggcgaagagctgatcaaggaaaatatgcgtatgaaggtggtcatggaaggttcggtcaacggccaccaattcaaatgcacaggtgaaggagaaggcagaccgtacgagggagtgcaaaccatgaggatcaaagtcatcgagggaggacccctgccatttgcctttgacattcttgccacgtcgttcatgtatggcagccgtacctttatcaagtacccggccgacatccctgatttctttaaacagtcctttcctgagggttttacttgggaaagagttacgagatacgaagatggtggagtcgtcaccgtcacgcaggacaccagccttgaggatggcgagctcgtctacaacgtcaaggtcagaggggtaaactttccctccaatggtcccgtgatgcagaagaagaccaagggttgggagcctaatacagagatgatgtatccagcagatggtggtctgagaggatacactgacatcgcactgaaagttgatggtggtggccatctgcactgcaacttcgtgacaacttacaggtcaaaaaagaccgtcgggaacatcaagatgcccggtgtccatgccgttgatcaccgcctggaaaggatcgaggagagtgacaatgaaacctacgtagtgcaaagagaagtggcagttgccaaatacagcaaccttggtggtggcatggacgagctatataagtag

SEQ ID NO: 284 - DNA, 2532 bp, synthetic construct
  source 1..2532: mol_type = other DNA
atgagtagcgctatgttggtgacttgcctcccggaccccagcagcagcttccgtgaggatgccccgcggcccccagtgccaggggaagaaggggagaccccaccgtgtcagccaggggtgggcaagggccaggtcaccaaacccatgcctgtctcttccaacaccaggcggaacgaagatgggttgggggagccggaggggcgggcatctccggattcccctctgacccggtggaccaagtccttacactccttattgggcgatcaagacggtgcttacctgttccgaactttcctggagagggagaaatgcgtggataccttagacttctggtttgcctgcaatggattcaggcagatgaacctgaaggataccaaaactttacgagtagccaaagcgatctacaaaaggtacattgagaacaacagcattgtctccaagcagctgaagcctgccaccaagacctacataagagatggcatcaagaagcagcagattgattccatcatgtttgaccaggcgcagaccgagatccagtcggtgatggaggaaaatgcctaccagatgtttttgacttctgatatatacctcgaatatgtgaggagtgggggagaaaacacagcttacatgagtaatgggggactcgggagcctaaaggtcgtgtgtggctatctccccaccttgaatgaagaagaggagtggacttgtgccgacttcaagtgcaaactttcgccaaccgtggttggcttgtccagcaaaactctgagggccacggcgagtgtgaggtccacggaaactgttgacagtggatacaggtccttcaagaggagcgatcctgttaatccttatcacataggttctggctatgtctttgcaccagccaccagcgccaacgacagtgagatatccagtgatgcgctgacggatgattccatgtccatgacggacagcagtgtagatggaattcctccttatcgtgtgggcagtaagaaacagctccagagagaaatgcatcgcagtgtgaaggccaatggccaagtgtctctacctcatttcccgagaacccaccgcctgcccaaggagatgacccccgtggaacccgccacctttgcagctgagctgatctcgaggctggaaaagctgaagctggagttggagagccgccacagcctggaggagcgcctgcagcagatccgagaggatgaagagagagagggctccgagctcacactcaattcgcgggagggggcgcccacgcagcaccccctctccctactgccctccggcagctacgaggaagacccgcagacgatactggacgatcacctgtccagggtcctcaagacccctggctgccagtctccaggcgtaggccgctatagcccccgctcccgctccccggaccaccaccaccaccaccattcgcagtaccactccctgctcccgcccggtggcaagctgcctcccgcggccgcctcgccgggcgcctgccccctcctcgggggcaaaggctttgtgaccaagcagacgacgaagcatgtccaccaccactacatccaccaccatgccgtccccaagaccaaggaggagatcgaggcggaggccacgcagcgggtgcactgcttctgccctgggggcagcgagtattactgctactcgaaatgcaaaagccactccaaggctccggaaaccatgcccagcgagcagtttggcggcagcagaggcagtaccttgcccaaacgcaatgggaaaggcacggagccgggcctggccctgcccgccagggaaggaggggcccccggcggagctggggccctgcagcttccccgggaggaaggagacaggtcgcaggatgtctggcagtggatgctggagagtgagcggcagagcaagcccaagccccatagtgcccaaagcacaaaaaaggcctaccccttggagtctgcccgctcgtctccaggcgaacgagccagccggcaccatctgtgggggggcaacagcgggcacccccgcaccaccccccgtgcccacctgttcacccaggaccctgcgatgcctcccctgaccccacccaacacgctggctcagctggaggaggcctgtcgcaggctagctgaggtgtcgaagcccccaaagcagcggtgctgtgtggccagtcagcagagggacaggaatcattcggccactgttcagacgggagccacacccttctccaatccaagcctggctccagaagatcacaaagagccaaagaaactggcaggtgtccacgcgctccaggccagtgagttggttgtcacttactttttctgtggggaagaaattccataccggaggatgctgaaggctcagagcttgaccctgggccactttaaagagcagctcagcaaaaagggaaattataggtattacttcaaaaaagcaagcgatgagtttgcctgtggagcggtgtttgaggagatctgggaggatgagacggtgctcccgatgtatgaaggccggattctgggcaaagtggagcggatcgattag

SEQ ID NO: 285 - DNA, 300 bp, synthetic construct
  source 1..300: mol_type = other DNA
atgacagtgaccctgaacatggaaagacaccacttcctgggcatcagcattgtgggccagtccaatgacagaggcgatggaggaatctacatcggctctatcatgaagggcggcgccgtcgccgctgatggcagaatcgagcctggcgacatgctgctgcaggtgaacgacgtgaacttcgagaacatgagcaacgacgacgccgtgcgggtgctgcgggaaggcggtggacggagcaaactgaatccacccgatgagtctggacccggttgtatgtcatgtaaatgtgtgttgagctag

SEQ ID NO: 286 - DNA, 237 bp, synthetic construct
  source 1..237: mol_type = other DNA
atgacagtgaccctgaacatggaaagacaccacttcctgggcatcagcattgtgggccagtccaatgacagaggcgatggaggaatctacatcggctctatcatgaagggcggcgccgtcgccgctgatggcagaatcgagcctggcgacatgctgctgcaggtgaacgacgtgaacttcgagaacatgagcaacgacgacgccgtgcgggtgctgcgggaaggaagcgagggatag

SEQ ID NO: 287 - DNA, 249 bp, synthetic construct
  source 1..249: mol_type = other DNA
atgacagtgaccctgaacatggaaagacaccacttcctgggcatcagcattgtgggccagtccaatgacagaggcgatggaggaatctacatcggctctatcatgaagggcggcgccgtcgccgctgatggcagaatcgagcctggcgacatgctgctgcaggtgaacgacgtgaacttcgagaacatgagcaacgacgacgccgtgcgggtgctgcgggaaggaagcgagggaagcggctctgaatag

SEQ ID NO: 288 - DNA, 1341 bp, synthetic construct
  source 1..1341: mol_type = other DNA
atgaccgtgacactgaatatggaaagacaccacttcctgggaattagcatcgtgggccagagcaacgaccggggcgacggcggcatctacatcggcagcatcatgaaaggcggcgccgtggccgctgatggcagaatcgagcctggcgatatgctgctgcaggtgaatgacgtgaacttcgagaacatgagcaacgacgacgccgtgcgggtgctgagagagatcgtgtcccagaccggccctatcagcctgacagtcgccaagtgctgggaccccacccctagatcctacttcaccgtgcctagggctgaccctgtgcgccccatcgaccccgccgcctggctgagccacaccgccgctctgaccggagcacttcctagatacggcacaagcccttgtagctctgccgtgaccagaaccagctccagcagcctgacatctagcgtgcctggcgccccacagctcgaggaagcccctctgaccgtgaagtccgacatgagcgccgttgtgagggtgatgcagctgcctgattctggcctggaaatccgggaccggatgtggctgaaaatcaccatcgccaacgccgtgatcggcgccgatgtggtggattggctgtatacccacgtcgagggcttcaaggaacggagagaggccagaaagtacgcctcttccctgctgaagcacggctttctgcggcacacagttaacaagatcaccttcagcgagcagtgctactacgtgttcggcgacctgtgcagcaatctggccacactgaacctgaacagcggcagcagcggaacaagcgatcaggacaccctggcccctctgccacatcccgctgctccttggcctctgggccaaggctacccctaccagtaccctggccctccaccttgcttccctccagcttatcaagatcctggatttagctacggctctggctctaccggcagccagcagagcgagggcagcaagagcagcggatctacaagaagcagccggcgggcccctgggagagagaaagagagaagagccgccggagccggcggaagtggctccgagagcgaccacacagcccccagcggcgtgggatctagctggcgggaaagacctgctggccagctgagcagaggctctagccccagatcccaggccagcgccactgcccctggactgccccccccacaccctaccaccaaggcctacaccgtggtgggcggaccacctggcggccccccggtcagagaactggctgctgtgcctcctgagctgaccggcagtagacagtcttttcagaaggccatgggcaacccctgtgaattcttcgtggacatcatgtag

SEQ ID NO: 289 - DNA, 333 bp, synthetic construct
  source 1..333: mol_type = other DNA
atggccgagacaaagatcatctaccacatggacgaggaagaaaccccttacctggtgaaactgccagtggcccctgagcgggtgaccctggctgattttaagaacgtgctgagcaatagacctgtgcacgcctacaagttctttttcaaaagcatggatcaggacttcggcgtggtcaaggaagagatcttcgacgacaacgccaagctgccctgcttcaacggcagagtggtgtcttggctggttctggccgagggcggtggacggagcaaactgaatccacccgatgagtctggacccggttgtatgtcatgtaaatgtgtgttgagctag

SEQ ID NO: 290 - DNA, 258 bp, synthetic construct
  source 1..258: mol_type = other DNA
atggccgagacaaagatcatctaccacatggacgaggaagaaaccccttacctggtgaaactgccagtggcccctgagcgggtgaccctggctgattttaagaacgtgctgagcaatagacctgtgcacgcctacaagttctttttcaaaagcatggatcaggacttcggcgtggtcaaggaagagatcttcgacgacaacgccaagctgccctgcttcaacggcagagtggtgtcttggctggttctggccgagtag

SEQ ID NO: 291 - DNA, 330 bp, synthetic construct
  source 1..330: mol_type = other DNA
atgtgtgacagcatcgtggtcgcctactacttctgcggcgagcctatcccctacagaacactggttagaggcagagctgtgaccctgggccagttcaaggaactgctgaccaagaagggcagctaccggtactattttaagaaagtgtctgatgagttcgactgcggagtggtgttcgaggaggtgcgggaagatgaggccgtgctgcctgtgttcgaggaaaagatcatcggcaaggtggaaaaagtggacggcggtggacggagcaaactgaatccacccgatgagtctggacccggttgtatgtcatgtaaatgtgtgttgagctag

SEQ ID NO: 292 - DNA, 255 bp, synthetic construct
  source 1..255: mol_type = other DNA
atgtgtgacagcatcgtggtcgcctactacttctgcggcgagcctatcccctacagaacactggttagaggcagagctgtgaccctgggccagttcaaggaactgctgaccaagaagggcagctaccggtactattttaagaaagtgtctgatgagttcgactgcggagtggtgttcgaggaggtgcgggaagatgaggccgtgctgcctgtgttcgaggaaaagatcatcggcaaggtggaaaaagtggactag

SEQ ID NO: 293 - DNA, 375 bp, synthetic construct
  source 1..375: mol_type = other DNA
atggacatccacgtggaccctgagaagttcgccgccgagctgatcagcagactggaaggcgtgctgagagatcgggaaggcggtggacggagcaaactgaatccacccgatgagtctggacccggttgtatgtcatgtaaatgtgtgttgagcggcagcggcgaaggtcgggggagtttgcttacctgtggcgatgtcgaggagaatccagggcccggatccgacatccacgtggaccctgagaagttcgccgccgagctgatcagcagactggaaggcgtgctgagagatcgggaaggcggtggacggagcaaactgaatccacccgatgagtctggacccggttgtatgtcatgtaaatgtgtgttgagctag

SEQ ID NO: 294 - DNA, 255 bp, synthetic construct
  source 1..255: mol_type = other DNA
atggacatccacgtggaccctgagaagttcgccgccgagctgatcagcagactggaaggcgtgctgagagatcgggaaggcggtggacggagcaaactgaatccacccgatgagtctggacccggttgtatgtcatgtaaatgtgtgttgagcggcagcggcgaaggtcgggggagtttgcttacctgtggcgatgtcgaggagaatccagggcccggatccggaagcgagggaagcggctctgaaggaagctag

SEQ ID NO: 295 - protein, 392 aa, synthetic construct
EEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 296 - protein, 392 aa, synthetic construct
EEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRHCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 297 - protein, 392 aa, synthetic construct
EEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPCEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 298 - protein, 392 aa, synthetic construct
EEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNQRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 299 - protein, 781 aa, synthetic construct
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 300 - protein, 781 aa, synthetic construct
MATQADLMELDMAMEPDRKAAVSHWQQQSYLDYGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 301 - protein, 445 aa, synthetic construct
MTMFENVTRALARQLNPRGDLTPLDSLIDFKRFHPFCLVLRKRKSTLFWGARYVRTDYTLLDVLEPGSSPSDPTDTGNFGFKNMLDTRVEGDVDVPKTVKVKGTAGLSQNSTLEVQTLSVAPKALETVQERKLAADHPFLKEMQDQGENLYVVMEVVETVQEVTLERAGKAEACFSLPFFAPLGLQGSINHKEAVTIPKGCVLAFRVRQLMVKGKDEWDIPHICNDNMQTFPPGEKSGEEKVILIQASDVGDVHEGFRTLKEEVQRETQQVEKLSRVGQSSLLSSLSKLLGKKKELQDLELALEGALDKGHEVTLEALPKDVLLSKEAVGAILYFVGALTELSEAQQKLLVKSMEKKILPVQLKLVESTMEQNFLLDKEGVFPLQPELLSSLGDEELTLTEALVGLSGLEVQRSGPQYMWDPDTLPRLCALYAGLSLLQQLTKAS

SEQ ID NO: 302 - protein, 416 aa, synthetic construct
MFSVFEEITRIVVKEMDAGGDMIAVRSLVDADRFRCFHLVGEKRTFFGCRHYTTGLTLMDILDTDGDKWLDELDSGLQGQKAEFQILDNVDSTGELIVRLPKEITISGSFQGFHHQKIKISENRISQQYLATLENRKLKRELPFSFRSINTRENLYLVTETLETVKEETLKSDRQYKFWSQISQGHLSYKHKGQREVTIPPNRVLSYRVKQLVFPNKETMNIHFRGKTKSFPEEKDGASSCLGKSLGSEDSRNMKEKLEDMESVLKDLTEEKRKDVLNSLAKCLGKEDIRQDLEQRVSEVLISGELHMEDPDKPLLSSLFNAAGVLVEARAKAILDFLDALLELSEEQQFVAEALEKGTLPLLKDQVKSVMEQNWDELASSPPDMDYDPEARILCALYVVVSILLELAEGPTSVSS

SEQ ID NO: 303 - protein, 508 aa, synthetic construct
MPSMLERISKNLVKEIGSKDLTPVKYLLSATKLRQFVILRKKKDSRSSFWEQSDYVPVEFSLNDILEPSSSVLETVVTGPFHFSDIMIQKHKADMGVNVGIEVSVSGEASVDHGCSLEFQIVTIPSPNLEDFQKRKLLDPEPSFLKECRRRGDNLYVVTEAVELINNTVLYDSSSVNILGKIALWITYGKGQGQGESLRVKKKALTLQKGMVMAYKRKQLVIKEKAILISDDDEQRTFQDEYEISEMVGYCAARSEGLLPSFHTISPTLFNASSNDMKLKPELFLTQQFLSGHLPKYEQVHILPVGRIEEPFWQNFKHLQEEVFQKIKTLAQLSKDVQDVMFYSILAMLRDRGALQDLMNMLELDSSGHLDGPGGAILKKLQQDSNHAWFNPKDPILYLLEAIMVLSDFQHDLLACSMEKRILLQQQELVRSILEPNFRYPWSIPFTLKPELLAPLQSEGLAITYGLLEECGLRMELDNPRSTWDVEAKMPLSALYGTLSLLQQLAEA

SEQ ID NO: 304 - protein, 484 aa, synthetic construct
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPH

SEQ ID NO: 305 - protein, 496 aa, synthetic construct
MFAKATRNFLREVDADGDLIAVSNLNDSDKLQLLSLVTKKKRFWCWQRPKYQFLSLTLGDVLIEDQFPSPVVVESDFVKYEGKFANHVSGTLETALGKVKLNLGGSSRVESQSSFGTLRKQEVDLQQLIRDSAERTINLRNPVLQQVLEGRNEVLCVLTQKITTMQKCVISEHMQVEEKCGGIVGIQTKTVQVSATEDGNVTKDSNVVLEIPAATTIAYGVIELYVKLDGQFEFCLLRGKQGGFENKKRIDSVYLDPLVFREFAFIDMPDAAHGISSQDGPLSVLKQATLLLERNFHPFAELPEPQQTALSDIFQAVLFDDELLMVLEPVCDDLVSGLSPTVAVLGELKPRQQQDLVAFLQLVGCSLQGGCPGPEDAGSKQLFMTAYFLVSALAEMPDSAAALLGTCCKLQIIPTLCHLLRALSDDGVSDLEDPTLTPLKDTERFGIVQRLFASADISLERLKSSVKAVILKDSKVFPLLLCITLNGLCALGREHS

SEQ ID NO: 306 - skipped

SEQ ID NO: 307 - protein, 690 aa, synthetic construct
MGKRPAATKKAGQAKKKKGSGGSIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGEDLVVTHGDACLPNIMVENGRFSGFIDCGRLGVADRYQDIALATRDIAEELGGEWADRFLVLYGIAAPDSQRIAFYRLLDEFFGGGSGGGGSGRNTFAASVVVPYEPPGSEGSGSEGSGSEGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTSGSEGSGSEGVSKGEELIKENMRMKVVMEGSVNGHQFKCTGEGEGRPYEGVQTMRIKVIEGGPLPFAFDILATSFMYGSRTFIKYPADIPDFFKQSFPEGFTWERVTRYEDGGVVTVTQDTSLEDGELVYNVKVRGVNFPSNGPVMQKKTKGWEPNTEMMYPADGGLRGYTDIALKVDGGGHLHCNFVTTYRSKKTVGNIKMPGVHAVDHRLERIEESDNETYVVQREVAVAKYSNLGGGMDELYK

SEQ ID NO: 308 - protein, 843 aa, synthetic construct
MSSAMLVTCLPDPSSSFREDAPRPPVPGEEGETPPCQPGVGKGQVTKPMPVSSNTRRNEDGLGEPEGRASPDSPLTRWTKSLHSLLGDQDGAYLFRTFLEREKCVDTLDFWFACNGFRQMNLKDTKTLRVAKAIYKRYIENNSIVSKQLKPATKTYIRDGIKKQQIDSIMFDQAQTEIQSVMEENAYQMFLTSDIYLEYVRSGGENTAYMSNGGLGSLKVVCGYLPTLNEEEEWTCADFKCKLSPTVVGLSSKTLRATASVRSTETVDSGYRSFKRSDPVNPYHIGSGYVFAPATSANDSEISSDALTDDSMSMTDSSVDGIPPYRVGSKKQLQREMHRSVKANGQVSLPHFPRTHRLPKEMTPVEPATFAAELISRLEKLKLELESRHSLEERLQQIREDEEREGSELTLNSREGAPTQHPLSLLPSGSYEEDPQTILDDHLSRVLKTPGCQSPGVGRYSPRSRSPDHHHHHHSQYHSLLPPGGKLPPAAASPGACPLLGGKGFVTKQTTKHVHHHYIHHHAVPKTKEEIEAEATQRVHCFCPGGSEYYCYSKCKSHSKAPETMPSEQFGGSRGSTLPKRNGKGTEPGLALPAREGGAPGGAGALQLPREEGDRSQDVWQWMLESERQSKPKPHSAQSTKKAYPLESARSSPGERASRHHLWGGNSGHPRTTPRAHLFTQDPAMPPLTPPNTLAQLEEACRRLAEVSKPPKQRCCVASQQRDRNHSATVQTGATPFSNPSLAPEDHKEPKKLAGVHALQASELVVTYFFCGEEIPYRRMLKAQSLTLGHFKEQLSKKGNYRYYFKKASDEFACGAVFEEIWEDETVLPMYEGRILGKVERID

SEQ ID NO: 309 - protein, 99 aa, synthetic construct
MTVTLNMERHHFLGISIVGQSNDRGDGGIYIGSIMKGGAVAADGRIEPGDMLLQVNDVNFENMSNDDAVRVLREGGGRSKLNPPDESGPGCMSCKCVLS

SEQ ID NO: 310 - protein, 78 aa, synthetic construct
MTVTLNMERHHFLGISIVGQSNDRGDGGIYIGSIMKGGAVAADGRIEPGDMLLQVNDVNFENMSNDDAVRVLREGSEG

SEQ ID NO: 311 - protein, 82 aa, synthetic construct
MTVTLNMERHHFLGISIVGQSNDRGDGGIYIGSIMKGGAVAADGRIEPGDMLLQVNDVNFENMSNDDAVRVLREGSEGSGSE

SEQ ID NO: 312 - protein, 446 aa, synthetic construct
MTVTLNMERHHFLGISIVGQSNDRGDGGIYIGSIMKGGAVAADGRIEPGDMLLQVNDVNFENMSNDDAVRVLREIVSQTGPISLTVAKCWDPTPRSYFTVPRADPVRPIDPAAWLSHTAALTGALPRYGTSPCSSAVTRTSSSSLTSSVPGAPQLEEAPLTVKSDMSAVVRVMQLPDSGLEIRDRMWLKITIANAVIGADVVDWLYTHVEGFKERREARKYASSLLKHGFLRHTVNKITFSEQCYYVFGDLCSNLATLNLNSGSSGTSDQDTLAPLPHPAAPWPLGQGYPYQYPGPPPCFPPAYQDPGFSYGSGSTGSQQSEGSKSSGSTRSSRRAPGREKERRAAGAGGSGSESDHTAPSGVGSSWRERPAGQLSRGSSPRSQASATAPGLPPPHPTTKAYTVVGGPPGGPPVRELAAVPPELTGSRQSFQKAMGNPCEFFVDIM

SEQ ID NO: 313 - protein, 110 aa, synthetic construct
MAETKIIYHMDEEETPYLVKLPVAPERVTLADFKNVLSNRPVHAYKFFFKSMDQDFGVVKEEIFDDNAKLPCFNGRVVSWLVLAEGGGRSKLNPPDESGPGCMSCKCVLS

SEQ ID NO: 314 - protein, 85 aa, synthetic construct
MAETKIIYHMDEEETPYLVKLPVAPERVTLADFKNVLSNRPVHAYKFFFKSMDQDFGVVKEEIFDDNAKLPCFNGRVVSWLVLAE

SEQ ID NO: 315 - protein, 109 aa, synthetic construct
MCDSIVVAYYFCGEPIPYRTLVRGRAVTLGQFKELLTKKGSYRYYFKKVSDEFDCGVVFEEVREDEAVLPVFEEKIIGKVEKVDGGGRSKLNPPDESGPGCMSCKCVLS

SEQ ID NO: 316 - protein, 84 aa, synthetic construct
MCDSIVVAYYFCGEPIPYRTLVRGRAVTLGQFKELLTKKGSYRYYFKKVSDEFDCGVVFEEVREDEAVLPVFEEKIIGKVEKVD

SEQ ID NO: 317 - protein, 124 aa, synthetic construct
MDIHVDPEKFAAELISRLEGVLRDREGGGRSKLNPPDESGPGCMSCKCVLSGSGEGRGSLLTCGDVEENPGPGSDIHVDPEKFAAELISRLEGVLRDREGGGRSKLNPPDESGPGCMSCKCVLS

SEQ ID NO: 318 - protein, 84 aa, synthetic construct
MDIHVDPEKFAAELISRLEGVLRDREGGGRSKLNPPDESGPGCMSCKCVLSGSGEGRGSLLTCGDVEENPGPGSGSEGSGSEGS

SEQ ID NO: 319 - DNA, 1227 bp, synthetic construct
  source 1..1227: mol_type = other DNA
atggggaaaagacctgccgccacaaagaaagccggccaggccaagaagaagaaaggctctggcggaagcggaagcgagggaagcggctctgaaggaagcggaagcgagggctctggttccgaaggaagcaagagcatgagcagcatggtctccgacacaagctgtaccttcccatctagcgacggcattttctggaagcactggatccagaccaaggacggccagtgcggctctcctctggtgagcaccagagacggcttcatcgtgggcatccactctgccagcaactttacaaacacaaacaattacttcaccagcgtgccaaagaacttcatggaactgctgaccaaccaggaggctcagcagtgggtgtccggctggcggctgaacgccgatagcgtgctgtggggcggccacaaagtgttcatggtgaagccagaggagcctttccaacctgtgaaggaggctacccagctgatgaacagcggctctgagggcagcggcagcgagggatcagtgtccaagggcgaagaactgattaaggagaacatgcacatgaaactctatatggaaggcacagtggacaaccaccacttcaaatgcactagcgagggcgagggcaagccctacgagggcacccagaccatgcggatcaaagtggtggaaggcggacctctaccttttgccttcgacatcctggccaccagctttctgtacggctccaagacctttatcaaccatacccagggcatccccgacttcttcaagcagtctttccctgagggctttacatgggagagggtgaccacctatgaagatggaggcgtgctgacagccacacaggacaccagcctgcaagacggttgtctgatctacaacgtgaagatccggggcgtgaacttcacatctaatggccctgtgatgcagaaaaagacactgggatgggaggccttcaccgagacactgtaccctgccgacggaggtcttgaaggcagaaacgacatggccctgaaactggtgggaggaagccacctcatcgccaacgctaagacaacataccggagcaagaagcctgccaagaacctgaaaatgcccggcgtgtactacgtggactacagactggaaagaatcaaggaagctaataacgaaacctacgtggaacagcacgaggtggccgtcgccagatactgcgacctgcctagcaagctgggccacaagctgaactag

SEQ ID NO: 320 - DNA, 1998 bp, synthetic construct
  source 1..1998: mol_type = other DNA
atggggaaaagacctgccgccacaaagaaagccggccaggccaagaagaagaaaggctctggcggaagcattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttcggaagcgagggaagcggctctgaaggaagcggaagcgagggctctggcgagtctctgtttaagggccccagagattacaaccctatcagcagcaccatttgccacctgaccaacgagagcgatggacatacaaccagcctgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagaccagcggaagcgagggcagcggatccgaaggcgtgtctaagggcgaagagctgatcaaggaaaatatgcgtatgaaggtggtcatggaaggttcggtcaacggccaccaattcaaatgcacaggtgaaggagaaggcagaccgtacgagggagtgcaaaccatgaggatcaaagtcatcgagggaggacccctgccatttgcctttgacattcttgccacgtcgttcatgtatggcagccgtacctttatcaagtacccggccgacatccctgatttctttaaacagtcctttcctgagggttttacttgggaaagagttacgagatacgaagatggtggagtcgtcaccgtcacgcaggacaccagccttgaggatggcgagctcgtctacaacgtcaaggtcagaggggtaaactttccctccaatggtcccgtgatgcagaagaagaccaagggttgggagcctaatacagagatgatgtatccagcagatggtggtctgagaggatacactgacatcgcactgaaagttgatggtggtggccatctgcactgcaacttcgtgacaacttacaggtcaaaaaagaccgtcgggaacatcaagatgcccggtgtccatgccgttgatcaccgcctggaaaggatcgaggagagtgacaatgaaacctacgtagtgcaaagagaagtggcagttgccaaatacagcaaccttggtggtggcatggacgagctatataagtag

SEQ ID NO: 321 - DNA, 1284 bp, synthetic construct
  source 1..1284: mol_type = other DNA
atgggaaagcgccccgctgctacaaagaaggctggacaagctaagaagaagaagggaagcggtggcagtggcggcggcagcggtcgcaatacatttcgccattccgtcgtcgtcccatatgaaccaccaggggggggatccggcgggtcagaaggttctgggagcgagggctcagggagtgaaggaagtggcgagtctctgtttaagggccccagagattacaaccctatcagcagcaccatttgccacctgaccaacgagagcgatggacatacaaccagcctgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagaccagcggaagcgagggcagcggatccgaaggcgtgtctaagggcgaagagctgatcaaggaaaatatgcgtatgaaggtggtcatggaaggttcggtcaacggccaccaattcaaatgcacaggtgaaggagaaggcagaccgtacgagggagtgcaaaccatgaggatcaaagtcatcgagggaggacccctgccatttgcctttgacattcttgccacgtcgttcatgtatggcagccgtacctttatcaagtacccggccgacatccctgatttctttaaacagtcctttcctgagggttttacttgggaaagagttacgagatacgaagatggtggagtcgtcaccgtcacgcaggacaccagccttgaggatggcgagctcgtctacaacgtcaaggtcagaggggtaaactttccctccaatggtcccgtgatgcagaagaagaccaagggttgggagcctaatacagagatgatgtatccagcagatggtggtctgagaggatacactgacatcgcactgaaagttgatggtggtggccatctgcactgcaacttcgtgacaacttacaggtcaaaaaagaccgtcgggaacatcaagatgcccggtgtccatgccgttgatcaccgcctggaaaggatcgaggagagtgacaatgaaacctacgtagtgcaaagagaagtggcagttgccaaatacagcaaccttggtggtggcatggacgagctatataagtag

SEQ ID NO: 322 - DNA, 2073 bp, synthetic construct
  source 1..2073: mol_type = other DNA
atggggaaaagacctgccgccacaaagaaagccggccaggccaagaagaagaaaggctctggcggaagcattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttcggcggaggcagtggcggtggcggatctggacggaacaccttcagacactctgtggtggtgccctacgagcctcctggaagcgagggaagcggctctgaaggaagcggaagcgagggctctggcgagtctctgtttaagggccccagagattacaaccctatcagcagcaccatttgccacctgaccaacgagagcgatggacatacaaccagcctgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagaccagcggaagcgagggcagcggatccgaaggcgtgtctaagggcgaagagctgatcaaggaaaatatgcgtatgaaggtggtcatggaaggttcggtcaacggccaccaattcaaatgcacaggtgaaggagaaggcagaccgtacgagggagtgcaaaccatgaggatcaaagtcatcgagggaggacccctgccatttgcctttgacattcttgccacgtcgttcatgtatggcagccgtacctttatcaagtacccggccgacatccctgatttctttaaacagtcctttcctgagggttttacttgggaaagagttacgagatacgaagatggtggagtcgtcaccgtcacgcaggacaccagccttgaggatggcgagctcgtctacaacgtcaaggtcagaggggtaaactttccctccaatggtcccgtgatgcagaagaagaccaagggttgggagcctaatacagagatgatgtatccagcagatggtggtctgagaggatacactgacatcgcactgaaagttgatggtggtggccatctgcactgcaacttcgtgacaacttacaggtcaaaaaagaccgtcgggaacatcaagatgcccggtgtccatgccgttgatcaccgcctggaaaggatcgaggagagtgacaatgaaacctacgtagtgcaaagagaagtggcagttgccaaatacagcaaccttggtggtggcatggacgagctatataagtag

SEQ ID NO: 323 - DNA, 1494 bp, synthetic construct
  source 1..1494: mol_type = other DNA
atggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaacgttcttgtacgacggtattgaaattcaagctgatcagacccctgaagatttggacatggaggataacgatattattgaggctcacagagaacagattggaggtggcgggggaggaagcggaggagggggctctggaggaggcggcagcggaagcgagggaagcggctctgaaggaagcggaagcgagggctctggttccgaaggaagcaagagcatgagcagcatggtctccgacacaagctgtaccttcccatctagcgacggcattttctggaagcactggatccagaccaaggacggccagtgcggctctcctctggtgagcaccagagacggcttcatcgtgggcatccactctgccagcaactttacaaacacaaacaattacttcaccagcgtgccaaagaacttcatggaactgctgaccaaccaggaggctcagcagtgggtgtccggctggcggctgaacgccgatagcgtgctgtggggcggccacaaagtgttcatggtgaagccagaggagcctttccaacctgtgaaggaggctacccagctgatgaacagcggctctgagggcagcggcagcgagggatcagtgtccaagggcgaagaactgattaaggagaacatgcacatgaaactctatatggaaggcacagtggacaaccaccacttcaaatgcactagcgagggcgagggcaagccctacgagggcacccagaccatgcggatcaaagtggtggaaggcggacctctaccttttgccttcgacatcctggccaccagctttctgtacggctccaagacctttatcaaccatacccagggcatccccgacttcttcaagcagtctttccctgagggctttacatgggagagggtgaccacctatgaagatggaggcgtgctgacagccacacaggacaccagcctgcaagacggttgtctgatctacaacgtgaagatccggggcgtgaacttcacatctaatggccctgtgatgcagaaaaagacactgggatgggaggccttcaccgagacactgtaccctgccgacggaggtcttgaaggcagaaacgacatggccctgaaactggtgggaggaagccacctcatcgccaacgctaagacaacataccggagcaagaagcctgccaagaacctgaaaatgcccggcgtgtactacgtggactacagactggaaagaatcaaggaagctaataacgaaacctacgtggaacagcacgaggtggccgtcgccagatactgcgacctgcctagcaagctgggccacaagctgaactag

SEQ ID NO: 324 - DNA, 2319 bp, synthetic construct
  source 1..2319: mol_type = other DNA
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagacggaagcgagggaagcggctctgaaggaagcggaagcgagggctctggcgagtctctgtttaagggccccagagattacaaccctatcagcagcaccatttgccacctgaccaacgagagcgatggacatacaaccagcctgtacggcatcggcttcggccctttcatcatcaccaacaagcacctgtttcgcagaaacaacggcaccctgctggtgcagagcctgcacggcgttttcaaggtgaaaaataccacaaccctgcagcaacacctgatcgacggaagagatatgatcatcatcagaatgcctaaggatttcccccccttcccccagaagctgaagttcagagagcctcagcgggaagagagaatctgcctggtcaccaccaatttccagaccagcggaagcgagggcagcggatccgaaggcgtgtctaagggcgaagagctgatcaaggaaaatatgcgtatgaaggtggtcatggaaggttcggtcaacggccaccaattcaaatgcacaggtgaaggagaaggcagaccgtacgagggagtgcaaaccatgaggatcaaagtcatcgagggaggacccctgccatttgcctttgacattcttgccacgtcgttcatgtatggcagccgtacctttatcaagtacccggccgacatccctgatttctttaaacagtcctttcctgagggttttacttgggaaagagttacgagatacgaagatggtggagtcgtcaccgtcacgcaggacaccagccttgaggatggcgagctcgtctacaacgtcaaggtcagaggggtaaactttccctccaatggtcccgtgatgcagaagaagaccaagggttgggagcctaatacagagatgatgtatccagcagatggtggtctgagaggatacactgacatcgcactgaaagttgatggtggtggccatctgcactgcaacttcgtgacaacttacaggtcaaaaaagaccgtcgggaacatcaagatgcccggtgtccatgccgttgatcaccgcctggaaaggatcgaggagagtgacaatgaaacctacgtagtgcaaagagaagtggcagttgccaaatacagcaaccttggtggtggcatggacgagctatataagtag

SEQ ID NO: 325 - protein, 408 aa, synthetic construct
MGKRPAATKKAGQAKKKKGSGGSGSEGSGSEGSGSEGSGSEGSKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNSGSEGSGSEGSVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 326 - protein, 665 aa, synthetic construct
MGKRPAATKKAGQAKKKKGSGGSIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGEDLVVTHGDACLPNIMVENGRFSGFIDCGRLGVADRYQDIALATRDIAEELGGEWADRFLVLYGIAAPDSQRIAFYRLLDEFFGSEGSGSEGSGSEGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTSGSEGSGSEGVSKGEELIKENMRMKVVMEGSVNGHQFKCTGEGEGRPYEGVQTMRIKVIEGGPLPFAFDILATSFMYGSRTFIKYPADIPDFFKQSFPEGFTWERVTRYEDGGVVTVTQDTSLEDGELVYNVKVRGVNFPSNGPVMQKKTKGWEPNTEMMYPADGGLRGYTDIALKVDGGGHLHCNFVTTYRSKKTVGNIKMPGVHAVDHRLERIEESDNETYVVQREVAVAKYSNLGGGMDELYK

SEQ ID NO: 327 - protein, 427 aa, synthetic construct
MGKRPAATKKAGQAKKKKGSGGSGGGSGRNTFRHSVVVPYEPPGGGSGGSEGSGSEGSGSEGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTSGSEGSGSEGVSKGEELIKENMRMKVVMEGSVNGHQFKCTGEGEGRPYEGVQTMRIKVIEGGPLPFAFDILATSFMYGSRTFIKYPADIPDFFKQSFPEGFTWERVTRYEDGGVVTVTQDTSLEDGELVYNVKVRGVNFPSNGPVMQKKTKGWEPNTEMMYPADGGLRGYTDIALKVDGGGHLHCNFVTTYRSKKTVGNIKMPGVHAVDHRLERIEESDNETYVVQREVAVAKYSNLGGGMDELYK

SEQ ID NO: 328 - protein, 16 aa, synthetic construct
RNTFRHSVVVPYEPPG

SEQ ID NO: 329 - protein, 690 aa, synthetic construct
MGKRPAATKKAGQAKKKKGSGGSIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKARMPDGEDLVVTHGDACLPNIMVENGRFSGFIDCGRLGVADRYQDIALATRDIAEELGGEWADRFLVLYGIAAPDSQRIAFYRLLDEFFGGGSGGGGSGRNTFRHSVVVPYEPPGSEGSGSEGSGSEGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTSGSEGSGSEGVSKGEELIKENMRMKVVMEGSVNGHQFKCTGEGEGRPYEGVQTMRIKVIEGGPLPFAFDILATSFMYGSRTFIKYPADIPDFFKQSFPEGFTWERVTRYEDGGVVTVTQDTSLEDGELVYNVKVRGVNFPSNGPVMQKKTKGWEPNTEMMYPADGGLRGYTDIALKVDGGGHLHCNFVTTYRSKKTVGNIKMPGVHAVDHRLERIEESDNETYVVQREVAVAKYSNLGGGMDELYK

SEQ ID NO: 330 - protein, 502 aa, synthetic construct
MDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGGGGGSGGGGSGGGGSGSEGSGSEGSGSEGSGSEGSGSEGSKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMNSGSEGSGSEGSVSKGEELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANAKTTYRSKKPAKNLKMPGVYYVDYRLERIKEANNETYVEQHEVAVARYCDLPSKLGHKLN

SEQ ID NO: 331 - protein, 772 aa, synthetic construct
MEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDGSEGSGSEGSGSEGSGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTSGSEGSGSEGVSKGEELIKENMRMKVVMEGSVNGHQFKCTGEGEGRPYEGVQTMRIKVIEGGPLPFAFDILATSFMYGSRTFIKYPADIPDFFKQSFPEGFTWERVTRYEDGGVVTVTQDTSLEDGELVYNVKVRGVNFPSNGPVMQKKTKGWEPNTEMMYPADGGLRGYTDIALKVDGGGHLHCNFVTTYRSKKTVGNIKMPGVHAVDHRLERIEESDNETYVVQREVAVAKYSNLGGGMDELYK

SEQ ID NO: 332 - protein, 22 aa, synthetic construct
RSKLNPPDESGPGCMSCKCVLS

SEQ ID NO: 333 - skipped

SEQ ID NO: 334 - protein, 7 aa, synthetic construct
GGSGGSG

SEQ ID NO: 335 - protein, 6 aa, synthetic construct
GGSGGS

SEQ ID NO: 336 - protein, 5 aa, synthetic construct
GSGSS

SEQ ID NO: 337 - protein, 5 aa, synthetic construct
GGGGS

SEQ ID NO: 338 - protein, 5 aa, synthetic construct
GGSGS

SEQ ID NO: 339 - protein, 20 aa, synthetic construct
GSEGSGSEGSGSEGSGSEGS

SEQ ID NO: 340 - protein, 15 aa, synthetic construct
GSEGSGSEGSGSEGS

SEQ ID NO: 341 - protein, 18 aa, synthetic construct
EGSGSEGSGSEGSGSEGS

SEQ ID NO: 342 - protein, 5 aa, synthetic construct
SGGGG

SEQ ID NO: 343 - protein, 12 aa, synthetic construct
GSEGSGSEGSGS

SEQ ID NO: 344 - protein, 6 aa, synthetic construct
GGSGGG

SEQ ID NO: 345 - protein, 6 aa, synthetic construct
GSSGSG

SEQ ID NO: 346 - protein, 9 aa, synthetic construct
GSENLYFQS

SEQ ID NO: 347 - protein, 14 aa, synthetic construct
GGSSGSGENLYFQS

SEQ ID NO: 348 - protein, 11 aa, synthetic construct
SGSEGSGSEGS

SEQ ID NO: 349 - protein, 5 aa, synthetic construct
GSGSG

SEQ ID NO: 350 - protein, 5 aa, synthetic construct
GSGGS

SEQ ID NO: 351 - protein, 5 aa, synthetic construct
GGGSG

SEQ ID NO: 352 - protein, 5 aa, synthetic construct
GGSGG

SEQ ID NO: 353 - protein, 5 aa, synthetic construct
GSEGS

SEQ ID NO: 354 - protein, 10 aa, synthetic construct
GGGSGGGGSG

SEQ ID NO: 355 - protein, 30 aa, synthetic construct
GSGSGSGSGSGSGSGSGSGSGSGSGSGSGS

SEQ ID NO: 356 - protein, 10 aa, synthetic construct
GSEGSGSEGS

SEQ ID NO: 357 - protein, 6 aa, synthetic construct
KKKKKK

SEQ ID NO: 358 - protein, 6 aa, synthetic construct
KQKQKQ

SEQ ID NO: 359 - protein, 6 aa, synthetic construct
GSGSGS

SEQ ID NO: 360 - protein, 15 aa, synthetic construct
GGGGSGGGGSGGGGS

SEQ ID NO: 361 - protein, 5 aa, synthetic construct
FRHSV

SEQ ID NO: 362 - protein, 5 aa, synthetic construct
FAASV

(19) United States
(12) Patent Application Publication
(10) Pub. No.: US 2026/0327337 A1
(43) Pub. Date: Oct. 22, 2026

(54) ENGINEERED PROTEIN CIRCUITS FOR CANCER THERAPY

(71) Applicant: California Institute of Technology, Pasadena, CA (US)

(72) Inventors: Andrew C. Lu, Pasadena, CA (US); Lukas Moeller, Pasadena, CA (US); Michael B. Elowitz, Pasadena, CA (US)

(21) Appl. No.: 19/643,550

(22) Filed: Apr. 9, 2026

Related U.S. Application Data
(60) Provisional application No. 63/786,835, filed on Apr. 10, 2025.
(60) Provisional application No. 63/975,138, filed on Feb. 3, 2026.

(51) Int. Cl. C07K 16/32; A61K 38/00; A61K 48/00; A61P 35/00; C07K 14/00; C12N 9/50; G01N 33/575

(52) U.S. Cl. CPC C07K 16/32; A61K 48/005; A61P 35/00; C07K 14/001; C12N 9/506; C12Y 304/22044; G01N 33/575; G01N 33/57575; A61K 38/00; C07K 2317/92; C07K 2319/33; C07K 2319/61

(57) ABSTRACT

Disclosed herein include methods, compositions, and kits suitable for use in treating and/or determining cancer status in a subject. Compositions (e.g., nucleic acid compositions, synthetic protein circuits) provided herein can comprise a sensor module and an effector module. In some embodiments, expression of a synthetic protein circuit of the disclosure can induce, e.g., apoptosis and/or pyroptosis in a cell.

### RELATED APPLICATIONS

The present application claims priority to U.S. Provisional Application No. 63/786,835, filed Apr. 10, 2025; and U.S. Provisional Application No. 63/975,138, filed Feb. 3, 2026. The entire contents of these applications are hereby expressly incorporated by reference in their entireties.

### STATEMENT REGARDING FEDERALLY SPONSORED R&D

This invention was made with government support under Grant No. EB030015 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-810024-US_SequenceListing, created Apr. 9, 2026, which is 459,997 bytes in size. The information in the electronic format of the Sequence Listing is incorporated herein by reference in its entirety.

### BACKGROUND

### Field

The present disclosure relates generally to the field of synthetic biology, protein engineering, targeted therapy, oncology, Ras, programmed cell death, including apoptosis and pyroptosis, and nanoparticle delivery, including mRNA-LNP delivery.

### Description of the Related Art

Current targeted therapies for cancer rely on pharmacological inhibition, a mechanism susceptible to drug resistance. Pharmacological inhibition indirectly induces cancer cell death, requiring oncogenic addiction for efficacy and thus failing in cancers that lack this dependency or develop alternative resistance mechanisms. Consequently, there remains a substantial unmet need for therapies that directly and selectively eliminate cancer cells with minimal off-target effects and reduced potential for resistance. This unmet need is particularly pronounced in the context of Ras-driven cancers, affecting roughly one in four cancer patients. Ras-targeting therapies such as the small molecule inhibitors Sotorasib and RMC-7977 (as well as related compounds) have limitations in achieving complete and durable cancer cell elimination, or only work against a subset of Ras-mutations.

Oncogenic Ras mutations (including KRAS, NRAS, and HRAS) drive multiple human cancers. Existing targeted therapies frequently operate by inhibiting signaling through the Ras/RAF/MEK/ERK pathway. While such inhibitors may reduce pathway activity and slow tumor growth, clinical and preclinical limitations can include incomplete tumor clearance, cytostatic rather than cytotoxic responses, pathway rebound or compensatory signaling, on-pathway off-tumor toxicities in normal tissues, and acquisition of resistance.

There remains a need for therapies that (i) selectively eliminate Ras-mutant tumor cells by inducing active cell death, (ii) do not require oncogene addiction to function, (iii) remain effective in settings of acquired resistance to Ras-pathway inhibitors, (iv) enable programmable targeting of specific Ras alleles, and (v) reduce toxicity in normal tissues that are susceptible to on-pathway off-tumor effects.

### SUMMARY

Disclosed herein include synthetic protein circuits. In some embodiments, the synthetic protein circuit comprises: (a) a sensor module comprising: a first polypeptide comprising a first oncoprotein binding domain and a first part of a first orthogonal protease domain, wherein the first oncoprotein binding domain is capable of binding a first oncoprotein to form a first oncoprotein-bound polypeptide; a second polypeptide comprising a second oncoprotein binding domain and a second part of the first orthogonal protease domain, wherein the second oncoprotein binding domain is capable of binding a second oncoprotein to form a second oncoprotein-bound polypeptide, and wherein the first part of the first orthogonal protease domain and the second part of the first orthogonal protease domain are capable of associating with each other to constitute a first orthogonal protease capable of being in a first orthogonal protease active state when the first oncoprotein and the second oncoprotein are in close proximity at a proximity site, wherein the first oncoprotein and/or the second oncoprotein comprise: (i) a monomer having at least two distinct binding sites; (ii) a dimer having at least two binding sites per dimer in sufficient proximity to promote association; (iii) an oligomer having at least two binding sites per oligomer in sufficient proximity to promote association; (iv) an aggregate having at least two binding sites per aggregate in sufficient proximity to promote association; (v) a multi-protein complex in which two or more constituent proteins collectively provide at least two binding sites in sufficient proximity for association; and/or (vi) an onco-fusion protein, and (b) an effector module, wherein the first orthogonal protease in the first orthogonal protease active state is capable of activating the effector module in a cell.

Disclosed herein include synthetic protein circuits. In some embodiments, the synthetic protein circuit comprises: (a) a sensor module comprising: a first polypeptide comprising a first oncoprotein binding domain and a first part of a first orthogonal protease domain, wherein the first oncoprotein binding domain is capable of binding a first oncoprotein to form a first oncoprotein-bound polypeptide; a second polypeptide comprising a second oncoprotein binding domain and a second part of the first orthogonal protease domain, wherein the second oncoprotein binding domain is capable of binding a second oncoprotein to form a second oncoprotein-bound polypeptide, and wherein the first part of the first orthogonal protease domain and the second part of the first orthogonal protease domain are capable of associating with each other to constitute a first orthogonal protease capable of being in a first orthogonal protease active state when the first oncoprotein and the second oncoprotein are in close proximity at a proximity site, optionally the first and/or second oncoprotein binding domain comprise a sequence at least 85% identical to the sequence of SEQ ID NO: 109 or SEQ ID NO: 111; and (b) an effector module capable of inducing apoptosis or pyroptosis in a cell, wherein the first orthogonal protease in the first orthogonal protease active state is capable of activating the effector module, thereby inducing apoptosis or pyroptosis in the cell.

In some embodiments, the first oncoprotein and/or the second oncoprotein comprise a monomer having at least two distinct binding sites. In some embodiments, the first oncoprotein and/or the second oncoprotein comprise a dimer having at least two binding sites per dimer in sufficient proximity to promote association. In some embodiments, the first oncoprotein and/or the second oncoprotein comprise an oligomer having at least two binding sites per oligomer in sufficient proximity to promote association. In some embodiments, the first oncoprotein and/or the second oncoprotein comprise an aggregate having at least two binding sites per aggregate in sufficient proximity to promote association. In some embodiments, the first oncoprotein and/or the second oncoprotein comprise a multi-protein complex in which two or more constituent proteins collectively provide at least two binding sites in sufficient proximity for association. In some embodiments, the first oncoprotein and/or the second oncoprotein comprise an onco-fusion protein. In some embodiments, the first part of the first orthogonal protease domain and the second part of the first orthogonal protease domain have weak association affinity.

In some embodiments, the first oncoprotein binding domain and the second oncoprotein binding domain are identical. In some embodiments, the first oncoprotein binding domain and the second oncoprotein binding domain are different. In some embodiments, the first oncoprotein binding domain, the second oncoprotein binding domain, or both are specific to the first and/or second oncoprotein and do not bind or weakly bind a wild type version of the first and/or second oncoprotein. In some embodiments, the first oncoprotein binding domain and/or the second oncoprotein binding domain have up to 400-fold greater affinity for the first and/or second oncoprotein relative to a wild type version of the first and/or second oncoprotein.

In some embodiments, the first oncoprotein binding domain, the second oncoprotein binding domain, or both are capable of binding to a cryptic epitope of the first and/or second oncoprotein. In some embodiments, the cryptic epitope is not available for binding in a wild type version of the first and/or second oncoprotein. In some embodiments, the first oncoprotein binding domain, the second oncoprotein binding domain, or both are capable of binding a cryptic epitope within a p53 oncoprotein. In some embodiments, the first oncoprotein binding domain, the second oncoprotein binding domain, or both comprise a RAS binding domain (RBD) and/or RAS association domain (RAD).

In some embodiments, the first oncoprotein binding domain, the second oncoprotein binding domain, or both comprise an antibody, a nanobody, a monobody, a peptide, an engineered protein, a de novo protein, a natural protein, an aptamer, or any variant or fragment thereof. In some embodiments, the first oncoprotein binding domain, the second oncoprotein binding domain, or both comprise a nanobody, a Camel Ig, Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), single-domain antibody (sdAb), or any combination thereof.

In some embodiments, the first polypeptide and the second polypeptide are expressed in the cell on a single molecule as a proprotein. In some embodiments, the proprotein comprises one or more proprotein protease cleavage sites. In some embodiments, the first polypeptide and the second polypeptide are separated on the proprotein by at least one of the one or more proprotein protease cleavage sites. In some embodiments, the first polypeptide and the second polypeptide are separated on the proprotein by a first proprotein protease cleavage site, or a first proprotein protease cleavage site and a second proprotein protease cleavage site. In some embodiments, the proprotein comprises: the first polypeptide comprising the first oncoprotein binding domain and the first part of the first orthogonal protease domain and the second polypeptide comprising the second oncoprotein binding domain and the second part of the first orthogonal protease domain; a first proprotein protease cleavage site cleavable by the first orthogonal protease in the first orthogonal protease active state, or a first proprotein protease cleavage site and a second proprotein protease cleavage site each cleavable by the first orthogonal protease in the first orthogonal protease active state; and an optional linker. In some embodiments, the proprotein comprises, from N-terminus to C-terminus: the first polypeptide comprising the first oncoprotein binding domain and the first part of the first orthogonal protease domain; a first proprotein protease cleavage site cleavable by the first orthogonal protease in the first orthogonal protease active state; an optional linker; a second proprotein protease cleavage site cleavable by the first orthogonal protease in the first orthogonal protease active state; and the second polypeptide comprising the second oncoprotein binding domain and the second part of the first orthogonal protease domain. In some embodiments, the first part of the first orthogonal protease domain and the second part of the first orthogonal protease domain of the proprotein are capable of associating with each other to form the first orthogonal protease in the first orthogonal protease active state, wherein the first orthogonal protease in the first orthogonal protease active state is capable of cleaving the proprotein at the one or more proprotein protease cleavage sites, e.g., the first and/or second proprotein protease cleavage site, to release the first polypeptide and the second polypeptide as separate molecules in the cell. In some embodiments, the linker: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof, and/or comprises 3 repeating amino acid subunits or more.

In some embodiments, the first polypeptide, the second polypeptide, or both comprise at least one nuclear localization signal. In some embodiments, the first oncoprotein binding domain, the second oncoprotein binding domain, or both, comprise the sequence of SEQ ID NO: 109, SEQ ID NO: 110, or SEQ ID NO: 111. In some embodiments, the first oncoprotein binding domain, the second oncoprotein binding domain, or both, comprise the sequence of any one of SEQ ID NOs: 141-163. In some embodiments, the first polypeptide, the second polypeptide, or both comprise the sequence of any one of SEQ ID NOs: 5, 7, 116-117, and 178-179. In some embodiments, the first proprotein protease cleavage site, the second proprotein protease cleavage site, or both, comprise the sequence of any one of SEQ ID NOs: 247, 253, and 255-258 or a sequence having one or two mismatches relative to any one of SEQ ID NOs: 247, 253, and 255-258.

In some embodiments, the proprotein comprises the sequence of any one of SEQ ID NOs: 112-113 or a sequence at least 85% identical to the sequence of any one of SEQ ID NOs: 112-113. In some embodiments, the first oncoprotein, the second oncoprotein, or both comprise at least one mutation associated with cancer. In some embodiments, the first oncoprotein, the second oncoprotein, or both comprise a conformation, post-translational modification, interaction state, abundance, oligomerization state, multimerization state, and/or subcellular localization state associated with a disease or disorder. In some embodiments, the disease or disorder is cancer. In some embodiments, the first oncoprotein and the second oncoprotein are identical. In some embodiments, the first oncoprotein, the second oncoprotein, or both are endogenous proteins. In some embodiments, the first oncoprotein, the second oncoprotein, or both are over-expressed and/or mis-folded in the cell relative to a wild type version of the first and/or second oncoprotein. In some embodiments, the first oncoprotein, the second oncoprotein, or both are mis-folded, exposing a cryptic epitope. In some embodiments, the first oncoprotein, the second oncoprotein, or both comprise AKT, PI3K, MAPK, p44/42 MAP kinase, TYK2, p38 MAP kinase, PKC, PKA, SAPK, ELK, INK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCy, NF-kB, FAK, CREB, αIIIβ3, FcεRI, BAD, p70S6K, STAT1, STAT2, STAT3, STAT5, STAT6, or any combination thereof. In some embodiments, the first oncoprotein, the second oncoprotein, or both are capable of regulating cell survival, cell growth, cell proliferation, cell adhesion, cell migration, cell metabolism, cell morphology, cell differentiation, apoptosis, or any combination thereof.

In some embodiments: (i) the first oncoprotein and/or the second oncoprotein comprising a monomer having at least two distinct binding sites comprise: p53, β-catenin, e.g., CTNNB1, MYC, e.g., c-Myc, Cyclin D1, any variant thereof, or any combination thereof, (ii) the first oncoprotein and/or the second oncoprotein comprising a dimer having at least two binding sites per dimer in sufficient proximity to promote association comprise: MYC:MAX heterodimer, STAT3 constitutive homodimerization, RAF dimers, e.g., BRAF:CRAF or BRAF:BRAF, HIF-1α:HIF-1β, e.g., ARNT, any variants thereof, or any combination thereof; (iii) the first oncoprotein and/or the second oncoprotein comprising an oligomer having at least two binding sites per oligomer in sufficient proximity to promote association comprise: RAS nanoclusters, e.g., KRAS, NRAS, and/or HRAS nanoclusters, p53 tetramers, EML4-ALK trimerization, BCR-ABL tetramers, EGFR intracellular domain clusters, Nucleophosmin (NPM1) oligomers, or any combination thereof, (iv) the first oncoprotein and/or the second oncoprotein comprising an aggregate having at least two binding sites per aggregate in sufficient proximity to promote association comprise: mutant p53 amyloid aggregates, PTEN aggregates, or both; (v) the first oncoprotein and/or the second oncoprotein comprising a multi-protein complex in which two or more constituent proteins collectively provide at least two binding sites in sufficient proximity for association comprise: MDM2:p53, KRAS:RAF-RBD, β-catenin:TCF/LEF, Cyclin E:CDK2, or any combination thereof, and/or (vi) wherein the first oncoprotein and/or the second oncoprotein comprising an onco-fusion protein comprise: BCR-ABL, EML4-ALK, EWSR1-FLI1, PML-RARα, SS18-SSX, or any combination thereof.

In some embodiments, the first oncoprotein, the second oncoprotein, or both comprise a RAS protein. In some embodiments, the RAS protein is KRAS, NRAS, HRAS, or any combination thereof. In some embodiments, the RAS protein comprises at least one mutation associated with cancer. In some embodiments, the at least one mutation comprises a mutation in KRAS, NRAS, and/or HRAS. In some embodiments, the KRAS mutation is selected from the group consisting of G12C, G12V, G12S, G12A, G12D, G12R, G13V, G13C, G13D, Q61P, Q61L, Q61H, Q61K, Q61E, Q61R, A146V, A146P, and A146T. In some embodiments, the HRAS mutation is selected from the group consisting of G12V, G12C, G12S, G12A, G12D, G12R, G13V, G13dup, G13C, G13S, G13D, Q61L, Q61H, Q61K, and Q61R. In some embodiments, the NRAS mutation is selected from the group consisting of G12A, G12C, G12D, G12R, G12S, G12V, G13A, G13C, G13D, G13R, G13S, G13V, Q61E, Q61H, Q61K, Q61L, Q61P, and Q61R.

In some embodiments, the effector module comprises a protease activatable reporter. In some embodiments, the protease activatable reporter comprises a fluorescent protein comprising green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mScarlet, mApple, mCitrine, mCherry, mruby3, rsCherry, rsCherryRev, derivatives thereof, or any combination thereof. In some embodiments, the effector module comprises a synthetic apoptosis circuit or a synthetic pyroptosis circuit.

In some embodiments, the effector module comprises a synthetic apoptosis circuit comprising: an apoptosis polypeptide comprising a large subunit of an apoptotic effector protein and a small subunit of an apoptotic effector protein separated by a first orthogonal protease cleavage site, wherein two apoptosis polypeptides are capable of associating with each other to constitute an apoptotic protein complex in an apoptotic protein complex inactive state, wherein the first orthogonal protease in a first orthogonal protease active state is capable of cleaving the first orthogonal protease cleavage site of the apoptosis polypeptide, and wherein the first orthogonal protease cleavage site of the apoptosis polypeptide being cleaved changes the apoptotic protein complex from the apoptotic protein complex inactive state to an apoptotic protein complex active state, thereby activating the effector module, wherein the apoptotic protein complex in the apoptotic protein complex active state is capable of inducing apoptosis in the cell. In some embodiments, the apoptotic effector protein comprises caspase-3, caspase-6, caspase-7, or any variant, portion, or derivative thereof.

In some embodiments, the apoptosis polypeptide comprises one or more linkers. In some embodiments, any of the one or more linkers: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof, and/or comprises 3 repeating amino acid subunits or more. In some embodiments, the first orthogonal protease cleavage site comprises the sequence of any one of SEQ ID NOs 247, 253, and 255-258 or a sequence having one or two mismatches relative to any one of SEQ ID NOs 247, 253, and 255-258. In some embodiments, the apoptosis polypeptide comprises the sequence of any one of SEQ ID NOs: 220-221 or a sequence at least 85% identical to any one of SEQ ID NOs: 220-221.

In some embodiments, the effector module comprises a synthetic pyroptosis circuit comprising: a pyroptosis polypeptide comprising a pyroptosis effector domain and an inhibitory domain separated by a first orthogonal protease cleavage site, wherein the inhibitory domain is capable of inhibiting activity of the pyroptosis effector domain, thereby the pyroptosis polypeptide is in a pyroptosis polypeptide inactive state, wherein the first orthogonal protease in a first orthogonal protease active state is capable of cleaving the first orthogonal protease cleavage site of the pyroptosis polypeptide, and wherein the first orthogonal protease cleavage site of the pyroptosis polypeptide being cleaved changes the pyroptosis polypeptide from the pyroptosis polypeptide inactive state to a pyroptosis polypeptide active state, thereby activating the effector module, wherein the pyroptosis polypeptide in the pyroptosis polypeptide active state is capable of inducing pyroptosis in the cell.

In some embodiments, the inhibitory domain comprises a protein domain configured to reduce or abrogate the activity of the pyroptosis effector domain, e.g., a bulky domain. In some embodiments, the inhibitory domain comprises an auto-inhibitory domain of a gasdermin family of pore-forming proteins or maltose-binding protein. In some embodiments, the pyroptosis effector domain comprises an N-terminal domain of a gasdermin (GSDM) protein. In some embodiments, the GSDM protein is GSDMA, GSDMB, GSDMC, GSDMD, GSDME, or DFNB59. In some embodiments, the pyroptosis polypeptide comprises one or more linkers, optionally any of the one or more linkers: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; and/or comprises 3 repeating amino acid subunits or more. In some embodiments, inducing pyroptosis in the cell causes the cell to release one or more inflammatory cytokines. In some embodiments, the one or more inflammatory cytokines comprise IL-18, IL-1β, IL-6, IL-8, interferon gamma (IFN-γ), and/or tumor necrosis factor-alpha (TNF-α).

In some embodiments, the first orthogonal protease cleavage site comprises the sequence of any one of SEQ ID NOs 247, 253, and 255-258 or a sequence having one or two mismatches relative to any one of SEQ ID NOs 247, 253, and 255-258. In some embodiments, the pyroptosis polypeptide comprises the sequence of any one of SEQ ID NOs: 237-241 or a sequence at least 85% identical to any one of SEQ ID NOs: 237-241.

In some embodiments, the first orthogonal protease comprises or is derived from a viral protease. In some embodiments, the first orthogonal protease comprises tobacco etch virus (TEV) protease, tobacco vein mottling virus (TVMV) protease, hepatitis C virus protease (HCVP), derivatives thereof, or any combination thereof. In some embodiments, the first orthogonal protease comprises the sequence of any one of SEQ ID NOs: 211, 213, and 215 or a sequence having at least 85% identity to any one of SEQ ID NOs: 211, 213, and 215. In some embodiments, the first orthogonal protease comprises one or more mutations that boost catalytic efficiency. In some embodiments, the first orthogonal protease comprises the sequence of any one of SEQ ID NOs: 216-217 or a sequence having at least 85% identity to any one of SEQ ID NOs: 216-217.

The synthetic protein circuit can comprise a synthetic amplifier circuit. In some embodiments, the synthetic amplifier circuit comprises: (i) an amplifier protein comprising a first part of a second orthogonal protease domain, a first dimerization domain, a first orthogonal protease cleavage site the first orthogonal protease in the first orthogonal protease active state is capable of cleaving, a second dimerization domain, a second orthogonal cleavage site the first orthogonal protease in the first orthogonal protease active state is capable of cleaving, and a first caging domain; (ii) a companion amplifier protein comprising a second part of a second orthogonal protease domain, a third dimerization domain, a third orthogonal cleavage site the first orthogonal protease in the first orthogonal protease active state is capable of cleaving, a fourth dimerization domain, a fourth orthogonal cleavage site the first orthogonal protease in the first orthogonal protease active state is capable of cleaving, and a second caging domain.

In some embodiments, the amplifier protein and the companion amplifier protein separately do not comprise a second orthogonal protease capable of being in a second orthogonal protease active state. In some embodiments: the first caging domain is a catalytically inactive version of the second part of the second orthogonal protease domain. In some embodiments, the first caging domain has between about 50% to about 99% sequence identity to the second part of the second orthogonal protease domain. In some embodiments, the second caging domain is a catalytically inactive version of the first part of the second orthogonal protease domain. In some embodiments, the second caging domain has between about 50% to about 99% sequence identity to the first part of the second orthogonal protease domain. In some embodiments: the first orthogonal cleavage site and the second orthogonal cleavage site flank the second dimerization domain; and/or the third orthogonal cleavage site and the fourth orthogonal cleavage site flank the fourth dimerization domain. In some embodiments: the first dimerization domain is capable of binding the second dimerization domain; the first dimerization domain is capable of binding the third dimerization domain; and/or the third dimerization domain is capable of binding the fourth dimerization domain. In some embodiments, the affinity of the first dimerization domain for the second dimerization domain is weaker than the affinity of the first dimerization domain for the third dimerization domain. In some embodiments, the affinity of the third dimerization domain for the fourth dimerization domain is weaker than the affinity of the third dimerization domain for the first dimerization domain. In some embodiments, intramolecular binding between the first dimerization domain and the second dimerization domain of the amplifier protein is capable of preventing the first part of the second orthogonal protease domain from associating with the second part of the second orthogonal protease domain of the companion amplifier protein to form the second orthogonal protease in the second orthogonal protease active state. In some embodiments, intramolecular binding between the third dimerization domain and the fourth dimerization domain of the companion amplifier protein is capable of preventing the second part of the second orthogonal protease domain from associating with the first part of the second orthogonal protease domain of the amplifier protein to form the second orthogonal protease in the second orthogonal protease active state.

In some embodiments, the first orthogonal protease in the first orthogonal protease active state is capable of: cleaving the first orthogonal cleavage site and/or second orthogonal cleavage site of the amplifier protein, thereby forming a cleaved amplifier protein; and/or cleaving the third orthogonal cleavage site and/or fourth orthogonal cleavage site of the companion amplifier protein, thereby forming a cleaved companion amplifier protein. In some embodiments, the cleaved amplifier protein and the cleaved companion amplifier protein are capable of associating via intermolecular binding of the first dimerization domain and the third dimerization domain to form a first complex. In some embodiments, the first complex comprises the second orthogonal protease capable of being in the second orthogonal protease active state.

In some embodiments, one or more of the first dimerization domain, second dimerization domain, third dimerization domain, and fourth dimerization domain is selected from the group comprising DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD1117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, portions thereof, derivatives thereof, or any combination thereof.

In some embodiments, one or more of the first dimerization domain, second dimerization domain, third dimerization domain, and fourth dimerization domain comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, one or more of the first dimerization domain, second dimerization domain, third dimerization domain, and fourth dimerization domain is a homodimerization domain or a multimerization domain, e.g., a homo- or hetero-dimerizing or multimerizing leucine zipper, a PDZ domains, a SH3 domain, aGBD domain, or any combination thereof.

In some embodiments, the amplifier protein and/or the companion amplifier protein comprise a linker. In some embodiments, the linker: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; and/or comprises 3 repeating amino acid subunits or more. In some embodiments, the amplifier protein comprises the sequence of SEQ ID NO: 244 or a sequence at least 85% identical to SEQ ID NO: 244, and/or the companion amplifier protein comprises the sequence of SEQ ID NO: 245 or a sequence at least 85% identical to SEQ ID NO: 245.

In some embodiments, the effector module comprises a synthetic apoptosis circuit comprising: an apoptosis polypeptide comprising a large subunit of an apoptotic effector protein and a small subunit of an apoptotic effector protein separated by a fifth orthogonal protease cleavage site, wherein two apoptosis polypeptides are capable of associating with each other to constitute an apoptotic protein complex in an apoptotic protein complex inactive state, wherein the second orthogonal protease in a second orthogonal protease active state is capable of cleaving the fifth orthogonal protease cleavage site of the apoptosis polypeptide, and wherein the fifth orthogonal protease cleavage site of the apoptosis polypeptide being cleaved changes the apoptotic protein complex from the apoptotic protein complex inactive state to an apoptotic protein complex active state, thereby activating the effector module, wherein the apoptotic protein complex in the apoptotic protein complex active state is capable of inducing apoptosis in the cell.

In some embodiments, the apoptotic effector protein comprises caspase-3, caspase-6, caspase-7, or any variant, portion, or derivative thereof. In some embodiments, the apoptosis polypeptide comprises one or more linkers. In some embodiments, any of the one or more linkers: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; and/or comprises 3 repeating amino acid subunits or more. In some embodiments, the first orthogonal protease cleavage site, the second orthogonal cleavage site, the third orthogonal protease cleavage site, the fourth orthogonal protease cleavage site, the fifth orthogonal protease cleavage site, or any combination thereof, comprise the sequence of any one of SEQ ID NOs 247, 253, and 255-258 or a sequence having one or two mismatches relative to any one of SEQ ID NOs 247, 253, and 255-258. In some embodiments, the apoptosis polypeptide comprises the sequence of any one of SEQ ID NOs: 220-221 or a sequence at least 85% identical to any one of SEQ ID NOs: 220-221.

In some embodiments, the effector module comprises a synthetic pyroptosis circuit comprising: a pyroptosis polypeptide comprising a pyroptosis effector domain and an inhibitory domain separated by a fifth orthogonal protease cleavage site, wherein the inhibitory domain is capable of inhibiting activity of the pyroptosis effector domain, thereby the pyroptosis polypeptide is in a pyroptosis polypeptide inactive state, wherein the second orthogonal protease in a second orthogonal protease active state is capable of cleaving the fifth orthogonal protease cleavage site of the pyroptosis polypeptide, and wherein the fifth orthogonal protease cleavage site of the pyroptosis polypeptide being cleaved changes the pyroptosis polypeptide from the pyroptosis polypeptide inactive state to a pyroptosis polypeptide active state, thereby activating the effector module, wherein the pyroptosis polypeptide in the pyroptosis polypeptide active state is capable of inducing pyroptosis in the cell.

In some embodiments, the inhibitory domain comprises a protein domain configured to reduce or abrogate the activity of the pyroptosis effector domain, e.g., a bulky domain. In some embodiments, the inhibitory domain comprises an auto-inhibitory domain of a gasdermin family of pore-forming proteins or maltose-binding protein. In some embodiments, the pyroptosis effector domain comprises an N-terminal domain of a gasdermin (GSDM) protein. In some embodiments, the GSDM protein is GSDMA, GSDMB, GSDMC, GSDMD, GSDME, or DFNB59. In some embodiments, the pyroptosis polypeptide comprises one or more linkers. In some embodiments, any of the one or more linkers: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; and/or comprises 3 repeating amino acid subunits or more. In some embodiments, inducing pyroptosis in the cell causes the cell to release one or more inflammatory cytokines. In some embodiments, the one or more inflammatory cytokines comprise IL-18, IL-10, IL-6, IL-8, interferon gamma (IFN-γ), and/or tumor necrosis factor-alpha (TNF-α).

In some embodiments, the first orthogonal protease cleavage site, the second orthogonal cleavage site, the third orthogonal protease cleavage site, the fourth orthogonal protease cleavage site, the fifth orthogonal protease cleavage site, or any combination thereof, comprise the sequence of any one of SEQ ID NOs 247, 253, and 255-258 or a sequence having one or two mismatches relative to any one of SEQ ID NOs 247, 253, and 255-258 In some embodiments, the pyroptosis polypeptide comprises the sequence of any one of SEQ ID NOs: 237-241 or a sequence at least 85% identical to any one of SEQ ID NOs: 237-241.

In some embodiments, the first orthogonal protease and/or the second orthogonal protease comprises or is derived from a viral protease. In some embodiments, the first orthogonal protease and/or the second orthogonal protease comprises tobacco etch virus (TEV) protease, tobacco vein mottling virus (TVMV) protease, hepatitis C virus protease (HCVP), derivatives thereof, or any combination thereof. In some embodiments, the first orthogonal protease and/or the second orthogonal protease comprises the sequence of any one of SEQ ID NOs: 211, 213, and 215 or a sequence having at least 85% identity to any one of SEQ ID NOs: 211, 213, and 215. In some embodiments, the first orthogonal protease and/or the second orthogonal protease comprise one or more mutations that boost catalytic efficiency. In some embodiments, the first orthogonal protease and/or the second orthogonal protease comprise the sequence of any one of SEQ ID NOs: 216-217 or a sequence having at least 85% identity to any one of SEQ ID NOs: 216-217.

In some embodiments, the first oncoprotein, the second oncoprotein, or both, are capable of being localized at the proximity site. In some embodiments, the first oncoprotein when in a first oncoprotein active state, the second oncoprotein when in a second oncoprotein active state, or both, are capable of being localized at the proximity site. In some embodiments, the first oncoprotein when in a first inactive state, the second oncoprotein when in a second inactive state, or both, are capable of being localized at the proximity site. In some embodiments, the first oncoprotein binding domain of the first polypeptide is capable of binding to the first oncoprotein, the second oncoprotein binding domain of the second polypeptide is capable of binding to the second oncoprotein, or both. In some embodiments, the first oncoprotein binding domain of the first polypeptide is capable of binding to the first oncoprotein in a first oncoprotein active state, the second oncoprotein binding domain of the second polypeptide is capable of binding to the second oncoprotein in a second oncoprotein active state, or both. In some embodiments, the first oncoprotein binding domain of the first polypeptide is capable of binding to the first oncoprotein in a first inactive state, the second oncoprotein binding domain of the second polypeptide is capable of binding to the second oncoprotein in a second inactive state, or both. In some embodiments, the first oncoprotein binding domain of the first polypeptide is capable of binding the first oncoprotein to form the first oncoprotein-bound polypeptide at the proximity site, the second oncoprotein binding domain of the second polypeptide is capable of binding the second oncoprotein to form the second oncoprotein-bound polypeptide at the proximity site, or both. In some embodiments, the proximity site comprises one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, sarcomere, focal contact, ribosome, microsome, plasma membrane, nuclear membrane, or any combination thereof.

In some embodiments, a first concentration of the first oncoprotein-bound polypeptide is at least two-fold higher at the proximity site as compared to a first cellular location other than the proximity site when the first oncoprotein is in a first oncoprotein active state. In some embodiments, a second concentration of the second oncoprotein-bound polypeptide is at least two-fold higher at the proximity site as compared a second cellular location other than the proximity site when the second oncoprotein is in a second oncoprotein active state. In some embodiments, a first concentration of the first orthogonal protease in the first orthogonal protease active state is at least two-fold higher at the proximity site as compared to a cellular location other than the proximity site when the first oncoprotein is in a first oncoprotein active state and/or when the second oncoprotein is in a second oncoprotein active state. In some embodiments, the first part of the first orthogonal protease domain and the second part of the first orthogonal protease domain have the weak association affinity when the first oncoprotein is in a first oncoprotein inactive state and/or the second oncoprotein inactive state. In some embodiments, the first part of the first orthogonal protease domain and the second part of the first orthogonal protease domain are incapable of associating to form the first orthogonal protease in the first orthogonal protease active state when the first oncoprotein is in a first oncoprotein inactive state and/or the second oncoprotein is in a second oncoprotein inactive state. In some embodiments, a first concentration of the first oncoprotein-bound polypeptide and a second concentration of the second oncoprotein-bound polypeptide at the proximity site are insufficient for the first part of the first orthogonal protease domain and the second part of the first orthogonal protease domain to form an active first orthogonal protease when the first oncoprotein is in a first oncoprotein inactive state and/or the second oncoprotein is in a second oncoprotein inactive state. In some embodiments, a first concentration of the first oncoprotein-bound polypeptide at the proximity site is comparable to a first cellular location other than the proximity site when the first oncoprotein is in a first oncoprotein inactive state. In some embodiments, a second concentration of the second oncoprotein-bound polypeptide at the proximity site is comparable to a second cellular location other than the proximity site when the second oncoprotein is in a second oncoprotein inactive state. In some embodiments, the first part of the first orthogonal protease domain and the second part of the first orthogonal protease domain are capable of associating with each other to form the first orthogonal protease in the first orthogonal protease active state at a threshold first polypeptide concentration and a threshold second polypeptide concentration at the proximity site. In some embodiments, the threshold first polypeptide concentration and the threshold second polypeptide concentration at the proximity site are reached at a threshold oncoprotein activation level of the first and/or second oncoprotein.

In some embodiments, the presence and/or activation of the synthetic protein circuit in the cell does not inhibit ERK signaling in the cell. In some embodiments, the presence and/or activation of the synthetic protein circuit in the cell does not cause a significant reduction in the levels of phospho-ERK (pERK) in the cell. In some embodiments, the presence and/or activation of the synthetic protein circuit in the cell does not cause significant transcriptional perturbation in the cell. In some embodiments, the cell is a eukaryotic cell. In some embodiments, the cell is a mammalian cell. In some embodiments, the cell is a cancer cell.

Disclosed herein include nucleic acid compositions. In some embodiments, the nucleic acid composition comprises: one or more polynucleotides encoding a synthetic protein circuit of the disclosure, optionally the one or more polynucleotides comprise: one or more first polynucleotides encoding the first polypeptide, the second polypeptide, the proprotein, or any combination thereof, and/or one or more second polynucleotides encoding the protease activatable reporter, the apoptosis polypeptide and/or the pyroptosis polypeptide; optionally the one or more polynucleotides further comprise one or more third polynucleotides encoding the amplifier protein, the companion amplifier protein, or any combination thereof.

In some embodiments, at least two of the one or more polynucleotides are operably linked to a tandem gene expression element. In some embodiments, the one or more polynucleotides comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof, and/or a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof. In some embodiments, the one or more polynucleotides are operably connected to a promoter selected from the group comprising: a minimal promoter, e.g., TATA, miniCMV, and/or miniPromo; a tissue-specific promoter and/or a lineage-specific promoter; and/or a ubiquitous promoter, e.g., a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof.

In some embodiments, the nucleic acid composition is configured to enhance stability, durability, and/or expression level, optionally a 5′ untranslated region (UTR), a 3′ UTR, and/or a 5′ cap; optionally one or more modified nucleotides, further optionally selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine; and/or optionally a modified nucleotide in place of one or more uridines, optionally the modified nucleoside is selected from pseudouridine (ψ), N 1-methyl-pseudouridine (m 1Ψ), and 5-methyl-uridine (m5U).

In some embodiments, the nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. In some embodiments, at least one of the one or more vectors is a viral vector, a plasmid, a transposable element, a naked DNA vector, a bacteriophage, or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon. In some embodiments, the one or more polynucleotides are comprised in the one or more vectors. In some embodiments, the one or more polynucleotides are comprised in the same vector and/or different vectors. In some embodiments, any of the one or more polynucleotides are situated on the same nucleic acid and/or different nucleic acids.

In some embodiments, the nucleic acid composition comprises circular mRNA, circular DNA, self-amplifying RNA, self-amplifying RNA, and/or mRNA. In some embodiments, the nucleic acid composition is or comprises mRNA. In some embodiments, the mRNA is formulated in a lipid nanoparticle (LNP). In some embodiments, the mRNA comprises a 5′ untranslated region (UTR), a 3′ UTR, and/or a cap. In some embodiments, the mRNA comprises one or more modified nucleotides selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine. In some embodiments, the mRNA comprises a modified nucleotide in place of one or more uridines. In some embodiments, the modified nucleoside is selected from pseudouridine (ψ), N 1-methyl-pseudouridine (m 1Ψ), and 5-methyl-uridine (m5U).

In some embodiments, the LNP comprises one or more of an ionizable cationic lipid, a non-cationic lipid, a sterol, and a PEG-modified lipid. In some embodiments, the non-cationic lipid is a neutral lipid. In some embodiments, the LNP comprises 0.5-15 mol % PEG-modified lipid, 5-25 mol % non-cationic lipid, 25-55 mol % sterol, and 20-60 mol % ionizable cationic lipid. In some embodiments, the LNP comprises: 40-55 mol % ionizable cationic lipid, 5-15 mol % neutral lipid, 35-45 mol % sterol, and 1-5 mol % PEG-modified lipid. In some embodiments, the LNP comprises: 47 mol % ionizable cationic lipid, 11.5 mol % neutral lipid, 38.5 mol % sterol, and 3.0 mol % PEG-modified lipid; 48 mol % ionizable cationic lipid, 11 mol % neutral lipid, 38.5 mol % sterol, and 2.5 mol % PEG-modified lipid; 49 mol % ionizable cationic lipid, 10.5 mol % neutral lipid, 38.5 mol % sterol, and 2.0 mol % PEG-modified lipid; 50 mol % ionizable cationic lipid, 10 mol % neutral lipid, 38.5 mol % sterol, and 1.5 mol % PEG-modified lipid; or 51 mol % ionizable cationic lipid, 9.5 mol % neutral lipid, 38.5 mol % sterol, and 1.0 mol % PEG-modified lipid. In some embodiments: the ionizable cationic lipid is heptadecan-9-yl 8 ((2 hydroxyethyl)(6 oxo 6-(undecyloxy)hexyl)amino)octanoate; the neutral lipid is 1,2 distearoyl sn glycero-3 phosphocholine (DSPC); the sterol is cholesterol; and/or the PEG-modified lipid is 1-monomethoxypolyethyleneglycol-2,3-dimyristylglycerol with polyethylene glycol of average molecular weight 2000 (PEG2000 DMG).

In some embodiments, the wt/wt ratio of lipid to mRNA is from about 1:100 to about 100:1. In some embodiments, the one or more first polynucleotides, the one or more second polynucleotides, the one or more third polynucleotides, or any combination thereof, are formulated in separate compositions or the same composition.

Disclosed herein include pharmaceutical compositions. In some embodiments, the pharmaceutical composition comprises a nucleic acid composition of the disclosure, and at least one pharmaceutically acceptable excipient. Disclosed herein include nucleic acid compositions and pharmaceutical compositions for use as a medicament. Disclosed herein include nucleic acid compositions and pharmaceutical compositions for use in a method of treating cancer.

Disclosed herein include methods of treating a disease or disorder characterized by aberrant signaling of one or more oncoproteins. In some embodiments, the method comprises: expressing a synthetic protein circuit of the disclosure in a cell of a subject in need thereof.

Disclosed herein include methods of treating a disease or disorder characterized by aberrant signaling of one or more oncoproteins. In some embodiments, the method comprises: administering any of the nucleic acid compositions or the pharmaceutical compositions of the disclosure to a subject in need thereof.

In some embodiments, the disease or disorder is associated with expression of an oncoprotein. In some embodiments, the disease associated with expression of an oncoprotein is selected from the group consisting of a proliferative disease, a precancerous condition, a cancer, and a non-cancer related indication associated with expression of the oncoprotein. In some embodiments, the disease is cancer. In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia. In some embodiments, the cancer is a β-catenin associated cancer, a p53 associated cancer and/or a Myc associated cancer.

In some embodiments, the cancer is a RAS-associated cancer. In some embodiments, at least a portion of cancer cells of the subject comprise one or more RAS mutations. In some embodiments, the one or more RAS mutations comprises a mutation in KRAS, NRAS, and/or HRAS. In some embodiments, the KRAS mutation is selected from the group consisting of G12C, G12V, G12S, G12A, G12D, G12R, G13V, G13C, G13D, Q61P, Q61L, Q61H, Q61K, Q61E, Q61R, A146V, A146P, and A146T. In some embodiments, the HRAS mutation is selected from the group consisting of G12V, G12C, G12S, G12A, G12D, G12R, G13V, G13dup, G13C, G13S, G13D, Q61L, Q61H, Q61K, and Q61R. In some embodiments, the NRAS mutation is selected from the group consisting of G12A, G12C, G12D, G12R, G12S, G12V, G13A, G13C, G13D, G13R, G13S, G13V, Q61E, Q61H, Q61K, Q61L, Q61P, and Q61R. In some embodiments, the at least a portion of cancer cells of the subject comprising one or more RAS mutations do not require RAS and/or RAS signaling for survival. In some embodiments, the subject has not received a prior cancer treatment. In some embodiments, the subject has not received a prior cancer treatment comprising administration of a RAS-targeting drug. In some embodiments, the subject has received at least one prior cancer treatment. In some embodiments, the prior cancer treatment comprises administration of a RAS-targeting drug. In some embodiments, the subject has developed resistance to a RAS-targeting drug. In some embodiments, the RAS targeting drug comprises Sotorasib, RMC-7977, or both.

In some embodiments, the method comprises administration of at least one additional anti-cancer agent to the subject. In some embodiments, the additional anti-cancer agent comprises a chemotherapeutic agent, an immunomodulatory drug, an immune metabolism modifying drug, a targeted therapy, radiation, an anti-angiogenesis agent, an agent that reduces immune-suppression, or any combination thereof. In some embodiments, the chemotherapeutic agent comprises an alkylating agent, a topoisomerase inhibitor, an anti-metabolite, an anti-mitotic agent, or any combination thereof. In some embodiments, the administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

In some embodiments, a tumor volume of the subject after treatment with the synthetic protein circuit, the composition, or the pharmaceutical composition decreases at least 50% relative to the tumor volume with no treatment or a previous treatment with the RAS targeting drug. In some embodiments, the subject achieves a complete response. In some embodiments, the subject is a mammal. In some embodiments, the mammal is human.

Disclosed herein include methods of detecting an oncoprotein in a cell. In some embodiments, the method comprises: expressing a synthetic protein circuit of the disclosure in the cell or contacting the cell with a nucleic acid composition of the disclosure, wherein the activation of the effector module in the cell is an indication of the presence and/or amount of the oncoprotein in the cell.

In some embodiments, the method is performed in vitro, in vivo, or ex vivo. In some embodiments, the method comprises administration of the nucleic acid composition to a subject that has or is suspected of having cancer, and wherein activation of the effector module in one or more cells of the subject is an indication of the cancer status of the subject. In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia. In some embodiments, the cancer is a β-catenin associated cancer, a p53 associated cancer and/or a Myc associated cancer.

In some embodiments, the cancer is a RAS-associated cancer. In some embodiments, at least a portion of cancer cells of the subject comprise one or more RAS mutations. In some embodiments, the one or more RAS mutations comprises a mutation in KRAS, NRAS, and/or HRAS. In some embodiments, the KRAS mutation is selected from the group consisting of G12C, G12V, G12S, G12A, G12D, G12R, G13V, G13C, G13D, Q61P, Q61L, Q61H, Q61K, Q61E, Q61R, A146V, A146P, and A146T. In some embodiments, the HRAS mutation is selected from the group consisting of G12V, G12C, G12S, G12A, G12D, G12R, G13V, G13dup, G13C, G13S, G13D, Q61L, Q61H, Q61K, and Q61R. In some embodiments, the NRAS mutation is selected from the group consisting of G12A, G12C, G12D, G12R, G12S, G12V, G13A, G13C, G13D, G13R, G13S, G13V, Q61E, Q61H, Q61K, Q61L, Q61P, and Q61R.

In some embodiments, the administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. In some embodiments, the subject is a mammal. In some embodiments, the mammal is human.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 displays an exemplary schematic showing the disclosed engineered protein circuits selectively target cancer cells. This schematic illustration represents idealized therapeutic circuit behavior. (1) Therapeutic circuits composed of sensor and effector modules can be delivered to cells as mRNA in lipid nanoparticles (LNPs). (2) Sensors activate selectively upon binding mutant Ras clusters. More specifically, Ras clustering co-recruits complementary split protease sensor halves to the cell membrane, leading to protease reconstitution (lower panel). (3) Active sensors trigger cell-death effectors and initiate apoptotic (left) or pyroptotic (right) cell death in Ras-mutant cells. Pyroptosis can further stimulate the immune system to kill non-transfected cells (arrows). (4) Circuits eliminate Ras-driven tumors without harming healthy tissue.

FIG. 2A-FIG. 2G display non-limiting exemplary schematics and data showing engineered sensors specifically respond to mutant Ras. As shown in FIG. 2A, an ideal sensor should reconstitute a split protease effector in response to mutant but not wild-type Ras. In FIG. 2B, to screen for sensitive and specific Ras sensors, HEK293 cells were transiently transfected with ectopic Ras variants (marked by mRuby3), candidate sensors (marked by mTagBFP2, labeled BFP), and a protease-activatable fluorescent reporter (IFP/GFP ratio denotes reporter activation). This setup enabled quantitative analysis of sensor performance across a multi-dimensional space of sensor and Ras expression levels. As shown in FIG. 2C, the responses of candidate Ras sensors were characterized against mutant (KRAS^{G12c}) and wild-type Ras (KRAS^{WT}). Data points represent median reporter activation in high Ras expression regimes (Methods). Sensors are color-coded by the type of Ras binding domain (legend). Horizontal and vertical dotted lines denote reporter activation in the absence of a sensor (negative control). FIG. 2D displays data and schematics showing Sensor v2 (12VC1-based) maintains high sensitivity to mutant Ras across a broad range of ectopic Ras expression levels, with minimal background activation in wild-type Ras-expressing cells. Data points represent median reporter activation (Methods). Negative control line represents the activity of a sensor containing GSGSGS (SEQ ID NO: 359) peptides as binders. Structures depict binders in complex with KRAS (RAF1-1: structure generated by AlphaFold3, K13: structure from PDB entry 6H46, 12VC1: structure from PDB entry 7LOG). FIG. 2E shows exemplary data related to v2 sensor detects a broad range of oncogenic RAS variants. Top: v2 (purple) exhibits enhanced sensitivity to a wide range of oncogenic KRAS (left) and HRAS (right) mutations compared to v1 (gray). Bars show the fold-change sensor activation in response to mutant relative to wild-type Ras after gating for high Ras expression (Methods). Bottom: v1 exhibited more uniform response to diverse Ras mutations compared to v2. Data points correspond to the fold-change sensor activation to mutant relative to wild-type Ras. Boxplots show median (center line), quartiles (box), and 1.5× interquartile range (line). Dotted line corresponds to equal responses to mutant and wild-type Ras. FIG. 2F displays non-limiting exemplary data showing at the single-cell level, the v2 sensor discriminated between mutant and wild-type Ras-expressing cells. Receiver operating characteristic (ROC) analysis was performed by varying the protease reporter threshold, generating a confusion matrix, and plotting the true positive rate (Ras-mutant cells above reporter threshold) against the false positive rate (wild-type Ras cells above reporter threshold) at matched Ras expression levels. Dotted line represents the performance of a random classifier. FIG. 2G displays a chart showing the v2 sensor detects endogenous mutant Ras levels. Sensors and fluorescent protease reporter were transiently transfected into cancer cell lines with endogenous levels of Ras and analyzed by flow cytometry. The human cancer cell lines MIA PaCa-2 (KRAS^{G12C}), NCI-H358 (KRAS^{G12C}), and NCI-H441 (KRAS^{G12v}) express mutant Ras, while HEK293, PLC/PRF/5, SNU-423, SNU-449, and SNU-475 express wild-type KRAS. Negative control line represents the activity of a sensor containing GSGSGS (SEQ ID NO: 359) peptides as binders. Positive control line represents a sensor containing the P3/P4 coiled-coil domains as binders. Error bars and shaded error regions in panels FIG. 2D, FIG. 2E, and FIG. 2G were computed as bootstrap 95% confidence intervals of the median (Methods). FIG. 2C, FIG. 2D-FIG. 2F were computed in cells with intermediate sensor expression levels.

FIG. 3A-FIG. 3D display non-limiting exemplary schematics and data showing cis and trans protease regulation increases sensitivity. FIG. 3A displays an exemplary schematic showing single-protein sensor designs facilitate 1:1 stoichiometry between sensor components to improve sensor performance. As shown in FIG. 3B, Sensor v2-s improves mutant Ras discrimination through “self-cleaving” mechanism (cis regulation) inspired by natural viral mechanisms. Some sensor designs incorporated mutant TEV protease variants (eTEV, uTEV3) with elevated catalytic efficiencies. Median sensor responses (IFP/GFP ratio) were measured in on-target (KRAS^{G12C}-expressing MIA PaCa-2) and off-target (wild-type KRAS-expressing HEK293) cells. As shown in FIG. 3C, amplifier proteases (TVMVP) can be activated by TEVP cleavage. In this design, both halves of split TVMVP are fused to their complementary, catalytically dead halves and held together by a weak coiled-coil interaction, thus intramolecularly caging and inhibiting TVMVP activity. Cleavage by TEVP removes the inactive fragments, allowing the active halves to displace the weak interaction with a stronger coiled-coil pair, reconstituting TVMV protease. FIG. 3D displays exemplary graphs showing protease amplifier increases output compared to non-amplified circuits when expressed at different levels (individual plots). Response curves are shown across low (gating for co-transfected Ruby between 10³-10⁴ a.u.), medium (104-10′ a.u.), and high (10⁵-10⁶ a.u.) amplifier expression bins.

FIG. 4A-FIG. 4E display non-limiting exemplary schematics and data showing therapeutic circuits potently and selectively eliminate Ras-mutant cancer cells. Shown in FIG. 4A is an in vitro treatment scheme for therapeutic circuits. mRNA-encoded sensors and effectors were encapsulated in lipid nanoparticles (LNPs) and delivered to cancer cells. Cell viability was measured 3-5 days post-treatment using CellTiter-Glo. As shown in FIG. 4B, engineered sensor v2-s conditionally activates apoptosis (via engineered Casp-3) or pyroptosis (via engineered Gasdermin A, GSDMA) effectors in cells harboring mutant Ras. Heatmaps show selective killing of mutant Ras-expressing (MIA PaCa-2) vs. wild-type (HEK293) cells across different sensor and effector mRNA-LNP concentrations (n=3 replicates, mean cell viability depicted). FIG. 4C displays exemplary data showing a circuit composed of sensor v2-s and engineered Casp-3 selectively eliminates cancer cells harboring mutant Ras (MIA PaCa-2, NCI-H358, SW1573, OV56; all KRAS^{G12C}), but spares wild-type Ras-expressing control cell lines (SNU398, SNU475, HEK293; all KRAS^{WT}). Viability expressed as luminescence normalized to no sensor controls; error bars represent mean±sd (n=3 replicates). FIG. 4D shows a graph of circuit responsiveness to endogenous Ras signaling induced by EGF stimulation. Ras activation was quantified as the percentage of pERK-positive cells measured by antibody staining and flow cytometry. Error bars for pERK measurements represent 95% confidence intervals derived from bootstrapping (1,000 iterations). Cell viability was assayed with CellTiter-Glo; error bars represent mean±sd (n=2 replicates). FIG. 4E shows the results of RNA-seq on HEK293 cells treated with RMC-7977 or circuit at day 1 or day 4 post-treatment.

FIG. 5A-FIG. 5D display non-limiting exemplary schematics and data showing therapeutic circuits prevent and treat liver tumors in vivo. FIG. 5A displays a schematic of an experiment showing circuit composed of v1 sensor, amplifier, and engineered Casp-3 prevents tumor formation in vivo. Tumors were generated by co-administration of plasmids encoding NRAS^{G12V}, TP53 shRNA, and a sleeping beauty transposase via hydrodynamic tail vein injection (HDT). Liver tumor burden was quantified using two independent metrics: (1) surface tumor area, manually annotated following blinded evaluation, and (2) liver-to-body weight ratio, a standard quantitative measure in hydrodynamic liver tumor models. FIG. 5B displays exemplary data showing apoptosis circuits prevent tumor formation in vivo. Top: mouse livers (n=13-16 female mice per condition) extracted 36-40 days after tumor induction. Liver images and manually annotated surface tumor nodules (red) are displayed side-by-side. Bottom left: quantification of total surface tumor area (blinded manual annotation of tumor nodules). Bars depict mean tumor surface area (px)±se (error bars). Individual data points denote accumulated surface tumor area of individual animals from anterior and posterior views. Adjusted p value=4.1×10⁻³ for PBS vs. no amplifier; 3.2×10⁻⁶ for PBS vs. full circuit; 3.2×10⁻⁶ for no amplifier vs. full circuit (pairwise Mann-Whitney U tests with Benjamini-Hochberg (BH) correction, two-sided). Bottom middle: bars depict mean liver/body weight ratio (%)±se (error bars). Data points denote liver/body weight ratio of individual animals. Adjusted p value=2.8×10⁻³ for PBS vs. no amplifier; 3.5×10⁻⁷ for PBS vs. full circuit; 1.5×10⁻⁵ for no amplifier vs. full circuit (pairwise Mann-Whitney U tests with BH correction, two-sided). Bottom right: overall survival depicted as Kaplan-Meier curves (group 2; n=7-10 male mice per condition). Median survival 42 days for PBS; 59 days for no amplifier; 145 days (termination of experiment) for full circuit. P value=7×10⁴ (***) for PBS vs. no amplifier; 1×10⁴ (****) for PBS vs. full circuit; 1×10⁻⁴ (****) for no amplifier vs. full circuit (log-rank test, all p values significant after Bonferroni correction). FIG. 5C displays an in vivo treatment scheme for therapeutic circuits. mRNA-encoded sensors and effectors were encapsulated in lipid nanoparticles (LNPs) and intravenously administered on days 5, 6, and 7 following tumor induction. On day 26, mice were sacrificed and livers were collected for evaluation of tumor burden. FIG. 5D displays non-limiting exemplary data related to systemic intravenous delivery of pyroptosis circuit v1 (v1 sensor, amplifier, sensor-activatable gasdermin D; GSDMD) induced robust anti-tumor activity (n=10-11 female mice per condition). P value=0.00055 when comparing distribution of surface tumor areas between PBS and circuit v1; 0.00078 when comparing distribution of liver/body weight ratios (Mann-Whitney U test, two-sided). Bars depict mean±se (error bars). Individual data points denote mean surface tumor area (px) and liver/body weight ratios (%), respectively.

FIG. 6A-FIG. 6E display non-limiting exemplary schematics and data showing circuits are less susceptible to intrinsic and acquired resistance compared to Ras-targeting drugs. FIG. 6A displays a schematic showing circuits can directly induce cancer cell death, potentially minimizing susceptibility to resistance mechanisms that limit targeted therapies. Circuits used in this figure were composed of sensor v2-s and Casp-3. If not specified otherwise, circuit concentrations denote total amounts of sensor v2-s and Casp-3 added at a 1:1 ratio. FIG. 6B displays exemplary schematics and data related to dose-response curves for Ras inhibitors (Sotorasib, RMC-7977) and circuit. Viability measured after 3 days (CellTiter-Glo, mean±sd, n=3-4). Curves were fitted with a four-parameter log-logistic model (Methods). Shown in FIG. 6C are exemplary images from co-culture of MIA PaCa-2 (KRAS^{G12C}) and HEK293 (wild-type KRAS) cells stably expressing GFP and mRuby3, respectively. Cells were treated with saturating concentrations of Paclitaxel (30 μM), RMC-7977 (1 μM), Sotorasib (1 μM), or the therapeutic circuit (150 pg/μL Casp-3 and 100 pg/μL sensor v2-s). Fluorescence images were acquired on day 5. Scale bar, 200 μm. FIG. 6D shows graphs of efficacy comparison between circuits and Ras inhibitors in cell lines that differ in Ras addiction. Dashed lines represent 0% and 100% cell viability, respectively. For each cell line/RAS status, shown from left to right is: Sotorasib (1 μM), RMC (1 μM), Circuit (320 pg/μL). As shown in FIG. 6E, repeated exposure to Sotorasib or the therapeutic circuit alters subsequent treatment responsiveness in MIA PaCa-2 cells. Cells were treated every 5 days for 3 weeks, then challenged with both Sotorasib and circuit components. Changes in EC₃₀ values reflect acquired resistance or sensitization. Data are presented as fold-change in EC₃₀ (mean±sd, n=4 replicates). The sum-of-squares F-test was used to determine if selection treatment yields significantly altered dose-response curves from cells that did not undergo selection treatment: p value≈0, F value=141 for Sotorasib selection and challenge treatment; p value=5×10⁻¹⁴, F value=43 for circuit selection and challenge treatment; p value≈0, F value=235 for Sotorasib selection and circuit challenge treatment; p value=0.11, F value=2 for circuit selection and Sotorasib challenge treatment.

FIG. 7 displays a graph of data showing previous RAF1-based Ras sensor (v1) insufficiently discriminated mutant and wild-type Ras. When highly expressed (co-transfected mTagBFP2 between 10⁵-10⁶ a.u.), the v1 sensor activates in a non-specific fashion due to sensor multimerization. In a medium sensor expression regime (co-transfected mTagBFP2 between 10⁴-10⁵ a.u.), the v1 sensor shows low sensitivity and specificity. Sensor performance was evaluated following the experimental workflow outlined in FIG. 2B. High Ras expression corresponds to mRuby3 fluorescence (co-transfected with ectopic Ras) of 10^{5.5}-10^{6.5} a.u.; medium Ras expression to 10^{4.5}-10^{5.5} a.u. Bar plots represent median sensor protease activities; error bars denote 95% confidence intervals calculated by bootstrapping (1,000 iterations). Shown from left to right for each condition is KRAS G12C, KRAS WT, and No Ras (Neg. Ctrl.).

FIG. 8A-FIG. 8C display non-limiting exemplary schematics and data related to a Ras binder screen to identify Ras-mutant-specific sensors. FIG. 8A shows a workflow used for sensor screening and quantitative analysis of flow cytometry data. After generation of binder library and experimental screening by flow cytometry, sensor activation was determined as follows: cells that received the sensor (as marked by mTagBFP2 fluorescence between 10⁴-10⁶ a.u.; in the following referred to as median sensor expression) and expressed high levels of ectopic Ras (as marked by mRuby3 fluorescence between 10⁵-10⁶ a.u.) were selected. Further, we gated for cells with protease reporter expression between 10⁵-10⁶ a.u. (as marked by reporter GFP fluorescence), corresponding to the regime with the highest reporter dynamic range. Finally, median protease activity was calculated as the fraction of activated protease reporter proteins (IFP/GFP). Confidence estimates were obtained by bootstrapping. As shown in FIG. 8B, a library screen enables characterization of sensor activity against mutant KRAS^{G12C} compared to a no Ras control. Data points represent median reporter activation in high Ras and medium sensor expression regime. Sensors are color-coded by the category of Ras binding domain. Text denotes binding domain used for each sensor. Negative control line denotes reporter activation in the absence of a sensor. As shown in FIG. 8C, library screen enables characterization of sensor activity against wild-type KRAS compared to a no Ras control. Data points represent median reporter activation in high Ras and medium sensor expression regime. Sensors are color-coded by the category of Ras binding domain. Text denotes binding domain used for each sensor. Negative control line denotes reporter activation in the absence of a sensor.

FIG. 9A-FIG. 9C display non-limiting exemplary data showing Ras expression dependency of individual sensor responses. FIG. 9A shows graphs of sensor responses of natural Ras binder-based sensors. Shown in FIG. 9B are graphs of responses of sensors incorporating de novo Ras binders (DNB). Shown in FIG. 9C are graphs of responses of sensors utilizing previously published synthetic Ras binders, nanobodies, or monobodies. In FIG. 9A-FIG. 9C, headings specify binding domains used for each sensor module. Data points represent median reporter activation in medium sensor expression regime. Negative control line represents the activity of a sensor containing GSGSGS (SEQ ID NO: 359) peptides as binders. Shaded areas depict 95% confidence intervals from bootstrapping (1,000 iterations). Arrows are included in some graphs to denote KRAS G12C.

FIG. 10A-FIG. 10C display non-limiting exemplary data related to characterization of Ras sensors v1 and v2. Shown in FIG. 10A is a comparison of sensor v1 and v2 responses across a broad spectrum of NRAS oncogenic mutations. Top: Bars show the fold-change sensor activation in response to mutant relative to wild-type Ras after gating for high Ras and intermediate sensor expression. Bottom: v1 exhibited more uniform response to diverse Ras mutations compared to v2. Data points correspond to the fold-change sensor activation to mutant relative to wild-type Ras. Boxplots show median (center line), quartiles (box), and 1.5× interquartile range (line). Dotted line corresponds to equal responses to mutant and wild-type Ras. FIG. 10B displays graphs showing sensor v2 is sensitive to a range of KRAS, HRAS, and NRAS oncogenic mutations at different sensor and Ras expression levels. Data points represent median reporter activation. Negative control line represents the activity of a sensor containing GSGSGS (SEQ ID NO: 359) peptides as binders. Shaded areas depict 95% confidence intervals from bootstrapping (1,000 iterations). As shown in FIG. 10C, sensors can classify the Ras mutational status of single cells. Area under curve values are obtained by ROC analysis (Methods).

FIG. 11 displays an exemplary schematic and data related to the design and characterization of amplifiers. Schematic overview is shown of rationally designed candidate constructs built from modular components, including split TVMVP, catalytically inactive TVMVP, coiled-coil zipper domains, and input protease cleavage sites. Design details are provided in Methods. Most candidates showed TEVP-inducible activation with varying levels of background activity. Candidates 15-18, which employed a double-caged TVMVP architecture, achieved low background and high activation.

FIG. 12A-FIG. 12C display non-limiting exemplary schematics and data showing mRNA delivery via LNP and treatment effects on transcriptome. As shown in FIG. 12A, LNPs enable titratable, near-complete transfection of MIA PaCa-2 cells with Citrine mRNA (as measured by flow cytometry). FIG. 12B displays schematics and data showing LNPs enable correlated transfection of multiple proteins. Co-encapsulation of mRuby3 and Citrine mRNAs in a single LNP results (administration of 300 pg/μL mRNA-LNP per well) in comparable correlation between the two proteins (R²=0.79) compared to co-transfection of separately LNP-encapsulated mRuby3 and Citrine mRNAs (R²=0.67, administration of 150 pg/μL mRNA-LNP per mRNA; as measured by flow cytometry). R² denotes squared correlation coefficient. FIG. 12C displays a volcano plot of bulk RNA-sequencing from HEK293 cells treated with drugs and circuit for 1 or 4 days. RMC-7977 induced more differential gene expression than Sotorasib or the circuit at day 1. By day 4, transcriptomic profiles largely returned to baseline.

FIG. 13 displays non-limiting exemplary images and data related to in vivo tumor prevention. In vivo co-delivery of tumor inducing agents and circuits for evaluation of tumor prevention. Hydrodynamic tail vein injection was used to efficiently transfect hepatocytes in mice. Tumor-inducing agents were co-delivered with circuit plasmid DNA. Mice receiving no circuit developed multifocal, aggressive liver tumors. Inclusion of Sensor v1 and Casp-3 provided partial inhibition of tumor initiation. The full circuit, including the amplifier module, completely blocked tumor formation. Surface tumor nodules were manually annotated. View 1 and 2 denote front and back view of livers.

FIG. 14A-FIG. 14D display non-limiting exemplary graphs of quantification of effects in tumor prevention and mRNA-LNP treatment experiments. Distribution of liver tumor burden across treatment groups is shown in FIG. 14A and FIG. 14C. Top: liver-to-body weight ratio. Bottom: total tumor surface area. Shown in FIG. 14B and FIG. 14D are empirical cumulative distribution function (ECDF) of tumor burden for each treatment group.

FIG. 15 displays gross images of mouse livers after mRNA-LNP treatment. Mice were first induced with tumor-initiating agents, and tumors were allowed to develop before treatment. PBS-treated animals developed more extensive tumors than those receiving the full circuit, indicating therapeutic efficacy. Surface tumor nodules were manually annotated. View 1 and 2 denote front and back view of livers.

FIG. 16A-FIG. 16C display graphs related to comparison of circuit and Ras inhibitors Sotorasib and RMC-7977. As shown in FIG. 16A, killing efficacy of the mRNA-LNP circuit (v2-s sensor, Casp-3) is independent of plating density in MIA PaCa-2 (KRAS^{G12C}) cells stably expressing GFP. Viability (mean±sd, n=3 replicates) was determined by flow cytometry; live cells were defined as GFP high (>10⁵ a.u.) and Annexin V low (<10⁴ a.u.). The circuit induces sustained depletion of SW1573 cells over 7 days, while Sotorasib- and RMC-7977-treated populations rebound after 3-5 days (FIG. 16B). Viability was measured using CellTiter-Glo (mean±sd, n=2 replicates). As shown in FIG. 16C, transient Ras overexpression enhances circuit sensitivity and reduces Sotorasib efficacy. MIA PaCa-2 cells were co-treated with 200 pg/μL KRAS^{G12C} or NeoR (control) mRNA-LNP and either Sotorasib or the circuit (v2-s sensor, Casp-3). Viability (mean, n=4 replicates) was assessed; dotted lines indicate EC₃₀ values, and fold-change in response due to Ras overexpression is annotated.

FIG. 17A-FIG. 17C display non-limiting exemplary data related to cytotoxicity of engineered protein circuits. FIG. 17A shows a graph of cell viability, normalized to cell counts at treatment (day 0), as measured by CellTiter-Glo. Points show mean viability, error bars show standard deviation across replicates. Treatment concentrations: 750 pg/μL sensor+125 pg/μL Caspase-3 (circuit), 1 μM Sotorasib, 1 μM RMC-7977. FIG. 17B displays a graph of the fraction of treated MIA PaCa-2 cells with activated caspase-3 (cc3 antibody, clone C92-605.rMAb) as measured 12 h after treatment. Treatment concentrations: 400 pg/μL circuit, 1 μM RMC-7977. Apoptosis induction in MIA PaCa-2 cells quantified by Annexin V staining and flow cytometry (FIG. 17C). Flow cytometry was performed 24 h after treatment with 400 pg/μL circuit, 1 μM Sotorasib, and 1 μM RMC-7977.

FIG. 18A-FIG. 18C display non-limiting exemplary data related to ERK-independence of Ras-sensing protein circuits. Shown are graphs of change in fraction of cells that are pERK+ or active Casp3+ from DMSO negative control or LNP negative control containing filler mRNA. Assays are performed via “Cell staining with intracellular antibodies and flow cytometry” protocol. Drug concentrations are either denoted on plot, or treated at 1 μM concentration. Circuits are treated at 400 pg/μL total.

FIG. 19 displays a graph showing sensor modularity enables retargeting to user-defined mutations. v2-s and modified v2-s sensors were expressed in HEK293FT cells with wild-type or mutant Ras. The iProtease reporter system was used to read out sensor activation. Flow cytometry was performed 48 h after DNA transfection, as described in Methods below.

FIG. 20 displays graph showing membrane localization plays a key role in the activation of Ras-targeting circuits. v2-s sensors were expressed in HEK293FT cells with Ras variants as described in the panel. The iProtease reporter system was used to read out sensor activation. Flow cytometry was performed 48 h after DNA transfection, as described in Methods below.

FIG. 21 displays a graph of number of TEV protease cleavage site occurrences in the human proteome.

FIG. 22A-FIG. 22C display non-limiting exemplary data showing circuits do not perturb Caco-2 cells. RNAseq captures biological changes expected from circuit expression and drug treatment (FIG. 22A). Circuits are less perturbative than drugs (FIG. 22B). Circuits avoided Ras-MAPK pathway suppression and proliferation inhibition caused by RMC-7977 (FIG. 22C).

FIG. 23 displays non-limiting exemplary data showing sensors remain effective against clinically relevant inhibitor-resistant Ras mutations. v2-s sensors were expressed in HEK293FT cells with Ras variants as described in the panel. The iProtease reporter system was used to read out sensor activation. Flow cytometry was performed 48 h after DNA transfection, as described in Methods below.

FIG. 24 displays non-limiting exemplary data showing split TEV protease does not spontaneously reconstitute. v2-s and modified v2-s sensors were expressed in HEK293FT cells with Ras variants as described in the panel. The iProtease reporter system was used to read out sensor activation. Flow cytometry was performed 48 h after DNA transfection, as described in Methods below.

FIG. 25 displays non-limiting exemplary data showing sensor does not activate in response to GDP-bound KRAS, and concurrent Sotorasib administration abolishes sensor activity. v2-s sensors were expressed in HEK293FT cells with Ras variants as described in the panel. The iProtease reporter system was used to read out sensor activation. Sotorasib was used at a treatment concentration of 1 μM. Flow cytometry was performed 48 h after DNA transfection, as described in Methods below.

FIG. 26 displays non-limiting exemplary data showing cell viability of human wild-type Ras cell lines in response to circuit and RMC-7977 treatment. Cell viability was measured by CTG 3 days after treatment. A seeding concentration of 2 k cells/well was used. Treatment was administered immediately after seeding. Bars denote mean cell viability; error bars denote standard deviation across replicates.

FIG. 27 displays non-limiting exemplary data showing Z-VAD-FMK administration rescues circuit-induced cell death. Active caspase-3 was measured by intracellular staining and flow cytometry 12 hours after treatment in MIA PaCa-2 cells, using the protocol as described below. For each LNP concentration, Z-VAD-FML at 0 μM and 80 μM is shown from left to right.

FIG. 28 displays non-limiting exemplary data showing in vivo NRAS tumor suppression with IV-delivered mRNA-LNP protein circuits. 12 8-week-old female FVB mice were subjected to NRAS hydrodynamic tail vein injection (HTVi) on Day 0 and subsequently dosed intravenously with mRNA-LNP formulations encoding the indicated circuits (1.5 mg/kg total mRNA per dose) according to the dosing schedule shown. At Day 21, animals were euthanized, and livers were harvested for assessment of tumor burden and organ mass. Representative gross liver images are shown from both anterior and posterior views; the five livers exhibiting the highest visible tumor burden are depicted. Liver-to-body weight ratio was calculated at necropsy (day 21) and plotted for each treatment group, with statistical significance indicated on the graph. On the graph, shown from left to right: Pyroptosis circuit, Apoptosis circuit, N/C TEVP Only LNP, and PBS.

FIG. 29 displays non-limiting exemplary data showing in vivo KRAS tumor suppression with IV-delivered mRNA-LNP protein circuits. 11 8-week-old female FVB mice were subjected to KRAS hydrodynamic tail vein injection (HTVi) on Day 0 and subsequently dosed intravenously with mRNA-LNP formulations encoding the indicated circuits (1.5 mg/kg total mRNA per dose) according to the dosing schedule shown. At Day 18, animals were euthanized, and livers were harvested for assessment of tumor burden and organ mass. Representative gross liver images are shown from both anterior and posterior views. Liver-to-body weight ratio was calculated at necropsy (day 18) and plotted for each treatment group, with statistical significance indicated on the graph.

FIG. 30 displays exemplary schematics related to catalytic execution vs. stoichiometric inhibition.

FIG. 31 displays a graph of KRAS G12C occupancy vs. cell viability.

FIG. 32 displays non-limiting exemplary data related to sensitivity of the enclosed circuits and methods as compared to RAS targeting drugs.

FIG. 33 displays non-limiting exemplary data related to effects on ERK signaling.

FIG. 34 displays non-limiting exemplary data related to oncogene addiction.

FIG. 35 displays non-limiting exemplary data related to oncogene addiction. For each Ras status/addicted point, shown from left to right is: Sotorasib—Ras G12C inhibitor (1 μM), RMC-7977—Pan-Ras inhibitor (1 μM), and Circuit (320 pg/μl).

FIG. 36 displays non-limiting exemplary data related to reduced susceptibility of the disclosed circuits to resistance.

FIG. 37 displays non-limiting exemplary data showing sensors detect overexpressed beta-catenin. Sensors composed of two beta-catenin binding domains (CDBs, x-axis) fused to split TVMV protease halves were transfected into cells overexpressing beta-catenin (blue dots: WT beta-catenin; orange dots: S33Y beta-catenin) or a negative control (gray dots, NeoR). The dimerizing domains P3/P4 serve as positive controls. Sensor activation was measured by tracking iProtease reporter activation (as implemented in Methods below) with flow cytometry 48 h after DNA transfection into HEK293FT cells. Data is displayed after gating for similar beta-catenin and sensor expression levels, as determined by co-transfection markers. Unless annotated as “full length”, CDBs are derived from beta-catenin-binding domains of the corresponding proteins. “NB” refers to nanobodies introduced in Traenkle et al, Mol. Cell. Proteomics 14, 707-723 (2015).

FIG. 38 displays non-limiting exemplary data showing sensors can discriminate between beta-catenin wild-type and mutant cancer cell lines. Sensors composed of two beta-catenin binding domains (blue (left data point): ECAD+AXIN1; orange (middle data point): ICAT+AXIN1; green (right data point): TCF7+BCL9; binding domains were derived from beta-catenin-binding domains of the corresponding proteins) fused to split TVMV protease halves. Cancer cell lines were transfected with sensor DNA, and sensor activation was measured by tracking iProtease reporter activation (as implemented in Methods below) with flow cytometry 48 h after DNA transfection into HEK293FT cells. Data is displayed after gating for similar sensor expression levels, as determined by co-transfected BFP. P.C. and N.C. denote positive (P3/P4) and negative (NeoR) controls, respectively.

FIG. 39A-FIG. 39B display non-limiting exemplary data showing sensor activation depends on both beta-catenin and sensor concentration. Shown in FIG. 39A is data related to sensor (same sensors and experimental layout as in FIG. 37) response to different beta-catenin levels (x-axis). Hook effect is visible at high beta-catenin concentrations. FIG. 39B shows data related to sensor (same sensors and experimental layout as in FIG. 38) response depends on sensor levels. P.C. and N.C. denote positive (P3/P4) and negative (NeoR) controls, respectively.

FIG. 40 displays a graph showing protein domains inhibit Wnt signaling. Transfection of protein domains (subplot titles) into HEK293FT cells can inhibit Wnt signaling (orange bar, 2^{nd} data point on the x-axis) compared to no Wnt ligand control (blue bar, 1^{st} data point on the x-axis) without interfering with beta-catenin degradation machinery in general (green bar, 3^{rd} data point on the x-axis). Beta-catenin levels were read out with TOP/FOP luminescence reporter system.

FIG. 41 displays non-limiting exemplary data showing sensors detect p53 concentration. Sensors composed of p53-binding nanobody NB139 fused to split TEV protease halves (orange, gray, black lines) were transfected into cells overexpressing p53 (blue, orange, green: WT p53; others: mutant p53 as annotated in legend). NeoR and P3/P4 fused to TEVP halves were used as negative and positive controls, respectively, instead of p53 sensors. Sensor activation was measured by tracking iProtease reporter activation (as implemented in Methods below) with flow cytometry 48 h after DNA transfection into HEK293FT cells. Data is displayed after gating for similar sensor expression levels across conditions, as determined by the co-transfection marker BFP. The x-axis depicts p53 concentrations as determined by co-transfected Ruby.

FIG. 42A-FIG. 42B display data related to detection of protein mutations. FIG. 42A displays results of an initial screen of de novo binders against the p53 cryptic epitope. Approximately 90 de novo protein binders designed against the cryptic epitope of the p53 DNA-binding domain were each fused to one half of a split protease, while a construct presenting the cryptic epitope was fused to the complementary half. Sensor activation was measured by tracking iProtease reporter activation (as implemented in Methods) with flow cytometry 48 h after DNA transfection into HEK293FT cells. The blue dashed line indicates the response threshold; approximately 20 binders exceeding this threshold were selected for further characterization. FIG. 42B displays results of specificity testing of top de novo binders against cryptic epitope variants. Top binders from the initial screen (FIG. 42A), each fused to one half of a split protease, were co-expressed with target constructs fused to the complementary split protease half: a negative control (NLS-NeoR, gray, 1^{st} bar for each binder), the wild-type cryptic epitope (NLS-NeoR-FRHSV, blue, 2^{nd} bar for each binder), or a mutated epitope variant (NLS-NeoR-FAASV, red, 3^{rd} bar for each binder). Known p53 nanobodies (NLS-NB139 variants) served as reference controls. Several de novo binders exhibited high activation against the cryptic epitope with low activation against both control conditions, demonstrating conformational epitope specificity. Sensor activation was measured by tracking iProtease reporter activation (as implemented in Methods below) with flow cytometry 48 h after DNA transfection into HEK293FT cells. Data is displayed after gating for similar sensor expression levels, as determined by a co-transfection marker.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include synthetic protein circuits. In some embodiments, the synthetic protein circuit comprises: (a) a sensor module comprising: a first polypeptide comprising a first oncoprotein binding domain and a first part of a first orthogonal protease domain, wherein the first oncoprotein binding domain is capable of binding a first oncoprotein to form a first oncoprotein-bound polypeptide; a second polypeptide comprising a second oncoprotein binding domain and a second part of the first orthogonal protease domain, wherein the second oncoprotein binding domain is capable of binding a second oncoprotein to form a second oncoprotein-bound polypeptide, and wherein the first part of the first orthogonal protease domain and the second part of the first orthogonal protease domain are capable of associating with each other to constitute a first orthogonal protease capable of being in a first orthogonal protease active state when the first oncoprotein and the second oncoprotein are in close proximity at a proximity site, wherein the first oncoprotein and/or the second oncoprotein comprise: (i) a monomer having at least two distinct binding sites; (ii) a dimer having at least two binding sites per dimer in sufficient proximity to promote association; (iii) an oligomer having at least two binding sites per oligomer in sufficient proximity to promote association; (iv) an aggregate having at least two binding sites per aggregate in sufficient proximity to promote association; (v) a multi-protein complex in which two or more constituent proteins collectively provide at least two binding sites in sufficient proximity for association; and/or (vi) an onco-fusion protein, and (b) an effector module, wherein the first orthogonal protease in the first orthogonal protease active state is capable of activating the effector module in a cell.

Disclosed herein include synthetic protein circuits. In some embodiments, the synthetic protein circuit comprises: (a) a sensor module comprising: a first polypeptide comprising a first oncoprotein binding domain and a first part of a first orthogonal protease domain, wherein the first oncoprotein binding domain is capable of binding a first oncoprotein to form a first oncoprotein-bound polypeptide; a second polypeptide comprising a second oncoprotein binding domain and a second part of the first orthogonal protease domain, wherein the second oncoprotein binding domain is capable of binding a second oncoprotein to form a second oncoprotein-bound polypeptide, and wherein the first part of the first orthogonal protease domain and the second part of the first orthogonal protease domain are capable of associating with each other to constitute a first orthogonal protease capable of being in a first orthogonal protease active state when the first oncoprotein and the second oncoprotein are in close proximity at a proximity site, optionally the first and/or second oncoprotein binding domain comprise a sequence at least 85% identical to the sequence of SEQ ID NO: 109 or SEQ ID NO: 111; and (b) an effector module capable of inducing apoptosis or pyroptosis in a cell, wherein the first orthogonal protease in the first orthogonal protease active state is capable of activating the effector module, thereby inducing apoptosis or pyroptosis in the cell.

Disclosed herein include nucleic acid compositions. In some embodiments, the nucleic acid composition comprises: one or more polynucleotides encoding a synthetic protein circuit of the disclosure, optionally the one or more polynucleotides comprise: one or more first polynucleotides encoding the first polypeptide, the second polypeptide, the proprotein, or any combination thereof, and/or one or more second polynucleotides encoding the protease activatable reporter, the apoptosis polypeptide and/or the pyroptosis polypeptide; optionally the one or more polynucleotides further comprise one or more third polynucleotides encoding the amplifier protein, the companion amplifier protein, or any combination thereof.

Disclosed herein include pharmaceutical compositions. In some embodiments, the pharmaceutical composition comprises a nucleic acid composition of the disclosure, and at least one pharmaceutically acceptable excipient.

Disclosed herein include nucleic acid compositions and pharmaceutical compositions for use as a medicament. Disclosed herein include nucleic acid compositions and pharmaceutical compositions for use in a method of treating cancer.

Disclosed herein include methods of treating a disease or disorder characterized by aberrant signaling of one or more oncoproteins. In some embodiments, the method comprises: expressing a synthetic protein circuit of the disclosure in a cell of a subject in need thereof. In some embodiments, the method comprises: administering any of the nucleic acid compositions or the pharmaceutical compositions of the disclosure to a subject in need thereof.

Disclosed herein include methods of detecting an oncoprotein in a cell. In some embodiments, the method comprises: expressing a synthetic protein circuit of the disclosure in the cell or contacting the cell with a nucleic acid composition of the disclosure, wherein the activation of the effector module in the cell is an indication of the presence and/or amount of the oncoprotein in the cell.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “about” means plus or minus 5% of the provided value.

As used herein, the term “vector” refers to a polynucleotide construct, typically a plasmid or a virus, used to transmit genetic material to a host cell (e.g., a target cell). Vectors can be, for example, viruses, plasmids, cosmids, or phage. A vector can be a viral vector. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “construct,” as used herein, refers to a recombinant nucleic acid that has been generated for the purpose of the expression of a specific nucleotide sequence(s), or that is to be used in the construction of other recombinant nucleotide sequences.

The terms “polynucleotide” and “nucleic acid” are used interchangeably herein and refer to a polymeric form of nucleotides of any length, either ribonucleotides or deoxyribonucleotides. A polynucleotide can be single-, double-, or multi-stranded DNA or RNA, genomic DNA, cDNA, DNA-RNA hybrids/triple helices, or a polymer including purine and pyrimidine bases or other natural, chemically or biochemically modified, non-natural, or derivatized nucleotide bases. Any of the RNA sequences disclosed herein may also be DNA (either single-stranded or double-stranded), e.g., wherein “U” is converted to “T.” Any of the DNA sequences disclosed herein may also be RNA, e.g., wherein “T” is converted to “U.”

The term “regulatory element” and “expression control element” are used interchangeably and refer to nucleic acid molecules that can influence the expression of an operably linked coding sequence in a particular organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a cell.

As used herein, 2A sequences or elements refer to small peptides introduced as a linker between two proteins, allowing autonomous intraribosomal self-processing of polyproteins (See e.g., de Felipe. Genetic Vaccines and Ther. 2: 13 (2004); deFelipe et al. Traffic 5:616-626 (2004)). These short peptides allow co-expression of multiple proteins from a single vector. Many 2A elements are known in the art. Examples of 2A sequences that can be used in the methods and system disclosed herein, without limitation, include 2A sequences from the foot-and-mouth disease virus (F2A), equine rhinitis A virus (E2A), Thosea asigna virus (T2A), and porcine tescho virus-1 (P2A).

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “variant” refers to a polynucleotide (or polypeptide) having a sequence substantially similar to a reference polynucleotide (or polypeptide). In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

As used herein, the term “effective amount” refers to an amount sufficient to effect beneficial or desirable biological and/or clinical results.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. For example, in some embodiments treatment may reduce the level of RAS signaling in the subject, thereby to reduce, alleviate, or eradicate the symptom(s) of the disease(s). As used herein, the term “prevention” refers to any activity that reduces the burden of the individual later expressing those RAS-related disease symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, ammo acids, carbohydrates including glucose, mannose, or dextrins, chelating agents such as EDTA, sugar alcohols such as mannitol or sorbitol, salt-forming counterions such as sodium, and nonionic surfactants such as Tween, polyethylene glycol (PEG), and Pluronics. Auxiliary, stabilizer, emulsifier, lubricant, binder, pH adjuster controller, isotonic agent and other conventional additives may also be added to the carriers.

Described herein is a set of engineered protein circuits for therapy, e.g., cancer therapy. The circuits share the core feature that they have the ability to directly rewire cancer-associated protein-level signatures to diverse forms of cancer cell death. This direct rewiring of target detection to cell death activation is demonstrated herein using Ras-mutant cancers as an example, but note that this is a general therapeutic strategy to target diverse human cancers and other diseases.

Described herein are engineered protein circuits that specifically sense cancer-associated signals and conditionally kill corresponding cancer cells. In particular, there are provided circuits that directly sense mutant Ras proteins using a protease-based detection system, effectively distinguishing cancerous from non-cancerous cells, and directly triggering apoptosis or immunogenic pyroptosis. The circuits consist of three main modules: (1) a highly sensitive and specific protease-based sensor using the 12VC1-monobody and other natural, synthetic, and de novo designed Ras binding domains that are responsive to a wide range of clinically relevant Ras isoforms and mutation; (2) an optional protease-activatable protease module for signal amplification; and (3) protease-triggered effector modules that induce either non-inflammatory apoptosis or immunogenic pyroptosis.

Delivered transiently as mRNA encapsulated within lipid nanoparticles (LNPs), these sense-kill circuits have demonstrated robust efficacy against Ras-mutant cancer cell lines in vitro and significantly reduced tumor burden in immunocompetent mouse models with aggressive, multifocal Ras-driven liver tumors. The circuits exhibit robust specificity and minimal activation in Ras wild-type cells, ensuring safety and efficacy. These circuits offer a potent, specific, and resistance-avoiding alternative to existing Ras-targeting therapies, bypassing limitations associated with pharmacological inhibition, overcoming intrinsic and acquired resistance, and potentially synergistically enhancing existing treatments such as checkpoint inhibitors and other immunotherapies.

It is noted that while some Ras-binding domains, including the 12VC1 monobody, were previously publicly available, their therapeutic utility when expressed individually in cancer cells was minimal. Unexpectedly, extensive empirical screening from a broad and diverse library of Ras-binding domains revealed that the incorporation of the 12VC1 monobody specifically into the presently disclosed engineered protease-based sensor conferred strong specificity and sensitivity for mutant Ras. Additionally, a strong interaction of the 12VC1-based Ras sensor was observed with engineered protease-triggered cell death effector proteins described herein. More specifically, delivery of the Ras sensor and engineered cell death effectors achieved nearly complete dose-dependent elimination of Ras-mutant cancer cells, while notably sparing cells expressing wild-type Ras. This unpredictable discovery highlights the inventive step and significant non-obviousness associated with these novel engineered protein circuits.

Synthetic biology allows for rational design of circuits that confer new functions in living cells. For example, CHOMP (circuits of hacked orthogonal modular proteases) enables design of composable protein circuit components. Many natural cellular functions can be implemented by protein-level circuits, in which proteins specifically modify each other's activity, localization, or stability. Synthetic protein circuits have been described in, Gao, Xiaojing J., et al. “Programmable protein circuits in living cells.” Science 361.6408 (2018): 1252-1258; and WO2019/147478; the content of each of these, including any supporting or supplemental information or material, is incorporated herein by reference in its entirety. In some embodiments, synthetic protein circuits respond to inputs only above or below a certain tunable threshold concentration, such as those provided in US2020/0277333, the content of which is incorporated herein by reference in its entirety. In some embodiments, synthetic protein circuits comprise one or more synthetic protein circuit design components and/or concepts of US2020/0071362, the content of which is incorporated herein by reference in its entirety. In some embodiments, synthetic protein circuits comprise rationally designed circuits, including miRNA-level and/or protein-level incoherent feed-forward loop circuits, that maintain the expression of a protein, e.g., a polynucleotide of the disclosure at an efficacious level, such as those provided in US2021/0171582, the content of which is incorporated herein by reference in its entirety. The compositions, methods, systems and kits provided herein can be employed in concert with those described in International Patent Application No. PCT/US2021/048100, entitled “Synthetic Mammalian Signaling Circuits For Robust Cell Population Control” filed on Aug. 27, 2021, the content of which is incorporated herein by reference in its entirety. The systems, methods, compositions, and kits provided herein can, in some embodiments, be employed in concert with the systems, methods, compositions, and kits described in PCT Patent Application Publication No. WO2022/125590, entitled, “A synthetic circuit for cellular multistability,” the content of which is incorporated herein by reference in its entirety. The systems, methods, compositions, and kits provided herein can, in some embodiments, be employed in concert with the systems, methods, compositions, and kits described in U.S. Patent Application No. 2018/0142307 and 2020/0172968, the contents of which are incorporated herein by reference in their entirety. The systems, methods, compositions, and kits provided herein can, in some embodiments, be employed in concert with the systems, methods, compositions, and kits for described in U.S. Patent Publication No. 2023/0076395, entitled, “CELL-TO-CELL DELIVERY OF RNA CIRCUITS,” and in U.S. Patent Publication No. 2023/0071834, entitled, “EXPORTED RNA REPORTERS FOR LIVE-CELL MEASUREMENT,” the contents of which are incorporated herein by reference in their entirety. The systems, methods, compositions, and kits provided herein can, in some embodiments, be employed in concert with the systems, methods, compositions, and kits described in PCT Application No. PCT/US23/69663, entitled, “A SYNTHETIC PROTEIN-LEVEL NEURAL NETWORK IN MAMMALIAN CELLS,” filed Jul. 5, 2023, the content of which is incorporated herein by reference in its entirety. The systems, methods, compositions, and kits provided herein can, in some embodiments, be employed in concert with the systems, methods, compositions, and kits described in PCT Application Publication No. WO2020117713A1, entitled, “In situ readout of dna barcodes,” the content of which is incorporated herein by reference in its entirety. The systems, methods, compositions, and kits provided herein can, in some embodiments, be employed in concert with the systems, methods, compositions, and kits described in U.S. patent application Ser. Nos. 17/820,232, 17/820,235, and 18/757,460, the contents of which are incorporated herein by reference in their entireties. The systems, methods, compositions, and kits provided herein can, in some embodiments, be employed in concert with the systems, methods, compositions, and kits described in PCT Application Publication No. WO2024081912A1, entitled, “PROTEIN-BASED SIGNAL AMPLIFICATION,” the content of which is incorporated herein by reference in its entirety. The systems, methods, compositions, and kits provided herein can, in some embodiments, be employed in concert with the systems, methods, compositions, and kits described in U.S. patent application Ser. No. 18/799,870, entitled, “MOLECULAR RECORDING METHODS AND SYSTEMS TO CAPTURE LINEAGE RELATIONSHIPS IN DIFFERENTIATING STEM CELLS,” filed Aug. 9, 2024, the content of which is incorporated herein by reference in its entirety. The systems, methods, compositions, and kits provided herein can, in some embodiments, be employed in concert with the systems, methods, compositions, and kits described in PCT Patent Application No. PCT/US24/48247, entitled, “MIRNA CIRCUITS FOR CONTROLLED GENE EXPRESSION,” filed Sep. 24, 2024, the content of which is incorporated herein by reference in its entirety. The systems, methods, compositions, and kits provided herein can, in some embodiments, be employed in concert with the systems, methods, compositions, and kits described in Vlahos A E, Kang J, Aldrete C A, Zhu R, Chong L S, Elowitz M B, Gao X J. Protease-controlled secretion and display of intercellular signals. Nat Commun. 2022 Feb. 17; 13(1):912. doi: 10.1038/s41467-022-28623-y. PMID: 35177637; PMCID: PMC8854555, the content of which is hereby incorporated by reference in its entirety. The systems, methods, compositions, and kits provided herein can, in some embodiments, be employed in concert with the systems, methods, compositions, and kits described in Xia et al. (“Synthetic protein circuits for programmable control of mammalian cell death.” Cell (2024): 2785-2800), the content of which, including its star methods, supplementary materials and supplemental data, is incorporated herein by reference in its entirety. The systems, methods, compositions, and kits provided herein can, in some embodiments, be employed in concert with the systems, methods, compositions, and kits described in U.S. patent application Ser. No. 18/945,412, entitled “Synpoptosis circuits for programmable cell death control,” which is hereby incorporated by reference in its entirety. Some of the methods and compositions disclosed herein are also disclosed in Lu et al., “Engineered protein circuits for cancer therapy”, bioRxiv, 2025.04.16.647665, which is hereby incorporated by reference in its entirety.

### Generalizability

While the data presented herein are focused on Ras-mutant cancers, the general approach is not limited to Ras. The finding that engineered protein circuits can directly rewire the presence of a cancer-associated protein signature (e.g., the presence of mutant Ras) to cancer cell death potently and specifically enough to enable robust function in cancer cells and in vivo is of broad relevance to the field of cancer.

Replacing the sensor in the current circuits may immediately retarget our circuits to other cancer drivers such as MYC, β-catenin, or other oncogenes. In fact, circuits do not need to respond to oncogenes at all, but they can be designed to sense any protein or combination of proteins that provide an accurate signature of disease state and are compatible with a protein-based sensing mechanism. Similarly, circuit output can also be broadened to include the release of cytokines or the activation of other therapeutic responses. Engineered protein circuits are thus a versatile next-generation therapeutic platform for diverse human cancers. Thus, an oncoprotein of the disclosure can include protein associated with a cancer state of a cell, but not causative for said cancer.

Further, protein circuits with direct sense-kill capabilities are of relevance beyond cancer. For instance, using the same principles described here, circuits can be generalized to eliminate senescent cells, fibrotic tissue, or autoimmune cells. Further embodiments of the protein circuits, compositions, and methods of the disclosure are described below.

In some embodiments, the invention comprises a circuit composed of a sensor of cancer cell state (e.g., protease-based Ras activity sensor) and a sensor-activatable cell death effector (e.g., TEV protease-activatable caspase-3) as well as the method of eliminating cancer cells with this circuit. In some embodiments, a circuit is composed of a sensor of cancer cell state (e.g., protease-based Ras activity sensor) and an activatable pyroptosis effector (gasdermin module) to enhance immune-mediated tumor clearance in vivo, significantly reducing liver tumor burden in immunocompetent mouse models. It is further confirmed herein the hypothesis of the pyroptosis field that the combination of a conditional gasdermin activator and an activatable gasdermin has therapeutic relevance. Some embodiments involve mRNA-LNP-delivered circuits and their therapeutic use.

Disclosed herein are programmable therapeutic circuits that detect mutant Ras proteins and trigger amplified intracellular execution programs that induce active tumor cell death without requiring sustained inhibition of the Ras-ERK signaling axis. In some embodiments, the circuits are nucleic-acid encoded and delivered via mRNA-lipid nanoparticle (mRNA-LNP), viral vectors, plasmids, or other delivery formats. The circuits may comprise a mutant Ras sensor module, an optional amplifier/logic module, and an effector module that executes cell death (e.g., apoptosis and/or pyroptosis).

In some embodiments, the disclosed circuits and methods provide one or more of the following advantages. The circuits can exhibit cytotoxic versus cytostatic behavior, e.g., the induction of active cell death, distinguishing cytotoxic elimination from cytostatic growth arrest. The circuits can exhibit ERK-independence, e.g., execution of tumor cell death while phospho-ERK signaling is not substantially suppressed, distinguishing “sense-and-kill” circuits from stoichiometric pathway inhibition. In some embodiments, the circuits can function without oncogene addiction, e.g., they can display activity in Ras-mutant tumor contexts that are poorly responsive to Ras-pathway inhibitors or otherwise do not exhibit oncogene addiction. The circuits can involve catalytic execution versus stoichiometric inhibition, e.g., amplified cascade behavior in which detection of mutant Ras triggers lethal output without requiring stoichiometric blockade of signaling complexes. In some embodiments, the disclosed circuits display programmability to specific Ras alleles, e.g., sensor substitution enables targeting of defined KRAS variants, including KRAS G12D, KRAS G12C, and others. In some embodiments, the disclosed circuits are characterized by safety and reduced off-tumor toxicity, e.g., reduced transcriptional perturbation, preserved signaling signatures, and/or reduced viability effects in representative normal tissue models that are susceptible to on-pathway toxicity. In some embodiments, the disclosed circuits exhibit efficacy in drug-resistant settings, e.g., maintained killing of tumor cells exhibiting acquired resistance to KRAS inhibitors, including Sotorasib and/or RMC-7977, including resistance-associated mutations or phenotypes. Further discussion of the properties of the disclosed circuits are described below.

ERK-Independent Killing (“Sense-and-Kill” without Pathway Suppression)

In some embodiments, the disclosed circuits induce cell death without requiring suppression of ERK phosphorylation. This distinguishes the disclosed circuits from pathway inhibitors and supports a mechanism in which mutant Ras functions as a trigger marker rather than a target for stoichiometric inhibition.

### Temporal Interpretation of ERK Phosphorylation During Circuit-Mediated Killing

In some embodiments, “ERK-independent” operation is evaluated at early timepoints, before widespread loss of viability, because later-stage dying cells can show reduced total ERK and pERK as a secondary consequence of cellular collapse. Accordingly, in some embodiments, ERK phosphorylation (pERK) is measured shortly after circuit exposure (e.g., minutes to a few hours, prior to commitment to death as assessed by viability/apoptosis markers) so that the readout reflects upstream signaling rather than degradation associated with death. In this framework, pathway inhibitors typically reduce pERK as a proximate mechanism leading to growth arrest and/or death, whereas any pERK decrease observed in circuit-treated cells at later timepoints may be downstream of cytotoxicity rather than its cause. Thus, the disclosed circuits can initiate cytotoxic execution while upstream ERK phosphorylation remains substantially preserved at early timepoints, distinguishing “sense-and-kill” execution from stoichiometric pathway suppression.

### Cytotoxicity Versus Cytostasis: Active Cell Death

In some embodiments, the disclosed circuits are cytotoxic rather than cytostatic. Cytotoxicity is demonstrated by (i) reduction of viable cell number below day-0 baseline and/or (ii) induction of active apoptosis markers (e.g., Annexin V positivity, cleaved caspase) and/or (iii) rescue by apoptosis inhibitors such as pan-caspase inhibitors.

### Mechanistic Basis for Cytotoxicity and Threshold Behavior

In some embodiments, the circuits described herein induce active cell death when executioner caspase activity exceeds a functional threshold in the target cell. Without intending to be bound by theory, apoptosis is triggered when (i) a sufficient number of executioner caspase molecules (e.g., caspase-3 and/or caspase-7) are activated to drive substrate cleavage at a rate that commits the cell to death, and (ii) the activated caspase activity exceeds endogenous anti-apoptotic buffering capacity, including inhibition by XIAP and related inhibitors of apoptosis proteins. In some embodiments, the disclosed amplifier/effector architectures are configured to generate a burst or sustained level of executioner caspase activation sufficient to overcome XIAP-mediated suppression, thereby producing net cytotoxicity rather than growth arrest. In some embodiments, operation below this activation threshold produces partial pathway engagement or sublethal signaling without cytotoxic commitment, consistent with thresholded apoptosis.

### Activity in Non-Addicted Ras-Mutant Contexts

In some embodiments, the disclosed circuits are effective in Ras-mutant tumor contexts that are not strongly dependent on Ras signaling for proliferation and/or are poorly responsive to Ras-pathway inhibitors. In such contexts, inhibitors may provide only limited benefit, whereas the disclosed circuits provide potent cytotoxicity. Non-addicted contexts may be defined operationally by weak response to pathway inhibitors at specified conditions and/or by alternative dependency metrics.

### Operational Definition and Tested Non-Addicted Contexts

In some embodiments, circuit efficacy does not depend on classical “oncogene addiction” to Ras signaling. In this disclosure, non-addicted Ras-mutant contexts are operationally defined as Ras-mutant tumor cells and/or tumors that exhibit limited response to Ras-pathway inhibitors under defined conditions (e.g., reduced growth inhibition or incomplete response at clinically relevant inhibitor exposures), and/or are characterized by low Ras dependency by an appropriate dependency metric. In such contexts, pathway inhibitors may produce modest cytostatic effects without durable elimination, despite the presence of a Ras mutation. The disclosed circuits, in contrast, can induce cytotoxic cell death in these non-addicted contexts because execution is triggered by mutant Ras as a marker and amplified through downstream execution modules, rather than relying on sustained suppression of Ras-ERK signaling.

### Catalytic Execution Versus Stoichiometric Inhibition

In some embodiments, the disclosed circuits operate by catalytic execution rather than stoichiometric inhibition. Under this model, limited sensor engagement can trigger amplified downstream activation sufficient to cross cell-death thresholds, without requiring suppression of ERK signaling. Catalytic execution can be supported by data demonstrating cytotoxicity at circuit levels that do not measurably suppress pERK and/or by amplifier behavior demonstrating increased gain or left-shifted activation.

Regime-Dependent Transition from Catalytic Execution to Stoichiometric Inhibition

In some embodiments, the disclosed circuits exhibit distinct functional regimes depending on sensor abundance, localization, and target engagement. In a low-sensor/sub-saturating engagement regime, the sensor functions primarily as a trigger that initiates an enzymatic cascade (e.g., protease activation and amplification), culminating in effector activation and cytotoxic commitment. In this regime, tumor cell death can be achieved without blanket occupancy of mutant Ras and without substantial suppression of downstream ERK phosphorylation at early timepoints. In a high-sensor/saturating engagement regime, the sensor may occupy a larger fraction of mutant Ras molecules and partially interfere with Ras effector interactions, potentially producing an inhibitor-like effect on signaling. Accordingly, in some embodiments, the disclosure emphasizes operation in the low-sensor regime, where lethal output arises from catalytic amplification rather than stoichiometric blockade. Dose-response and/or expression-response relationships are provided herein to illustrate the regime dependence and to guide the selection of delivery and expression parameters that favor catalytic execution.

### Programmability to Specific KRAS Alleles (Including G12D)

In some embodiments, sensor modules are swapped to target specific KRAS alleles. In some embodiments, a KRAS G12D-specific sensor drives activation of the same effector modules used in other allele contexts. In some embodiments, selectivity is demonstrated by preferential killing of cells harboring the targeted allele relative to wild-type controls or alternative alleles.

### Safety and Reduced On-Pathway Off-Tumor Toxicity

In some embodiments, safety is evaluated by: (a) transcriptomic profiling (e.g., RNA-seq) in representative normal tissue models, (b) pathway signature scoring (e.g., KRAS signaling signature, cell cycle/proliferation signatures), and/or (c) viability assays (e.g., CellTiter-Glo) in tissue models that commonly experience on-pathway off-tumor toxicity (e.g., intestinal epithelium, skin, hematopoietic lineages). In various embodiments, circuit treatment produces reduced transcriptional perturbation and/or reduced suppression of pathway signatures in normal tissues compared to Ras-pathway inhibitors under matched conditions, while maintaining potent cytotoxicity in Ras-mutant tumor cells.

### Efficacy in Drug-Resistant Settings

In some embodiments, circuit efficacy is maintained in tumor cells that are resistant to Ras inhibitors, including Sotorasib and/or RMC-7977. Resistance may be modeled by selection under inhibitor pressure to generate acquired resistance populations, or by introduction of resistance-associated mutations/phenotypes. In some embodiments, drug-resistant populations remain sensitive to circuit-induced cell death and, in some embodiments, exhibit greater sensitivity to the circuit than parental populations.

### Combination Therapy, Sequencing, and Regimens

In some embodiments, circuits are used: (a) as salvage therapy after progression on inhibitors, (b) as first-line therapy to reduce the emergence of resistance, (c) in combination with inhibitors, and/or (d) in defined sequences (inhibitor→circuit; circuit→inhibitor; alternating regimens). In some embodiments, dosing regimens are designed to exploit rapid cytotoxicity and reduce continuous selective pressure.

The sensing paradigm described herein uses target-binding moieties fused to complementary portions of a split effector, such as a split protease. In certain embodiments, the target-binding moieties comprise mutant-selective Ras binders, and the sensor specificity and activity arise from: (i) a conformational difference between mutant Ras and wild-type Ras; (ii) nanoclustering of mutant Ras at the cell membrane, which promotes productive sensor colocalization and/or dimerization; and/or (iii) membrane localization of Ras, which increases the likelihood of productive encounters between the sensor components and the target.

Importantly, the sensing architecture is not limited to Ras. More generally, the disclosed systems can be adapted to detect or discriminate target states that differ based on one or more of the following: protein conformation, mutation status, post-translational modification, interaction state, abundance, oligomerization state, complex formation, and/or subcellular localization.

In some embodiments, successful implementation of the sensor design is facilitated by one or more of the following target properties.

### Target Binding.

The target (e.g., an oncoprotein) preferably permits binding by one or more binding moieties, such as antibodies, antibody fragments, nanobodies, monobodies, peptides, engineered proteins, de novo proteins, natural proteins, aptamers, other affinity reagents, or domains of the former proteins.

Target Interactions and/or Oligomerization.

In some embodiments, the target (e.g., an oncoprotein) may be: (i) a monomer having at least two distinct binding sites; (ii) a dimer having at least two binding sites per dimer in sufficient proximity to promote split-effector complementation; (iii) an oligomer having at least two binding sites per oligomer in sufficient proximity; (iv) an aggregate having at least two binding sites per aggregate in sufficient proximity; (v) a multi-protein complex in which two or more constituent proteins collectively provide at least two binding sites in sufficient proximity for complementation; and/or (vi) an onco-fusion protein. Additional proteins may be present in such complexes, provided they do not substantially prevent productive interaction between the target and the sensor components.

### Expression Level and Turnover.

In principle, the sensing paradigm is not subject to a strict lower or upper bound on target expression level or turnover rate. However, higher target abundance and/or slower target turnover may improve detectability in some implementations.

### Subcellular Localization.

In some embodiments, the target (e.g., an oncoprotein) is intracellular or includes at least one intracellular domain. The target may be localized in the cytoplasm, plasma membrane, nucleus, organelles, or other intracellular compartments. Sensor localization signals, such as nuclear localization sequences, membrane-localizing motifs, organelle-targeting sequences, export sequences, or combinations thereof, may optionally be used to enrich sensor components in the relevant compartment. Example 4 illustrates the application of these principles beyond Ras.

Disclosed herein include synthetic protein circuits. In some embodiments, the synthetic protein circuit comprises: (a) a sensor module comprising: a first polypeptide comprising a first oncoprotein binding domain and a first part of a first orthogonal protease domain, wherein the first oncoprotein binding domain is capable of binding a first oncoprotein to form a first oncoprotein-bound polypeptide; a second polypeptide comprising a second oncoprotein binding domain and a second part of the first orthogonal protease domain, wherein the second oncoprotein binding domain is capable of binding a second oncoprotein to form a second oncoprotein-bound polypeptide, and wherein the first part of the first orthogonal protease domain and the second part of the first orthogonal protease domain are capable of associating with each other to constitute a first orthogonal protease capable of being in a first orthogonal protease active state when the first oncoprotein and the second oncoprotein are in close proximity at a proximity site, wherein the first oncoprotein and/or the second oncoprotein comprise: (i) a monomer having at least two distinct binding sites; (ii) a dimer having at least two binding sites per dimer in sufficient proximity to promote association; (iii) an oligomer having at least two binding sites per oligomer in sufficient proximity to promote association; (iv) an aggregate having at least two binding sites per aggregate in sufficient proximity to promote association; (v) a multi-protein complex in which two or more constituent proteins collectively provide at least two binding sites in sufficient proximity for association; and/or (vi) an onco-fusion protein, and (b) an effector module, wherein the first orthogonal protease in the first orthogonal protease active state is capable of activating the effector module in a cell.

Disclosed herein include synthetic protein circuits. In some embodiments, the synthetic protein circuit comprises: (a) a sensor module comprising: a first polypeptide comprising a first oncoprotein binding domain and a first part of a first orthogonal protease domain, wherein the first oncoprotein binding domain is capable of binding a first oncoprotein to form a first oncoprotein-bound polypeptide; a second polypeptide comprising a second oncoprotein binding domain and a second part of the first orthogonal protease domain, wherein the second oncoprotein binding domain is capable of binding a second oncoprotein to form a second oncoprotein-bound polypeptide, and wherein the first part of the first orthogonal protease domain and the second part of the first orthogonal protease domain are capable of associating with each other to constitute a first orthogonal protease capable of being in a first orthogonal protease active state when the first oncoprotein and the second oncoprotein are in close proximity at a proximity site, optionally the first and/or second oncoprotein binding domain comprise a sequence at least 85% identical to the sequence of SEQ ID NO: 109 or SEQ ID NO: 111; and (b) an effector module capable of inducing apoptosis or pyroptosis in a cell, wherein the first orthogonal protease in the first orthogonal protease active state is capable of activating the effector module, thereby inducing apoptosis or pyroptosis in the cell.

The first oncoprotein and/or the second oncoprotein can comprise a monomer having at least two distinct binding sites. The first oncoprotein and/or the second oncoprotein can comprise a dimer having at least two binding sites per dimer in sufficient proximity to promote association. The first oncoprotein and/or the second oncoprotein can comprise an oligomer having at least two binding sites per oligomer in sufficient proximity to promote association. The first oncoprotein and/or the second oncoprotein can comprise an aggregate having at least two binding sites per aggregate in sufficient proximity to promote association. The first oncoprotein and/or the second oncoprotein can comprise a multi-protein complex in which two or more constituent proteins collectively provide at least two binding sites in sufficient proximity for association. The first oncoprotein and/or the second oncoprotein can comprise an onco-fusion protein. In some embodiments, the first part of the first orthogonal protease domain and the second part of the first orthogonal protease domain have weak association affinity.

The first oncoprotein binding domain and the second oncoprotein binding domain can be identical. The first oncoprotein binding domain and the second oncoprotein binding domain can be different.

In some embodiments, the first oncoprotein binding domain, the second oncoprotein binding domain, or both are specific to the first and/or second oncoprotein and do not bind or weakly bind a wild type version of the first and/or second oncoprotein. In some embodiments, the first oncoprotein binding domain and/or the second oncoprotein binding domain have up to 400-fold (e.g., 1.5-fold, 2-fold, 10-fold, 20-fold, 30-fold, 40-fold, 50-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 310-fold, 320-fold, 330-fold, 340-fold, 350-fold, 360-fold, 370-fold, 380-fold, 390-fold, and 400-fold, or a number or a range between any two of these values) greater affinity for the first and/or second oncoprotein relative to a wild type version of the first and/or second oncoprotein.

The first oncoprotein binding domain, the second oncoprotein binding domain, or both can be capable of binding to a cryptic epitope of the first and/or second oncoprotein. In some embodiments, the cryptic epitope is not available for binding in a wild type version of the first and/or second oncoprotein. The first oncoprotein binding domain, the second oncoprotein binding domain, or both can be capable of binding a cryptic epitope within a p53 oncoprotein. For examples of binding domains capable of binding within a cryptic epitope of a p53 oncoprotein, see, Table 11.

The first oncoprotein binding domain, the second oncoprotein binding domain, or both can comprise a RAS binding domain (RBD) and/or RAS association domain (RAD). The first oncoprotein binding domain, the second oncoprotein binding domain, or both can comprise an antibody, a nanobody, a monobody, a peptide, an engineered protein, a de novo protein, a natural protein, an aptamer, or any variant or fragment thereof. The first oncoprotein binding domain, the second oncoprotein binding domain, or both can comprise a nanobody, a Camel Ig, Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), single-domain antibody (sdAb), or any combination thereof.

There are provided, in some embodiments, antigen-binding moieties (e.g., monobodies). In some embodiments, the first and/or second oncoprotein binding domain comprise an antigen binding moiety. The antigen-binding moiety can be configured to bind any of the oncoproteins contemplated herein. The antigen-binding moiety can be configured to bind a oncoprotein in an active and/or inactive state as described herein. Antigen-binding moieties can comprise antibodies, antibody fragments, and variants. In some embodiments, antibody fragments and variants may comprise antigen binding regions from intact antibodies. Examples of antibody fragments and variants may include, but are not limited to Fab, Fab′, F(ab′)2, and Fv fragments; diabodies; linear antibodies; single-chain antibody molecules such as single chain variable fragment (scFv); and multi specific antibodies formed from antibody fragments.

For the purposes herein, an “antibody” may comprise a heavy and light variable domain as well as an Fc region. As used herein, the term “native antibody” usually refers to a heterotetrameric glycoprotein of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end: the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FVV) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments based on comparisons with other antibodies can also be used. Determining residues that make up CDRs may include the use of numbering schemes including, but not limited to, those taught by Kabai, Chothia, and Honegger.

H and VL domains have three CDRs each. VL CDRs are referred to herein as CDR-L1, CDR-L2 and CDR-L3, in order of occurrence when moving from N- to C-terminus along the variable domain polypeptide. VH CDRs are referred to herein as CDR-H1, CDR-H2 and CDR-H3, in order of occurrence when moving from N- to C-terminus along the variable domain polypeptide. Each of CDRs has favored canonical structures with the exception of the CDR-H3, which comprises amino acid sequences that may be highly variable in sequence and length between antibodies resulting in a variety of three-dimensional structures in antigen-binding domains. In some cases, CDR-H3s may be analyzed among a panel of related antibodies to assess antibody diversity. Various methods of determining CDR sequences are known in the art and may be applied to known antibody sequences.

As used herein, the term “Fv” refers to an antibody fragment comprising the minimum fragment on an antibody needed to form a complete antigen-binding site. These regions consist of a dimer of one heavy chain and one light chain variable domain in tight, non-covalent association. Fv fragments can be generated by proteolytic cleavage, but are largely unstable. Recombinant methods are known in the art for generating stable Fv fragments, typically through insertion of a flexible linker between the light chain variable domain and the heavy chain variable domain (to form a single chain Fv (scFv) or through the introduction of a disulfide bridge between heavy and light chain variable domains.

As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

As used herein, the term “single chain Fv” or “scFv” refers to a fusion protein of VH and VL antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen. Using molecular genetics, two scFvs can be engineered in tandem into a single polypeptide, separated by a linker domain, called a “tandem scFv” (tascFv). Construction of a tascFv with genes for two different scFvs yields a “bispecific single-chain variable fragments” (bis-scFvs).

As used herein, the term “bispecific antibody” refers to an antibody capable of binding two different antigens. Such antibodies typically comprise regions from at least two different antibodies. As used herein, the term “diabody” refers to a small antibody fragment with two antigen-binding sites. Diabodies are functional bispecific single-chain antibodies (bscAb). Diabodies comprise a heavy chain variable domain VH connected to a light chain variable domain VL in the same polypeptide chain. By using a linker that is too short to allow pairing between the two domains on the same chain, the domains are forced to pair with the complementary domains of another chain and create two antigen-binding sites.

The term “intrabody” can refer to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling and cell division. In some embodiments, methods provided herein may include intrabody-based therapies. In some such embodiments, variable domain sequences and/or CDR sequences disclosed herein may be incorporated into one or more constructs for intrabody-based therapy.

As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen.

The modifier “monoclonal” can indicate the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity. In one embodiment, the antibody may be a humanized full-length antibody.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition or structure as compared to a native antibody. Antibody variants may include, but are not limited to, antibodies with altered isotypes (e.g., IgA, IgD, IgE, IgG1, IgG2, IgG3, IgG4, or IgM), humanized variants, optimized variants, multi-specific antibody variants (e.g., bispecific variants), and antibody fragments.

In some embodiments, the antigen-binding moieties provided herein comprise antibody mimetics (e.g., monobodies). As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affitins, anticalins, avimers, Centyrins, DARPINS™, Fynomers and Kunitz and domain peptides. In other embodiments, antibody mimetics may include one or more non-peptide regions.

In some embodiments, the antigen-binding moieties provided herein comprise multispecific antibodies that bind more than one epitope. As used herein, the terms “multibody” or “multispecific antibody” refer to an antibody wherein two or more variable regions bind to different epitopes. The epitopes may be on the same or different targets. In some embodiments, a multi-specific antibody is a “bispecific antibody” which recognizes two different epitopes on the same or different antigens. In one aspect, bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen. New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

In some embodiments, the antigen-binding moieties provided herein comprise antibodies comprising a single antigen-binding domain (e.g., nanobodies). These molecules are extremely small, with molecular weights approximately one-tenth of those observed for full-sized mAbs. Further antibodies may include “nanobodies” derived from the antigen-binding variable heavy chain regions (VHHs) of heavy chain antibodies found m camels and llamas, which lack light chains (Nelson, A. L., MAbs. 2010. January-February; 2(1):77-83).

In some embodiments, the antibody may be “miniaturized”. Among the best examples of mAb miniaturization are the small modular immunopharmaceuticals (SMIPs) from Trubion Pharmaceuticals. These molecules, which can be monovalent or bivalent, are recombinant single-chain molecules containing one VL, one VH antigen-binding domain, and one or two constant “effector” domains, all connected by linker domains. One example of miniaturized antibodies is called “unibody” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo.

In some embodiments, the antigen-binding moieties provided herein comprise single-domain antibodies (sdAbs, or nanobodies) which are antibody fragment consisting of a single monomelic variable antibody domain. In some embodiments, it is able to bind selectively to a specific antigen (e.g., like a whole antibody). In one aspect, a sdAb may be a “Camel Ig or “camelid VHH”. As used herein, the term “camel Ig” refers to the smallest known antigen-binding unit of a heavy chain antibody (Koch-No lte, et al, FASEB J., 2007, 21: 3490-3498). A “heavy chain antibody” or a “camelid antibody” refers to an antibody that contains two VH domains and no light chains (Riechmann L. et al, J. Immunol. Methods, 1999, 231: 25-38; international patent publication NOs. WO 1994/04678 and WO 1994/025591; and U.S. Pat. No. 6,005,079). In another aspect, a sdAb may be a “immunoglobulin new antigen receptor” (IgNAR). As used herein, the term “immunoglobulin new antigen receptor” refers to class of antibodies from the shark immune repertoire that consist of homodimers of one variable new antigen receptor (VNAR) domain and five constant new antigen receptor (CNAR) domains. IgNARs represent some of the smallest known immunoglobulin-based protein scaffolds and are highly stable and possess efficient binding characteristics. The inherent stability can be attributed to both (i) the underlying Ig scaffold, which presents a considerable number of charged and hydrophilic surface exposed residues compared to the conventional antibody VH and VL domains found in murine antibodies; and (ii) stabilizing structural features in the complementary determining region (CDR) loops including inter-loop disulphide bridges, and patterns of intra-loop hydrogen bonds.

In some embodiments, the antigen-binding moieties provided herein comprise intrabodies. Intrabodies are a form of antibody that is not secreted from a cell in which it is produced, but instead targets one or more intracellular proteins. Intrabodies are expressed and function intracellularly, and may be used to affect a multitude of cellular processes including, but not limited to intracellular trafficking, transcription, translation, metabolic processes, proliferative signaling and cell division. Sequences from donor antibodies may be used to develop intrabodies. Intrabodies are often recombinantly expressed as single domain fragments such as isolated VH and VL domains or as a single chain variable fragment (scFv) antibody within the cell. For example, intrabodies are often expressed as a single polypeptide to form, a single chain antibody comprising the variable domains of the heavy and light chains joined by a flexible linker polypeptide, intrabodies typically lack disulfide bonds and are capable of modulating the expression or activity of target genes through their specific binding activity. Single chain intrabodies are often expressed from a recombinant nucleic acid molecule and engineered to be retained intracellularly (e.g., retained in the cytoplasm, endoplasmic reticulum, or periplasm).

The first and second polypeptides can be expressed in the cell as separate molecules. The first polypeptide and the second polypeptide can be expressed in the cell on a single molecule as a proprotein. The proprotein can comprise one or more proprotein protease cleavage sites. The first polypeptide and the second polypeptide can be separated on the proprotein by at least one of the one or more proprotein protease cleavage sites. The first polypeptide and the second polypeptide can be separated on the proprotein by a first proprotein protease cleavage site, or a first proprotein protease cleavage site and a second proprotein protease cleavage site. The proprotein can comprise: the first polypeptide comprising the first oncoprotein binding domain and the first part of the first orthogonal protease domain and the second polypeptide comprising the second oncoprotein binding domain and the second part of the first orthogonal protease domain; a first proprotein protease cleavage site cleavable by the first orthogonal protease in the first orthogonal protease active state, or a first proprotein protease cleavage site and a second proprotein protease cleavage site each cleavable by the first orthogonal protease in the first orthogonal protease active state; and an optional linker.

The components of a proprotein can be arranged in any order. For example, from N-terminus to C-terminus: the first or second oncoprotein binding domain—nTEVP—first and/or second orthogonal cleavage site—the first or second oncoprotein binding domain—cTEVP, the first or second oncoprotein binding domain—cTEVP—first and/or second orthogonal cleavage site—the first or second oncoprotein binding domain—nTEVP, cTEVP—binder—first and/or second orthogonal cleavage site the first or second oncoprotein binding domain—nTEVP. The proprotein can comprise, from N-terminus to C-terminus: the first polypeptide comprising the first oncoprotein binding domain and the first part of the first orthogonal protease domain; a first proprotein protease cleavage site cleavable by the first orthogonal protease in the first orthogonal protease active state; an optional linker; a second proprotein protease cleavage site cleavable by the first orthogonal protease in the first orthogonal protease active state; and the second polypeptide comprising the second oncoprotein binding domain and the second part of the first orthogonal protease domain.

In some embodiments, the linker: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; and/or comprises 3 repeating amino acid subunits or more. The first polypeptide, the second polypeptide, or both can comprise at least one nuclear localization signal.

In some embodiments, the first part of the first orthogonal protease domain and the second part of the first orthogonal protease domain of the proprotein are capable of associating with each other to form the first orthogonal protease in the first orthogonal protease active state, wherein the first orthogonal protease in the first orthogonal protease active state is capable of cleaving the proprotein at the one or more proprotein protease cleavage sites, e.g., the first and/or second proprotein protease cleavage site, to release the first polypeptide and the second polypeptide as separate molecules in the cell.

The first oncoprotein binding domain, the second oncoprotein binding domain, or both, can comprise or consist of the sequence of SEQ ID NO: 109, SEQ ID NO: 110, or SEQ ID NO: 111. The first oncoprotein binding domain, the second oncoprotein binding domain, or both, can comprise a sequence having at least 85% (e.g., 85%, 86%, 87%, 88%, 89%, 90%, 91%, 92%, 93%, 94%, 95%, 96%, 97%, 98%, 99%, 100%, or a number or a range between any two of these values) identity to SEQ ID NO: 109, SEQ ID NO: 110, or SEQ ID NO: 111.

The first oncoprotein binding domain, the second oncoprotein binding domain, or both can be capable of binding to p53, for instance, a cryptic epitope in mutant p53. The first oncoprotein binding domain, the second oncoprotein binding domain, or both, can comprise or consist of the sequence of any one of SEQ ID NOs: 141-163. The first oncoprotein binding domain, the second oncoprotein binding domain, or both, can comprise a sequence having at least 85% (e.g., 85%, 86%, 87%, 88%, 89%, 90%, 91%, 92%, 93%, 94%, 95%, 96%, 97%, 98%, 99%, 100%, or a number or a range between any two of these values) identity to any one of SEQ ID NOs: 141-163.

Provided herein are first and/or second polypeptides, e.g., scaffolds thereof, in which the first and/or second oncoprotein binding domain can be modified as determined by one of skill in the art. The first polypeptide, the second polypeptide, or both can comprise the sequence of any one of SEQ ID NOs: 5, 7, 116-117, and 178-179 or a sequence having at least 85% identity (e.g., 85%, 86%, 87%, 88%, 89%, 90%, 91%, 92%, 93%, 94%, 95%, 96%, 97%, 98%, 99%, 100%, or a number or a range between any two of these values) identity to any one of SEQ ID NOs: 5, 7, 116-117, and 178-179.

The first proprotein protease cleavage site, the second proprotein protease cleavage site, or both can comprise any protease cleavage site known in the art. The first proprotein protease cleavage site, the second proprotein protease cleavage site, or both, can comprise the sequence of any one of SEQ ID NOs: 247, 253, and 255-258 or a sequence having one or two mismatches relative to any one of SEQ ID NOs: 247, 253, and 255-258.

The proprotein can comprise the sequence of any one of SEQ ID NOs: 112-113 or a sequence at least 85% (e.g., 85%, 86%, 87%, 88%, 89%, 90%, 91%, 92%, 93%, 94%, 95%, 96%, 97%, 98%, 99%, 100%, or a number or a range between any two of these values) identical to the sequence of any one of SEQ ID NOs: 112-113.

Any of the polypeptides disclosed herein can comprise a degron. A degron is a portion of a protein that is important in regulation of protein degradation rates. Known degrons include short amino acid sequences, structural motifs, and exposed amino acids (often Lysine or Arginine) located anywhere in the protein. In fact, some proteins can even contain multiple degrons. Degrons are present in a variety of organisms, from the N-degrons (see N-end Rule) first characterized in yeast to the PEST sequence of mouse ornithine decarboxylase. Degrons have been identified in prokaryotes as well as eukaryotes. While there are many types of different degrons, and a high degree of variability even within these groups, degrons are all similar for their involvement in regulating the rate of a protein's degradation. Much like protein degradation (see proteolysis) mechanisms are categorized by their dependence or lack thereof on Ubiquitin, a small protein involved in proteasomal protein degradation, degrons are also be referred to as “Ubiquitin-dependent” or “Ubiquitin-independent.

In some embodiments, the degron comprises an N-degron, a dihydrofolate reductase (DHFR) degron, a FKB protein (FKBP) degron, derivatives thereof, or any combination thereof. The degron can comprise an N-degron. Some degrons are ubiquitin-dependent or ubiquitin-independent. A degron can comprise DHFR degron, an N-degron, a phospho degron, a heat inducible degron, a photosensitive degron, an oxygen dependent degron, ornithine decarboxylase degron, estrogen receptor domain degrons, a ecDHFR degron, an FKBP degron, a UnaG degron, or any combination thereof. As a non-limiting example, the degron may be an ornithine decarboxylase degron. The degron can comprise a ecDHFR degron. The presence of a degron sequence in a polypeptide can result in the polypeptide being targeted for degradation, e.g., be in “a destabilized state.”

### Oncoproteins

The first oncoprotein, the second oncoprotein, or both can comprise at least one mutation associated with cancer. The first oncoprotein, the second oncoprotein, or both can comprise a conformation, post-translational modification, interaction state, abundance, oligomerization state, multimerization state, and/or subcellular localization state associated with a disease or disorder. The disease or disorder can be cancer.

The first oncoprotein and the second oncoprotein can be identical. The first oncoprotein, the second oncoprotein, or both can be endogenous proteins. The first oncoprotein, the second oncoprotein, or both can be over-expressed and/or mis-folded in the cell relative to a wild type version of the first and/or second oncoprotein. The first oncoprotein, the second oncoprotein, or both can be mis-folded, exposing a cryptic epitope.

The first oncoprotein, the second oncoprotein, or both can comprise AKT, PI3K, MAPK, p44/42 MAP kinase, TYK2, p38 MAP kinase, PKC, PKA, SAPK, ELK, INK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCy, NF-kB, FAK, CREB, αIIIβ3, FcεRI, BAD, p70S6K, STAT1, STAT2, STAT3, STAT5, STAT6, or any combination thereof. The first oncoprotein, the second oncoprotein, or both can be capable of regulating cell survival, cell growth, cell proliferation, cell adhesion, cell migration, cell metabolism, cell morphology, cell differentiation, apoptosis, or any combination thereof.

In some embodiments: (i) the first oncoprotein and/or the second oncoprotein comprising a monomer having at least two distinct binding sites comprise: p53, β-catenin, e.g., CTNNB1, MYC, e.g., c-Myc, Cyclin D1, any variant thereof, or any combination thereof, (ii) the first oncoprotein and/or the second oncoprotein comprising a dimer having at least two binding sites per dimer in sufficient proximity to promote association comprise: MYC:MAX heterodimer, STAT3 constitutive homodimerization, RAF dimers, e.g., BRAF:CRAF or BRAF:BRAF, HIF-1α:HIF-1β, e.g., ARNT, any variants thereof, or any combination thereof; (iii) the first oncoprotein and/or the second oncoprotein comprising an oligomer having at least two binding sites per oligomer in sufficient proximity to promote association comprise: RAS nanoclusters, e.g., KRAS, NRAS, and/or HRAS nanoclusters, p53 tetramers, EML4-ALK trimerization, BCR-ABL tetramers, EGFR intracellular domain clusters, Nucleophosmin (NPM1) oligomers, or any combination thereof, (iv) the first oncoprotein and/or the second oncoprotein comprising an aggregate having at least two binding sites per aggregate in sufficient proximity to promote association comprise: mutant p53 amyloid aggregates, PTEN aggregates, or both; (v) the first oncoprotein and/or the second oncoprotein comprising a multi-protein complex in which two or more constituent proteins collectively provide at least two binding sites in sufficient proximity for association comprise: MDM2:p53, KRAS:RAF-RBD, β-catenin:TCF/LEF, Cyclin E:CDK2, or any combination thereof, and/or (vi) wherein the first oncoprotein and/or the second oncoprotein comprising an onco-fusion protein comprise: BCR-ABL, EML4-ALK, EWSR1-FLI1, PML-RARα, SS18-SSX, or any combination thereof.

RAS genes are the most frequently mutated oncogenes and play critical roles in the development and progression of malignancies. The first oncoprotein, the second oncoprotein, or both can comprise a RAS protein. The RAS protein can be KRAS, NRAS, HRAS, or any combination thereof. The RAS protein can comprise at least one mutation associated with cancer. The at least one mutation can comprise a mutation in KRAS, NRAS, and/or HRAS. In some embodiments, the KRAS mutation is selected from the group consisting of G12C, G12V, G12S, G12A, G12D, G12R, G13V, G13C, G13D, Q61P, Q61L, Q61H, Q61K, Q61E, Q61R, A146V, A146P, and A146T. In some embodiments, the HRAS mutation is selected from the group consisting of G12V, G12C, G12S, G12A, G12D, G12R, G13V, G13dup, G13C, G13S, G13D, Q61L, Q61H, Q61K, and Q61R. In some embodiments, the NRAS mutation is selected from the group consisting of G12A, G12C, G12D, G12R, G12S, G12V, G13A, G13C, G13D, G13R, G13S, G13V, Q61E, Q61H, Q61K, Q61L, Q61P, and Q61R.

### Effector Modules

The effector module can comprise a protease activatable reporter. The reporter can provide a readout of oncoprotein activity in a cell. The protease activatable reporter can comprise a fluorescent protein comprising green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mScarlet, mApple, mCitrine, mCherry, mruby3, rsCherry, rsCherryRev, derivatives thereof, or any combination thereof. The effector module can comprise a synthetic apoptosis circuit or a synthetic pyroptosis circuit. The synthetic apoptosis circuit or synthetic pyroptosis circuit can induce cell death, e.g., of a cancer cell.

In some embodiments, the effector module comprises a synthetic apoptosis circuit comprising: an apoptosis polypeptide comprising a large subunit of an apoptotic effector protein and a small subunit of an apoptotic effector protein separated by a first orthogonal protease cleavage site, wherein two apoptosis polypeptides are capable of associating with each other to constitute an apoptotic protein complex in an apoptotic protein complex inactive state, wherein the first orthogonal protease in a first orthogonal protease active state is capable of cleaving the first orthogonal protease cleavage site of the apoptosis polypeptide, and wherein the first orthogonal protease cleavage site of the apoptosis polypeptide being cleaved changes the apoptotic protein complex from the apoptotic protein complex inactive state to an apoptotic protein complex active state, thereby activating the effector module, wherein the apoptotic protein complex in the apoptotic protein complex active state is capable of inducing apoptosis in the cell.

The apoptotic effector protein can comprise caspase-3, caspase-6, caspase-7, or any variant, portion, or derivative thereof. The apoptosis polypeptide can comprise one or more linkers. In some embodiments, any of the one or more linkers: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof, and/or comprises 3 repeating amino acid subunits or more.

The first orthogonal protease cleavage site can comprise any protease cleavage site known in the art. The first orthogonal protease cleavage site can comprise the sequence of any one of SEQ ID NOs 247, 253, and 255-258 or a sequence having one or two mismatches relative to any one of SEQ ID NOs 247, 253, and 255-258. The apoptosis polypeptide can comprise the sequence of any one of SEQ ID NOs: 220-221 or a sequence at least 85% (e.g., 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 220-221.

In some embodiments, the effector module comprises a synthetic pyroptosis circuit comprising: a pyroptosis polypeptide comprising a pyroptosis effector domain and an inhibitory domain separated by a first orthogonal protease cleavage site, wherein the inhibitory domain is capable of inhibiting activity of the pyroptosis effector domain, thereby the pyroptosis polypeptide is in a pyroptosis polypeptide inactive state, wherein the first orthogonal protease in a first orthogonal protease active state is capable of cleaving the first orthogonal protease cleavage site of the pyroptosis polypeptide, and wherein the first orthogonal protease cleavage site of the pyroptosis polypeptide being cleaved changes the pyroptosis polypeptide from the pyroptosis polypeptide inactive state to a pyroptosis polypeptide active state, thereby activating the effector module, wherein the pyroptosis polypeptide in the pyroptosis polypeptide active state is capable of inducing pyroptosis in the cell.

The inhibitory domain can comprise a protein domain configured to reduce or abrogate the activity of the pyroptosis effector domain, e.g., a bulky domain. The inhibitory domain can comprise an auto-inhibitory domain of a gasdermin family of pore-forming proteins or maltose-binding protein. The pyroptosis effector domain can comprise an N-terminal domain of a gasdermin (GSDM) protein. The GSDM protein can be GSDMA, GSDMB, GSDMC, GSDMD, GSDME, or DFNB59. The pyroptosis polypeptide can comprise one or more linkers. In some embodiments, any of the one or more linkers: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; and/or comprises 3 repeating amino acid subunits or more. In some embodiments, inducing pyroptosis in the cell causes the cell to release one or more inflammatory cytokines. The one or more inflammatory cytokines can comprise IL-18, IL-1β, IL-6, IL-8, interferon gamma (IFN-γ), and/or tumor necrosis factor-alpha (TNF-α). The first orthogonal protease cleavage site can comprise any protease cleavage site known in the art. The first orthogonal protease cleavage site can comprise the sequence of any one of SEQ ID NOs 247, 253, and 255-258 or a sequence having one or two mismatches relative to any one of SEQ ID NOs 247, 253, and 255-258.

The pyroptosis polypeptide can comprise the sequence of any one of SEQ ID NOs: 237-241 or a sequence at least 85% (e.g., 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 237-241.

In some embodiments, first orthogonal protease comprises or is derived from a viral protease. The first orthogonal protease can comprise tobacco etch virus (TEV) protease, tobacco vein mottling virus (TVMV) protease, hepatitis C virus protease (HCVP), derivatives thereof, or any combination thereof. The first orthogonal protease can comprise the sequence of any one of SEQ ID NOs: 211, 213, and 215 or a sequence having at least 85% (e.g., 85%, 86%, 87%, 88%, 89%, 90%, 91%, 92%, 93%, 94%, 95%, 96%, 97%, 98%, 99%, 100%, or a number or a range between any two of these values) identity to any one of SEQ ID NOs: 211, 213, and 215. The first orthogonal protease can comprise one or more mutations that boost catalytic efficiency. The first orthogonal protease can comprise the sequence of any one of SEQ ID NOs: 216-217 or a sequence having at least 85% (e.g., 85%, 86%, 87%, 88%, 89%, 90%, 91%, 92%, 93%, 94%, 95%, 96%, 97%, 98%, 99%, 100%, or a number or a range between any two of these values) identity to any one of SEQ ID NOs: 216-217.

### Amplifier Modules

The synthetic protein circuit can comprise a synthetic amplifier circuit. An amplifier circuit can, in some embodiments, enhance sensor performance. In some embodiments, the synthetic amplifier circuit comprises: (i) an amplifier protein comprising a first part of a second orthogonal protease domain, a first dimerization domain, a first orthogonal protease cleavage site the first orthogonal protease in the first orthogonal protease active state is capable of cleaving, a second dimerization domain, a second orthogonal cleavage site the first orthogonal protease in the first orthogonal protease active state is capable of cleaving, and a first caging domain; (ii) a companion amplifier protein comprising a second part of a second orthogonal protease domain, a third dimerization domain, a third orthogonal cleavage site the first orthogonal protease in the first orthogonal protease active state is capable of cleaving, a fourth dimerization domain, a fourth orthogonal cleavage site the first orthogonal protease in the first orthogonal protease active state is capable of cleaving, and a second caging domain.

In some embodiments, the amplifier protein and the companion amplifier protein separately do not comprise a second orthogonal protease capable of being in a second orthogonal protease active state. In some embodiments: the first caging domain is a catalytically inactive version of the second part of the second orthogonal protease domain. In some embodiments, the first caging domain has between about 50% to about 99% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values) sequence identity to the second part of the second orthogonal protease domain. In some embodiments, the second caging domain is a catalytically inactive version of the first part of the second orthogonal protease domain. In some embodiments, the second caging domain has between about 50% to about 99% (e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values) sequence identity to the first part of the second orthogonal protease domain. In some embodiments: the first orthogonal cleavage site and the second orthogonal cleavage site flank the second dimerization domain; and/or the third orthogonal cleavage site and the fourth orthogonal cleavage site flank the fourth dimerization domain.

In some embodiments: the first dimerization domain is capable of binding the second dimerization domain; the first dimerization domain is capable of binding the third dimerization domain; and/or the third dimerization domain is capable of binding the fourth dimerization domain. The affinity of the first dimerization domain for the second dimerization domain can be weaker than the affinity of the first dimerization domain for the third dimerization domain. The affinity of the third dimerization domain for the fourth dimerization domain can be weaker than the affinity of the third dimerization domain for the first dimerization domain. Intramolecular binding between the first dimerization domain and the second dimerization domain of the amplifier protein can be capable of preventing the first part of the second orthogonal protease domain from associating with the second part of the second orthogonal protease domain of the companion amplifier protein to form the second orthogonal protease in the second orthogonal protease active state. Intramolecular binding between the third dimerization domain and the fourth dimerization domain of the companion amplifier protein can be capable of preventing the second part of the second orthogonal protease domain from associating with the first part of the second orthogonal protease domain of the amplifier protein to form the second orthogonal protease in the second orthogonal protease active state.

The first orthogonal protease in the first orthogonal protease active state can be capable of: cleaving the first orthogonal cleavage site and/or second orthogonal cleavage site of the amplifier protein, thereby forming a cleaved amplifier protein; and/or cleaving the third orthogonal cleavage site and/or fourth orthogonal cleavage site of the companion amplifier protein, thereby forming a cleaved companion amplifier protein.

In some embodiments, the cleaved amplifier protein and the cleaved companion amplifier protein are capable of associating via intermolecular binding of the first dimerization domain and the third dimerization domain to form a first complex. The first complex can comprise the second orthogonal protease capable of being in the second orthogonal protease active state.

In some embodiments, one or more of the first dimerization domain, second dimerization domain, third dimerization domain, and fourth dimerization domain is selected from the group comprising DHD9 heterodimer a, DHD13_XAAA heterodimer a, DHD13_XAXA heterodimer a, DHD13_XAAX heterodimer a, DHD13_2:341 heterodimer a, DHD13_AAAA heterodimer a, DHD13_BAAA heterodimer a, DHD13_4:123 heterodimer a, DHD13_1:234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34_XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3:124 heterodimer a, DHD37_1:234 heterodimer a, DHD37_AXBB heterodimer a, DHD37_XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3:214 heterodimer a, DHD94_2:143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102_1:243 heterodimer a, DHD103 heterodimer a, DHD103_1:423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD144 heterodimer a, DHD9 heterodimer b, DHD13_XAAA heterodimer b, DHD13_XAXA heterodimer b, DHD13_XAAX heterodimer b, DHD13_2:341 heterodimer b, DHD13_AAAA heterodimer b, DHD13_BAAA heterodimer b, DHD13_4:123 heterodimer b, DHD13_1:234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37_BBBB heterodimer b, DHD37_XBXB heterodimer b, DHD37_AXXB heterodimer b, DHD37_3:124 heterodimer b, DHD37_1:234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3:214 heterodimer b, DHD94_2:143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1:243 heterodimer b, DHD103 heterodimer b, DHD103_1:423 heterodimer b, DHD104 heterodimer b, DHD105 heterodimer b, DHD106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD17 heterodimer b, DHD131 heterodimer b, DHD132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, portions thereof, derivatives thereof, or any combination thereof.

In some embodiments, one or more of the first dimerization domain, second dimerization domain, third dimerization domain, and fourth dimerization domain comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof.

One or more of the first dimerization domain, second dimerization domain, third dimerization domain, and fourth dimerization domain can be a homodimerization domain or a multimerization domain, e.g., a homo- or hetero-dimerizing or multimerizing leucine zipper, a PDZ domains, a SH3 domain, aGBD domain, or any combination thereof.

The amplifier protein and/or the companion amplifier protein can comprise a linker. In some embodiments, the linker: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; and/or comprises 3 repeating amino acid subunits or more.

In some embodiments, the amplifier protein comprises the sequence of SEQ ID NO: 244 or a sequence at least 85% (e.g., 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 244, and/or the companion amplifier protein comprises the sequence of SEQ ID NO: 245 or a sequence at least 85% (e.g., 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 245.

An amplifier can amplify the activity of an effector module (e.g., any of the effector described herein). In some embodiments, the effector module comprises a synthetic apoptosis circuit comprising: an apoptosis polypeptide comprising a large subunit of an apoptotic effector protein and a small subunit of an apoptotic effector protein separated by a fifth orthogonal protease cleavage site, wherein two apoptosis polypeptides are capable of associating with each other to constitute an apoptotic protein complex in an apoptotic protein complex inactive state, wherein the second orthogonal protease in a second orthogonal protease active state is capable of cleaving the fifth orthogonal protease cleavage site of the apoptosis polypeptide, and wherein the fifth orthogonal protease cleavage site of the apoptosis polypeptide being cleaved changes the apoptotic protein complex from the apoptotic protein complex inactive state to an apoptotic protein complex active state, thereby activating the effector module, wherein the apoptotic protein complex in the apoptotic protein complex active state is capable of inducing apoptosis in the cell.

The apoptotic effector protein can comprise caspase-3, caspase-6, caspase-7, or any variant, portion, or derivative thereof. The apoptosis polypeptide can comprise one or more linkers. In some embodiments, any of the one or more linkers: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof, and/or comprises 3 repeating amino acid subunits or more.

In some embodiments, the first orthogonal protease cleavage site, the second orthogonal cleavage site, the third orthogonal protease cleavage site, the fourth orthogonal protease cleavage site, the fifth orthogonal protease cleavage site, or any combination thereof, comprise the sequence of any one of SEQ ID NOs 247, 253, and 255-258 or a sequence having one or two mismatches relative to any one of SEQ ID NOs 247, 253, and 255-258. The apoptosis polypeptide can comprise the sequence of any one of SEQ ID NOs: 220-221 or a sequence at least 85% (e.g., 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 220-221.

In some embodiments, the effector module comprises a synthetic pyroptosis circuit comprising: a pyroptosis polypeptide comprising a pyroptosis effector domain and an inhibitory domain separated by a fifth orthogonal protease cleavage site, wherein the inhibitory domain is capable of inhibiting activity of the pyroptosis effector domain, thereby the pyroptosis polypeptide is in a pyroptosis polypeptide inactive state, wherein the second orthogonal protease in a second orthogonal protease active state is capable of cleaving the fifth orthogonal protease cleavage site of the pyroptosis polypeptide, and wherein the fifth orthogonal protease cleavage site of the pyroptosis polypeptide being cleaved changes the pyroptosis polypeptide from the pyroptosis polypeptide inactive state to a pyroptosis polypeptide active state, thereby activating the effector module, wherein the pyroptosis polypeptide in the pyroptosis polypeptide active state is capable of inducing pyroptosis in the cell.

The inhibitory domain can comprise a protein domain configured to reduce or abrogate the activity of the pyroptosis effector domain, e.g., a bulky domain. The inhibitory domain can comprise an auto-inhibitory domain of a gasdermin family of pore-forming proteins or maltose-binding protein. The pyroptosis effector domain can comprise an N-terminal domain of a gasdermin (GSDM) protein. The GSDM protein can be GSDMA, GSDMB, GSDMC, GSDMD, GSDME, or DFNB59. The pyroptosis polypeptide can comprise one or more linkers. In some embodiments, any of the one or more linkers: is a flexible linker, a rigid linker, or a hybrid linker; is hydrophilic or hydrophobic; is between 1 and 250 amino acids; comprises one or more flexible amino acid residues, optionally about 1 to about 18 flexible amino acid residues, further optionally the flexible amino acid residues comprise glycine, serine, or a combination thereof; and/or comprises 3 repeating amino acid subunits or more. In some embodiments, inducing pyroptosis in the cell causes the cell to release one or more inflammatory cytokines. The one or more inflammatory cytokines can comprise IL-18, IL-1β, IL-6, IL-8, interferon gamma (IFN-γ), and/or tumor necrosis factor-alpha (TNF-α).

An orthogonal protease cleavage site can comprise any protease cleavage site known in the art. In some embodiments, the first orthogonal protease cleavage site, the second orthogonal cleavage site, the third orthogonal protease cleavage site, the fourth orthogonal protease cleavage site, the fifth orthogonal protease cleavage site, or any combination thereof, comprise the sequence of any one of SEQ ID NOs 247, 253, and 255-258 or a sequence having one or two mismatches relative to any one of SEQ ID NOs 247, 253, and 255-258. The pyroptosis polypeptide can comprise the sequence of any one of SEQ ID NOs: 237-241 or a sequence at least 85% (e.g., 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 237-241.

In some embodiments, the first orthogonal protease and/or the second orthogonal protease comprises or is derived from a viral protease. The first orthogonal protease and/or the second orthogonal protease can comprise tobacco etch virus (TEV) protease, tobacco vein mottling virus (TVMV) protease, hepatitis C virus protease (HCVP), derivatives thereof, or any combination thereof. The first orthogonal protease and/or the second orthogonal protease can comprise the sequence of any one of SEQ ID NOs: 211, 213, and 215 or a sequence having at least 85% (e.g., 85%, 86%, 87%, 88%, 89%, 90%, 91%, 92%, 93%, 94%, 95%, 96%, 97%, 98%, 99%, 100%, or a number or a range between any two of these values) identity to any one of SEQ ID NOs: 211, 213, and 215. The first orthogonal protease and/or the second orthogonal protease can comprise one or more mutations that boost catalytic efficiency. The first orthogonal protease and/or the second orthogonal protease can comprise the sequence of any one of SEQ ID NOs: 216-217 or a sequence having at least 85% (e.g., 85%, 86%, 87%, 88%, 89%, 90%, 91%, 92%, 93%, 94%, 95%, 96%, 97%, 98%, 99%, 100%, or a number or a range between any two of these values) identity to any one of SEQ ID NOs: 216-217.

### Localization and Concentration

The first oncoprotein, the second oncoprotein, or both, can be capable of being localized at the proximity site. The first oncoprotein when in a first oncoprotein active state, the second oncoprotein when in a second oncoprotein active state, or both, can be capable of being localized at the proximity site. The first oncoprotein when in a first inactive state, the second oncoprotein when in a second inactive state, or both, can be capable of being localized at the proximity site.

In some embodiments, the first oncoprotein binding domain of the first polypeptide is capable of binding to the first oncoprotein, the second oncoprotein binding domain of the second polypeptide is capable of binding to the second oncoprotein, or both. In some embodiments, the first oncoprotein binding domain of the first polypeptide is capable of binding to the first oncoprotein in a first oncoprotein active state, the second oncoprotein binding domain of the second polypeptide is capable of binding to the second oncoprotein in a second oncoprotein active state, or both. In some embodiments, the first oncoprotein binding domain of the first polypeptide is capable of binding to the first oncoprotein in a first inactive state, the second oncoprotein binding domain of the second polypeptide is capable of binding to the second oncoprotein in a second inactive state, or both. In some embodiments, the first oncoprotein binding domain of the first polypeptide is capable of binding the first oncoprotein to form the first oncoprotein-bound polypeptide at the proximity site, the second oncoprotein binding domain of the second polypeptide is capable of binding the second oncoprotein to form the second oncoprotein-bound polypeptide at the proximity site, or both.

The proximity site can comprise one or more of a cell membrane, lipid raft, mitochondrion, peroxisome, cytosol, vesicle, lysosome, plasma membrane, nucleus, nucleolus, inner mitochondrial matrix, inner mitochondrial membrane, intermembrane space, outer mitochondrial membrane, secretory vesicle, endoplasmic reticulum, golgi body, phagosome, endosome, exosome, microtubule, microfilament, intermediate filament, sarcomere, focal contact, ribosome, microsome, plasma membrane, nuclear membrane, or any combination thereof.

A first concentration of the first oncoprotein-bound polypeptide can be at least two-fold higher at the proximity site as compared to a first cellular location other than the proximity site when the first oncoprotein is in a first oncoprotein active state. A second concentration of the second oncoprotein-bound polypeptide can be at least two-fold higher at the proximity site as compared a second cellular location other than the proximity site when the second oncoprotein is in a second oncoprotein active state. A first concentration of the first orthogonal protease in the first orthogonal protease active state can be at least two-fold higher at the proximity site as compared to a cellular location other than the proximity site when the first oncoprotein is in a first oncoprotein active state and/or when the second oncoprotein is in a second oncoprotein active state.

In some embodiments, the first part of the first orthogonal protease domain and the second part of the first orthogonal protease domain have the weak association affinity when the first oncoprotein is in a first oncoprotein inactive state and/or the second oncoprotein inactive state. In some embodiments, the first part of the first orthogonal protease domain and the second part of the first orthogonal protease domain are incapable of associating to form the first orthogonal protease in the first orthogonal protease active state when the first oncoprotein is in a first oncoprotein inactive state and/or the second oncoprotein is in a second oncoprotein inactive state. In some embodiments, a first concentration of the first oncoprotein-bound polypeptide and a second concentration of the second oncoprotein-bound polypeptide at the proximity site are insufficient for the first part of the first orthogonal protease domain and the second part of the first orthogonal protease domain to form an active first orthogonal protease when the first oncoprotein is in a first oncoprotein inactive state and/or the second oncoprotein is in a second oncoprotein inactive state. A first concentration of the first oncoprotein-bound polypeptide at the proximity site can be comparable to a first cellular location other than the proximity site when the first oncoprotein is in a first oncoprotein inactive state. A second concentration of the second oncoprotein-bound polypeptide at the proximity site can be comparable to a second cellular location other than the proximity site when the second oncoprotein is in a second oncoprotein inactive state.

In some embodiments, the first part of the first orthogonal protease domain and the second part of the first orthogonal protease domain are capable of associating with each other to form the first orthogonal protease in the first orthogonal protease active state at a threshold first polypeptide concentration and a threshold second polypeptide concentration at the proximity site. The threshold first polypeptide concentration and the threshold second polypeptide concentration at the proximity site can be reached at a threshold oncoprotein activation level of the first and/or second oncoprotein.

### Cells

The cell can be a eukaryotic cell. The cell can be a mammalian cell. The cell can be a cancer cell. The cell can be of any cell type. For example, the mammalian cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof, further optionally the stem cell comprises an embryonic stem cell, an induced pluripotent stem cell (iPSC), a hematopoietic stem/progenitor cell (HSPC), or any combination thereof.

In some embodiments, the presence and/or activation of the synthetic protein circuit in the cell does not inhibit ERK signaling in the cell. In some embodiments, the presence and/or activation of the synthetic protein circuit in the cell does not cause a significant reduction in the levels of phospho-ERK (pERK) in the cell. In some embodiments, the presence and/or activation of the synthetic protein circuit in the cell does not cause significant transcriptional perturbation in the cell.

### Compositions

Disclosed herein include nucleic acid compositions. In some embodiments, the nucleic acid composition comprises: one or more polynucleotides encoding a synthetic protein circuit of the disclosure, optionally the one or more polynucleotides comprise: one or more first polynucleotides encoding the first polypeptide, the second polypeptide, the proprotein, or any combination thereof, and/or one or more second polynucleotides encoding the protease activatable reporter, the apoptosis polypeptide and/or the pyroptosis polypeptide; optionally the one or more polynucleotides further comprise one or more third polynucleotides encoding the amplifier protein, the companion amplifier protein, or any combination thereof.

At least two of the one or more polynucleotides can be operably linked to a tandem gene expression element. In some embodiments, the one or more polynucleotides comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof, and/or a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

The one or more polynucleotides can be operably connected to a promoter selected from the group comprising: a minimal promoter, e.g., TATA, miniCMV, and/or miniPromo; a tissue-specific promoter and/or a lineage-specific promoter; and/or a ubiquitous promoter, e.g., a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof.

In some embodiments, the nucleic acid composition is configured to enhance stability, durability, and/or expression level, optionally a 5′ untranslated region (UTR), a 3′ UTR, and/or a 5′ cap; optionally one or more modified nucleotides, further optionally selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine; and/or optionally a modified nucleotide in place of one or more uridines, optionally the modified nucleoside is selected from pseudouridine (ψ), N 1-methyl-pseudouridine (m 1Ψ), and 5-methyl-uridine (m5U).

In some embodiments, the nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. At least one of the one or more vectors can be a viral vector, a plasmid, a transposable element, a naked DNA vector, a bacteriophage, or any combination thereof. The viral vector can be an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. The transposable element can be piggybac transposon or sleeping beauty transposon. The one or more polynucleotides can be comprised in the one or more vectors. The one or more polynucleotides can be comprised in the same vector and/or different vectors.

Any of the one or more polynucleotides can be situated on the same nucleic acid and/or different nucleic acids. The nucleic acid composition can comprise circular mRNA, circular DNA, self-amplifying RNA, self-amplifying RNA, and/or mRNA. The nucleic acid composition can be or can comprise mRNA. The mRNA can be formulated in a lipid nanoparticle (LNP). The mRNA can comprise a 5′ untranslated region (UTR), a 3′ UTR, and/or a cap. The mRNA can comprise one or more modified nucleotides selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine. The mRNA can comprise a modified nucleotide in place of one or more uridines. The modified nucleoside is selected from pseudouridine (ψ), N 1-methyl-pseudouridine (m 1Ψ), and 5-methyl-uridine (m5U).

The LNP can comprise one or more of an ionizable cationic lipid, a non-cationic lipid, a sterol, and a PEG-modified lipid. The non-cationic lipid can be a neutral lipid. The LNP can comprise 0.5-15 mol % PEG-modified lipid, 5-25 mol % non-cationic lipid, 25-55 mol % sterol, and 20-60 mol % ionizable cationic lipid. The LNP can comprise: 40-55 mol % ionizable cationic lipid, 5-15 mol % neutral lipid, 35-45 mol % sterol, and 1-5 mol % PEG-modified lipid. The LNP can comprise: 47 mol % ionizable cationic lipid, 11.5 mol % neutral lipid, 38.5 mol % sterol, and 3.0 mol % PEG-modified lipid; 48 mol % ionizable cationic lipid, 11 mol % neutral lipid, 38.5 mol % sterol, and 2.5 mol % PEG-modified lipid; 49 mol % ionizable cationic lipid, 10.5 mol % neutral lipid, 38.5 mol % sterol, and 2.0 mol % PEG-modified lipid; 50 mol % ionizable cationic lipid, 10 mol % neutral lipid, 38.5 mol % sterol, and 1.5 mol % PEG-modified lipid; or 51 mol % ionizable cationic lipid, 9.5 mol % neutral lipid, 38.5 mol % sterol, and 1.0 mol % PEG-modified lipid. The ionizable cationic lipid can be heptadecan-9-yl 8 ((2 hydroxyethyl)(6 oxo 6-(undecyloxy)hexyl)amino)octanoate; the neutral lipid can be 1,2 distearoyl sn glycero-3 phosphocholine (DSPC); the sterol can be cholesterol; and/or the PEG-modified lipid can be 1-monomethoxypolyethyleneglycol-2,3-dimyristylglycerol with polyethylene glycol of average molecular weight 2000 (PEG2000 DMG). The wt/wt ratio of lipid to mRNA can be from about 1:100 to about 100:1. The one or more first polynucleotides, the one or more second polynucleotides, the one or more third polynucleotides, or any combination thereof, can be formulated in separate compositions or the same composition.

Disclosed herein include pharmaceutical compositions. In some embodiments, the pharmaceutical composition comprises a nucleic acid composition of the disclosure, and at least one pharmaceutically acceptable excipient. Disclosed herein include nucleic acid compositions and pharmaceutical compositions for use as a medicament. Disclosed herein include nucleic acid compositions and pharmaceutical compositions for use in a method of treating cancer. Also provided herein are kits comprising any of the nucleic acid compositions of the disclosure, and instructions for use.

### Exemplary Sequences

Provided below is additional information regarding the synthetic circuits of the disclosure, including exemplary sequence information.

### Ras Sensor

The Ras sensor uses a split protease complementation architecture. Two polypeptides, each comprising a Ras-binding domain fused to one half of, e.g., a split TEV protease (nTEVP or cTEVP), are co-expressed. When both binder copies engage the same RAS molecule, the split protease halves are brought into proximity and reconstitute active protease activity.

### Sensor Scaffolds

The sensor scaffolds define the constant regions of each polypeptide. The binding domain ([BINDER]) is the variable module each binder listed in Table 3-Table 6 can be inserted at this position. nTEVP Half Scaffold can have the following structure: [BINDER]-Linker-nTEVP-WT-IRES-BFP. Sequences include: DNA (constant region, excluding binder), SEQ ID NO: 1; Protein (constant region, excluding binder), SEQ ID NO: 5. Additional information is provided in Table 1A-Table 1B below.

TABLE 1A
nTEVP Half Scaffold DNA

| Region | Type | Position | Length | Notes |
|---|---|---|---|---|
| Linker | DNA | 1-21 | 21 bp | Flexible linker |
| nTEVP-WT + stop | CDS | 22-375 | 354 bp | N-terminal half of |
| | | | | TEV protease |

TABLE 1B
cTEVP Half Scaffold Protein

| Region | Type | Position | Length | Notes |
|---|---|---|---|---|
| Linker | peptide | 1-7 | 7 aa | GGSGGSG (SEQ ID |
| | | | | NO: 334) |
| nTEVP-WT | CDS | 8-124 | 117 aa | N-terminal half |
| | | | | of TEV protease |

The constructs can include an IRES (598 bp): Internal ribosome entry site, downstream of nTEVP scaffold (IRES, SEQ ID NO: 2). BFP (mTagBFP2, 732 bp/243 aa), Blue fluorescent protein, can be co-expressed via an IRES. Exemplary BFP DNA sequence is SEQ ID NO: 3 and exemplary protein sequence is SEQ ID NO: 6.

cTEVP Half Scaffold can have the following structure: [BINDER]-Linker-cTEVP-WT. Sequences include: DNA (constant region, excluding binder), SEQ ID NO: 4; Protein (constant region, excluding binder), SEQ ID NO: 7. Additional information is provided in Table 2A-Table 2B below.

TABLE 2A
cTEVP Half Scaffold DNA

| Region | Type | Position | Length | Notes |
|---|---|---|---|---|
| Linker | DNA | 1-18 | 18 bp | Flexible linker |
| cTEVP-WT + stop | CDS | 19-378 | 360 bp | C-terminal half of |
| | | | | TEV protease |

TABLE 2B
cTEVP Half Scaffold Protein

| Region | Type | Position | Length | Notes |
|---|---|---|---|---|
| Linker | peptide | 1-6 | 6 aa | GGSGGS (SEQ ID |
| | | | | NO: 335) |
| cTEVP-WT | CDS | 7-125 | 119 aa | C-terminal half |
| | | | | of TEV protease |

### Ras Binding Domains

The following binding domains are used in both nTEVP and cTEVP half configurations. Each binder is inserted at the [BINDER] position in the scaffolds described above.

TABLE 3
Nature Ras Binding Domains

| | DNA | | Protein | |
|---|---|---|---|---|
| Name/Description | SEQ ID NO: | SIZE (bp) | SEQ ID NO: | SIZE (AA) |
| ARAF (aa 1-98) | 8 | 297 bp | 38 | 99 aa |
| ARAF (aa 1-167) | 9 | 504 bp | 39 | 168 aa |
| BRAF (aa 149-227) | 10 | 243 bp | 40 | 81 aa |
| RAF1 (CRAF RBD, with | 11 | 243 bp | 41 | 81 aa |
| K108A mutation) | | | | |
| RAF1 (CRAF RBD, without | 12 | 243 bp | 42 | 81 aa |
| K108A mutation) | | | | |
| RAF1 (CRAF RBD, R59A | 13 | 243 bp | 43 | 81 aa |
| mutation) | | | | |
| RAF1 RBD + CID (CRAF RBD | 14 | 399 bp | 44 | 133 aa |
| and cysteine-rich domain) | | | | |
| PIK3CG (PI3K gamma RBD) | 15 | 282 bp | 45 | 94 aa |
| PIK3CA (PI3K alpha RBD) | 16 | 363 bp | 46 | 121 aa |
| PIK3CD (PI3K delta RBD) | 17 | 327 bp | 47 | 109 aa |
| PLCE1 (aa 2135-2242, with | 18 | 336 bp | 48 | 112 aa |
| GGS linker) | | | | |
| SOS1 (aa 761-1032) | 19 | 819 bp | 49 | 273 aa |
| RIN1 (aa 620-712) | 20 | 282 bp | 50 | 94 aa |
| RAP1GDS1 (aa 181-608) | 21 | 1287 bp | 51 | 429 aa |
| MAPKAP1 (aa 269-385, | 22 | 354 bp | 52 | 118 aa |
| mSIN1 CRIM domain) | | | | |
| RASSF5 (NORE1A) | 23 | 489 bp | 53 | 163 aa |
| TIAM1 (Ras-binding domain) | 24 | 690 bp | 54 | 230 aa |
| RGL1 (aa 662-771, RA | 25 | 333 bp | 55 | 111 aa |
| domain) | | | | |
| RGL1 (aa 260-543, REM | 26 | 855 bp | 56 | 285 aa |
| domain) | | | | |
| RGL1 (aa 260-543 + | 27 | 1212 bp | 57 | 404 aa |
| GGSGGSGGS + aa 662-771, | | | | |
| REM + RA domains) | | | | |
| RGL3 (aa 606-716) | 28 | 336 bp | 58 | 112 aa |
| RASA1 (p120 RasGAP) | 29 | 993 bp | 59 | 331 aa |
| NF1 (Neurofibromin GRD) | 30 | 840 bp | 60 | 280 aa |
| RASA4 (aa 712-785 + | 31 | 1149 bp | 61 | 383 aa |
| GGSGG + aa 263-565) | | | | |
| RASA4 (aa 263-565) | 32 | 912 bp | 62 | 304 aa |
| IQGAP1 (aa 1013-1276) | 33 | 801 bp | 63 | 267 aa |
| RASAL3 | 34 | 945 bp | 64 | 315 aa |
| CRAF (RAF1 RBD, with | 35 | 249 bp | 65 | 83 aa |
| K108A) | | | | |
| HRAS-raf-binding-peptide | 36 | 492 bp | 66 | 164 aa |
| fused to CRAF RBD | | | | |
| RASSF1 RA domain | 37 | 525 bp | 67 | 175 aa |

TABLE 4
De Novo Designed Ras Binders

| | DNA | | Protein | |
|---|---|---|---|---|
| Name/Description | SEQ ID NO: | SIZE (bp) | SEQ ID NO: | SIZE (bp) |
| DNRB1 (de novo Ras binder 1) | 68 | 156 bp | 81 | 52 aa |
| DNRB3 (de novo Ras binder 3) | 69 | 225 bp | 82 | 75 aa |
| DNRB4 (de novo Ras binder 4) | 70 | 195 bp | 83 | 65 aa |
| DNRB6 (de novo Ras binder 6) | 71 | 261 bp | 84 | 87 aa |
| DNRB7 (de novo Ras binder 7) | 72 | 180 bp | 85 | 60 aa |
| DNRB8 (de novo Ras binder 8) | 73 | 228 bp | 86 | 76 aa |
| DNRB9 (de novo Ras binder 9) | 74 | 252 bp | 87 | 84 aa |
| DNRB10 (de novo Ras binder 10) | 75 | 201 bp | 88 | 67 aa |
| DNRB11 (de novo Ras binder 11) | 76 | 180 bp | 89 | 60 aa |
| DNRB12 (de novo Ras binder 12) | 77 | 258 bp | 90 | 86 aa |
| DNRB13 (de novo Ras binder 13) | 78 | 204 bp | 91 | 68 aa |
| DNRB14 (de novo Ras binder 14) | 79 | 210 bp | 92 | 70 aa |
| DNRB15 (de novo Ras binder 15) | 80 | 183 bp | 93 | 61 aa |

TABLE 5
Synthetic Ras Binding Domains

| | DNA | | Protein | |
|---|---|---|---|---|
| Name/Description | SEQ ID NO: | SIZE (bp) | SEQ ID NO: | SIZE (AA) |
| NS1 monobody | 94 | 318 bp | 99 | 106 aa |
| JAM20 | 95 | 291 bp | 100 | 97 aa |
| K13 DARPin | 96 | 471 bp | 101 | 157 aa |
| K19 DARPin | 97 | 471 bp | 102 | 157 aa |
| R15m10 | 98 | 279 bp | 103 | 93 aa |

TABLE 6
12VC binders

| | DNA | | Protein | |
|---|---|---|---|---|
| Name/Description | SEQ ID NO: | SIZE (bp) | SEQ ID NO: | SIZE (AA) |
| 12VC1 | 104 | 288 bp | 109 | 96 aa |
| 12VC3 | 105 | 288 bp | 110 | 96 aa |
| 12D4 Monobody | 106 | 279 bp | 111 | 93 aa |

### Full Sensor Polyprotein Sequences

Complete polyprotein sequences for the two primary Ras sensor constructs are described below. Each polyprotein encodes two copies of the binder flanking split protease halves, with TEV cleavage sites serving as the protease substrate. 12VC1 Polyprotein can have the following structure: M-12VC1-GGSGGS-nTEVP(uTEV3)-GSGSS-TEVs-GGGGS-TEVs-GGSGS-12VC1-GGSGGS-cTEVP(uTEV3)*-S. Further details can be found in Table 7 below.

TABLE 7
12VC1 Polyprotein

| | | Position | | | |
|---|---|---|---|---|---|
| Domain | Type | (aa) | Length | | Notes |
| M | start | 1 | 1 | aa | Initiator methionine |
| G | residue | 2 | 1 | aa | N-terminal glycine |
| 12VC1 | CDS | 3-97 | 95 | aa | First copy; anti-RAS monobody |
| GGSGGS* | linker | 98-103 | 6 | aa | Flexible linker |
| nTEVP | CDS | 104-221 | 118 | aa | N-terminal half of uTEV3 protease |
| GSGSS* | linker | 222-226 | 5 | aa | Flexible linker |
| TEVs #1 | CDS | 227-233 | 7 | aa | ENLYFQS (SEQ ID NO: 247); TEV |
| | | | | | protease cleavage site |
| GGGGS* | linker | 234-238 | 5 | aa | Flexible linker |
| TEVs #2 | CDS | 239-245 | 7 | aa | ENLYFQS (SEQ ID NO: 247); TEV |
| | | | | | protease cleavage site |
| GGSGS* | linker | 246-250 | 5 | aa | Flexible linker |
| 12VC1 | CDS | 251-345 | 95 | aa | Second copy; anti-RAS monobody |
| GGSGGS* | linker | 346-351 | 6 | aa | Flexible linker |
| cTEVP | CDS | 352-469 | 118 | aa | C-terminal half of uTEV3 protease |
| S | residue | 470 | 1 | aa | C-terminal serine |

*GGSGGS, SEQ ID NO: 335 GSGSS, SEQ ID NO: 336 GGGGS, SEQ ID NO: 337 GGSGS, SEQ ID NO: 338

12VC1 polyprotein sequences include: 12VC1 polyprotein DNA (1413 bp), SEQ ID NO: 107. 12VC1 polyprotein Protein (470 aa), SEQ ID NO: 112.

12D4 Polyprotein can have the following structure: M-G-12D4-GGSGGS-nTEVP(uTEV3)-GSGSS-TEVs-GGGGS-TEVs-GGSGS-12D4-GGSGGS-cTEVP(uTEV3)-S. Further details can be found in Table 8 below.

TABLE 8
12D4 Polyprotein

| | | Position | | | |
|---|---|---|---|---|---|
| Domain | Type | (aa) | Length | | Notes |
| M | start | 1 | 1 | aa | Initiator methionine |
| G | residue | 2 | 1 | aa | N-terminal glycine |
| 12D4 | CDS | 3-94 | 92 | aa | First copy; thermostabilized anti- |
| | | | | | RAS monobody |
| GGSGGS* | linker | 95-100 | 6 | aa | Flexible linker |
| nTEVP | CDS | 101-218 | 118 | aa | N-terminal half of uTEV3 protease |
| GSGSS* | linker | 219-223 | 5 | aa | Flexible linker |
| TEVs #1 | CDS | 224-230 | 7 | aa | ENLYFQS (SEQ ID NO: 247); TEV |
| | | | | aa | protease cleavage site |
| GGGGS* | linker | 231-235 | 5 | aa | Flexible linker |
| TEVs #2 | CDS | 236-242 | 7 | aa | ENLYFQS (SEQ ID NO: 247); TEV |
| | | | | | protease cleavage site |
| GGSGS* | linker | 243-247 | 5 | aa | Flexible linker |
| 12D4 | CDS | 248-339 | 92 | aa | Second copy; anti-RAS monobody |
| GGSGGS* | linker | 340-345 | 6 | aa | Flexible linker |
| cTEVP | CDS | 346-463 | 118 | aa | C-terminal half of uTEV3 protease |
| S | residue | 464 | 1 | aa | C-terminal serine |

*GGSGGS, SEQ ID NO: 335 GSGSS, SEQ ID NO: 336 GGGGS, SEQ ID NO: 337 GGSGS, SEQ ID NO: 338 GGSGGS, SEQ ID NO: 335

### Architectural Notes

Both polyproteins above share identical architecture and differ only in the binder domain: 12VC1, 95 aa monobody; binds RAS via the Switch I/II pocket; 12D4, 92 aa thermostabilized monobody (12D1>I20V/P51S/W78F/Y79W); binds the same RAS pocket. The split protease halves (nTEVP and cTEVP) are from the uTEV3 variant, an ultra-active engineered TEV protease carrying mutations I138T, S153N, T180A, and S219V (cTEVP half). The dual TEV cleavage sites (TEVs TEVs) flanking the GGGGS (SEQ ID NO: 337) linker serve as the protease substrate. When the two binder copies engage the same RAS molecule, the split protease halves are brought into proximity and reconstitute active protease, which then cleaves these sites to release the downstream signal.

### p53 Sensor System

The p53 sensor system uses the same split protease complementation architecture as the Ras sensor. Two polypeptides, each comprising a p53-binding domain fused to one half of a split TEV protease, are co-expressed. p53 forms tetramers in the nucleus; when both binder-protease fusions engage the same p53 tetramer (or adjacent p53 molecules), the split protease halves are brought into proximity and reconstitute active protease. In some embodiments, binders target a cryptic epitope exposed selectively in conformationally destabilized mutant p53, enabling discrimination of mutant from wild-type p53 based on epitope accessibility rather than abundance alone.

### Sensor Scaffold

Each sensor polypeptide follows the generalized architecture: [TAG/LOCALIZATION]-[BINDER]-Linker-[PROTEASE HALF]-[MARKER].

Where TAG is an optional localization or solubility tag (e.g., bipartite NLS for nuclear targeting, SUMOstar for solubility), BINDER is a p53-binding domain (see Table 11-Table 12), PROTEASE HALF is nTEVP or cTEVP, and an optional MARKER is a fluorescent protein (e.g., mTagBFP2, mRuby3). The protease halves (nTEVP and cTEVP) are identical to those used in the Ras sensor system (see above). The constant regions (linker+protease half) are shown below with a [BINDER] placeholder; each binder from Table 11-Table 12 is inserted at this position.

cTEVP Half Scaffold can have the structure: [TAG]-[BINDER]-Linker-cTEVP-[MARKER]. Also see Table 9 below.

TABLE 9
cTEVP Half Scaffold

| Region | Type | Position | Length | Notes |
|---|---|---|---|---|
| Linker | peptide | variable | 20 aa | GSEGSGSEGSGSEGSG |
| (L20) | | | | SEGS (SEQ ID NO: |
| | | | | 339) |
| cTEVP | CDS | variable | 119 aa | C-terminal half |
| | | | | of TEV protease |

cTEVP Half Scaffold sequences can include: DNA (constant region, excluding binder) sequence, SEQ ID NO: 114; Protein (constant region, excluding binder) sequence, SEQ ID NO: 116.

nTEVP Half Scaffold can have the structure: [TAG]-[BINDER]-Linker-nTEVP-[MARKER]. Also see Table 10 below.

TABLE 10
nTEVP Half Scaffold

| Region | Type | Position | Length | Notes |
|---|---|---|---|---|
| Linker | peptide | variable | 15 aa | GSEGSGSEGSGSEGS |
| | | | | (representative, |
| | | | | SEQ ID NO: 340) |
| nTEVP | CDS | variable | 118 aa | N-terminal half |
| | | | | of TEV protease |

nTEVP Half Scaffold sequences can include: DNA (constant region, excluding binder) sequence, SEQ ID NO: 115; Protein (constant region, excluding binder) sequence, SEQ ID NO: 117.

### De Novo Designed p53 Binders (Cryptic Epitope)

The following 23 de novo designed protein binders target the cryptic epitope of the p53 DNA-binding domain (FRHSV region). This epitope is buried in correctly folded wild-type p53 and selectively exposed in conformationally destabilized mutant p53. Each binder is inserted at the [BINDER] position in the scaffolds described above.

TABLE 11
De Novo Designed p53 Binders

| | DNA | | Protein | |
|---|---|---|---|---|
| Name/Description | SEQ ID NO: | SIZE (bp) | SEQ ID NO: | SIZE (AA) |
| P53B_004 | 118 | 285 bp | 141 | 95 aa |
| P53B_006 | 119 | 297 bp | 142 | 99 aa |
| P53B_008 | 120 | 330 bp | 143 | 110 aa |
| P53B_014 | 121 | 246 bp | 144 | 82 aa |
| P53B_024 | 122 | 213 bp | 145 | 71 aa |
| P53B_026 | 123 | 216 bp | 146 | 72 aa |
| P53B_029 | 124 | 255 bp | 147 | 85 aa |
| P53B_032 | 125 | 285 bp | 148 | 95 aa |
| P53B_033 | 126 | 285 bp | 149 | 95 aa |
| P53B_039 | 127 | 312 bp | 150 | 104 aa |
| P53B_041 | 128 | 321 bp | 151 | 107 aa |
| P53B_042 | 129 | 216 bp | 152 | 72 aa |
| P53B_043 | 130 | 216 bp | 153 | 72 aa |
| P53B_048 | 131 | 291 bp | 154 | 97 aa |
| P53B_049 | 132 | 291 bp | 155 | 97 aa |
| P53B_051 | 133 | 273 bp | 156 | 91 aa |
| P53B_058 | 134 | 345 bp | 157 | 115 aa |
| P53B_063 | 135 | 273 bp | 158 | 91 aa |
| P53B_065 | 136 | 348 bp | 159 | 116 aa |
| P53B_066 | 137 | 357 bp | 160 | 119 aa |
| P53B_067 | 138 | 291 bp | 161 | 97 aa |
| P53B_070 | 139 | 264 bp | 162 | 88 aa |
| P53B_073 | 140 | 291 bp | 163 | 97 aa |

### p53 Binding Domains

The following binding domains target p53 through known biological interfaces. Each is inserted at the [BINDER] position in a scaffold as described herein.

Nb139 Nanobody (P-0561): Anti-p53 VHH nanobody (single-domain antibody) targeting p53. scFV_ME1 (P-0560): Single-chain variable fragment (scFv) derived from the ME1 anti-p53 antibody, contains VH and VL domains connected by a (GGGGS)3 (SEQ ID NO: 360) linker, followed by a short framework spacer. MDM2 p53-Binding Domain (no lid) (P-0562): the p53-binding domain of human MDM2 (murine double minute 2), with the N-terminal lid region removed, MDM2 is a major endogenous negative regulator of p53, binding the p53 transactivation domain. EP300 TAZ2/CH3 Domain (P-0563): the TAZ2 (transcriptional adaptor zinc-binding domain 2, also known as CH3) domain of human EP300 (p300 histone acetyltransferase); this zinc-finger domain mediates direct binding to the p53 activation domain. ASPP1 p53-Binding Domain (P-0564): the p53-binding domain of human ASPP1 (apoptosis-stimulating protein of p53-1). ASPP1 binds the DNA-binding domain of p53 and stimulates p53-dependent apoptosis by enhancing the transactivation of pro-apoptotic target genes. Top7 (Negative Control) (P-0559): top7 is a computationally designed protein (Kuhlman et al., Science, 2003) with a novel fold not found in nature; it has no known binding affinity for p53 and serves as a negative control to establish baseline signal in the split-TEV protease complementation assay. Further details are provided in Table 12 below.

TABLE 12
p53 Binding Domains

| | DNA | | Protein | |
|---|---|---|---|---|
| Name/Description | SEQ ID NO: | SIZE (bp) | SEQ ID NO: | SIZE (AA) |
| Nb139 Nanobody (P-0561). | 164 | 378 bp | 170 | 126 aa |
| Anti-p53 VHH nanobody | | | | |
| (single-domain antibody) | | | | |
| targeting p53 | | | | |
| scFV_ME1 (P-0560) | 165 | 774 bp | 171 | 258 aa |
| MDM2 p53-Binding Domain | 166 | 255 bp | 172 | 85 aa |
| (no lid) (P-0562) | | | | |
| EP300 TAZ2/CH3 Domain | 167 | 222 bp | 173 | 74 aa |
| (P-0563) | | | | |
| ASPP1 p53-Binding Domain | 168 | 684 bp | 174 | 228 aa |
| (P-0564) | | | | |
| Top7 (Negative Control) | 169 | 279 bp | 175 | 93 aa |
| (P-0559) | | | | |

### TP53 Target Sequences

Wild-type and mutant TP53 protein sequences (WT, R175H, Y220C, R248Q) used as endogenous sensor targets are provided in, e.g., Table 31. These are endogenous proteins detected by the sensor; they are not part of the sensor construct.

### Beta-Catenin Sensor System

The beta-catenin sensor system uses the same split protease complementation architecture, but with TVMV protease instead of TEV. Two polypeptides, each comprising a beta-catenin-binding domain fused to one half of a split TVMV protease (nTVMVP or cTVMVP-I40D), are co-expressed.

### Sensor Scaffolds

nTVMVP Half Scaffold can have the structure: [BINDER]-Linker-nTVMVP-GSG-T2A-GS-mTagBFP2.

TABLE 13
nTVMVP Half Scaffold

| Region | Type | Position | Length | Notes |
|---|---|---|---|---|
| Linker | linker | 1-18 | 18 aa | EGSGSEGSGSEGSGSEGS |
| | | | | (SEQ ID NO: 341) |
| nTVMVP | CDS | 19-136 | 118 aa | N-terminal half of TVMV |
| | | | | protease |
| GSG | linker | 137-139 | 3 aa | Spacer |
| T2A | CDS | 140-157 | 18 aa | Self-cleaving peptide |
| GS | linker | 158-159 | 2 aa | Spacer |
| mTagBFP2 | CDS | 160-395 | 236 aa | Blue fluorescent protein |

Sequences can include: DNA (constant region, 1188 bp), SEQ ID NO: 176; Protein (constant region, 395 aa), SEQ ID NO: 178.

cTVMVP Half Scaffold can have the structure: [BINDER]-Linker-cTVMVP-140D. The I40D mutation (isoleucine to aspartate at position 40 of the cTVMVP fragment) is a destabilizing mutation used to reduce basal protease activity.

TABLE 14
cTVMVP Half Scaffold

| Region | Type | Position | Length | Notes |
|---|---|---|---|---|
| Linker | linker | 1-18 | 18 aa | EGSGSEGSGSEGSGS |
| | | | | EGS (SEQ ID NO: |
| | | | | 341) |
| cTVMVP- | CDS | 19-136 | 118 aa | C-terminal half |
| 140D | | | | of TVMV protease |
| | | | | (140D |
| | | | | destabilizing |
| | | | | mutation) |

cTVMVP half scaffold sequences can include: DNA (constant region, 414 bp), SEQ ID NO: 177; Protein (constant region, 137 aa), SEQ ID NO: 179.

### Beta-Catenin Binding Domains

Table 15 provides sequences of natural beta-catenin binding domains. Table 16 below provides sequences of beta-catenin nanobodies.

TABLE 15
Natural Beta-Catenin Binding Domains

| | DNA | | Protein | |
|---|---|---|---|---|
| Name/Description | SEQ ID NO: | SIZE (bp) | SEQ ID NO: | SIZE (AA) |
| TCF7 (first 60 AA) | 180 | 189 bp | 187 | 63 aa |
| LEF1 (first 60 AA) | 181 | 189 bp | 188 | 63 aa |
| Axin1-CBD (long) | 182 | 201 bp | 189 | 67 aa |
| BCL9-CBD | 183 | 147 bp | 190 | 49 aa |
| E-cadherin cytoplasmic | 184 | 180 bp | 191 | 60 aa |
| domain (CDH1, short) | | | | |
| ICAT (CTNNBIP1) | 185 | 249 bp | 192 | 83 aa |
| alpha-Catenin (CTNNA1) | 186 | 630 bp | 193 | 210 aa |

TABLE 16
Beta-Catenin Nanobodies

| | DNA | | Protein | |
|---|---|---|---|---|
| Name/Description | SEQ ID NO: | SIZE (bp) | SEQ ID NO: | SIZE (AA) |
| CTNNB1 nanobody BC1 | 194 | 435 bp | 199 | 145 aa |
| CTNNB1 nanobody BC6 | 195 | 429 bp | 200 | 143 aa |
| CTNNB1 nanobody BC9 | 196 | 435 bp | 201 | 145 aa |
| CTNNB1 nanobody BC13 | 197 | 420 bp | 202 | 140 aa |

### TCF7Full-Length

TCF7 (full-length, codon-optimized) can have the following sequences: DNA sequence, SEQ ID NO: 198; protein sequence SEQ ID NO: 203.

### Proteases

### TEV Protease

Full-length TEV protease fused to mTagBFP2 reporter can have the following construct architecture: Met-TEV-protease-GSEGSGSEGS linker-mTagBFP2. Sequence elements are provided in Table 17 below.

TABLE 17
TEV Protease Construct

| Domain | Type | Position (aa) | Length | Notes |
|---|---|---|---|---|
| Met | start | 1 | 1 aa | Initiator methionine |
| TEV protease | CDS | 2-240 | 239 aa | Full TEV protease domain |
| GSEGSGSEGS | linker | 241-250 | 10 aa | Flexible linker |
| linker (SEQ ID | | | | |
| NO: 356) | | | | |
| mTagBFP2 | CDS | 251-486 | 236 aa | Blue fluorescent reporter |

TEV Protease Construct sequences include: full construct DNA (1461 bp): SEQ ID NO: 204; Full construct protein (486 aa): SEQ ID NO: 210; TEV protease domain only (239 aa): SEQ ID NO: 211.

### TVMV Protease

TVMV protease construct can have the following architecture: Met-GS-TVMV protease-GS. Sequence elements are provided in Table 18 below.

TABLE 18
TVMV Protease Construct

| Domain | Type | Position (aa) | Length | Notes |
|---|---|---|---|---|
| Met | start | 1 | 1 aa | Initiator methionine |
| GS | linker | 2-3 | 2 aa | Cloning-derived spacer |
| TVMV protease | CDS | 4-239 | 236 aa | Full TVMV protease domain |
| GS | linker | 240-241 | 2 aa | C-terminal spacer |

TVMV Protease Construct sequences include: full construct DNA (726 bp), SEQ ID NO: 205; Full construct protein (241 aa), SEQ ID NO: 212; TVMV protease domain only (236 aa), SEQ ID NO: 213.

### HCV NS3/4A Protease

Construct architecture can include: Met GS P1 peptide GGSGS NS4A cofactor-GSGS-HCV NS3 protease-SGSG-T2A-GS-mBeRFP. Table 19 below provides sequence elements. GGSGS linker, SEQ ID NO: 338.

TABLE 19
HCV NS3/4A Protease Construct

| Domain | Type | Position (aa) | Length | Notes |
|---|---|---|---|---|
| Met | start | 1 | 1 aa | Initiator methionine |
| P1 peptide | CDS | 4-36 | 33 aa | Heterodimerization domain |
| NS4A cofactor | CDS | 42-53 | 12 aa | Activating peptide for NS3 |
| HCV NS3 | CDS | 58-250 | 193 aa | Full catalytic domain |
| protease | | | | |
| T2A | CDS | 255-272 | 18 aa | Self-cleaving peptide |
| mBeRFP | CDS | 275-508 | 234 aa | Red fluorescent reporter |

HCV NS3/4A Protease Construct sequences include: full construct DNA (1527 bp), SEQ ID NO: 206; Full construct protein (508 aa), SEQ ID NO: 214; HCV NS3 protease domain only (193 aa), SEQ ID NO: 215.

### Enhanced TEV Protease Variants

Enhanced TEV proteases disclosed herein include uTEV3 and eTEV. uTEV3 mutations vs WT TEVP: I138T, S153N, T180A, S219V (all in cTEVP half). eTEV mutations vs WT TEVP: S3I, P8Q, S31T (nTEVP half); T173A, S219R, A231V (cTEVP half). Sequences are provided in Table 20 below. Mutations are listed in Table 21.

TABLE 20
Enhanced Protease Variants

| | DNA | | Protein | |
|---|---|---|---|---|
| Name/Description | SEQ ID NO: | SIZE (bp) | SEQ ID NO: | SIZE (AA) |
| uTEV3 (Original codons) | 207 | 708 bp | 216 | 236 aa |
| uTEV3 (Codon optimized) | 208 | 708 bp | 216 | 236 aa |
| eTEV | 209 | 708 bp | 217 | 236 aa |

TABLE 21
Mutation Comparison

| Position | WT TEVP | uTEV3 | eTEV | Region |
|---|---|---|---|---|
| 3 | S | S | I | nTEVP |
| 8 | P | P | Q | nTEVP |
| 31 | S | S | T | nTEVP |
| 138 | I | T | I | cTEVP |
| 153 | S | N | S | cTEVP |
| 173 | T | T | A | cTEVP |
| 180 | T | A | T | cTEVP |
| 219 | S | V | R | cTEVP |
| 231 | A | A | V | cTEVP |

### Effector Modules

### Engineered Caspase-3 Constructs

Caspase-3 with TVMV Release construct can have the following structure: M-Casp3te(large subunit)-TEVs-Casp3te(small subunit)-GS-TVMVs-SGGGG-CAAX (SGGGG linker, SEQ ID NO: 342).

Engineered caspase-3 with the natural inter-subunit linker replaced by a TEV protease cleavage site (TEVs). Tethered to the plasma membrane via a C-terminal CAAX motif. A TVMV protease cleavage site between the caspase and CAAX enables regulated membrane release. Sequences include: DNA (966 bp), SEQ ID NO: 218; Protein (321 aa), SEQ ID NO: 220.

Membrane-Tethered Caspase-3 can have the following structure: M-Casp3te(large subunit)-TEVs-Casp3te(small subunit)-20aa GSEGS linker-CAAX (4×GSEGS linker, SEQ ID NO: 339). Same engineered caspase-3 as disclosed above, but with the TVMV protease-releasable linker replaced by a constitutive 20-amino acid (GSEGS)x4 flexible linker (SEQ ID NO: 339) between the caspase and the CAAX membrane anchor. This membrane-tethered variant lacks the regulated membrane release mechanism, providing a comparison construct. Sequences include: DNA (981 bp), SEQ ID NO: 219; Protein (326 aa), SEQ ID NO: 221.

### Architectural Notes

Both caspase-3 effectors share the same core engineered enzyme (Casp3te, 283 aa including Met). The caspase-3 large subunit (p17, 173 aa) contains the active site. The TEV site (ENLYFQS, SEQ ID NO; 247) replaces the natural IETD inter-subunit linker. Cleavage at this site by reconstituted TEV protease separates the large and small subunits, activating the caspase.

Both use the same CAAX motif (RSKLNPPDESGPGCMSCKCVLS (SEQ ID NO: 332), 22 aa) derived from KRAS4B for plasma membrane localization via prenylation.

### Gasdermin N-Terminal Pore-Forming Domains

When released from their C-terminal autoinhibitory domains by protease cleavage, gasdermin N-terminal domains insert into the cell membrane to form pores, triggering pyroptosis. Table 22 below provides exemplary sequences for gasdermin N-terminal domains.

TABLE 22
Gasdermin N-Terminal Pore-Forming Domains

| | DNA | | Protein | |
|---|---|---|---|---|
| Name/Description | SEQ ID NO: | SIZE (bp) | SEQ ID NO: | SIZE (AA) |
| GSDMA N-terminus | 222 | 738 bp | 227 | 246 aa |
| GSDMB N-terminus | 223 | 732 bp | 228 | 244 aa |
| GSDMC N-terminus | 224 | 720 bp | 229 | 240 aa |
| GSDMD N-terminus | 225 | 825 bp | 230 | 275 aa |
| GSDME N-terminus | 226 | 810 bp | 231 | 270 aa |

### Full-Length Engineered Gasdermins (TEV-Activatable)

Full-length gasdermins with the natural cleavage site replaced by a TEV protease cleavage site (ENLYFQS, SEQ ID NO: 247). TEV protease activity releases the N-terminal pore-forming domain from C-terminal autoinhibition. Provided below in Table 23 are sequences for exemplary protease activatable gasdermins.

TABLE 23
Full-Length Engineered Gasdermins (TEV-Activatable)

| | DNA | | Protein | |
|---|---|---|---|---|
| Name/Description | SEQ ID NO: | SIZE (bp) | SEQ ID NO: | SIZE (AA) |
| GSDMA with TEV site | 232 | 1359 bp | 237 | 452 aa |
| (cleavage after position 246) | | | | |
| GSDMB with TEV site | 233 | 1272 bp | 238 | 423 aa |
| (cleavage after position 244) | | | | |
| GSDMC with TEV site | 234 | 1548 bp | 239 | 515 aa |
| (cleavage after position 240) | | | | |
| GSDMD with TEV site | 235 | 1476 bp | 240 | 491 aa |
| (cleavage after position 275) | | | | |
| GSDME with TEV site | 236 | 1512 bp | 241 | 503 aa |
| (cleavage after position 270) | | | | |

### Signal Amplifier

The amplifier construct encodes three polypeptides from a single mRNA via T2A and P2A ribosomal skipping peptides. Sequences include: full construct DNA (3081 bp), SEQ ID NO: 242; Full construct protein (1026 aa, pre-cleavage): SEQ ID NO: 243.

### Amplifier Protein (Polypeptide 1, 363 aa)

The amplifier protein can have the structure: N-TVMV(WT)-linker-P3-linker-TEVsite-linker-AP4 ms-TEVsite-C-TVMV(C151A)-T2A. Sequences include: Protein (363 aa), SEQ ID NO: 244. Sequence elements are provided below in Table 24.

TABLE 24
Amplifier Protein

| | Position | | | |
|---|---|---|---|---|
| Domain | (aa) | Length | | Notes |
| N-TVMV (WT) | 1-119 | 119 | aa | Active; H46, D81 intact |
| Linker | 120-130 | 11 | aa | GSEGSGSEGSGS (SEQ ID NO: 343) |
| P3 coiled-coil | 131-163 | 33 | aa | Binds AP3ms on Polypeptide 2 |
| Linker | 164-169 | 6 | aa | GGSGGG (SEQ ID NO: 344) |
| TEV site | 170-176 | 7 | aa | ENLYFQS (SEQ ID NO: 247) |
| Linker | 177-182 | 6 | aa | GSSGSG (SEQ ID NO: 345) |
| AP4ms coiled-coil | 183-215 | 33 | aa | Binds P4 on Polypeptide 2 |
| Linker + TEV site | 216-224 | 9 | aa | GS + ENLYFQS (SEQ ID NO: 346) |
| C-TVMV (C151A) | 225-343 | 119 | aa | Catalytically dead (C151A) |
| T2A | 344-363 | 20 | aa | Ribosomal skipping peptide |

### Companion Amplifier Protein (Polypeptide 2, 366 aa)

Companion amplifier protein can have the structure: N-TVMV(H46R,D81N)-TEVsite-AP3 ms-TEVsite-P4-C-TVMV(WT) P2A. Sequences include: Protein (366 aa). Companion amplifier protein can comprise the sequence of SEQ ID NO: 245. Sequence elements are provided below in Table 25.

TABLE 25
Companion Amplifier Protein

| | Position | | | |
|---|---|---|---|---|
| Domain | (aa) | Length | | Notes |
| N-TVMV (H46R, D81N) | 1-122 | 122 | aa | Catalytically dead |
| TEV site | 125-131 | 7 | aa | ENLYFQS (SEQ ID NO: 247) |
| AP3ms coiled-coil | 134-166 | 33 | aa | Binds P3 on Polypeptide 1 |
| Linker + TEV site | 167-179 | 13 | aa | GGSSGSG + ENLYFQS (SEQ ID |
| | | | | NO: 347) |
| P4 coiled-coil | 186-217 | 32 | aa | Binds AP4ms on Polypeptide 1 |
| Linker | 218-227 | 10 | aa | SGSEGSGSEGS (SEQ ID NO: 348) |
| C-TVMV (WT) | 228-345 | 118 | aa | Active; C151 intact |
| P2A | 346-366 | 21 | aa | Ribosomal skipping peptide |

### HaloTag Reporter (Polypeptide 3, 297 aa)

HaloTag reporter can have the following sequence: Protein (297 aa), SEQ ID NO: 246.

### Circuit Logic

Polypeptide 1 has an active N-TVMV half and an inactive C-TVMV half (C151A). Polypeptide 2 has an inactive N-TVMV half (H46R, D81N) and an active C-TVMV half. Coiled-coil pairs (P3/AP3 ms and P4/AP4 ms) tether the two polypeptides as a dimer. When TEV sites are cleaved by upstream sensor signal, the active halves (N-TVMV from Poly1+C-TVMV from Poly2) are liberated and reconstitute active split-TVMV protease, creating a positive feedback amplification loop.

### Cleavage Sites

### TEV Protease Cleavage Sites

Consensus: ENLYFQ/S (cleavage between Q and S), SEQ ID NO: 247.

All TEV cleavage sites encode the canonical ENLYFQS heptapeptide. Five distinct DNA codon variants were identified, reflecting different codon optimization strategies. The deliberate codon diversification between paired sites within the same construct prevents homologous recombination during plasmid propagation. Sequences are provided in Table 26 below.

TABLE 26
TEV Protease Cleavage Sites

| | DNA Sequence | Codons (E-N-L-Y-F-Q-S), |
|---|---|---|
| Variant | (21 bp) | SEQ ID NO: 247 |
| TEVs-v1 | SEQ ID NO: 248 | GAA-AAC-CTT-TAT-TTC-CAG-TCT |
| tevs-v1 | SEQ ID NO: 249 | GAG-AAT-CTC-TAC-TTT-CAG-AGT |
| TEVs-v2 | SEQ ID NO: 250 | GAG-AAC-TTG-TAC-TTC-CAG-TCC |
| TEVs-v3 | SEQ ID NO: 251 | GAG-AAC-TTG-TAC-TTC-CAG-AGT |
| TEVs-v4 | SEQ ID NO: 252 | GAA-AAC-CTT-TAC-TTC-CAA-TCA |

### TVMV Protease Cleavage Site

Consensus: ETVRFQ/S (cleavage between Q and S), SEQ ID NO: 253

TABLE 27
TVMV Protease Cleavage Sites

| | DNA Sequence | | |
|---|---|---|---|
| Variant | (21 bp) | Protein | Context |
| TVMVs-1 | SEQ ID NO: 254 | ETVRFQS | Between caspase-3 |
| | | (SEQ ID | C-terminus and |
| | | NO: 253) | CAAX membrane |
| | | | anchor |

### HCV Protease Cleavage Sites (Literature Reference)

HCV NS3/4A protease substrates from literature are shown below.

TABLE 28
HCV Protease Cleavage Sites

| Junction | Sequence | P1/P1′ | Notes |
|---|---|---|---|
| NS3/4A | DLEVVT/STWVL | T/S | Auto- |
| junction | (SEQ ID NO: 255) | | cleavage |
| NS4A/4B | DEMEEC/ASHL | C/A | |
| junction | (SEQ ID NO: 256) | | |
| NS4B/5A | DCSTPC/SGSW | C/S | |
| junction | (SEQ ID NO: 257) | | |
| NS5A/5B | EDVVCC/SMSYTW | C/S | |
| junction | (SEQ ID NO: 258) | | |

iProtease Reporter Constructs

Generalized iProtease reporters based on split GFP and split mIFP. The protease cleavage site is modular and can be swapped to make the reporter responsive to different proteases. The localization tag determines the subcellular compartment where the reporter operates.

Structure can include: GFP(1-10)-GGSGS-mIFP(N)-[CLEAVAGE SITE]-mIFP(C)-GGS-GFP11 [LOCALIZATION TAG](GGSGS linker, SEQ ID NO: 338).

TABLE 29
iProtease Reporter Constructs

| Localization | C-terminal Tag | Cleavage Site Options |
|---|---|---|
| Cytoplasmic | No tag | TEV: ENLYFQS¹/TVMV: ETVRFQS² |
| Nuclear | SV40-NLSx3 (34 aa) | TEV: ENLYFQS¹/TVMV: ETVRFQS² |
| Membrane | SGGG-CAAX (27 aa) | TEV: ENLYFQS¹/TVMV: ETVRFQS² |

¹SEQ ID NO: 247
²SEQ ID NO: 253

TABLE 30
iProtease Reporter Constructs - Sequences

| | DNA | | Protein | |
|---|---|---|---|---|
| Name/Description | SEQ ID NO: | SIZE (bp) | SEQ ID NO: | SIZE (AA) |
| iTEVP (cytoplasmic) | 259 | 1641 bp | 265 | 546 aa |
| iTEVP-NLSx3 (nuclear) | 260 | 1743 bp | 266 | 580 aa |
| iTEVP-CAAX (membrane) | 261 | 1722 bp | 267 | 573 aa |
| iTVMVP (cytoplasmic) | 262 | 1641 bp | 268 | 546 aa |
| iTVMVP-NLSx3 (nuclear) | 263 | 1743 bp | 269 | 580 aa |
| iTVMVP-CAAX (membrane) | 264 | 1722 bp | 270 | 573 aa |

### Additional Sequences

### TP53 Variant Sequences

Exemplary wild-type and mutant TP53 proteins used as targets in p53 native binder sensor constructs are provided in the table below.

TABLE 31
TP53 Variant Sequences

| | DNA | | Protein | |
|---|---|---|---|---|
| Name/Description | SEQ ID NO: | SIZE (bp) | SEQ ID NO: | SIZE (AA) |
| TP53 Wild-type | 271 | 1176 bp | 295 | 392 aa |
| TP53 R175H | 272 | 1176 bp | 296 | 392 aa |
| TP53 Y220C | 273 | 1176 bp | 297 | 392 aa |
| TP53 R248Q | 274 | 1176 bp | 298 | 392 aa |

TABLE 32
CTNNB1 Target Proteins

| | DNA | | Protein | |
|---|---|---|---|---|
| Name/Description | SEQ ID NO: | SIZE (bp) | SEQ ID NO: | SIZE (AA) |
| WT CTNNB1 (beta-catenin) | 275 | 2343 bp | 299 | 781 aa |
| S33Y CTNNB1 (constitutively | 276 | 2343 bp | 300 | 781 aa |
| active mutant) | | | | |

TABLE 33
Un-engineered Full-Length Gasdermins (Reference)

| | DNA | | Protein | |
|---|---|---|---|---|
| Name/Description | SEQ ID NO: | SIZE (bp) | SEQ ID NO: | SIZE (AA) |
| GSDMA (full-length, | 277 | 1338 bp | 301 | 445 aa |
| unengineered) | | | | |
| GSDMB (full-length, | 278 | 1251 bp | 302 | 416 aa |
| unengineered) | | | | |
| GSDMC (full-length, | 279 | 1527 bp | 303 | 508 aa |
| unengineered) | | | | |
| GSDMD (full-length, | 280 | 1455 bp | 304 | 484 aa |
| unengineered) | | | | |
| GSDME (full-length, | 281 | 1491 bp | 305 | 496 aa |
| unengineered) | | | | |

### Top7 (Negative Control Binder)

Top7 is a computationally designed protein with a novel fold not found in nature. It has no known binding affinity for p53 and serves as a negative control. Sequences include: DNA (279 bp): SEQ ID NO: 169; Protein (93 aa): SEQ ID NO: 175.

Cleavage-Impaired Control (pAL0609 Variant)

Identical to pAL0608 except for two amino acid substitutions in the junction peptide: R300A and H301A (RH->AA). This construct serves as a cleavage-impaired control. Sequences include: DNA (2073 bp): SEQ ID NO: 283; Protein (690 aa): SEQ ID NO: 307.

### NeoR Control

Neomycin resistance gene (NeoR) used as a negative control (non-functional filler) in sensor experiments. These sequences are well known in the art; the NeoR coding sequence appears within the nTEVP half constructs described herein.

### Wnt Pathway Non-Sensor Components

Individual Wnt pathway proteins expressed from separate constructs are provided in Table 34 below. These are NOT sensor components but rather pathway modulators or reporters used in experiments alongside the beta-catenin sensors.

TABLE 34
Wnt Pathway Non-Sensor Components

| | | DNA | | Protein | |
|---|---|---|---|---|---|
| | | SEQ ID | SIZE | SEQ ID | SIZE |
| NAME | Description | NO: | (bp) | NO: | (AA) |
| AXIN2 | Full-length Axin2 protein, a | 284 | 2532 bp | 308 | 843 aa |
| | negative regulator of the Wnt | | | | |
| | pathway and a transcriptional | | | | |
| | target of beta-catenin/TCF | | | | |
| | signaling. Used as a readout of | | | | |
| | pathway activity. | | | | |
| PDZ-CAAX | PDZ domain of Dishevelled fused | 285 | 300 bp | 309 | 99 aa |
| | to a CAAX membrane-targeting | | | | |
| | motif. This construct recruits the | | | | |
| | PDZ domain to the plasma | | | | |
| | membrane. | | | | |
| PDZ (L4 | PDZ domain of Dishevelled with | 286 | 237 bp | 310 | 78 aa |
| linker | L4 (4-residue) linker, without | | | | |
| variant) | CAAX motif. | | | | |
| PDZ (L8 | PDZ domain of Dishevelled with | 287 | 249 bp | 311 | 82 aa |
| linker | L8 (8-residue) linker, without | | | | |
| variant) | CAAX motif. | | | | |
| DVL1 | Dishevelled-1 protein lacking the | 288 | 1341 bp | 312 | 446 aa |
| (without DIX | DIX domain. DVL1 is a central | | | | |
| domain) | mediator of Wnt signaling that | | | | |
| | relays signals from Frizzled | | | | |
| | receptors. | | | | |
| DIX-CAAX | DIX domain of Dishevelled fused | 289 | 333 bp | 313 | 110 aa |
| | to a CAAX membrane-targeting | | | | |
| | motif. | | | | |
| DIX | DIX domain of Dishevelled, | 290 | 258 bp | 314 | 85 aa |
| | without membrane targeting. | | | | |
| DAX-CAAX | DAX domain fused to a CAAX | 291 | 330 bp | 315 | 109 aa |
| | membrane-targeting motif. | | | | |
| DAX | DAX domain, without membrane | 292 | 255 bp | 316 | 84 aa |
| | targeting. | | | | |
| GID_CAAX- | Two copies of GID_CAAX | 293 | 375 bp | 317 | 124 aa |
| T2A- | separated by a T2A self-cleaving | | | | |
| GID_CAAX | peptide. This produces two | | | | |
| | independent GID_CAAX proteins | | | | |
| | from a single transcript. | | | | |
| GID_CAAX- | GID_CAAX followed by T2A | 294 | 255 bp | 318 | 84 aa |
| T2A-L10 | self-cleaving peptide and a 10- | | | | |
| | residue linker. | | | | |

### p53 Binding Assay Constructs (Screening)

The following constructs were used in split-TEV protease complementation binding assays to validate p53-binding domains. In these assays, a candidate binder is fused to one half of a split protease, and a target construct presenting the binding epitope (or a control) is fused to the complementary half Productive binding brings the two protease halves into proximity, enabling reconstitution and reporter activation. These are screening tools, not sensor compositions.

Cryptic Epitope cTEVP Half (Binding Assay)

Structure can include: NLS(biNLS)-GSGSG-[BINDER]-L20-cTEVP-GSEGSx2-mTagBFP2. Reference plasmid is P-0675 (reference with P531B_004 binder).

cTEVP half construct was used in split-TEV binding assays to screen de novo binders against the p53 cryptic epitope. The binder is fused to cTEVP; the complementary nTEVP half presents the target epitope. Exemplary DNA sequence is DNA (1512 bp): SEQ ID NO: 319. Constant region excludes binder; actual insert length depends on binder choice. Sequence is provided with GSGSG (SEQ ID NO: 349) linker between NLS and [BINDER] placeholder. Exemplary protein sequence is Protein (503 aa), SEQ ID NO: 325. Constant region protein excludes binder (503 aa with P53B_004 reference binder, 95 aa).

nTEVP Half with NeoR Spacer (Binding Assay Control)

Structure can include: NLS (biNLS)-GSGSG-NeoR-L15-nTEVP-L10[v3]-mRuby3 (linker GSGSG, SEQ ID NO: 349). Reference plasmid is P-0664.

Complementary nTEVP half used in binding assays. The NeoR spacer provides steric bulk without p53-binding activity, serving as a non-targeting control. Paired with the cTEVP binder half above. Sequences include: DNA (1998 bp), SEQ ID NO: 320; Protein (665 aa), SEQ ID NO: 326.

nTEVP Half-Minimal (No NeoR Spacer)

Structure can include: NLS (biNLS)-GSGGS-GGGSG-RH peptide-GGSGG-GSEGS linker-nTEVP-L10[v3]-mRuby3 (linker GSGGS, SEQ ID NO: 350; linker GGGSG, SEQ ID NO: 351; linker GGSGG, SEQ ID NO: 352; linker GSEGS, SEQ ID NO: 353). Reference plasmid is pAL0605. Minimal nTEVP half construct for binding assays, lacking the NeoR spacer. The RH peptide (RNTFRHSVVVPYEPPG (SEQ ID NO: 328)) derives from the TEV protease recognition/hinge region. Sequences include: DNA (1284 bp), SEQ ID NO: 321; Protein (427 aa), SEQ ID NO: 327.

nTEVP Half-NeoR+RH Peptide (Cryptic Epitope Target)

Structure can include: NLS (biNLS)-GSGSG-NeoR-GGGSGx2-RH peptide-GSEGS-nTEVP-L10 [v3]-mRuby3 (linker GSGSG, SEQ ID NO: 349; linker GGGSGx2, SEQ ID NO: 354; linker GSEGS, SEQ ID NO: 353). Reference plasmid is pAL0608. nTEVP half presenting the wild-type cryptic epitope peptide (RNTFRHSVVVPYEPPG (SEQ ID NO: 328), the FRHSV region). Used as target construct in split-TEV binding assays to screen de novo binders for cryptic epitope specificity. Sequences include: DNA (2073 bp), SEQ ID NO: 322; Protein (690 aa), SEQ ID NO: 329.

Native Binder cTEVP Half (Binding Assay)

Structure can include: SUMOstar-GSx15-[BINDER]-L20-cTEVP-GSEGSx2-mTagBFP2. Reference plasmid is P-0561 (reference with Nb139 binder; linker GSx15 (SEQ ID NO: 355); linker GSEGSx2, (SEQ ID NO: 356)). cTEVP half construct used in binding assays with native p53-binding domains. SUMOstar provides cleavable solubility tag; GSx15 is a 15-aa flexible linker. DNA (1872 bp) sequence is SEQ ID NO: 323. Constant region excludes binder; length is with Nb139 reference binder (126 aa). Protein (623 aa) sequence is: SEQ ID NO: 330. Constant region protein excludes binder (623 aa with Nb139 reference, 126 aa).

Native Binder nTEVP Half-TP53 Target Construct

Structure can include: [TP53_VARIANT]-L15-nTEVP-L10 [v3]-mRuby3. Reference plasmids include P-0328 (TP53 WT), P-0329 (R175H), P-0330 (Y220C), P-0331 (R248Q). nTEVP half construct with full-length TP53 variant fused to nTEVP. Used as target construct in binding assays to validate native p53-binding domains. TP53 variant sequences are provided in Table 31. DNA (2319 bp) sequence is SEQ ID NO: 324. Sequence is provided with TP53 WT. For mutant variants (R175H, Y220C, R248Q), substitute the TP53 region per Table 31. Protein (772 aa) sequence is SEQ ID NO: 331. Sequence provided is with TP53 WT (392 aa). For mutant variants, see Table 31.

### Methods

Disclosed herein include methods of treating a disease or disorder characterized by aberrant signaling of one or more oncoproteins. In some embodiments, the method comprises: expressing a synthetic protein circuit of the disclosure in a cell of a subject in need thereof.

Disclosed herein include methods of treating a disease or disorder characterized by aberrant signaling of one or more oncoproteins. In some embodiments, the method comprises: administering any of the nucleic acid compositions or the pharmaceutical compositions of the disclosure to a subject in need thereof.

The disease or disorder can be associated with expression of an oncoprotein. In some embodiments, the disease associated with expression of an oncoprotein is selected from the group consisting of a proliferative disease, a precancerous condition, a cancer, and a non-cancer related indication associated with expression of the oncoprotein.

The disease can be cancer. In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia. The cancer can be a β-catenin associated cancer, a p53 associated cancer and/or a Myc associated cancer.

The cancer can be a RAS-associated cancer. In some embodiments, at least a portion of cancer cells of the subject comprise one or more RAS mutations. The one or more RAS mutations can comprise a mutation in KRAS, NRAS, and/or HRAS. In some embodiments, the KRAS mutation is selected from the group consisting of G12C, G12V, G12S, G12A, G12D, G12R, G13V, G13C, G13D, Q61P, Q61L, Q61H, Q61K, Q61E, Q61R, A146V, A146P, and A146T. In some embodiments, the HRAS mutation is selected from the group consisting of G12V, G12C, G12S, G12A, G12D, G12R, G13V, G13dup, G13C, G13S, G13D, Q61L, Q61H, Q61K, and Q61R. In some embodiments, the NRAS mutation is selected from the group consisting of G12A, G12C, G12D, G12R, G12S, G12V, G13A, G13C, G13D, G13R, G13S, G13V, Q61E, Q61H, Q61K, Q61L, Q61P, and Q61R.

In some embodiments, the at least a portion of cancer cells of the subject comprising one or more RAS mutations do not require RAS and/or RAS signaling for survival. In some embodiments, the subject has not received a prior cancer treatment. In some embodiments, the subject has not received a prior cancer treatment comprising administration of a RAS-targeting drug. In some embodiments, the subject has received at least one prior cancer treatment. The prior cancer treatment can comprise administration of a RAS-targeting drug. In some embodiments, the subject has developed resistance to a RAS-targeting drug. The RAS targeting drug can comprise Sotorasib, RMC-7977, or both.

The method can comprise administration of at least one additional anti-cancer agent to the subject. The additional anti-cancer agent can comprise a chemotherapeutic agent, an immunomodulatory drug, an immune metabolism modifying drug, a targeted therapy, radiation, an anti-angiogenesis agent, an agent that reduces immune-suppression, or any combination thereof. The chemotherapeutic agent can comprise an alkylating agent, a topoisomerase inhibitor, an anti-metabolite, an anti-mitotic agent, or any combination thereof.

The administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. In some embodiments, a tumor volume of the subject after treatment with the synthetic protein circuit, the composition, or the pharmaceutical composition decreases at least 50% relative to the tumor volume with no treatment or a previous treatment with the RAS targeting drug. In some embodiments, the subject achieves a complete response. The subject can be a mammal. The mammal can be human.

Disclosed herein include methods of detecting an oncoprotein in a cell. In some embodiments, the method comprises: expressing a synthetic protein circuit of the disclosure in the cell or contacting the cell with a nucleic acid composition of the disclosure, wherein the activation of the effector module in the cell is an indication of the presence and/or amount of the oncoprotein in the cell. The method can be performed in vitro, in vivo, or ex vivo. The method can comprise administration of the nucleic acid composition to a subject that has or is suspected of having cancer, and wherein activation of the effector module in one or more cells of the subject is an indication of the cancer status of the subject.

In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. The cancer can be a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia. The cancer can be a β-catenin associated cancer, a p53 associated cancer and/or a Myc associated cancer.

The cancer can be a RAS-associated cancer. In some embodiments, at least a portion of cancer cells of the subject comprise one or more RAS mutations. The one or more RAS mutations can comprise a mutation in KRAS, NRAS, and/or HRAS. In some embodiments, the KRAS mutation is selected from the group consisting of G12C, G12V, G12S, G12A, G12D, G12R, G13V, G13C, G13D, Q61P, Q61L, Q61H, Q61K, Q61E, Q61R, A146V, A146P, and A146T. In some embodiments, the HRAS mutation is selected from the group consisting of G12V, G12C, G12S, G12A, G12D, G12R, G13V, G13dup, G13C, G13S, G13D, Q61L, Q61H, Q61K, and Q61R. In some embodiments, the NRAS mutation is selected from the group consisting of G12A, G12C, G12D, G12R, G12S, G12V, G13A, G13C, G13D, G13R, G13S, G13V, Q61E, Q61H, Q61K, Q61L, Q61P, and Q61R.

The administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The subject can be a mammal. The mammal can be human.

### EXAMPLES

Some aspects of the embodiments discussed above are disclosed in further detail in the following examples, which are not in any way intended to limit the scope of the present disclosure.

### Methods

Provided below are exemplary materials and methods used in Examples 1-4.

### Plasmid Construction

Plasmids were generated via Gibson Assembly (NEBuilder HiFi DNA Assembly Master Mix; New England BioLabs) or KLD cloning (T4 Polynucleotide Kinase, T4 DNA Ligase, DpnI, T4 DNA Ligase Buffer; Thermo Scientific). Gene fragments were sourced from Twist Bioscience or Integrated DNA Technologies (IDT), or PCR-amplified from existing laboratory plasmid constructs. IDT synthesized all PCR primers. Plasmids were purified (QIAprep Spin Miniprep Kit or Qiacube machine; Qiagen), normalized (Thermo Scientific NanoDrop 8000), and sequence-verified (Plasmidsaurus or Genewiz) prior to experimental use.

### Tissue Culture and Cell Lines

Most cell lines were obtained from ATCC and cultured under standard conditions (37° C., 5% CO2, humidified Eppendorf CellXpert C170i incubator). Adherent cells were maintained in Dulbecco's Modified Eagle Media (DMEM; Thermo Fisher) supplemented with 10% fetal bovine serum (FBS; Avantor), penicillin (1 unit/ml), streptomycin (1 μg/ml), glutamine (2 mM), sodium pyruvate (1 mM), and 1×Minimal Essential Media Non-Essential Amino Acids (all Thermo Fisher). HEK293FT cells were cultured for up to 20 passages, and human cancer cell lines for up to 15 passages, maintaining 10%-90% confluency. Cells were passaged using 0.25% Trypsin-EDTA (Thermo Fisher). Viable cell numbers for seeding densities were determined using Trypan Blue (Invitrogen) and the Countess 3 automated cell counter (Thermo Fisher). Cells were routinely confirmed mycoplasma-free (MycoStrip kit; InvivoGen). Cell lines with stably integrated fluorescent proteins used for microscopy were purchased from FenicsBIO.

### Transient Transfection

Transient transfections were performed utilizing FuGENE HD (Promega), Lipofectamine 3000 (Thermo Fisher), or LNPs (described below) as transfection agents. Unless otherwise noted, we seeded 10 k cells (96-well plate), 200 k cells (24-well plate) for FuGENE or LNP transfections, and 275 k cells (24-well plate) for Lipofectamine transfections. Cells were reverse-transfected immediately after seeding following manufacturer instructions. For FuGENE transfections, we incubated 1 μg plasmid DNA with 6 μl transfection reagent. For FuGENE transfections, 200 k cells were transfected with up to 566 ng of DNA. For Lipofectamine transfections, 1.5 μl Lipofectamine 3000 and 1.5 μl reagent P3000 were used with a total of 500 ng DNA to transfect an equivalent of 275 k cells. The total amount of plasmid DNA applied in each transfection was scaled according to seeding densities.

### Flow Cytometry

Cells were collected two days post-transient transfection for flow cytometry, filtered through a 40-μm cell strainer, and analyzed by flow cytometry (CytoFLEX S flow cytometer, Beckman Coulter). Unless otherwise noted, we used FITC-A channel (GFP of iTEVP reporter; excitation 488 nm, emission 525/40 nm; gain of 1), ECD-A channel (mRuby3; excitation 561 nm, emission 610/20 nm; gain of 1), PB450-A channel (mTagBFP2; excitation 405 nm, emission 450/45 nm; gain of 1), and APC-A700-A channel (IFP of iTEVP reporter; excitation 638 nm, emission 712/25; gain of 100). Live cells and singlets were gated using FlowJo (version 10.10, BD Biosciences). iTEVP reporter activation was assessed by calculating the IFP (activation) to GFP (reporter expression) ratio for cells with GFP expression between 10⁵ and 10⁶ a.u., corresponding to the reporter expression regime with maximal dynamic range. As the reporter construct was always transfected as a separate polytransfection mix, selecting cells with high reporter expression did not bias subsequent analysis steps, i.e., the expression distribution of other constructs (e.g., reporter or ectopic Ras) remained unaffected.

### Computational Identification of Ras Binding Domains

To identify candidate Ras binding domains, human proteins known to physically associate with KRAS were first extracted from the STRING database (physical subnetwork, medium confidence). All proteins passing these filters were co-folded with wild-type KRAS-4B and GTP using AlphaFold3 (AlphaFold Server). Binders predicted to interact with the CAAX domain of KRAS-4B were manually removed. To identify candidate binders for experimental characterization, we filtered for binders with high-confidence Ras interactions (interface PAE score<10 and interface plDDT score>75) and manually extracted Ras-interacting domains from the full-length proteins. For the proteins ARAF, RAF1, RGL1, and RASA4, multiple candidate binder versions corresponding to different truncations or domains of the full-length protein were generated. Sequence regions known to act as nuclear localization signals (NLS) were also mutated. Furthermore, we used RFdiffusion (Base_ckpt and Complex_beta_ckpt model with default parameters; no hotspot residue selection) to de novo design Ras binders between 50 and 100 amino acids in length. Briefly, target structures (KRAS, HRAS, NRAS including GTP or analog nucleotide as ligand) were obtained from the Protein Data Bank (PDB). PDB assemblies were filtered and processed to provide a single Ras domain as input for RFdiffusion. Sequence design was performed with ProteinMPNN-FastRelax (dl_binder_design pipeline). After co-folding candidate binders with their corresponding target using AlphaFold2 (dl_binder_design pipeline), binders were filtered for high-confidence Ras interactions (interface PAE score<10 and interface plDDT score>80). Binders with high likelihood of dimerization in the absence of Ras were removed (co-folding of binder homodimers with AlphaFold3). In parallel, a list of synthetic Ras-binding domains were compiled such as monobodies, DARPins, synthetic proteins, and nanobodies from the available literature. All sequences were codon-optimized for expression in human cells (Genscript).

### Sensor Screening

To evaluate the sensitivity and specificity of candidate Ras sensors, we reverse transfected HEK293FT cells (275 k seeding density) with three separate plasmid mixes encoding (1) binder-cTEVP (120 ng), binder-nTEVP-IRES-mTagBFP2 (150 ng); (2) wild-type or mutant Ras (15 ng), mRuby3 (45 ng); and (3) iTEVP reporter fused to a CAAX membrane tether (170 ng). Transfections were performed with Lipofectamine 3000 according to the manufacturer's instructions. To characterize the binders, we gated for medium sensor (mTagBFP2 expression between 104 and 106 a.u.) and high Ras expression (mRuby3 expression between 105 and 106 a.u.). To analyze sensor responses to varying amounts of Ras, we gated for medium sensor expression (mTagBFP2 expression between 104 and 106 a.u.) and binned Ras expression as shown in plots. All plots show median reporter activation for the corresponding gate or bin. Confidence estimates for sensor screening experiments were obtained by bootstrapping (95% confidence intervals; 1,000 bootstrap iterations).

### Sensor Targeting Scope Characterization

To evaluate the targeting scope of Ras sensors, we tested the sensor performance against a list of Ras variants compiled by selecting all KRAS, HRAS, and NRAS variants from the TCGA pan-cancer database (accessed via the cbioportal server) that are present in at least 10 cancer patients55,56. Analogous to the presently described sensor screening procedure, HEK293FT cells were reverse transfected (200 k seeding density) with three separate plasmid mixes encoding (1) binder-cTEVP (133 ng), binder-nTEVP-IRES-mTagBFP2 (180 ng); (2) wild-type or mutant Ras (13 ng), mRuby3 (40 ng); and (3) iTEVP reporter fused to a CAAX membrane tether (200 ng). Transfections were performed with FuGENE HD according to the manufacturer's instructions. To calculate the fold-change of the response to mutant vs. wild-type Ras, we gated for medium sensor (mTagBFP2 expression between 104 and 105.5 a.u.) and high Ras expression (mRuby3 expression between 105 and 106 a.u.) and divided the median activation to a given mutant Ras variant to the median activation to the corresponding wild-type Ras isoform. Confidence estimates were obtained by bootstrapping (95% confidence intervals; 1,000 bootstrap iterations).

### Single-Cell Classification Performance of Sensors

To evaluate the ability of the presently disclosed sensors to discriminate between wild-type (WT) and mutant Ras proteins at the single-cell level, receiver operating characteristic (ROC) curve analysis was conducted. First, all cells with medium sensor (mTagBFP2 expression between 104 and 105.5 a.u.) and high Ras expression (mRuby3 expression between 105 and 106 a.u.) were selected. Next, for each sensor, normalized reporter activation between cells expressing mutant Ras variants and those expressing the corresponding wild-type proteins was compared. Normalized activation was calculated as the ratio of the iTEVP activation signal to the iTEVP expression level (IFP/GFP). ROC curves were generated for each sensor by setting thresholds across the range of observed normalized reporter values. At each threshold, cells were classified into true positives (mutant cells above threshold), false positives (wild-type cells above threshold), true negatives (wild-type cells below threshold), and false negatives (mutant cells below threshold). The true positive rate (sensitivity) and false positive rate (1−specificity) were calculated accordingly. The area under the ROC curve (AUC) was determined using the trapezoidal rule, providing a quantitative measure of each sensor's discriminatory performance. Higher AUC values indicated better sensor performance in distinguishing mutant from wild-type Ras proteins.

### Sensor Characterization in Human Cancer Cell Lines

To characterize the performance of sensors in human cancer cell lines, 250k cancer cells in a 24 well plate were reverse transfected with two separate plasmid mixes encoding (1) binder-cTEVP (100 ng), binder-nTEVP-IRES-mTagBFP2 (135 ng); and (2) iTEVP reporter fused to a CAAX membrane tether (150 ng). After 48h, cells were analyzed by flow cytometry. Median sensor activation was calculated as the ratio of the iTEVP activation signal to the iTEVP expression level (IFP/GFP). Confidence estimates were obtained by bootstrapping (95% confidence intervals; 1,000 bootstrap iterations). The same setup was used to characterize single-protein sensors. Median reporter activity was calculated after gating for all sensor-transfected cells (mTagBFP2 expression between 103.5 and 106 a.u.).

### Design and Screening of Protease-Activatable Proteases

A panel of candidate protease-activatable proteases based on diverse natural and synthetic protein activation mechanisms were designed. All constructs were expressed in HEK293 cells via transient transfection for characterization.

Candidates 1-3: To emulate autoinhibition-based activation mechanisms observed in proteins such as WASP, SNARE, and ERM, constructs were designed in which a non-cleavable, synthetic autoinhibitory peptide was tethered to the active site of TVMV protease (TVMVP). The tether included a cleavage site for an orthogonal protease (TEVP), enabling activation through proteolytic removal of the inhibitory domain. Constructs were tested with and without co-expression of the activating TEVP protease. To modulate inhibitory dynamics, a series of variants were generated with progressively shortened linkers between the autoinhibitory peptide and TVMVP.

Candidates 4-7: To improve autoinhibition, a tethering strategy was developed in which the autoinhibitory peptide was anchored at both termini via two TEVP cleavage sites. We circularly permuted the TVMVP sequence and rejoined the new termini using antiparallel leucine zippers to stabilize the conformation. To optimize access of TEVP to its cleavage sites, varied the length of the linker flanking the autoinhibitory domain was also varied.

Candidates 8-10: Strain-based inhibition was next explored, drawing inspiration from previous studies where circular permutation introduces conformational strain that disrupts protein function (e.g., CRISPR). Circularly permuted variants of TVMVP were engineered in which the old termini were joined by short linkers, embedding the TEVP cleavage site to allow strain release upon protease input. Additional constructs included deletions of disordered residues at the native termini to increase the distance between ends and enhance strain potential.

Candidates 11-13: To sterically inhibit the TVMVP active site, we fused bulky de novo designed heterodimers to the protease, positioned to occlude the catalytic cleft. These heterodimeric blocking domains were flanked by TEVP cleavage sites to enable conditional displacement and activation.

Candidates 14-18: A protease caging strategy was adopted using split TVMVP. One protease half was caged by fusion to a catalytically inactive TVMVP fragment via designed coiled coils. The coiled coil associated with the inactive half was flanked by TEVP cleavage sites, enabling conditional release. Additional constructs extended this strategy by caging both protease halves with inactive TVMVP fragments, each released via TEVP cleavage. To enable protease activation through strand displacement, we substituted the coiled coils with lower-affinity, sequence-matched variants previously reported to allow displacement by higher-affinity interactors. These designs were intended to facilitate TEVP-dependent exchange of inhibitory domains with active TVMVP halves.

mRNA Production

DNA templates containing a 5′ T7 promoter sequence followed by the dinucleotide sequence AG were linearized by PCR (Q5 High-Fidelity DNA Polymerase, NEB). A 3′ end 120-base pair polyA-tail was added using PCR. The linear DNA templates were purified by Ampure beads or gel purification (Zymoclean Gel DNA Recovery Kit, Zymo). mRNA was subsequently produced via in vitro transcription (IVT) using NEB's HiScribe T7 High Yield RNA Synthesis Kit. The IVT reaction mix contained 10×Reaction Buffer (1× final), 1 μg DNA template, 4 mM CleanCap AG (TriLink), 2 μl T7 RNA polymerase mix, and the nucleotides ATP, GTP, CTP, and N1-Methyl-Pseudouridine-5′-Triphosphate (TriLink) at a final concentration of 5 mM. After an incubation period of 2 hours at 37° C., DNase I (NEB) was added and reactions were incubated for another 15 minutes at 37° C. Finally, mRNA was purified using Zymo's RNA Clean and Concentrator Kit and concentrations were read out by Nanodrop or Qubit RNA High Sensitivity or Broad Range kits. The concentration normalized mRNAs were stored at −80° C.

Lipid Nanoparticle (LNP) mRNA Encapsulation

A previously described 4 component LNP formulation (4A3-SC8, DOPE, Cholesterol, DMG-PEG) that enables transfection of cells in culture and in vivo was adopted.

To prepare the lipid stock solutions, a full 25 mg tube of 4A3-SC8 compound was dissolved in 167 μL of pure ethanol to yield a 150 mg/mL stock solution. Separately, 10 mg of DOPE was dissolved in 1.0 mL of pure ethanol to produce a 10 mg/mL stock solution (alternatively, 100 mg in 10 mL ethanol). Similarly, 10 mg of cholesterol was dissolved in 1.0 mL of pure ethanol (or 50 mg in 5 mL ethanol), and 10 mg of DMG-PEG was dissolved in 1.0 mL of ethanol (or 50 mg in 5 mL ethanol), resulting in 10 mg/mL stock solutions for each. A 20 mM working lipid mixture was prepared by combining 6.7 μL of the 4A3-SC8 solution (23.8%, 4.76 mM), 50.7 μL of the DOPE/DSPC solution (23.8%, 4.76 mM), 52.7 μL of the cholesterol solution (47.6%, 9.52 mM), and 34.2 μL of the DMG-PEG solution (4.8%, 0.96 mM).

The working lipid mixture was used to encapsulate mRNAs in the lipid nanoparticle (LNP) formulation. Initially, the lipid mixture was equilibrated at room temperature for at least 5 minutes and then vortexed at speed 10 for 5 seconds. A lipid mastermix was subsequently prepared by mixing 12 μL of the lipid mixture with 18 μL of 200-proof ethanol, yielding 30 μL per reaction. This mastermix was distributed into individual tubes. Each RNA mixture was prepared by combining 40 μL of RNA solution (250 ng/μL; total 10 μg RNA input) with 32 μL of nuclease-free water and 18 μL of 50 mM citrate buffer, yielding a total volume of 90 μL per reaction. To assemble the LNPs, 30 μL of the lipid mastermix was placed on a Vortex-Genie 2 vortex mixer set at speed level 1. While vortexing, 90 μL of the RNA mixture was rapidly pipetted into the lipid mastermix in a single action, and vortexing was continued for 30 seconds.

The resulting dispersion was incubated at room temperature for 5 minutes, and dialysis was initiated within 15 minutes of mixing. Dialysis was performed using Pur-A-Lyzer Midi 3500 dialysis tubes. Each tube was preconditioned by adding 900 μL of water, incubating for 5 minutes, and then removing the water. Approximately 120 μL of each LNP sample was transferred into the dialysis tubes, which were placed into a styrofoam holder and submerged in 1×PBS within a beaker. Dialysis was conducted either for 1 hour at room temperature or overnight at 4° C. After dialysis, each sample was transferred into an RNase-free 1.5 mL microcentrifuge tube, and the final volume was measured. Samples were adjusted to a total volume of 500 μL by adding the appropriate volume of 1×PBS. All samples were stored at 4° C.

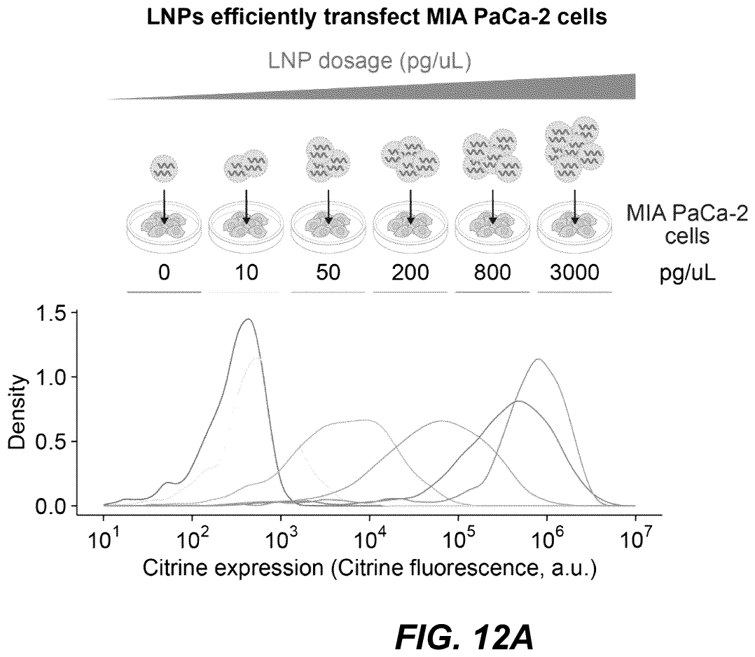

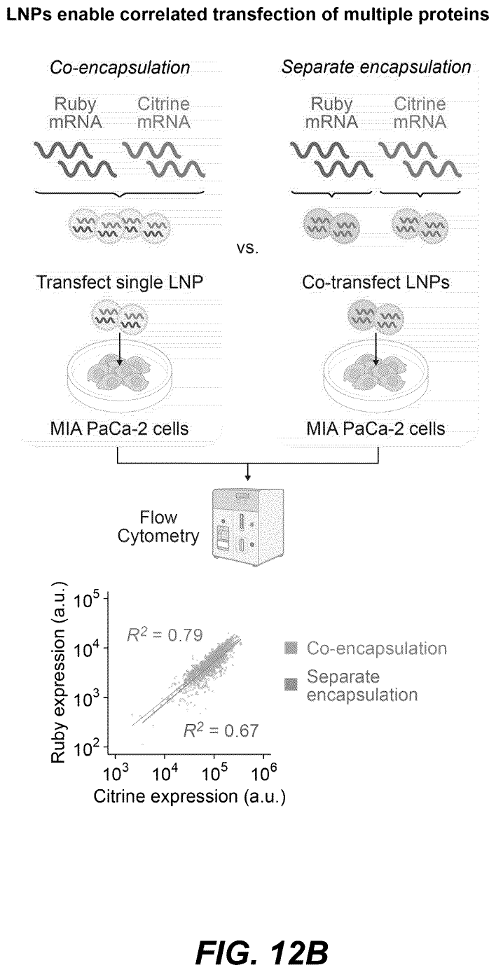

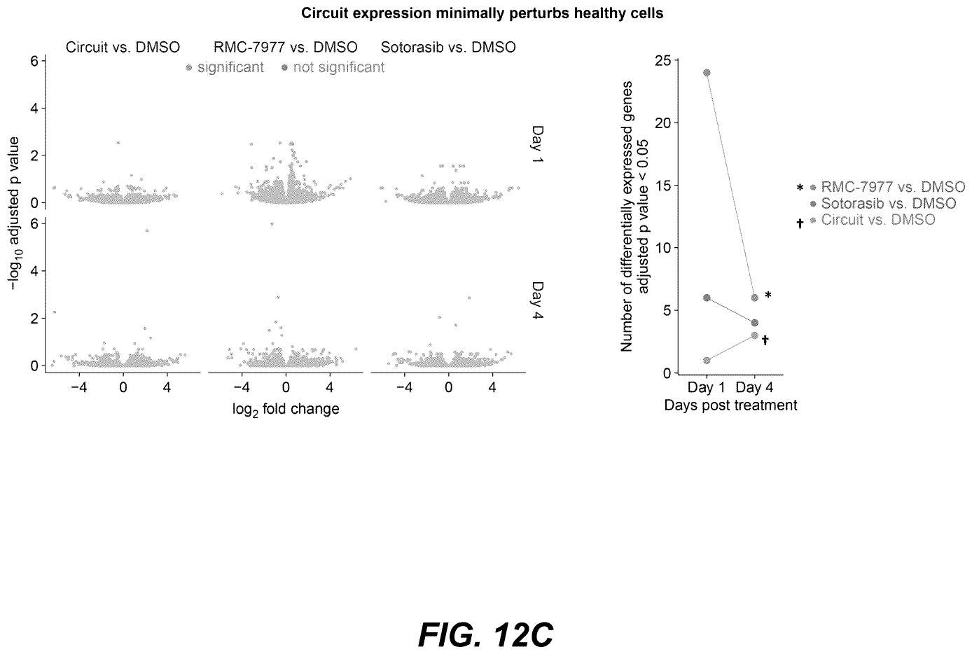

The resulting mRNA-LNP particles exhibited nearly complete transfection efficiency of human MIA PaCa-2 cells, and allowed titration of expression over four orders of magnitude (FIG. 12A). This approach was also compatible with multi-component delivery of separately encapsulated mRNAs (FIG. 12B).

mRNA-LNP Transfection of Human Cell Lines

LNPs were reverse transfected by first adding LNPs to culture plates, followed by the addition of cells on top. To maximize transfection efficiency in vitro, mRNA-containing LNPs were pre-complexed with ApoE, a naturally excreted liver protein that binds lipids in vivo and recruits lipid particles to cells expressing the low-density lipoprotein (LDL) receptor.

Cell Viability Assay with CellTiter-Glo

Cell viability was assessed using the CellTiter-Glo (CTG) Luminescent Cell Viability Assay (Promega). Cells were seeded into 96-well plates at 100 μL medium per well and incubated with treatments as described for each experiment. Nunc Edge 2.0 plates were used to minimize edge effects. On the day of the viability assay, plates were removed from the incubator and equilibrated at room temperature for 30 minutes prior to CTG reagent addition. Subsequently, 100 μL of CTG reagent was directly added into each well. Plates were then placed on an orbital shaker in the Promega GloMax instrument and shaken for 2 minutes to facilitate cell lysis and thorough mixing of the reagent. Following shaking, plates were incubated at room temperature for an additional 10 minutes to stabilize the luminescent signal. During this incubation, 180 μL of the mixture from each well was transferred into a white luminescence-compatible plate. Luminescence was measured using the GloMax according to the manufacturer's protocol. Cell viability was calculated by normalizing raw luminescence measurements of treated to untreated samples. Where applicable, dose-response curves were modeled with a four-parameter log-logistic model. The same model was used to calculate EC30, EC50, EC70, and EC90 concentrations. The goodness of fit between different models was compared with the sum-of-squares F-test. More specifically, we compared models fitting separate curves or a reduced four-parameter log-logistic model assuming a single curve.

Cell Viability Assay with Flow Cytometry

CellTiter-Glo (CTG) provides accurate measurements only within a specific range of cell densities. Thus, for experiments testing varying initial cell densities and their effects on drug versus circuit efficacy, we used flow cytometry to quantify cell viability. Annexin V staining was used to gate and exclude dead cells from analysis.

### Analysis of Ras Circuit Response to EGF

HEK293 cells (100 k cells per well) were seeded in a 24-well plate and treated with epidermal growth factor (EGF) at concentrations of 0, 0.1, 1, 10, and 100 ng/ml for 1 hour. Following treatment, cells were processed for phospho-ERK staining and analyzed by flow cytometry. Specifically, cells were fixed by adding an equal volume (600 μL) of 2× eBioscience™ IC Fixation Buffer directly to each well, followed by gentle vortexing to mix thoroughly. Samples were incubated for 20 minutes at room temperature in the dark. After fixation, cells were scraped thoroughly from each well using a pipette tip, collected, and centrifuged at 600×g for 5 minutes at room temperature, followed by removal of the supernatant. Cell pellets were then resuspended in the residual volume and permeabilized by adding 200 μL ice-cold 90-100% methanol (HPLC grade), followed by vortexing and incubation for at least 30 minutes on ice. Subsequently, cells were washed by adding 600 μL eBioscience™ Flow Cytometry Staining Buffer, centrifuged at 600×g for 5 minutes, and the supernatant discarded. The wash step was repeated, leaving approximately 100 μL of residual staining buffer. Cells were stained with 5 μL (0.125 μg) directly conjugated anti-phospho-ERK1/2 antibody (Invitrogen catalog #12-9109-42) for 30 minutes at room temperature in the dark. After staining, cells underwent two additional washes with 600 μL Flow Cytometry Staining Buffer, each followed by centrifugation at 400-600×g for 4-5 minutes, with final resuspension in 100 μL of staining buffer for analysis. Finally, cells were analyzed by flow cytometry using the entire remaining volume (100 μL). The fraction of pERK positive cells was calculated as the fraction of cells with ECD-A signal>103 a.u. Cell viability was measured as described above.

mRNA Sequencing and Analysis

mRNA was extracted from 96-well plates using Direct-zol-96 RNA Kits (Zymoresearch Cat #R2055). 50 ng of extracted mRNA from each sample was used as input for downstream NGS library preparation. mRNA-seq libraries were prepared in 96-well format using a modified 3′Pool-seq protocol. In brief, reverse transcription reactions were prepared by mixing input RNA with 1 μl Indexed RT Primer (10 μM), 1 μl 10 mM dNTP Mix (New England Biolabs Cat #N0447S), 1 μl diluted ERCC Spike-In Mix 1 (0.004 μL stock ERCC per μg RNA, ThermoFisher Cat #4456740), 3.6 μl of 5× RT buffer (ThermoFisher Cat #EP0752), 0.5 μl of RNase inhibitor (ThermoFisher Cat #EO0381), 1 μl Maxima RT H Minus (ThermoFisher Cat #EP0752), and 2.5 μl 10 μM Template Switching Oligo in an 18 μl reaction. Reverse transcription was carried out in a thermocycler using the program described in the 3′Pool-seq protocol. Samples from each row of the 96-well plate were pooled (column pooling) by mixing an equal volume of each reverse transcription reaction into a new well, for a total volume of 20 μl. Residual primers were degraded by adding 1 μl Exonuclease I (New England Biolabs) and incubating at 37° C. for 45 min, followed by denaturation at 92° C. for 15 min. Subsequent cDNA amplification, tagmentation, and row pooling were performed following the 3′Pool-seq protocol. Finally, 20 μl of pooled NGS library was subjected to gel-based size selection using E-Gel EX Agarose Gel (ThermoFisher Cat #G401001) to enrich for fragments in the 200-1000 bp range, and eluted in 15 μl. Eluted pooled NGS libraries were examined using an Agilent TapeStation 4200 (Agilent Technologies) to determine average fragment sizes. Library concentration was quantified using a Qubit 3.0 Fluorometer (Life Technologies). NGS libraries were diluted to 2 nM, denatured in 0.2 N NaOH, and loaded onto the Element AVITI sequencer following the Element Biosciences Cloudbreak Sequencing user guide. Read de-multiplexing was performed with Bases2Fastq, a standard software package used with the Element Biosciences system. De-multiplexed reads were aligned to the reference genome GRCh38.103 using STAR (2.7.8a) with the ENCODE standard options, except “-outFilterScoreMinOverLread 0.3-outFilterMatchNminOverLread 0.3-outFilterMultimapNmax 20”. Uniquely mapped reads that overlapped with genes were counted using HTSeq-count (0.13.5) with default settings, except “-m intersection-strict”. To normalize for differences in sequencing depth across samples, we rescaled gene counts to counts per million (CPM). Fold-changes and adjusted p-values were calculated using the R package DESeq2.

### Drug Treatment In Vitro

Cells were treated with Sotorasib/AMG-510 (10 mM, MedChemExpress, Cat. No.: HY-114277), RMC-7977 (10 mM, MedChemExpress, Cat. No.: HY-156498), or Paclitaxel (10 mM, MedChemExpress, Cat. No.: HY-B0015) at the indicated concentrations per experiment.

### Time-Lapse Microscopy

HEK293 and MIA PaCa-2 cell lines were engineered to stably express mRuby3 and GFP, respectively. A co-culture containing 50,000 total cells at a 3:1 ratio of HEK293 to MIA PaCa-2 was seeded into black-walled, glass-bottom 24-well plates (Ibidi) and incubated overnight. The following day, drug or circuit treatments were applied, and plates were imaged using an Olympus automated microscope controlled by MetaMorph software. Time-lapse image acquisition was performed every 2 hours. Images were processed using a custom pipeline in Python. Raw image tiles were first stitched with 10% overlap to reconstruct full fields of view. Stitched images underwent three main processing steps. First, Gaussian filtering (7×7 kernel) was applied to denoise background speckles. Second, image contrast was normalized using percentile-based intensity scaling, with intensities rescaled between the 1st and 99.9th percentiles to account for field-to-field variation. Finally, a dual-threshold segmentation strategy was used for cell detection and quantification. Pixels exceeding the high threshold (20th percentile of the normalized intensity range) were classified as healthy cells and assigned a fixed intensity value of 180. Pixels between the high (20th percentile) and low (15th percentile) thresholds were preserved with their scaled intensities to capture cells with reduced fluorescence signal, indicative of potential cell stress or death. This method enabled consistent detection of both bright and dim cells while preserving biologically relevant signal variation.

Sequential Resistance Selection with Drugs and Circuit

MIA PaCa-2 cells were plated in a 10 cm tissue culture plate (350 k cells/plate). Cells were treated with the IC70 concentrations that we previously determined, specifically 1 μM of Sotorasib and 60 pg/μL each of Ras sensor and Casp-3 mRNA-LNPs. After treatment for five days, cells were washed with PBS, trypsinized, replated at the same density (350 k cells), and re-treated identically as described above. In total, cells underwent three rounds of selection treatment. At two time points-before the first treatment (“no selection”) and after the final treatment-we collected cells and exposed them to concentration ranges of either Sotorasib or circuit. For Sotorasib, concentrations tested were 0.0001, 0.001, 0.01, 0.1, 1, and 10 μM. For circuit testing, cells were treated with mRNA-LNPs of Ras sensor/Casp-3 at concentrations of 10, 20, 40, 80, 160, and 320 μg/uL. Cell viability was read out by CellTiter-Glo three days post transfection.

### Analysis of Sotorasib and Circuit Response to Increased Ras Expression

10 k MIA PaCa-2 cells were transfected with either 200 pg/μL KRASG12C mRNA-LNP or 200 pg/μL NeoR mRNA-LNP as negative control. These mRNA-LNPs were co-delivered with 0.0001, 0.001, 0.01, 0.1, 1, and 10 UM of Sotorasib or 10, 20, 40, 80, 160, and 320 pg/uL of circuit mRNA-LNP (sensor v2-s+Casp-3). Cell viability was read out by CellTiter-Glo three days post transfection.

### Animal Studies

All animal handling, care, and treatment procedures were carried out in accordance with the applicable regulations and guidelines established by the relevant Institutional Animal Care and Use Committee (IACUC). Animals were housed in polycarbonate cages within an environmentally controlled, well-ventilated room maintained at a constant temperature of 20-26° C. and a relative humidity of 40-80%. Fluorescent lighting was provided on a 12-hour light/dark cycle.

### In Vivo Hydrodynamic Tail Vein Injection Liver Tumor Models

FVB/NJ mice (Jackson Laboratory, 001800) were used for HDT tumor models due to their enhanced susceptibility to tumor development. The FVB mice used in this study were ordered directly from Jackson Laboratory (001800). Hydrodynamic transfection was used to introduce transposable vectors expressing mutant NRAS-G12V, TP53 shRNA, and sleeping beauty transposase (SB100) into FVB mice. Mice were injected at 6-8 weeks of age, when their body weights were ~18-25 g. HDT plasmids were suspended at the noted concentrations in 2 mL of saline and administered via tail-vein injection over 7 seconds. A 10:1 mass ratio of combined HDT plasmids to SB100 transposase plasmid was used. In the genetic tumor rescue experiments PT2-NRAS-G12V was used, and 10 μg/mouse of each plasmid was added to the HDT volume. LNP studies substituted PT3-NRAS-G12V, and 1 μg of this plasmid/mouse was used due to increased tumor induction efficiency. In the in vivo mRNA-LNP experiments, LNPs were dosed at 1.5 mg total RNA per kg body weight dissolved in 0.2 mL PBS via the lateral tail vein at the noted time points.

### Liver and Body Weight Measurements

Animals were euthanized by cervical dislocation under isoflurane anesthesia. Livers were removed, weighed, and imaged at the time of sacrifice. Liver/body weight ratios were calculated as follows: (whole liver weight)/(intact body weight)×100. Extracted livers were imaged (anterior and posterior view) and corresponding images were used to assess the surface tumor burden of each animal. More specifically, tumor nodules were annotated manually in a blinded fashion. Surface tumor burden was calculated as accumulated surface tumor area over anterior and posterior liver views.

### Liver Surface Tumor Area Annotation and Masking

Liver images were manually annotated for nodules by an independent annotator with no involvement in the study. The annotation process was blinded: image files were assigned numeric identifiers, and the annotator was not provided with any information regarding experimental conditions. Tumors were manually segmented using NimbusImage on a total of 284 images of mouse livers. Each image received three separate masks: (1) “white nodes” for tumors that appeared lighter than surrounding tissue, (2) “black nodes” for tumors that appeared darker, and (3) a whole-liver mask outlining the total liver area. Only abnormalities larger than approximately 1 mm were included. Both raised masses and regions of distinct discoloration were classified as nodules. Non-tumor features such as fat deposits, blood vessels, bubbles, and specular reflections were excluded from annotation.

### Kaplan-Meier Curve

Tumors were induced in FVB mice as described above, with or without the noted circuit plasmids. Tumor development was allowed to proceed until reaching any of the three following clinical endpoints: a body condition score of 1, difficulty breathing, or decreased motility resulting in inability to obtain food or water, at which point mice were immediately euthanized at the recommendation of veterinary staff. Log-rank test was used to determine if differences in survival were significant.

### LNP Formulation and Physical Characterization for Mouse Studies

The following lipids were dissolved in 100% ethanol at a 15:30:15:3:7 molar ratio: 4A3-SC8 lipid (synthesized in-house), cholesterol (Sigma Aldrich Cat. No. C3045), DOPE (Avanti Polar Lipids Cat. No. 850725), DMG-PEG-2000 (Avanti Polar Lipids Cat. No. 880151), and DOTAP (Avanti, 890890P) with a total lipid: RNA mass ratio of 40:1. RNA was dissolved in 10 mM citrate buffer (pH 4.5) at a 3:1 v/v aqueous: organic phase ratio. LNPs were formed by microfluidic mixing of the lipid and RNA solutions using a Precision Nanosystems NanoAssemblr Benchtop Instrument, in accordance with the manufacturer's protocol. LNPs were dialyzed in PBS overnight at 4° C. and stored at 4° C. for up to 72 hours. Particle size and dispersity were measured by dynamic light scattering (DLS) using a Malvern Zetasizer DLS instrument.

### Software

The following software was used in this study for data collection: AlphaFold 3 (AlphaFold Server), Colabfold (v1.5.2), RFdiffusion (v1.1.0), ProteinMPNN-FastRelax (via dl_binder_design package v1.0.0), MetaMorph (version 6.2.6). The following software was used in this study for data analysis: PyMol (version 2.5.4), ChimeraX (version 1.7.1), FlowJo (version 10.10.0), SnapGene (version 8.0.2), Geneious (version 2023.0.4), Bases2Fastq (https://github.com/Elembio/bases2fastq-dx, Element Biosciences), NimbusImage, conda (v4.14.0), bioconductor-biomart (v2.58.0), bioconductor-biostrings (v2.70.1), bioconductor-deseq2 (v1.42.0), cairo (v1.18.0), imagemagick (v7.1.1_33), ipykernel (v6.29.5), ipython (v8.26.0), jupyter_server (v2.14.2), jupyterlab (v4.2.1). python (v3.12.3), r-base (v4.3.3), r-cowplot (v1.1.3), r-dplyr (v1.1.4), r-essentials (v4.3), r-ggplot2 (v3.5.1), r-ggpubr (v0.6.0), r-ggrepel (v0.9.5), r-jsonlite (v1.8.8), r-magick (v2.8.3), r-rcolorbrewer (v1.1_3), r-tidyverse (v2.0.0), pandas (v2.2.3), matplotlib (v3.10.1), numpy (v2.2.4), scipy (v1.15.2), scikit-learn (v1.6.1), opencv-python (v4.7.0.72), scikit-image (v0.20.0), pillow (v9.5.0)

### Statistics and Reproducibility

Unless otherwise noted, all experiments were conducted with three to four technical or biological replicates.

### Example 1

### Engineered Protein Circuits for Cancer Therapy

### Summary

Many targeted therapies indirectly suppress cancer cells by inhibiting oncogenic signaling pathways such as Ras. This renders them susceptible to resistance and limits their long-term clinical efficacy. Engineered protein circuits have been envisioned as an alternative to pharmacological inhibition that directly rewires oncogenic activity to cell death. However, it has remained unclear whether engineered protein circuits can potently and safely treat cancers. It is provided herein that Ras-targeting circuits can accurately discriminate between cancer and non-cancer cells, circumvent intrinsic and acquired resistance mechanisms that limit pharmacological inhibitors, and suppress cancer in vivo. These circuits combine three modules: a protease-based sensor that responds to a broad spectrum of clinically relevant Ras mutations, an optional protease amplifier, and protease-triggered cell death effectors. These effectors can flexibly trigger either non-inflammatory apoptosis or immunogenic pyroptosis, which has been shown to extend therapeutic effects beyond transfected cells. The resulting sense-kill circuits can be safely, efficiently, and transiently delivered to cells as mRNA in lipid nanoparticles (LNPs). The circuits exhibited potent efficacy against Ras-mutant human cancer cell lines with minimal off-target killing of wild-type Ras cells. In immunocompetent mice bearing aggressive, multifocal Ras-driven liver tumors, systemically-delivered mRNA-LNP circuits significantly reduced tumor burden. Further, therapeutic circuits provided more potent killing of Ras-mutant cancer cells than the Ras inhibitors Sotorasib and RMC-7977, and exhibited increased sensitivity to Sotorasib-resistant cells in vitro. These results establish a potent, specific, and programmable mechanism for treating cancer and other human diseases.

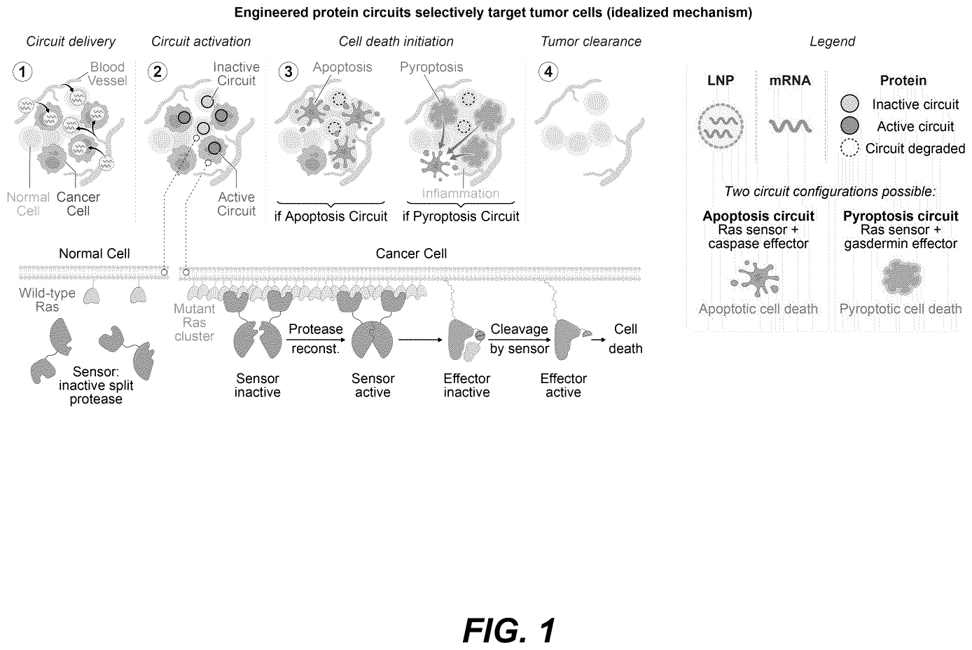

Therapeutic circuits comprise sets of engineered proteins that can be expressed in cells to sense disease-specific signals and conditionally trigger cell death or other responses (FIG. 1). In the context of cancer, they have the potential to offer higher efficacy and less opportunity for resistance than targeted therapies, greater molecular specificity than chemotherapies, and an expanded ability to sense intracellular proteins compared to CAR-T cells and antibody-drug conjugates. While aspects of the therapeutic circuit paradigm have been explored, fundamental questions have remained: Can engineered proteins sensitively and specifically detect mutant oncogenes in cancer cells? Can therapeutic circuits treat tumors in vivo? And, how do therapeutic circuits compare to targeted small molecule drugs in terms of treatment efficacy and resistance?

Described herein is the design, optimization, and characterization of protease-based therapeutic circuits that selectively kill Ras-mutant cancer cells. Proteases represent ideal building blocks for therapeutic circuits. They are modular, engineerable and can directly interface with endogenous pathways. Ras is explored herein because Ras mutations drive roughly one in four human cancers, yet durable and effective Ras-targeting therapies remain limited. More specifically, there are provided sensitive and broadly specific protease-based sensors of mutant Ras, engineer a protease-activated protease module that amplifies signals, and couple sensors and amplifiers to cell death effectors. Delivered as mRNA in lipid nanoparticles (LNPs), these circuits can target Ras-driven cancer cells in vitro as well as Ras-driven liver tumors in vivo. Finally, it is demonstrated herein that therapeutic circuits can provide advantages in efficacy and resistance compared to two state-of-the-art targeted pharmacological Ras inhibitors.

### Engineered Sensors Specifically Respond to Diverse Ras Mutations

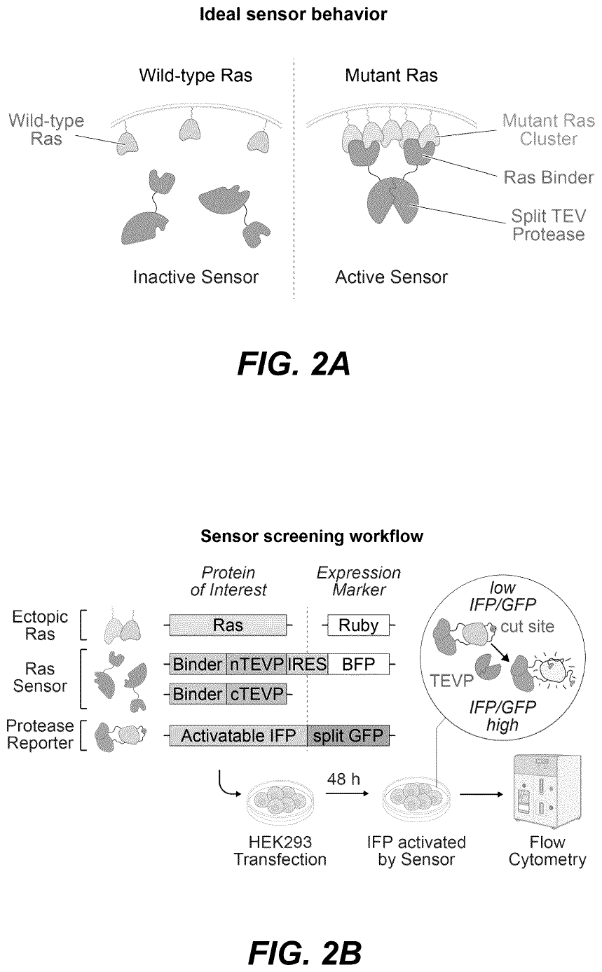

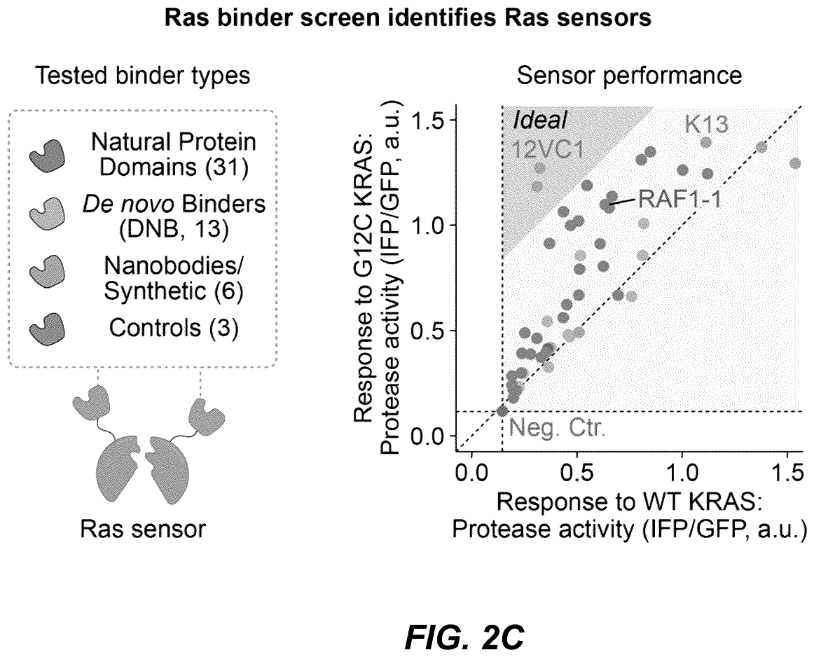

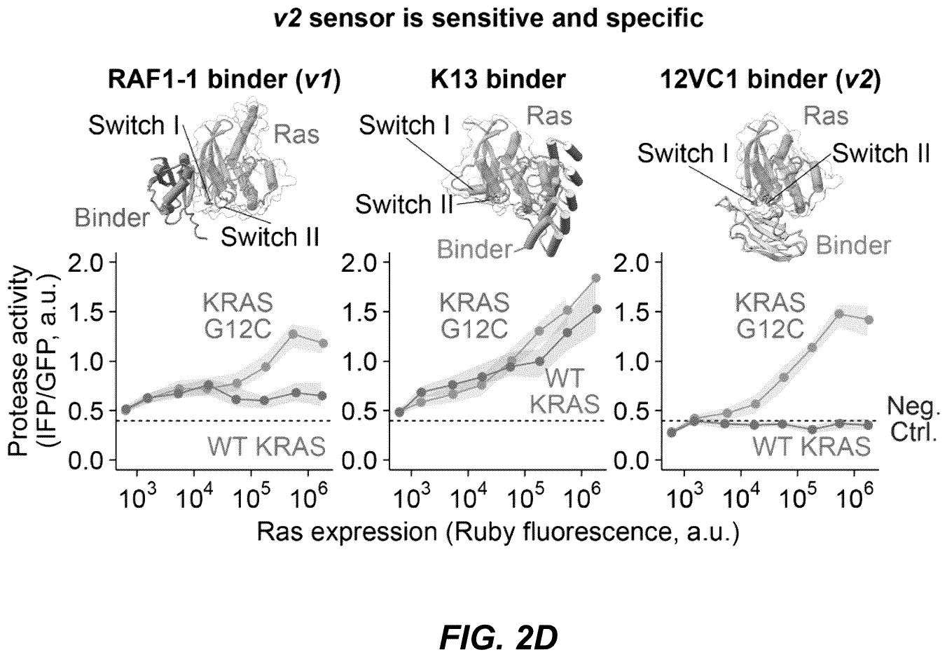

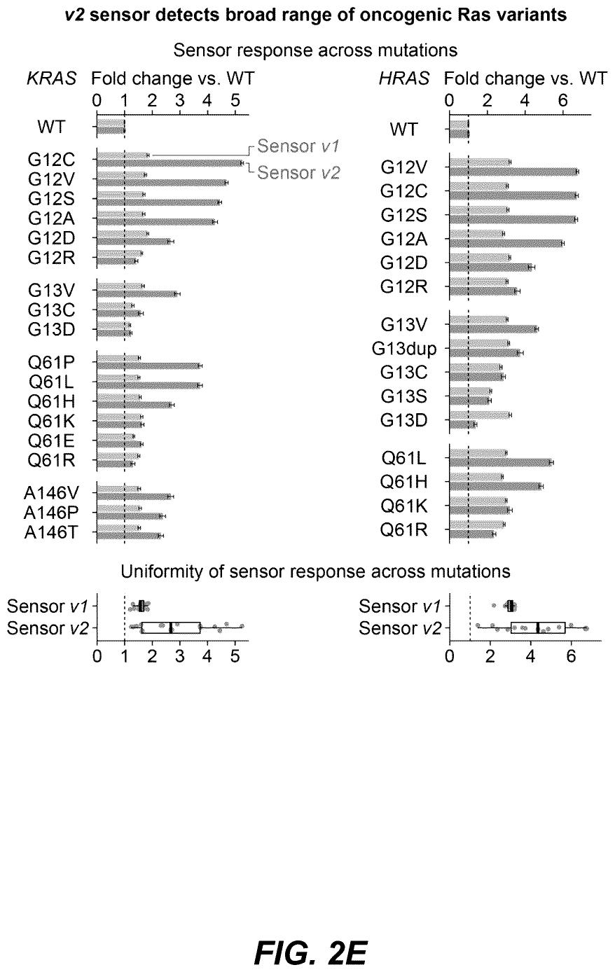

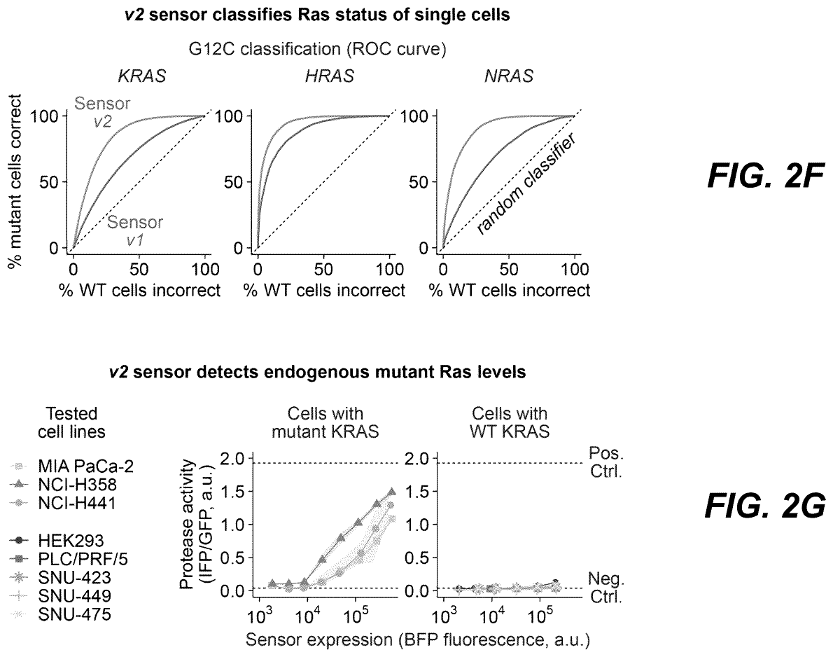

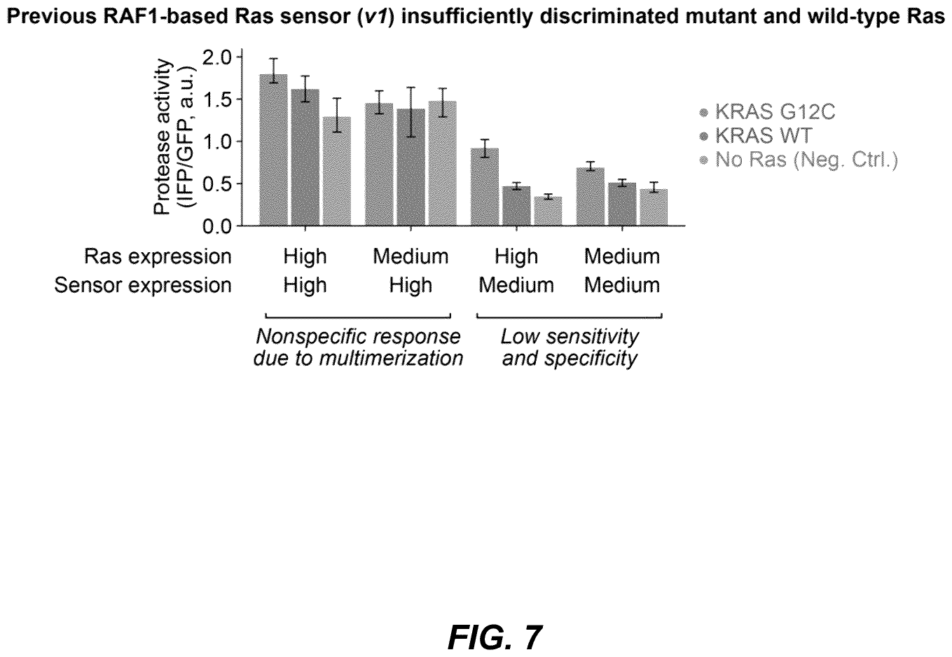

An ideal Ras sensor can strongly activate in the presence of diverse oncogenic Ras mutants, but not respond to wild-type Ras. A previous sensing mechanism used Ras binding domains attached to complementary halves of a split Tobacco Etch Virus (TEV) protease. Ras activation can then recruit split sensor proteins to Ras clusters, leading to protease reconstitution (FIG. 2A). In its initial design (v1), the sensor used the natural Ras-binding domain of RAF1. Despite responding to overexpressed Ras in HEK293 cells, this v1 sensor poorly discriminated mutant and wild-type Ras variants, due to limited specificity and undesired sensor multimerization (FIG. 7).

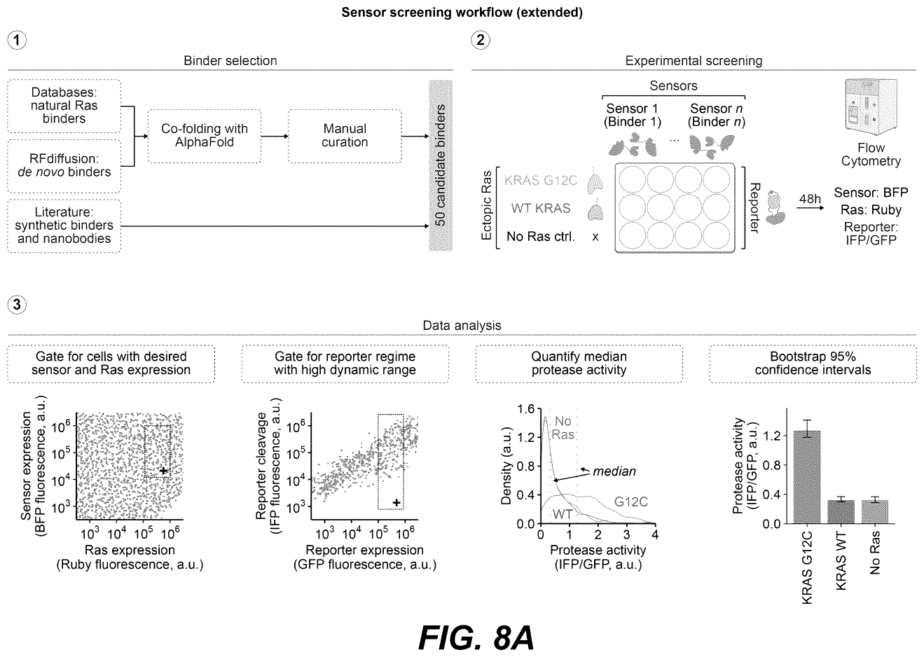

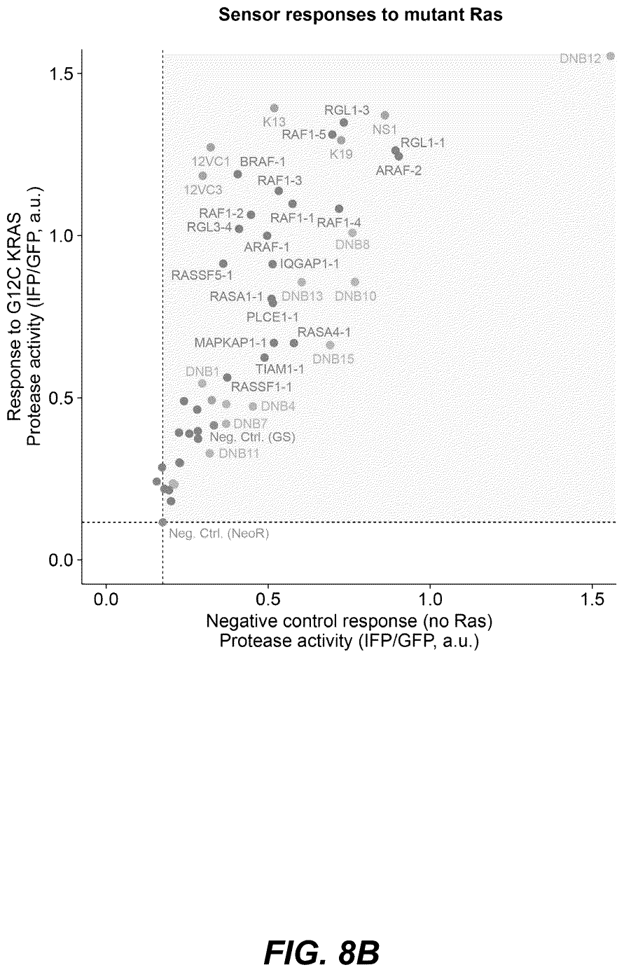

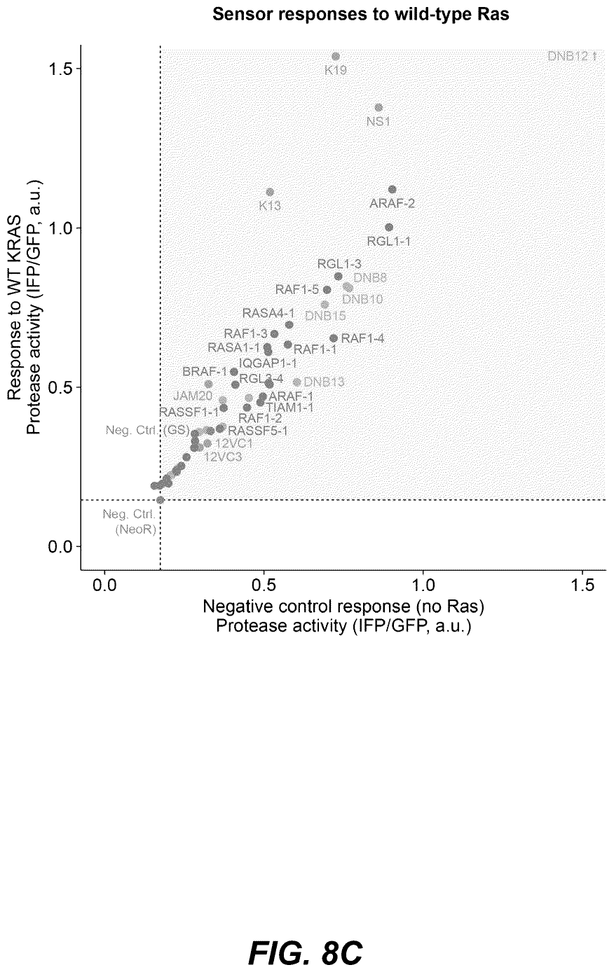

It was hypothesized that more specific and higher-affinity Ras binding domains may improve sensor function. To identify domains that would enable broad-spectrum mutant Ras sensing, a comprehensive library of human, de novo designed, and synthetic Ras binders was constructed. Specifically, a set of candidate human Ras binders was compiled by selecting proteins known to associate with KRAS, co-folding them with KRAS using AlphaFold3, and filtering for domains with high-confidence KRAS binding interfaces (See Methods). RFdiffusion was also used to design sensor variants that incorporate de novo Ras binders (See Methods). Finally, previously described Ras-binding monobodies, designed ankyrin repeat proteins (DARPins), synthetic proteins, and nanobodies were incorporated. To screen this library, each of these Ras binding domains was fused with complementary split TEVP domains and co-expressed with a panel of Ras variants as well as a fluorescent protease reporter in HEK293 cells (FIG. 2B, also see Methods). The reporter used a cleavage-activated IFP to measure protease activity and a GFP to track reporter expression, with the IFP/GFP ratio serving as normalized reporter readout. In the assay, additional fluorescent proteins were co-transfected to enable quantitative flow cytometry scanning of a broad, multi-dimensional expression space for each Ras mutant and candidate sensor (FIG. 2B, FIG. 8A).

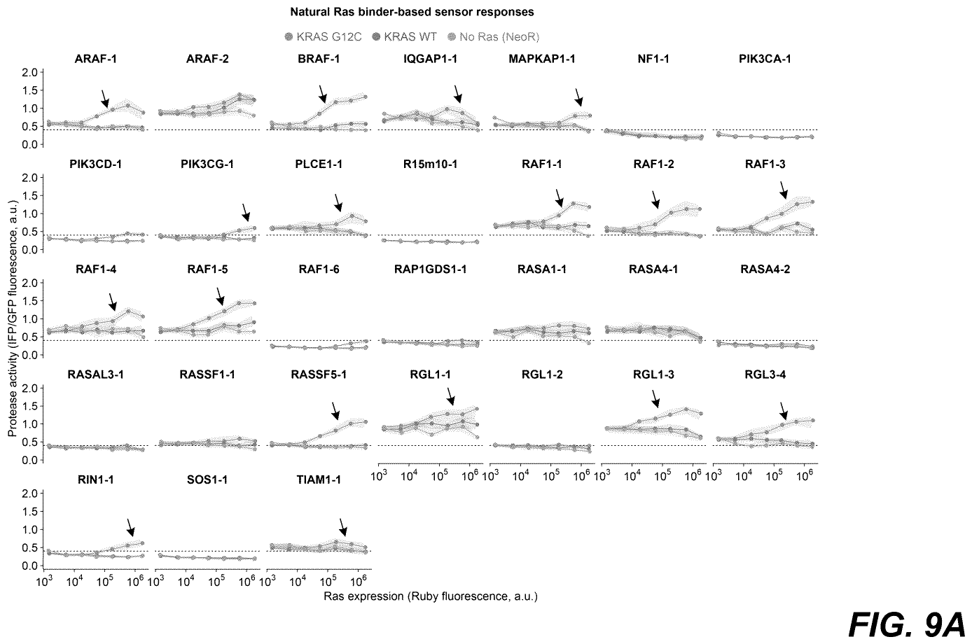

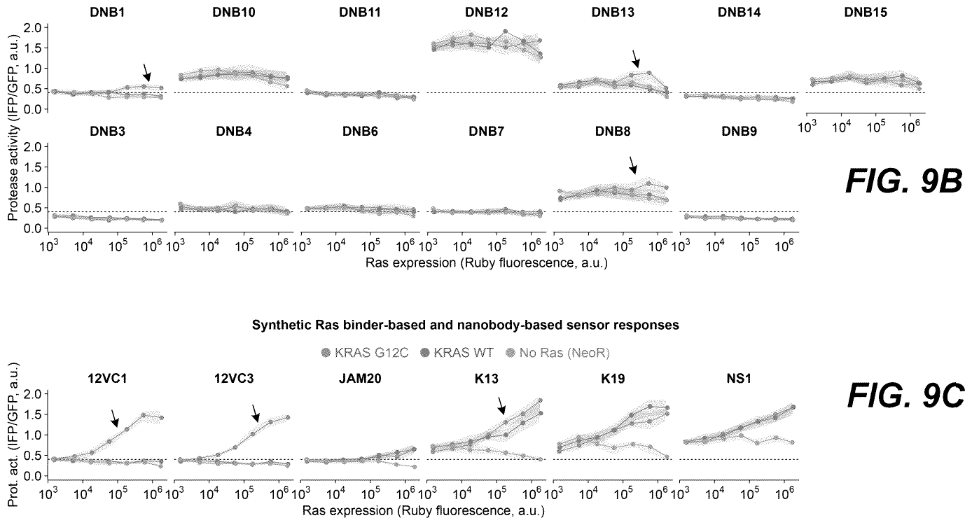

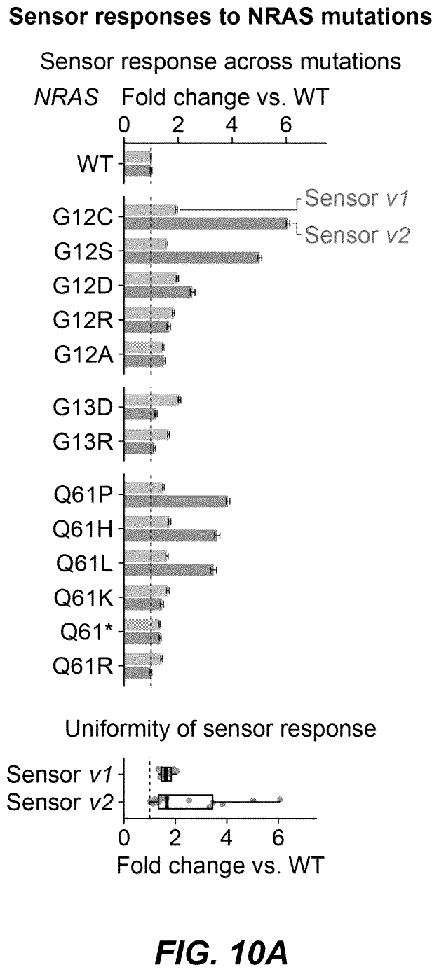

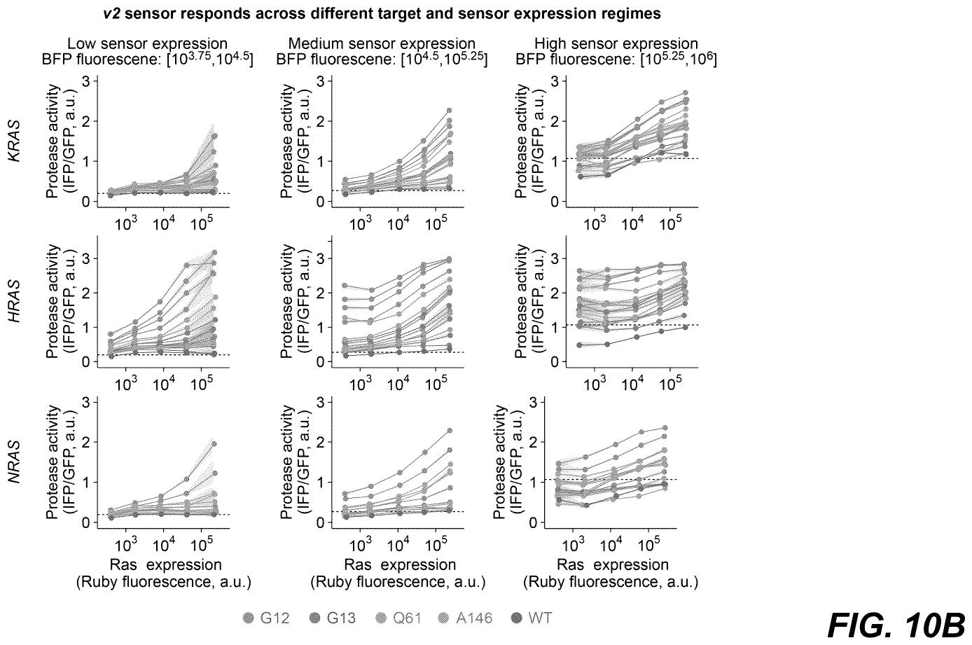

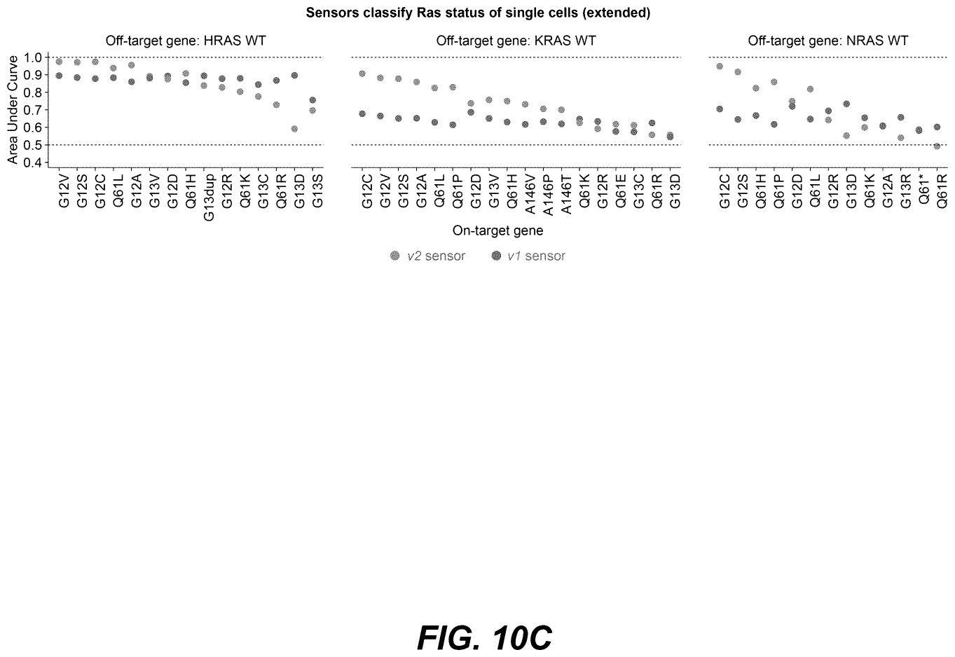

Candidate sensors varied in their responses to mutant (KRAS^{G12C}) and wild-type (KRAS^{WT}) Ras (FIG. 2C-FIG. 2D, FIG. 8B-FIG. 8C, FIG. 9A-FIG. 9C). Among the de novo designed binders, a small subset exhibited weak discrimination of mutant over wild-type Ras, providing a basis for potential further optimization (FIG. 9B). Sensors based on the synthetic monobody NS1 or the DARPins K13 and K19 were strongly Ras-responsive but did not discriminate between wild-type and mutant Ras variants (FIG. 2C-FIG. 2D). By contrast, sensors based on the previously described 12VC synthetic monobody (12VC1 and 12VC3) achieved sensitive and specific discrimination of mutant and wild-type Ras (FIG. 2C-FIG. 2D). This likely reflects the monobody selection process, which combined positive selection for KRAS^{G12C} binding with negative-selection against wild-type KRAS. Further, the 12VC1 sensor—henceforth referred to as v2—exhibited a sensor expression-dependent response across a broad range of Ras expression levels (FIG. 2D, FIG. 10B).

The v2 sensor detected a broad spectrum of clinically relevant oncogenic Ras variants. Following a similar experimental procedure as described above (See Methods), the response of v2 to a panel of 46 prevalent KRAS, HRAS, and NRAS mutations from the TCGA database was analyzed. At matched Ras expression levels, the v2 sensor strongly responded to most KRAS, HRAS, and NRAS mutants (FIG. 2E, FIG. 10A). The prevalent G12 mutations G12C, G12V, G12S, and G12A induced strong responses (FIG. 2E). Notably, the v2 sensor produced sufficient discrimination of wild-type and mutant Ras-expressing cells to enable accurate classification at the single-cell level (FIG. 2F, FIG. 10C).

To evaluate v2 sensor activity at physiologic Ras expression levels, human cancer cell lines were transfected with the sensor and a protease reporter. The sensor strongly responded in cell lines with mutant Ras, including MIA PaCa-2 (KRAS^{G12C}), NCI-H358 (KRAS^{G12C}), and NCI-H441 (KRASG12V; FIG. 2G). By contrast, it showed minimal response in cancer cell lines with wild-type Ras, including PLC/PRF/5, SNU-423, SNU-449, SNU-475. Taken together, these results indicate that the v2 sensor can sensitively and specifically respond to diverse, clinically-relevant Ras mutants expressed at endogenous levels in human cells.

### Sensor Optimization and Signal Amplification Improve Sensitivity

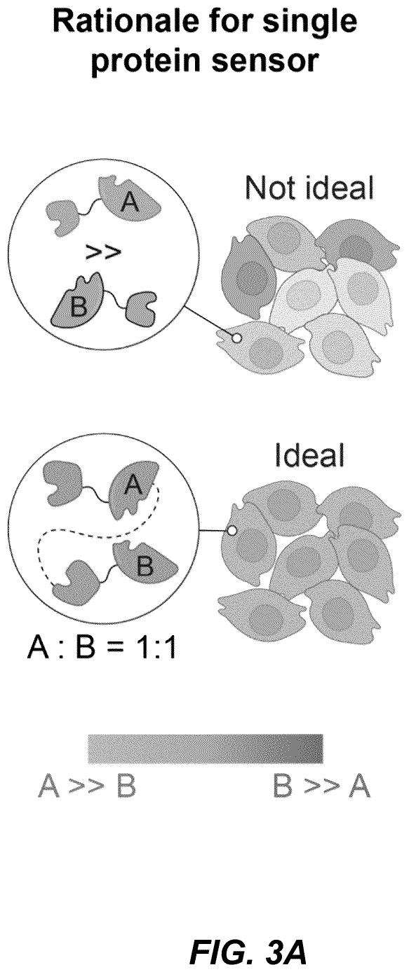

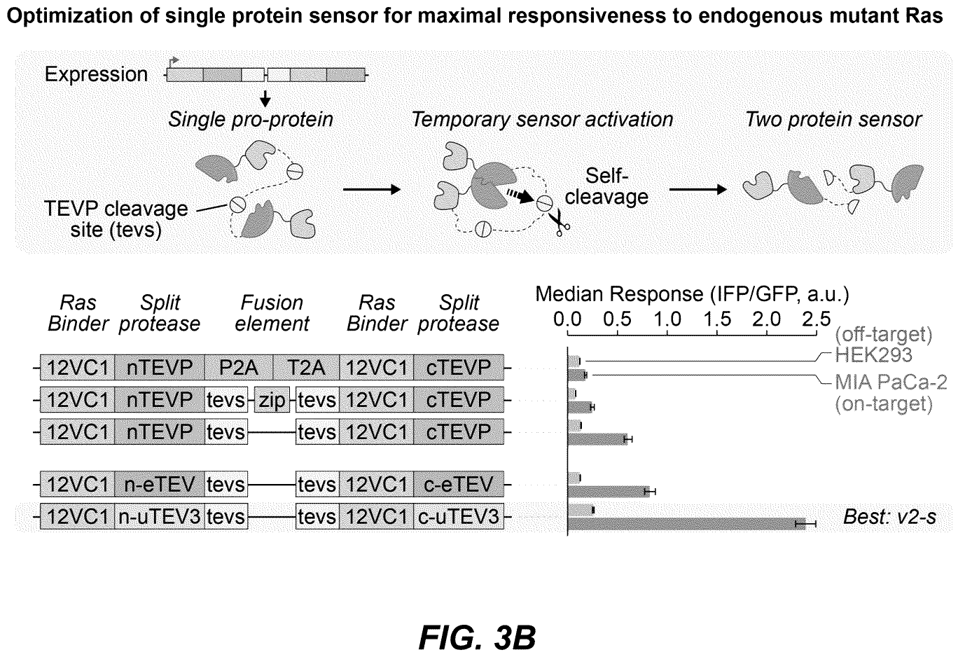

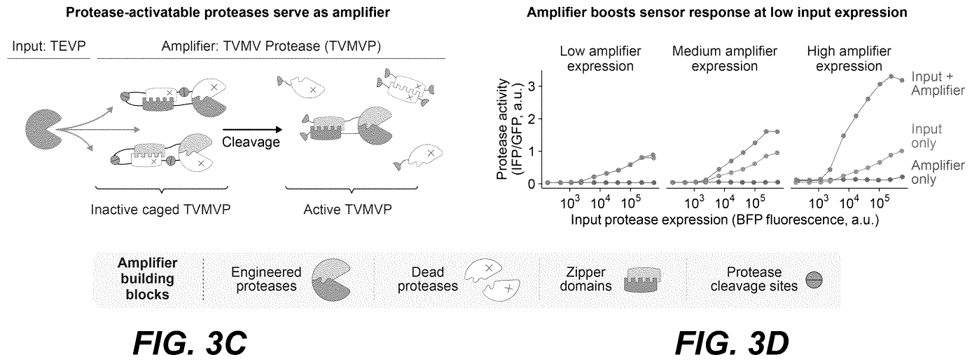

Anticipating variability of in vivo delivery, the two-protein sensor was re-designed as a single gene construct (FIG. 3A). First, we tried incorporating a T2A-P2A ribosomal skipping sequence. However, this modification degraded sensor performance (FIG. 3B). As an alternative, inspired by natural self-cleaving viral polyproteins, intramolecular cleavage sites for the sensor protease itself were introduced. In these designs, a polyprotein containing both sensor protein halves post-translationally cleaves itself into the two sensor components. These self-cleaving designs increased maximal activation. Additionally, incorporating TEV protease mutations further enhanced sensitivity (FIG. 3B). Based on these results, the self-cleaving sensor v2-s was adopted for subsequent experiments.

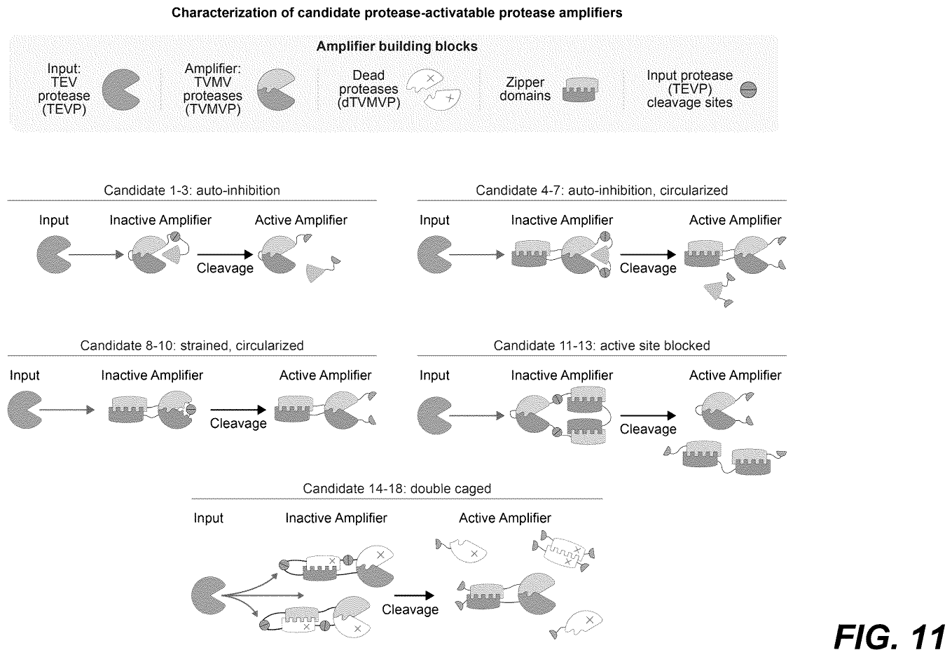

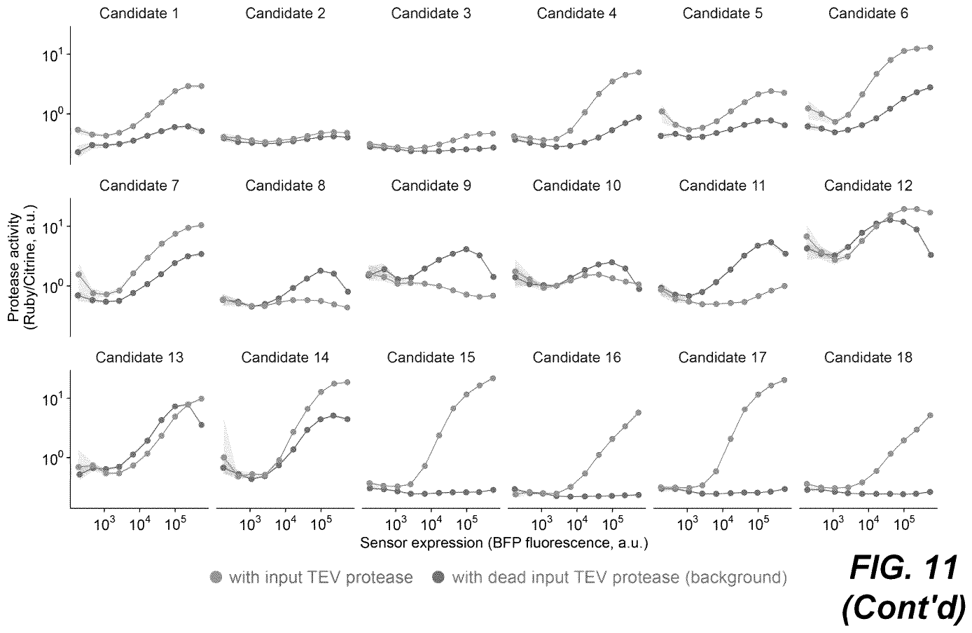

As a complementary approach, signal amplification to enhance sensor performance was also explored. Inspired by amplifying proteolytic cascades in cell death pathways, synthetic protease-activatable proteases were engineered (FIG. 11) using the tobacco vein mottling virus protease (TVMVP), which is orthogonal to the TEV protease. In the best design, split TVMVP halves were fused to caging domains consisting of catalytically dead complementary protease halves, which can be removed through TEVP cleavage (FIG. 3C). Once cleaved by TEVP, active TVMVP halves can reconstitute via attached coiled-coil domains (See Methods). This double-caged system amplified input signals compared to a constitutive TEVP alone, while maintaining minimal background activity in the absence of input TEVP signals (FIG. 3D). Together, these results establish optimized sensors and amplifiers for downstream in vivo applications.

### Circuits Potently and Selectively Eliminate Ras-Mutant Cancer Cells

To enable selective cancer cell killing in response to mutant Ras sensing, a complete therapeutic circuit must (1) be efficiently delivered to both healthy and cancer cells, and (2) couple Ras sensors to tightly controlled effectors of cell death. It was confirmed herein that an existing LNP formulation may efficiently deliver in vitro transcribed mRNA to human cells in culture, and that protein expression levels are quantitatively tunable by LNP concentrations (FIG. 12A-FIG. 12B, Methods).

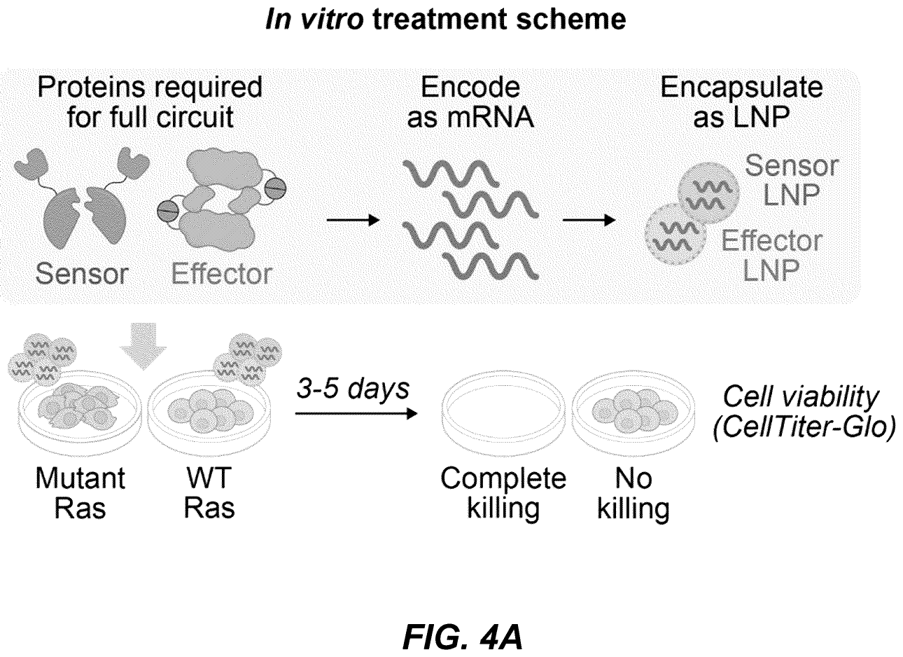

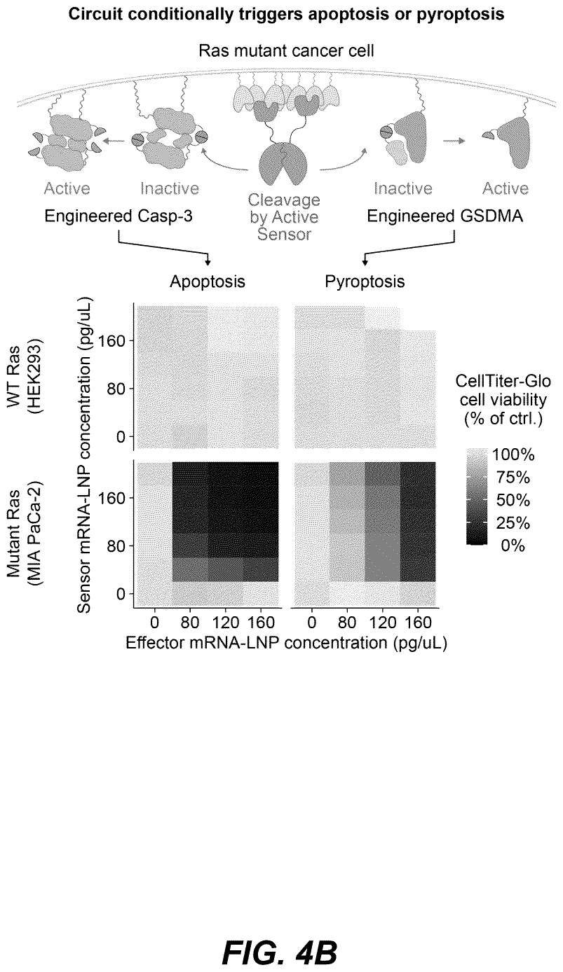

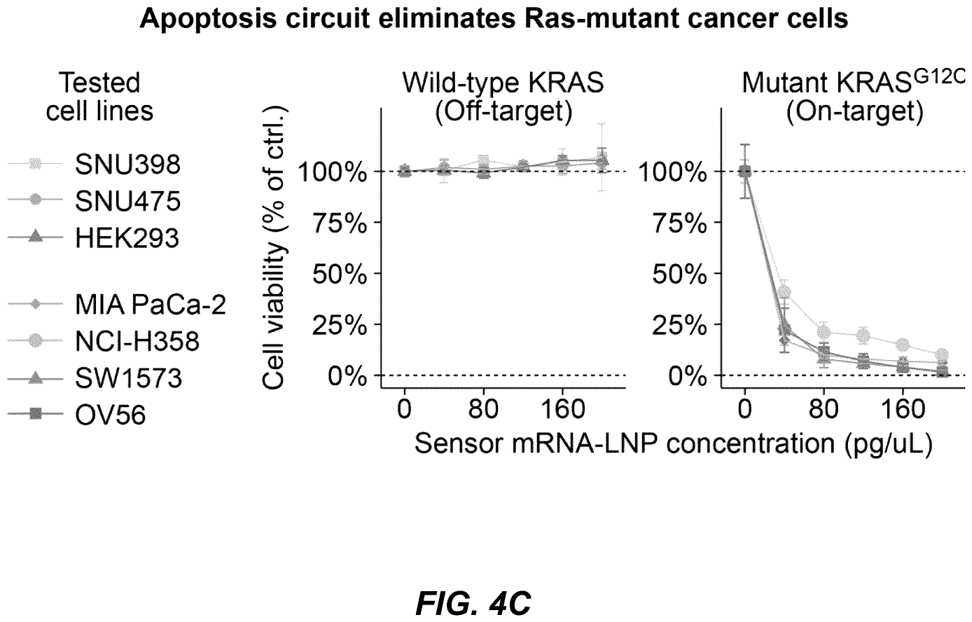

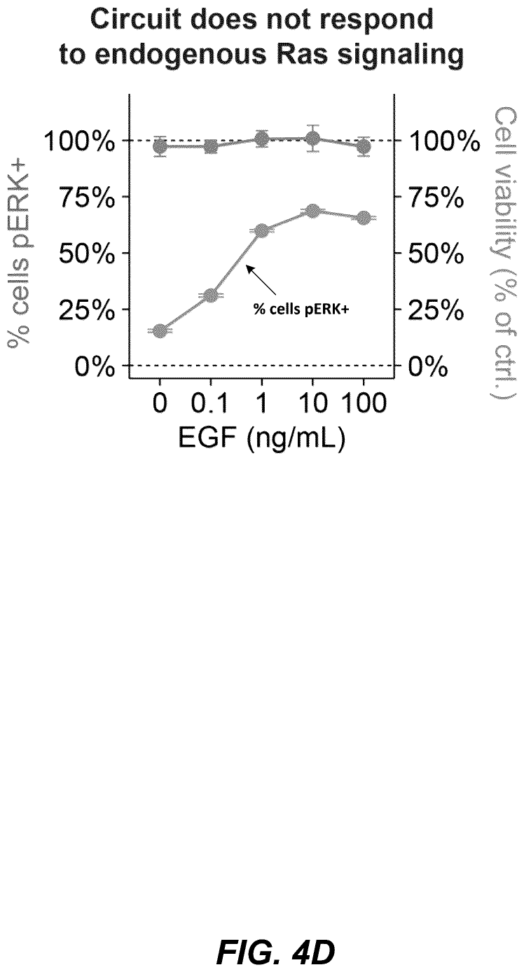

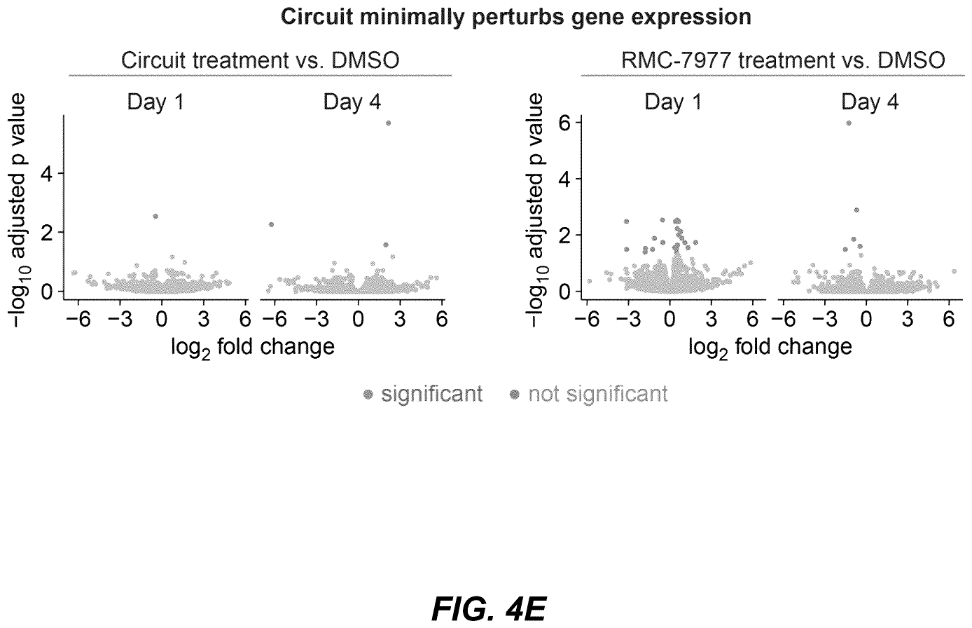

First, mutant Ras sensors were coupled to effectors of apoptosis, a precise and tightly regulated mode of cell death. Specifically, mRNAs encoding the v2-s sensor and a TEVP-activatable, membrane-localized caspase-3 effector (Casp-3) that conditionally triggers apoptosis (FIG. 4A) were separately encapsulated. Different concentrations of the two mRNA-LNP components were delivered to human cancer cell lines and analyzed cell viability 3-5 days post-treatment. Both the sensor and the effector were well tolerated across a wide dosage regime when delivered individually (FIG. 4B-FIG. 4C). Strikingly, co-delivery of the sensor and caspase effector induced potent, near-complete, and dose-dependent killing in Ras-mutant cancer cell lines, while sparing Ras wild-type cells (FIG. 4B-FIG. 4C). These results demonstrate that therapeutic circuits can selectively kill human Ras-mutant cancer cells.

To broaden the effects of therapeutic circuits beyond transfected cells, Ras sensing were also coupled to effectors of pyroptosis, an immunogenic form of cell death. Indeed, current mRNA delivery methods cannot penetrate all tumor cells in vivo. While partial tumor clearance may offer some therapeutic benefit, extending circuit-mediated killing to non-transfected cancer cells would be advantageous. Unlike apoptosis, pyroptosis releases inflammatory cytokines that can recruit the immune system, inducing non-cell autonomous tumor clearance even when triggered in a minority of cancer cells (FIG. 1). To test conditional activation of pyroptosis, the v2-s Ras sensor was co-delivered with a cleavage activatable gasdermin, the natural cell death effector of the pyroptosis program. As above, these components induced negligible background cell death when delivered alone, but potently and selectively eliminated Ras-mutant human cancer cells when co-delivered as a complete therapeutic circuit (FIG. 4B). Together, these results show that therapeutic circuits delivered as mRNA in LNPs can effectively target Ras-mutant cancer cells with two complementary cell death programs.

### Circuits do not Respond to Endogenous Ras Signaling or Perturb Gene Expression

A potential safety concern is off-target activation of circuits in wild-type cells engaged in normal Ras signaling. To test for such effects, mRNA-LNP circuits were transfected in HEK293 cells with or without Ras stimulation by epidermal growth factor (EGF). EGF treatment rapidly induced activation of the RAS-MEK-ERK pathway, with ERK phosphorylation detectable within 15 minutes, as expected (FIG. 4D). Nevertheless, the transfected cells exhibited no detectable cell death, even at saturating EGF concentrations. This ability to discriminate between wild-type and mutant Ras signaling may reflect a difference in Ras activation dynamics, with cancer cells exhibiting more sustained Ras activity than wild-type cells, where activation may be adaptive or oscillatory. These results demonstrate a safety feature of the sense-kill circuits and help explain their mutant specificity.

Another potential safety concern is that transient expression of circuit components could potentially perturb cell states. Using RNA-seq, the gene expression profile of HEK293 cells following treatment with either the mRNA-LNP circuit or the Ras targeting inhibitors Sotorasib and RMC-7977 was analyzed. 24 h after treatment, the mRNA-LNP circuit yielded only one differentially expressed gene (Benjamini-Hochberg-adjusted p<0.05; FIG. 4E, FIG. 12C). By contrast, RMC-7977 and Sotorasib differentially regulated 24 and 6 genes, respectively. In particular, RMC-7977 downregulated known transcriptional targets of the RAS-MAPK pathway such as DUSP6 (p=0.0041) or ETV5 (p=0.003), consistent with previous results and its role as a pan-Ras signaling inhibitor, while the circuit did not significantly affect these genes. By 4 days post-treatment, gene expression in all three conditions had largely returned to baseline. These results suggest that mRNA-LNP circuits minimally perturb healthy cells.

### Circuits Prevent and Treat Induced Liver Tumors In Vivo

Having demonstrated specific targeting in cell culture, it was next asked whether therapeutic circuits could function in vivo. The liver represents an ideal context to test circuits in vivo for two reasons. First, existing LNP formulations can efficiently transfect the liver. Second, the liver is the primary metastatic site for many Ras-driven cancers with high unmet clinical need, as well as the primary site for Ras-driven cholangiocarcinomas.

Liver tumors develop in complex microenvironments, with vasculature, immune cells, and extracellular matrix influencing cancer growth. To recapitulate these features, an established autochthonous liver cancer model in immunocompetent mice was used. In this model, hydrodynamic tail vein injection (HDT) is used to transfect hepatocytes with DNA encoding the oncogenic NRAS^{G12V} Ras variant, an shRNA targeting TP53 (shTP53), and the Sleeping Beauty (SB100) transposase for stable genomic integration and sustained expression. The model generates aggressive and multifocal liver cancers that advance to late-stage disease within five weeks after induction.

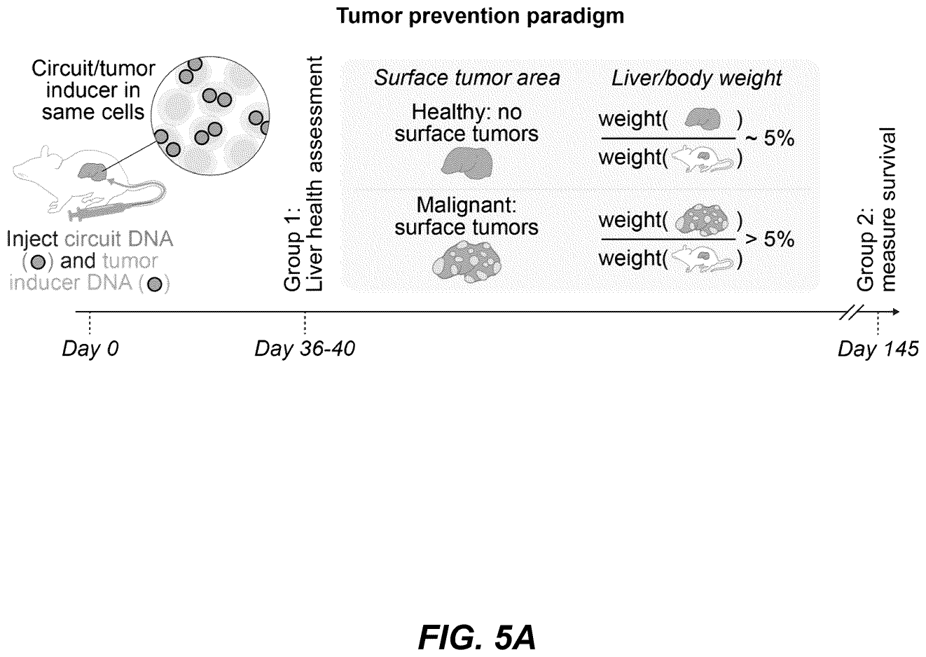

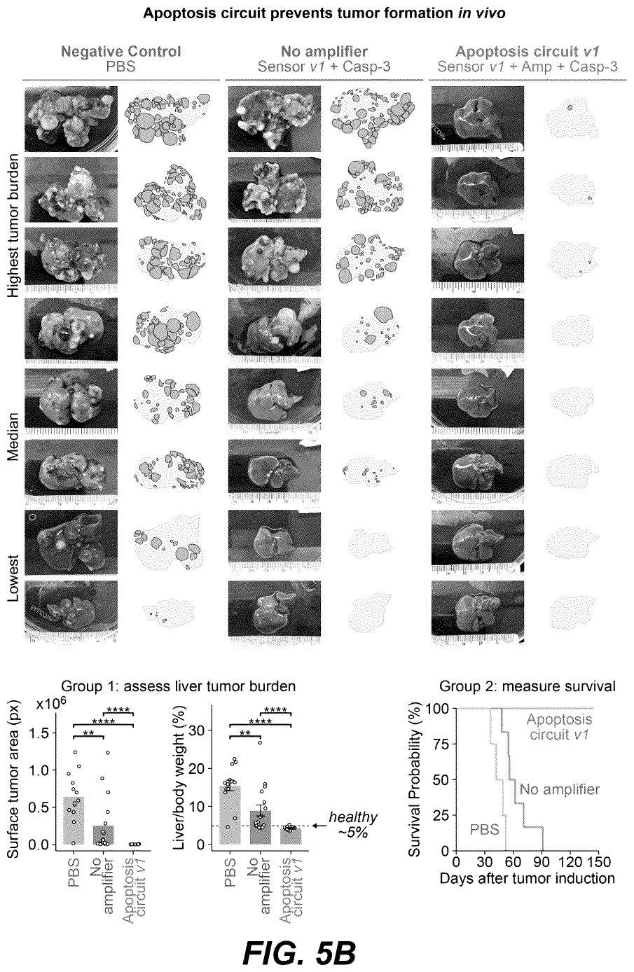

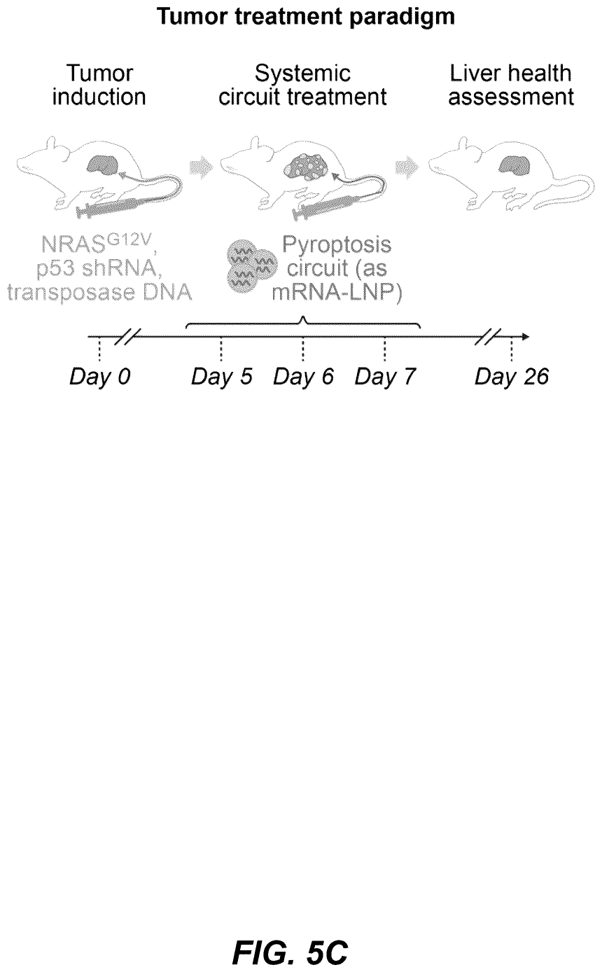

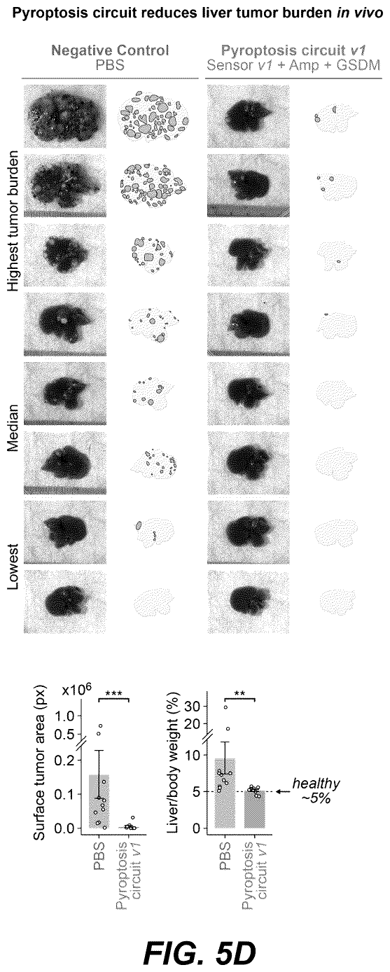

Using this model, it was first asked whether circuits could function in vivo, independently of potential delivery limitations. To address this question, DNA-encoded circuits were co-administered with the tumor-inducing agents to allow tumor initiation and circuit expression to occur largely in the same liver cells (FIG. 5A). We used circuits that incorporated the v1 sensor, as experiments were conducted before the improved sensor variants were developed, and the apoptosis cell death effector, as tumor induction was largely restricted to circuit-expressing cells. Further, circuit variants with or without the amplifier were included. In one mouse cohort, tumors were allowed to develop for five weeks, terminated the experiment, and measured total surface tumor areas and liver-to-body weight ratios, a standard measure of tumor burden in hydrodynamic liver tumor models (FIG. 5A, Methods). In a separate mouse cohort, survival was tracked to determine the durability of tumor suppression (FIG. 5A, Methods).

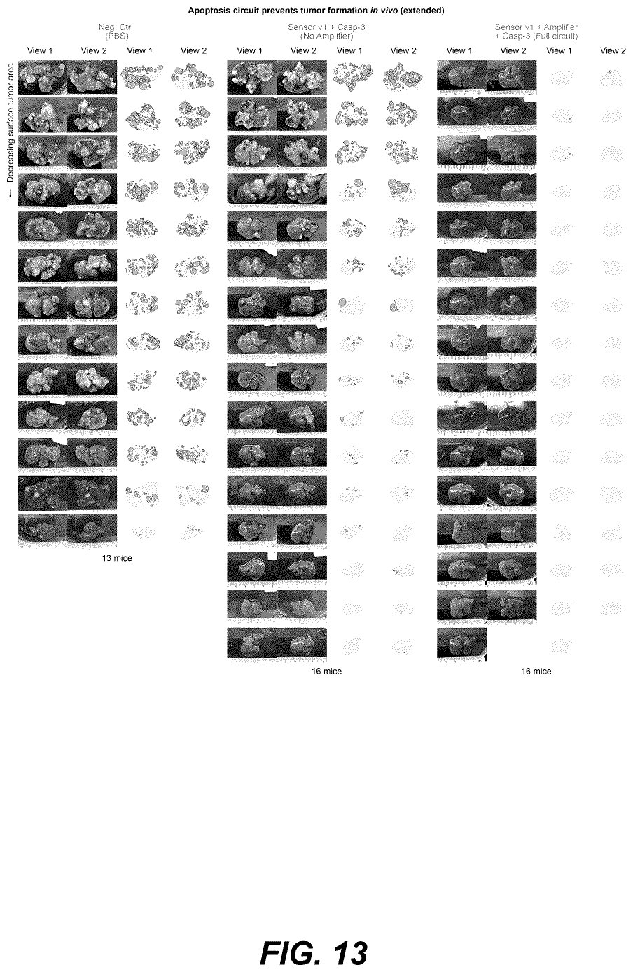

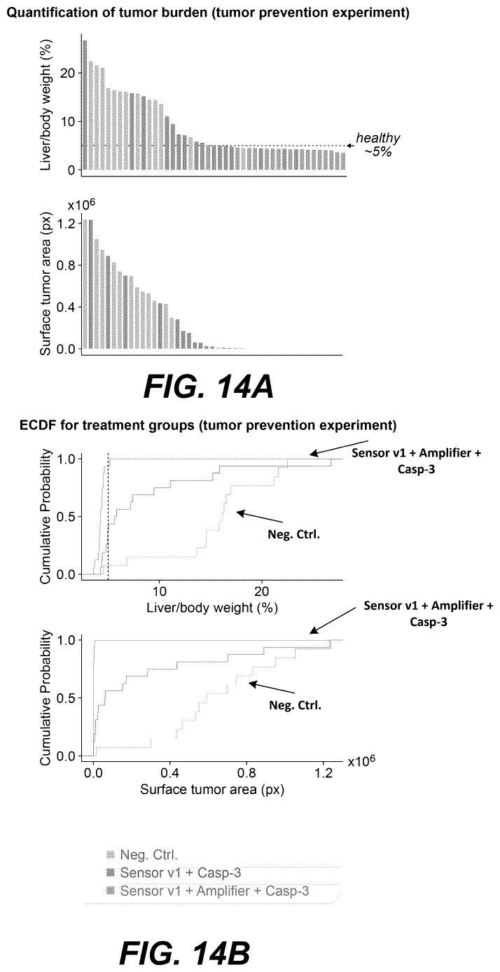

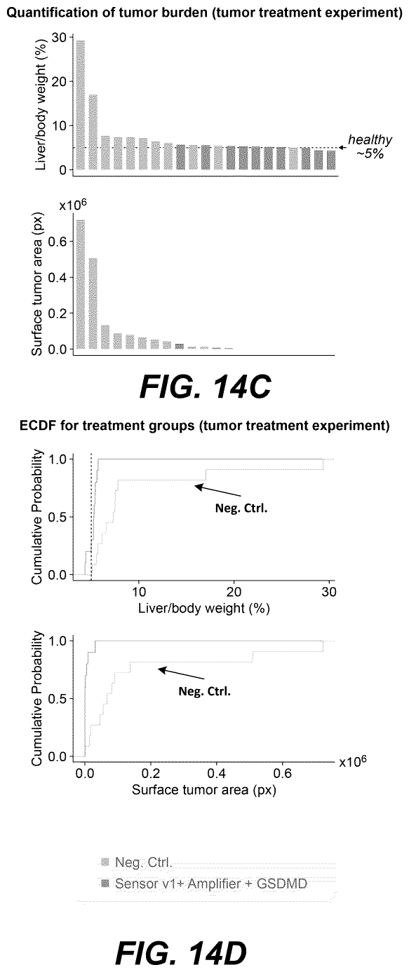

Mice that received no treatment showed robust, multifocal tumor formation (FIG. 5B, FIG. 13). Co-delivery of the sensor and cell death effector significantly, but incompletely, reduced tumor burden, as assessed by total surface tumor area (p=4.1×10⁻³) and liver-to-body weight ratios (p=2.8×10⁻³, FIG. 5B, FIG. 13, FIG. 14A-FIG. 14B). The same condition extended median mouse survival from 42 days (in untreated animals) to 59 days (p=7×10⁻⁴), but did not eliminate tumor development (FIG. 5B). Remarkably, inclusion of the amplifier module transformed this partial response into near-complete tumor suppression. Mice that received the complete sense-amplify-kill circuit showed no detectable surface tumors and exhibited liver-to-body weight ratios comparable to those of healthy animals (FIG. 5B, FIG. 13, FIG. 14A-FIG. 14B). In the parallel cohort, all mice treated with the sense-amplify-kill circuit survived beyond 145 days, at which time the experiment was terminated (FIG. 5B, p=1×10⁻⁴ for comparison to untreated). Overall, these results demonstrated that co-delivered therapeutic circuits can prevent the formation of induced mouse liver tumors in vivo.

Next, a more challenging treatment scenario was considered (FIG. 5C). Rather than co-delivering circuits with tumor-initiating agents, tumor formation (day 0) was initiated using the same procedure as above, and then tumors were allowed to develop for five days before the therapeutic circuits were systematically administered as mRNA-LNP in three doses, on days 5, 6, and 7. On day 25, we sacrificed mice and harvested livers for further analysis. In this case, because the circuits are unlikely to be delivered to the majority of cancer cells, the apoptosis effector was replaced with the pyroptosis effector as output. Thus, the circuits in this experiment consisted of the v1 sensor, amplifier, and gasdermin pyroptosis effector.

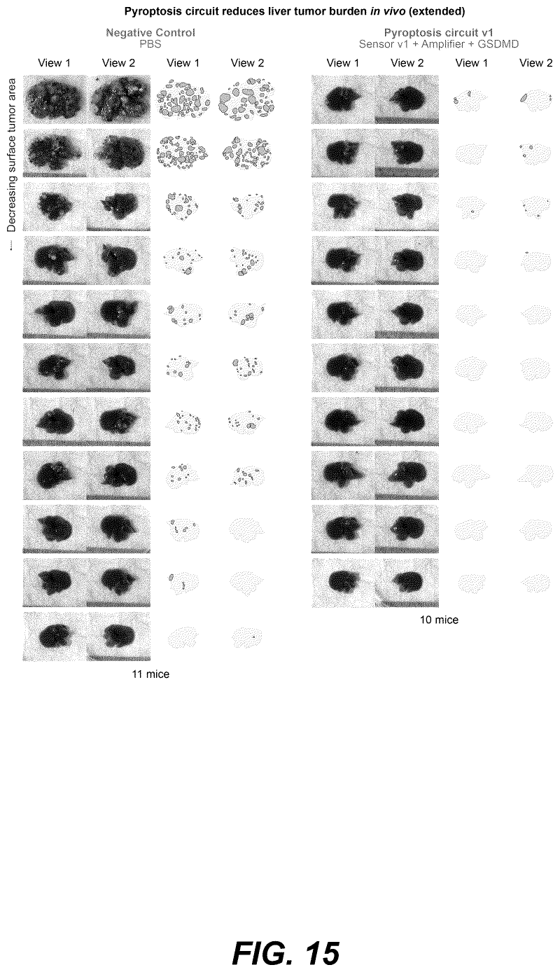

Compared to PBS-treated mice, circuit-treated animals showed near-complete tumor clearance FIG. 5D, FIG. 14C-FIG. 14D, FIG. 15). In fact, the livers of circuit-treated animals were almost completely free of surface tumor nodules (p value=0.00055 compared to the negative control group). The treated animals also exhibited liver-to-body weight ratios comparable to those of healthy animals (p value=0.00078 compared to negative control group). Thus, circuits delivered systemically using mRNA-LNPs are capable of eliminating previously initiated Ras-driven mouse liver tumors. This result was particularly striking given that this multifocal cancer model is known to be challenging to treat, and that this result was obtained using the less optimized sensor.

### Circuits Kill Drug-Resistant Cells

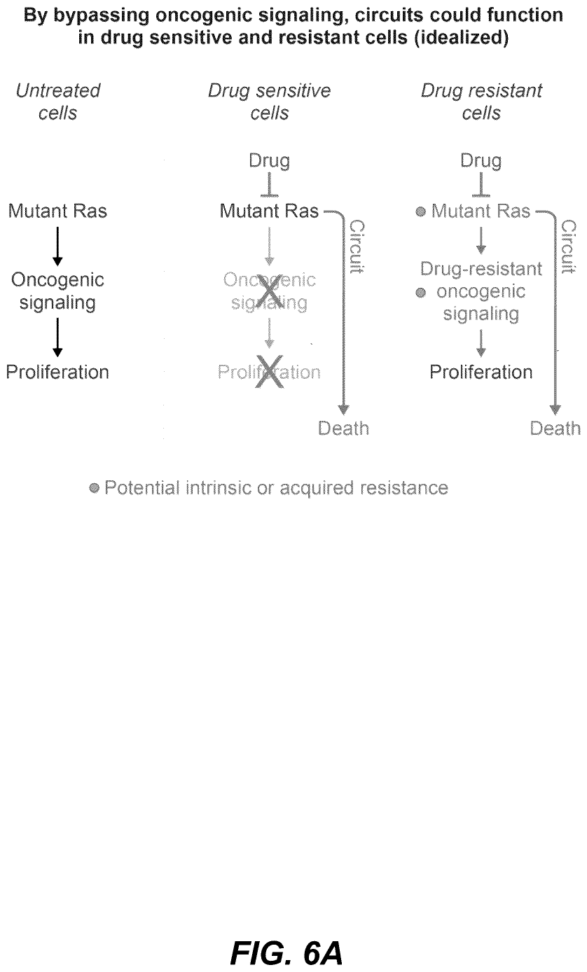

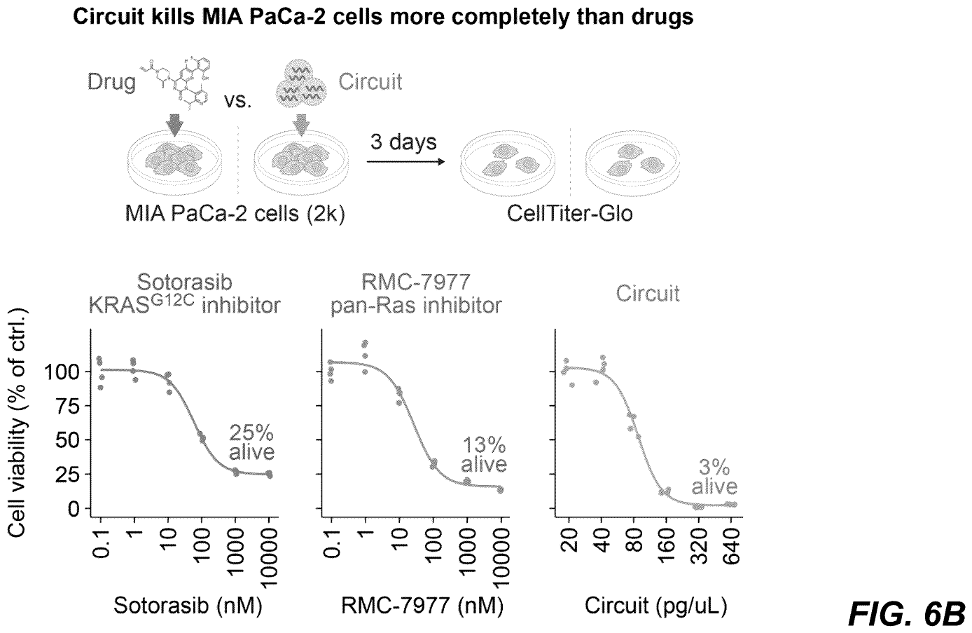

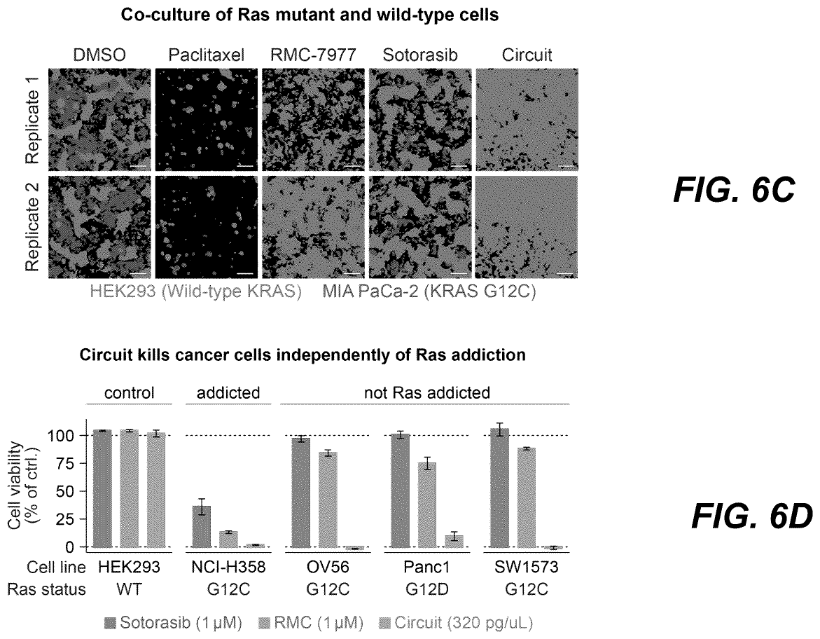

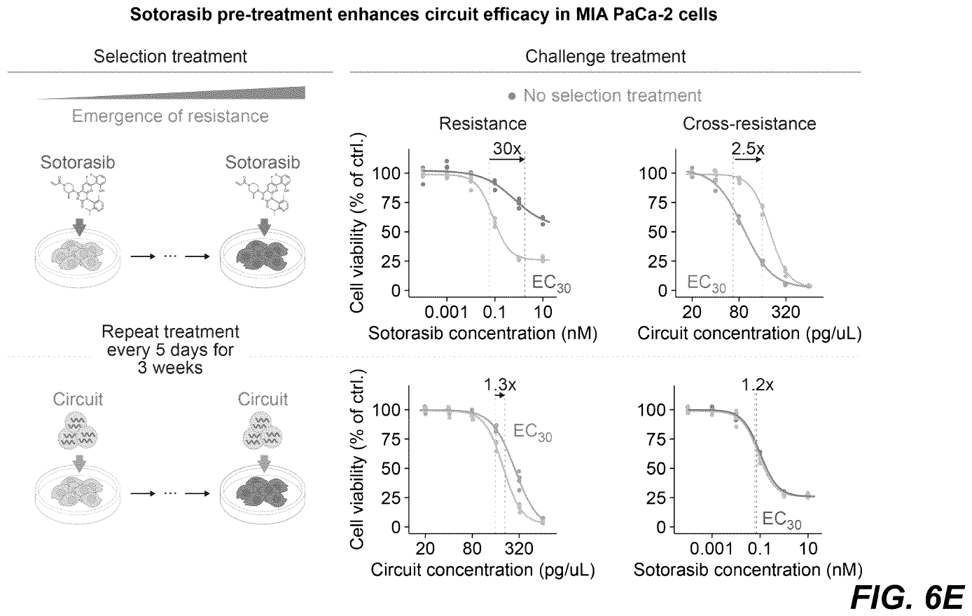

Both intrinsic and acquired resistance can limit the efficacy of Ras inhibitors (FIG. 6A). Intrinsic resistance can be assayed by the initial response of Ras-mutant cells to treatment. In contrast, acquired resistance arises when initially sensitive cells adapt over time, either by losing Ras dependency or by activating compensatory pathways to circumvent Ras inhibition. It can be observed through the gradual emergence of resistant cells after extended treatment. It was reasoned that therapeutic circuits, by directly rewiring mutant Ras sensing to cell death, would provide fewer opportunities for both types of resistance (FIG. 6A). To test this hypothesis, the performance of the mRNA-LNP-delivered apoptosis circuit (v2-s sensor, caspase effector) was directed compared to two clinically relevant small-molecule Ras inhibitors: Sotorasib, an approved KRAS^{G12C} inhibitor, and RMC-7977, a pan-Ras inhibitor with a related molecule currently under clinical evaluation.

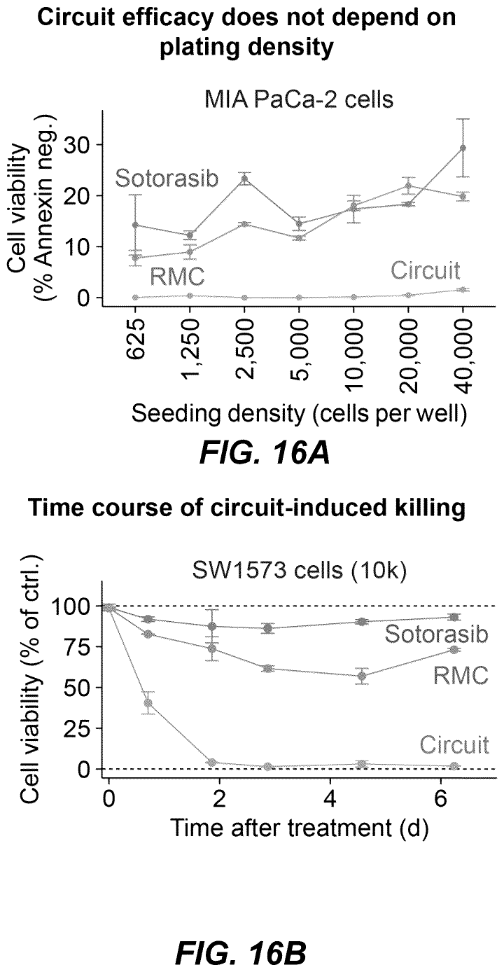

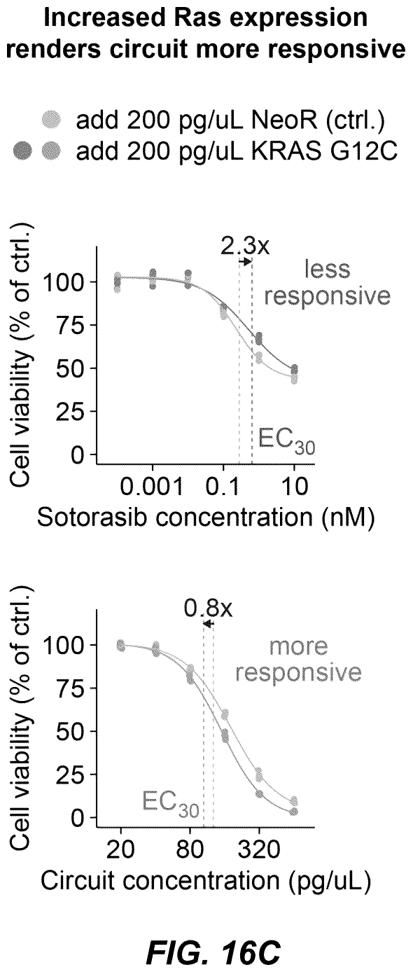

To assay for intrinsic resistance, the responses of KRAS^{G12C}-mutant MIA PaCa-2 cells were compared to either circuit or drugs. Sotorasib and RMC-7977 reduced cell viability in a dose-dependent manner by up to 74.8±0.5% or 86.8±0.3%, respectively, but neither drug achieved complete cell elimination, even at saturating concentrations (FIG. 6B). In contrast, the circuit achieved near complete elimination of all cells (97.1±0.1%, FIG. 6B). Also, while the potency of the drugs declined rapidly with increasing cell plating density, circuit potency remained constant (FIG. 16A).

In a complementary experiment, microscopy was used to compare drug and circuit effects on co-cultured target (MIA PaCa-2) and non-target (Ras wild-type HEK293) cells, engineered to stably express GFP and mRuby3 markers, respectively. Five days after treatment, the chemotherapeutic agent Paclitaxel produced potent but incomplete and non-specific clearance of both cell types (FIG. 6C). Sotorasib and RMC-7977 disproportionately reduced the size of the Ras-mutant cell population, but failed to eliminate them entirely, leaving behind visible colonies of Ras-mutant cells (FIG. 6C). By contrast, the circuit completely cleared Ras-mutant, but not wild-type Ras cells (FIG. 6C). Together, these results suggest that circuits can be more potent and specific than targeted therapies and chemotherapy.

Notably, circuits were effective against Ras mutant cells regardless of whether they exhibited oncogene addiction (FIG. 6D). Three Ras-mutant cell lines that do not exhibit Ras addiction were tested: SW1573 (lung, KRAS^{G12C}), OV56 (ovary, KRAS^{G12C}), and PANC-1 (pancreas, KRASG12D). While these cells exhibited weak responses to drugs, they remained fully sensitive to the circuit (FIG. 6C). The circuits also produced durable effects. The response of non-Ras addicted SW1573 cells to a single dose of drug or circuit over 1 week of continuous culture was analyzed. The circuit, but not the drugs, durably eliminated these cells and prevented their recovery within this period (FIG. 16B).

Acquired resistance limits the clinical efficacy of targeted therapies such as Sotorasib. To quantitatively compare the susceptibility of MIA PaCa-2 cells to acquired resistance under Sotorasib or circuit treatment, three rounds of five-day selection at the EC70 of each treatment was performed. Cells were then challenged with another round of the same respective treatment to assess resistance (FIG. 6E). Consistent with preclinical models and patient outcomes, Ras mutant MIA PaCa-2 cells rapidly acquired resistance, requiring 30-fold higher drug concentrations to reach the same cell killing efficiency (EC30) post-selection compared to pre-selection (FIG. 6E). In contrast, circuit selection increased circuit EC30 by only 1.3-fold. Moreover, Sotorasib's maximum killing efficiency dropped from ~75% in pre-selection cells to ~50% in post-selection cells, whereas the circuit maintained over 98% maximum efficiency post-selection (FIG. 6E). These results indicate that MIA PaCa-2 cells are less susceptible to acquired resistance to the circuit compared to Sotorasib.

Finally, given the clinical challenge of Sotorasib resistance, we wondered whether cells with acquired Sotorasib resistance (generated above) would also exhibit resistance to the circuit, or vice versa (FIG. 6E). Interestingly, Sotorasib-resistant cells required 2.5-fold lower concentrations of circuit mRNA-LNP to achieve the same killing efficiency (EC30) as non-resistant cells (FIG. 6E). Without being bound by any particular theory, it was reasoned that this effect may be related to the known resistance mechanisms of increased Ras expression (or amplification), either of which could increase sensitivity to the circuit. To test this hypothesis, KRAS^{G12C} was transiently overexpressed in MIA PaCa-2 cells and these cells were treated with either Sotorasib or the circuit. Indeed, transient KRAS^{G12C} overexpression slightly increased resistance to Sotorasib but enhanced sensitivity to the circuit (FIG. 16C). Together, these results highlight a fundamental difference in the resistance profile of drug and circuit-based therapies and suggest that future therapeutic circuits can help treat patients resistant to Ras-inhibiting drugs.

### Discussion

The results presented herein demonstrate end-to-end development of therapeutic protein circuits that kill Ras-mutant cancer cells and treat tumors in vivo. In fact, despite the induced tumors analyzed here being aggressive and extremely difficult to treat, circuits still showed significant therapeutic benefit. The circuits designed here achieved comparable specificity and superior potency compared to state-of-the-art drugs, while being considerably easier and faster to design. What accounts for these advantages? First, unlike drugs, the sense-and-kill mechanism does not require oncogenic addiction and is less sensitive to complex and poorly understood heterogeneity (outside of the Ras gene itself) in cancer cell populations. Second, the ability to use proteins as sensors allows for large binding interfaces that can detect molecular features such as mutations that are otherwise challenging to drug. Third, circuits allow precise control of cellular responses, in this case through activation of pre-defined cell death programs.

Circuit therapeutics have other advantages as well. The circuits are genetically compact (e.g., ~2 kb for the pyroptosis circuit v2-s) and easily deliverable by LNP. From a safety point of view, the use of mRNA allows circuits to function transiently without permanent genome modification. Circuits also offer benefits compared to pyroptosis-inducing gasdermin agonists. Unlike these drugs, which broadly activate pyroptosis and could induce hyper-inflammation, circuits restrict its activation to cancer cells. Additionally, since circuits ectopically deliver gasdermins, they can overcome the observed problem of gasdermin silencing in tumor cells, which would render drugs ineffective.

Perhaps the most interesting aspect of therapeutic circuits is their programmability. Replacing the sensor in the current circuits can retarget them to other cancer drivers such as MYC, β-catenin, or other oncogenes. In fact, circuits do not need to respond to oncogenes at all, but can be designed to sense any protein or combination of proteins that provide an accurate signature of disease state. Similarly, circuit outputs can also be broadened to include release of cytokines or activation of other therapeutic responses. Together, these results establish a potent, specific, and programmable mechanism for treating cancer and other human diseases.

### Example 2

### Engineered Protein Circuits for Cancer Therapy

Provided in this Example are additional studies related to the synthetic protein circuits of the disclosure.

### Circuits Enable Cytotoxic Cancer Cell Elimination

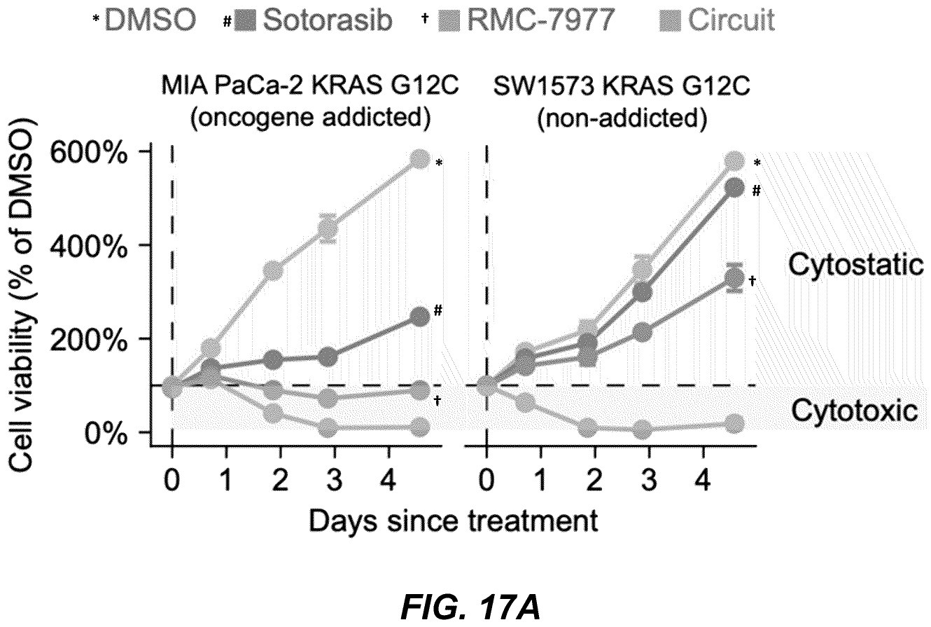

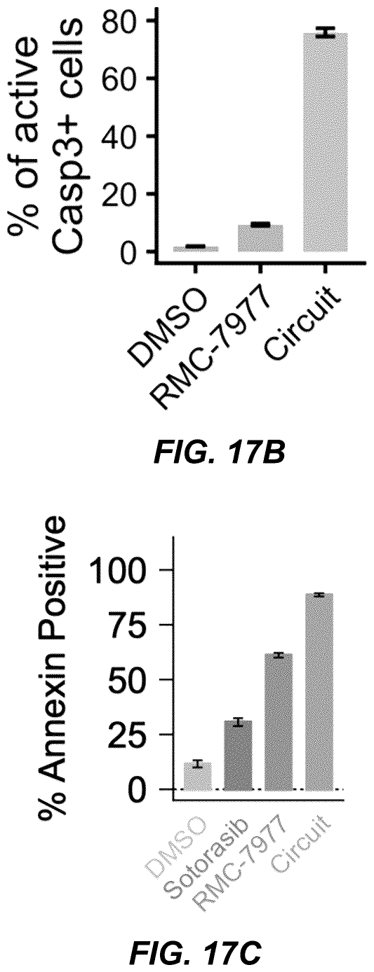

A time-course analysis was performed to quantify killing dynamics for the therapeutic circuits and the Ras-targeting inhibitors Sotorasib and RMC-7977 (FIG. 17A). Explicit Day 0 viability measurements were included to distinguish active cell death (cytotoxicity) from growth inhibition (cytostasis). Sotorasib is primarily cytostatic, slowing proliferation, whereas RMC-7977 produces a mixed cytotoxic and cytostatic response, consistent with prior reports (FIG. 17A-FIG. 17C). In contrast, the therapeutic circuits are strongly cytotoxic: they directly activate the caspase-3 cell-death program (positive active caspase-3 staining and Annexin V signal) and reduce viable cell populations to near zero, falling below the initial Day 0 baseline (FIG. 17A-FIG. 17C). This capacity to actively drive tumor regression is a key advantage of the circuit approach.

### Circuits Function Independently of MAPK Pathway Inhibition

Because prior characterization showed that the 12VC1 binder inhibits KRAS-driven MAPK signaling pathway signaling when expressed as a “naked” monobody, it was tested whether 12VC1-based circuits similarly suppress MAPK signaling or instead act through a mechanistically distinct pathway.

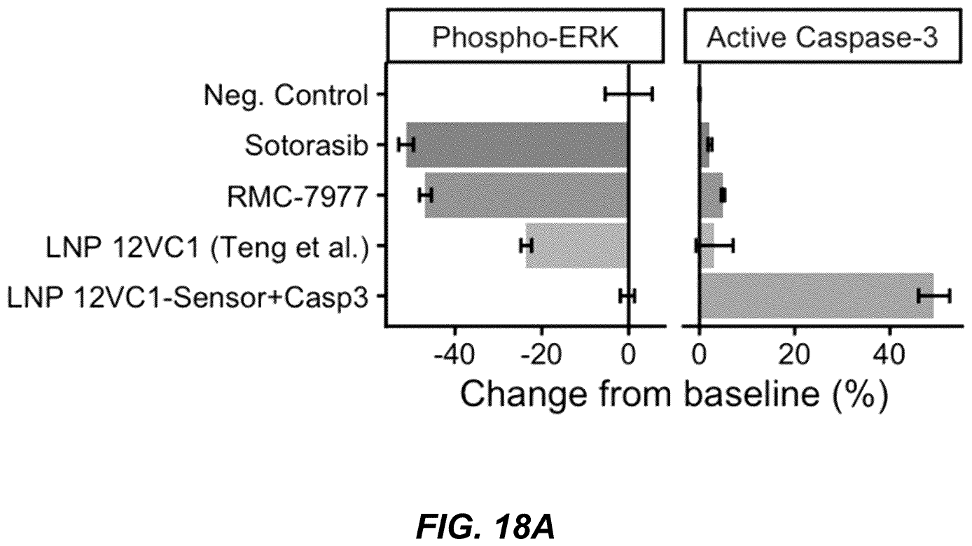

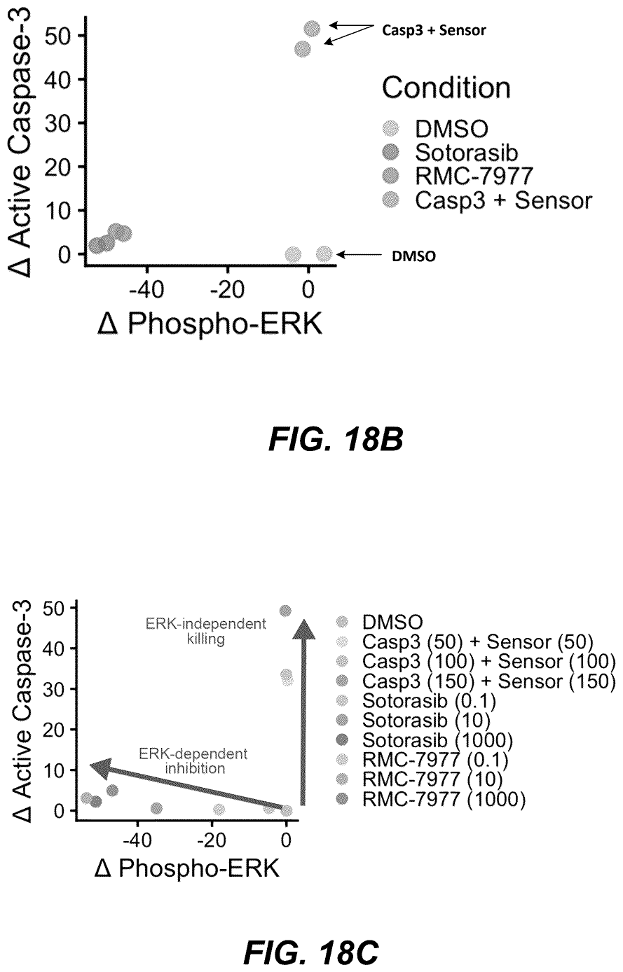

A head-to-head comparison was performed of (i) circuits, (ii) the naked 12VC1 monobody, and (iii) the Ras inhibitors Sotorasib and RMC-7977, measuring phospho-ERK (pERK) and active caspase-3 in MIA PaCa-2 cells after 24 hours (FIG. 18A-FIG. 18C). As expected, Sotorasib and RMC-7977 strongly reduced pERK. Consistent with prior reports, the naked 12VC1 monobody also reduced pERK. In contrast, the 12VC1-based circuit sensor did not measurably inhibit pERK, yet still triggered strong apoptosis.

Without being bound by any particular theory, two factors can contribute to the absence of measurable pERK inhibition by the circuit sensor. One is effective concentration. The v2 sensor is a large, complex, self-cleaving polyprotein encoded on a 1.5 kb mRNA and transiently expressed. It is unlikely to achieve as high of an effective concentration as the smaller (0.3 kb), naked monobody expressed in a sustained fashion from DNA, as in Teng et al “Selective and noncovalent targeting of RAS mutants for inhibition and degradation.” Nat. Commun. 12, 2656 (2021). A second factor is sterics. Fusing the small monobody to the bulky split-protease domains likely imposes steric constraints that prevent the high-occupancy binding required for sustained, stoichiometric blockade of downstream effectors like RAF.

This points to a fundamental difference in the circuit mechanism compared to inhibitors. Inhibitory drugs and monobodies physically block downstream effector binding and starve the cell of survival signals. On the contrary, circuits operate independently of ERK-signaling. Despite the lack of pERK suppression, circuits trigger high levels of active cell death. In this mechanism, mutant Ras functions merely as a marker to trigger an amplified enzymatic cascade.

Finally, while 12VC1 is exquisitely mutant specific, future sensors for other pathways may not achieve complete mutant specificity. In such cases, a lack of inhibition would prevent off-target modulation of biological pathways in healthy cells.

### Sensor Programmability Enables Retargeting to User-Defined Mutations

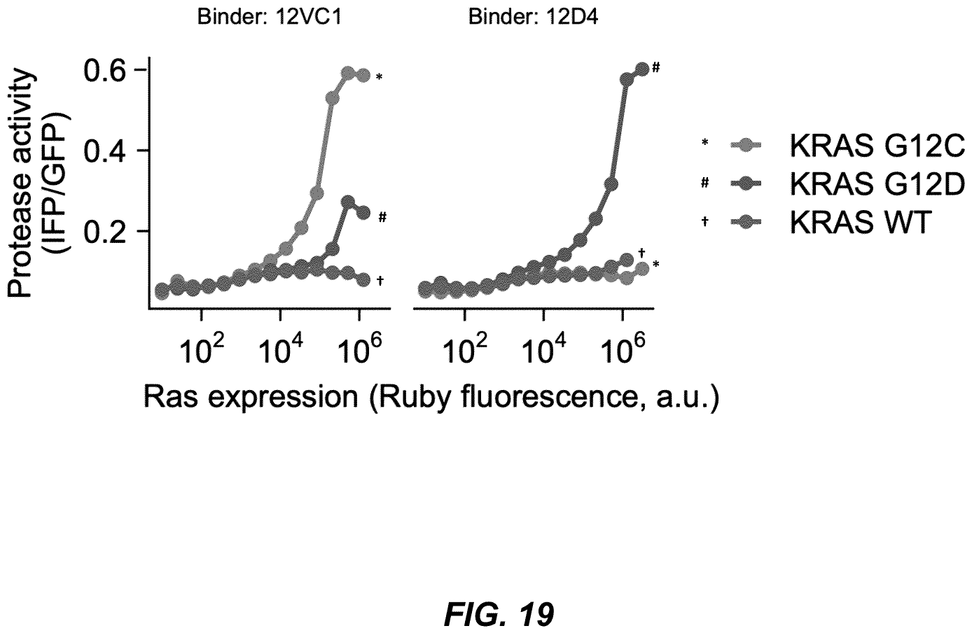

To demonstrate generalizability, a new circuit variant was engineered by swapping the 12VC1 binder, which primarily recognizes KRAS G12C, for the 12D4 monobody reported in a second pioneering paper from the same group, which is more selective for KRAS G12D. This new circuit potently detects G12D while discriminating against wild-type and G12C KRAS variants (FIG. 19). This result highlights the modular and extensible nature of the circuit platform and suggests the possibility of mixed circuits that co-express both G12C and G12D sensors.

### Activation of Ras-Targeting Sensor is Driven by Membrane Localization

To define biophysical requirements for sensor activation, a co-expression assay using engineered KRAS G12C variants that perturb membrane trafficking, anchoring, and oligomerization was performed.

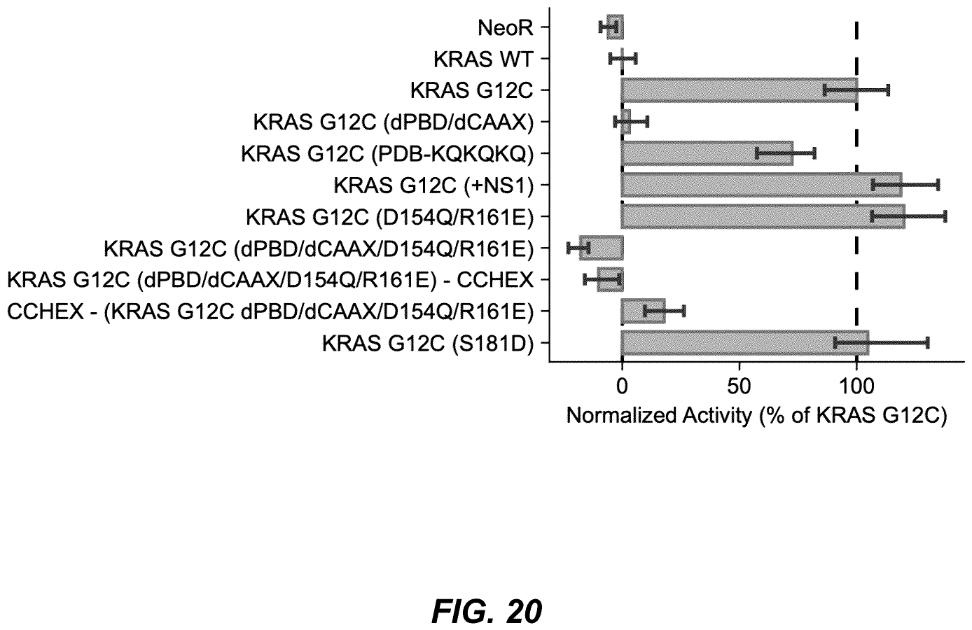

Deleting or altering the hypervariable region (HVR) that mediates membrane targeting, specifically the polybasic domain (dPBD) and CAAX motif (dCAAX), abolished activation. Weakening the polybasic domain (e.g., KKKKKK (SEQ ID NO: 357)→KQKQKQ (SEQ ID NO: 358)) reduced activity. Together, these data indicate that 2D membrane localization is required for robust sensing (FIG. 20).

To test if the sensor relies on Ras-Ras dimerization or clustering, three complementary approaches were used. The first was genetic disruption of dimerization (D154Q/R161E). Mutations were introduced that disrupt the salt bridges at the KRAS α4-α5 interface essential for KRAS homodimerization. The sensor retained full activity, suggesting that Ras dimerization is dispensable (FIG. 20). Second, steric disruption of dimerization (NS1 monobody at α4-α5 interface) was used. We co-expressed NS1, a monobody that binds the KRAS α4-α5 interface and physically blocks Ras dimerization. Consistent with the genetic data, NS1 co-expression did not inhibit sensor activation (FIG. 20). Finally, Synthetic cytosolic clustering was used. To test if clustering alone can drive activation in the absence of membrane localization, a cytosolic Ras mutant was fused to CCHEX, a hexameric protein ring that mimics the stoichiometry of Ras nanoclusters (~6 molecules) in the cytosol. This construct failed to activate the sensor (FIG. 20).

These data suggest that the circuit-sensing mechanism requires 2D membrane localization of mutant Ras, while indicating that clustering and dimerization may be insufficient for circuit sensitivity. This simple biophysical requirement (membrane-bound protein) suggests that the current sensor design can potentially be robust to heterogeneity in membrane clustering dynamics and potentially generalizable to other membrane-associated oncogenes.

Sensor does not Cleave Human Proteins

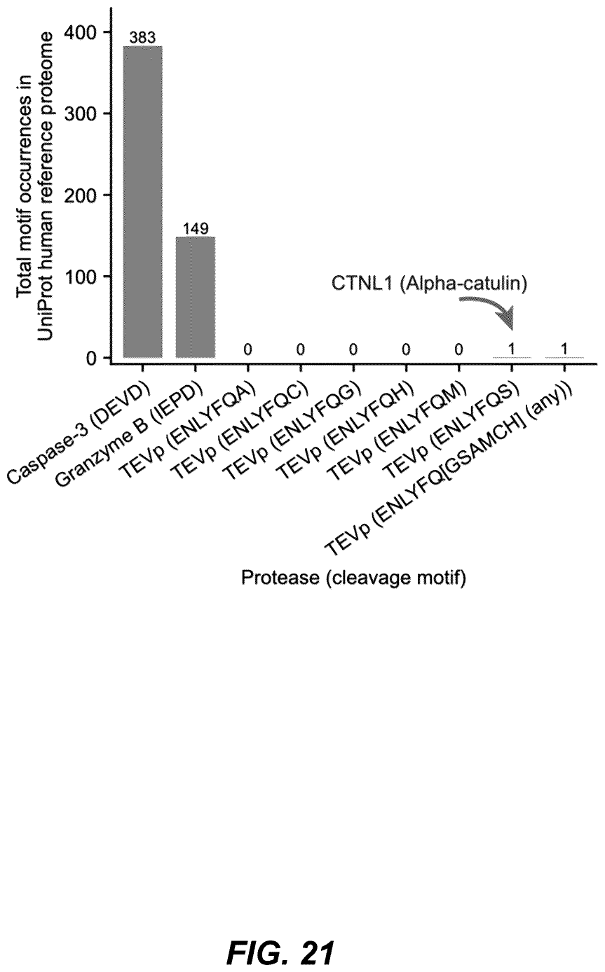

A bioinformatic search for canonical TEVP protease cleavage motifs (ENLYFS[G/S], SEQ ID NO: 247) within the Uniprot human reference proteome was performed. The TEVP protease motif is exceptionally rare, with only one predicted occurrence (in the CTNL1 gene) across the entire human proteome (FIG. 21).

As a control, recognition motifs for endogenous proteases, such as Caspase-3 and Granzyme B, occur hundreds of times. While it is acknowledged that this analysis may not fully capture non-canonical cleavage events or cryptic sites, this data suggests that the TEV protease is highly orthogonal to the human proteome. This specificity is consistent with our experimental observation that TEVP-based sensor expression minimally perturbs KRAS wild-type cells.

### Circuits do not Perturb Cells in Physiologically Relevant Contexts

To assess whether the circuits perturb wild-type Ras cells in a physiological context relevant to drug toxicity, RNA-seq was performed in Caco-2 intestinal epithelial cells treated with either the pan-Ras inhibitor RMC-7977 or the therapeutic circuits. Caco-2 cells were chosen because intestinal toxicity is a major dose-limiting adverse effect of Ras inhibitors, making this a clinically relevant model for detecting off-target effects in Ras-wild-type tissue.

### RNA-Seq Captures Expected Biological Changes in Caco-2 Cells

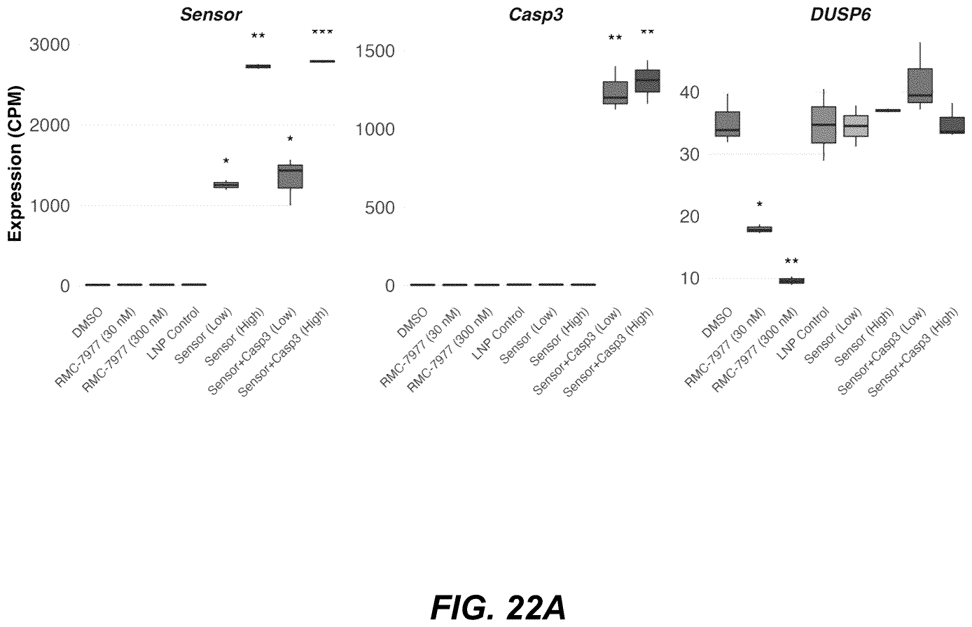

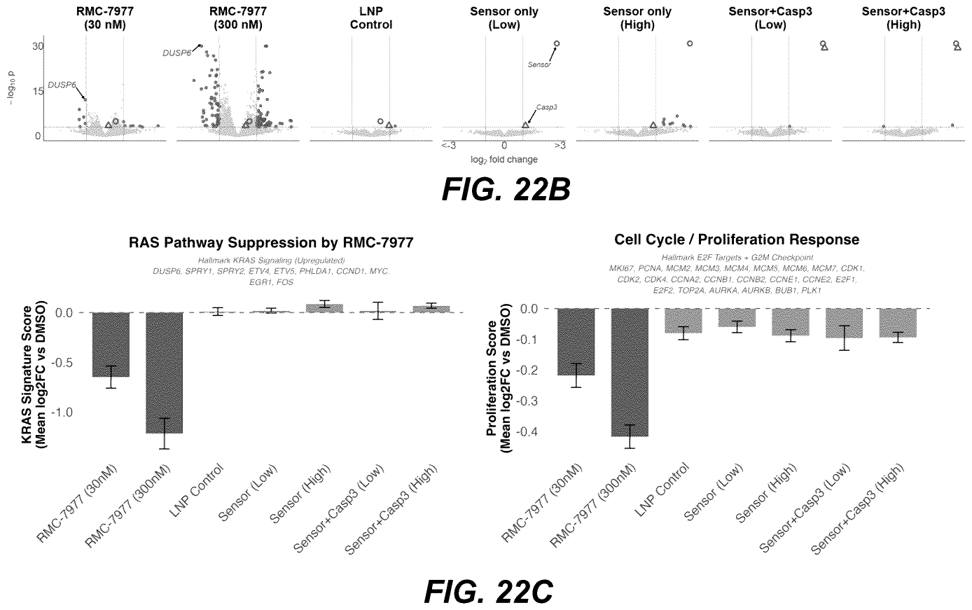

Consistent with the original RMC-7977 paper, treatment with RMC-7977 resulted in strong, dose-dependent knockdown of the canonical Ras marker DUSP6 (FIG. 22A). Gene ontology enrichment analysis confirmed that the top significantly enriched terms for RMC-7977 were “regulation of ERK1 and ERK2 cascade” (p<0.001) and “ERK1 and ERK2 cascade” (p<0.001). These results confirmed that our RNA-seq pipeline was sensitive to real biological effects.

### RMC-7977, but not Circuits, Induced Widespread Changes in Gene Expression

Robust expression of the circuit transgenes (Sensor and Casp3) exclusively in their respective transfected conditions was detected (FIG. 22A). Despite high expression levels, circuits induced negligible transcriptional perturbation compared to the drug (FIG. 22B). RMC-7977 caused an extensive, dose-dependent shift in global gene expression (6/6 upregulated/downregulated genes at 30 nM; 64/55 at 300 nM, p<0.001). In stark contrast, across all circuit conditions, gene expression profiles remained almost indistinguishable from the LNP control (FIG. 22A).

### Circuits Avoided Ras-MAPK Pathway Suppression and Proliferation Inhibition Caused by RMC-7977

To more rigorously quantify the gene expression impacts of drugs versus circuit, a “KRAS Signature Score” was calculated (HALLMARK_KRAS_SIGNALING_UP, M5953) and “Cell Cycle/Proliferation Score” (HALLMARK_E2F_TARGETS, M5925; HALLMARK G2M_CHECKPOINT, M5901) using gene sets from the Molecular Signatures Database (MSigDB v7) Hallmark Collection. RMC-7977 strongly suppressed both signatures in a dose-dependent manner, consistent with the known toxicity profile of Ras inhibitors (FIG. 22C). Conversely, the circuit had little to no effect on either signature, even at high expression levels (FIG. 22C).

Together, these data suggest that the v2 sensor behaves inertly in Ras wild-type cells. This is consistent with previous data showing that RMC-7977, but not 12VC1, binds and perturbs wild-type Ras. Consequently, circuits have the potential to avoid the broad transcriptional perturbations and associated toxicities observed with Ras small molecule inhibitors.

### Sensors Retain Activity Against Inhibitor-Resistant Mutations

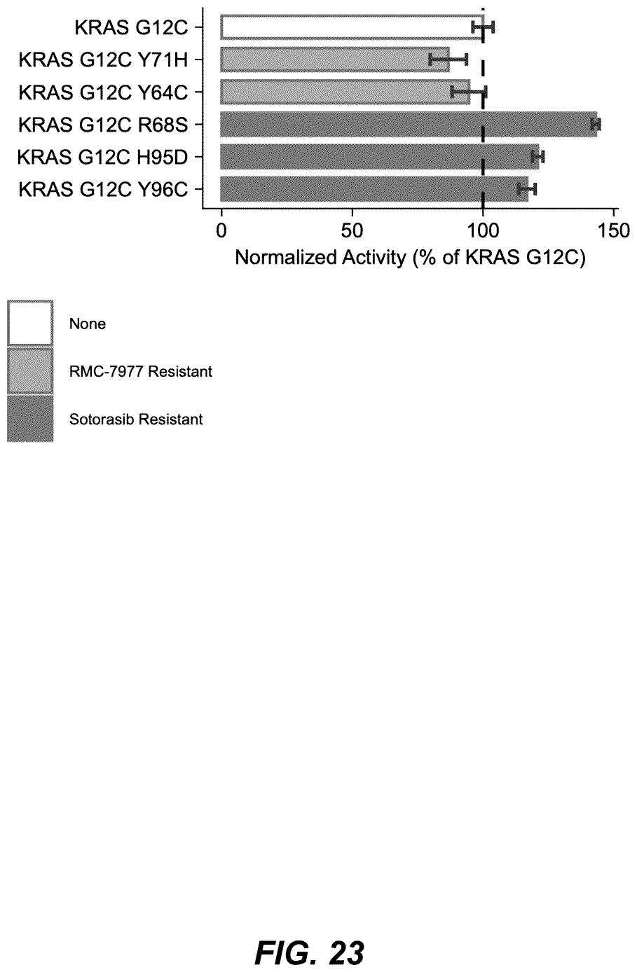

To address cis-mutations that confer resistance to KRAS inhibitors, the 12VC1 sensor was tested against KRAS G12C variants known to confer resistance to Sotorasib (e.g., H95D, Y96C, R68S) and to RMC-7977 (e.g., Y71H, Y64C). These mutations are of clinical significance. The sensor retained robust activity across all tested resistant variants, comparable to KRAS G12C (FIG. 23). This suggests that residues critical for drug binding are distinct from residues required for 12VC1 recognition.

More broadly, without being bound by any particular theory, this finding illustrates two distinct ways in which circuits provide resistance advantages over current inhibitors. One is secondary treatment. As the data above demonstrate, circuits can effectively target pre-treated, drug-resistant clones that have mutated the inhibitor-binding pocket, illustrating their potential as a next-line therapy. A second way is acquired resistance. It is acknowledged that mutations could theoretically arise at the Ras 12VC1 binding interface. However, this highlights the second advantage of circuits—the circuit's killing dynamics. As shown in the time-course analysis and acquired resistance experiments, circuits act as potent and rapid cytotoxic agents. This distinct mechanism minimizes the evolutionary time and population size available for resistance to emerge relative to cytostatic drugs.

Split TEV Protease does not Spontaneously Reconstitute

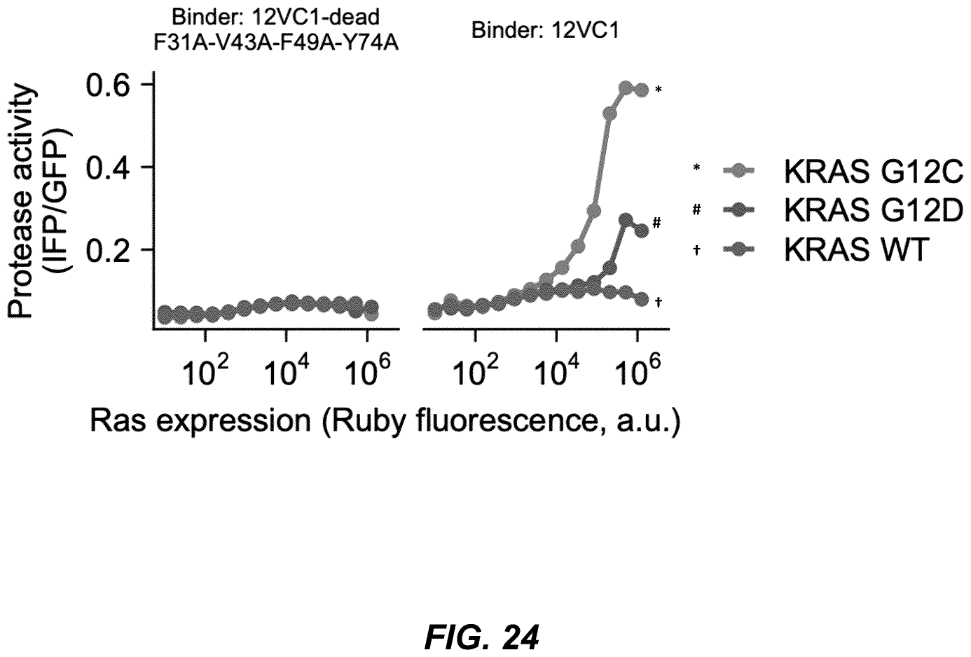

To distinguish background signal sources observed in earlier sensor configurations (v1), four point mutations in 12VC1 at the Ras-binding interface (F31A/V43A/F49A/Y74A) were introduced to abrogate Ras binding. The resulting “dead” sensor produced no measurable response, even at high expression levels, indicating that split-TEVp components do not spontaneously reconstitute to a detectable extent (FIG. 24). This supports the idea that prior background activity is more consistent with nonspecific self-association of the RAF1 RBD used in previous circuits rather than with spontaneous protease reconstitution.

### Circuits Operate Catalytically and can Amplify Weak Binding Events

While 12VC1 was designed to specifically act against G12C and G12V, they also noted that 12VC1 binds “marginally” to G12D. In the context of the original study, monobodies function via stoichiometric inhibition, so this marginal binding is functionally irrelevant as it cannot achieve the high receptor occupancy required to shut down MAPK signaling. Circuits, in contrast, operate catalytically. A single reconstituted TEVP sensor can cleave many substrate molecules, effectively amplifying “marginal” binding events into a robust output. Thus, the disclosed sensor can detect weak interactions that fail to inhibit signaling. Crucially, this signal is not an artifact, as shown in FIG. 24—the “dead” 12VC1 mutant (F31A/V43A/F49A/Y74A) produces no response to these mutants, confirming that the activity observed here is specifically driven by 12VC1-G12D engagement.

### Sensor Activation Depends on GTP-Bound Active Ras

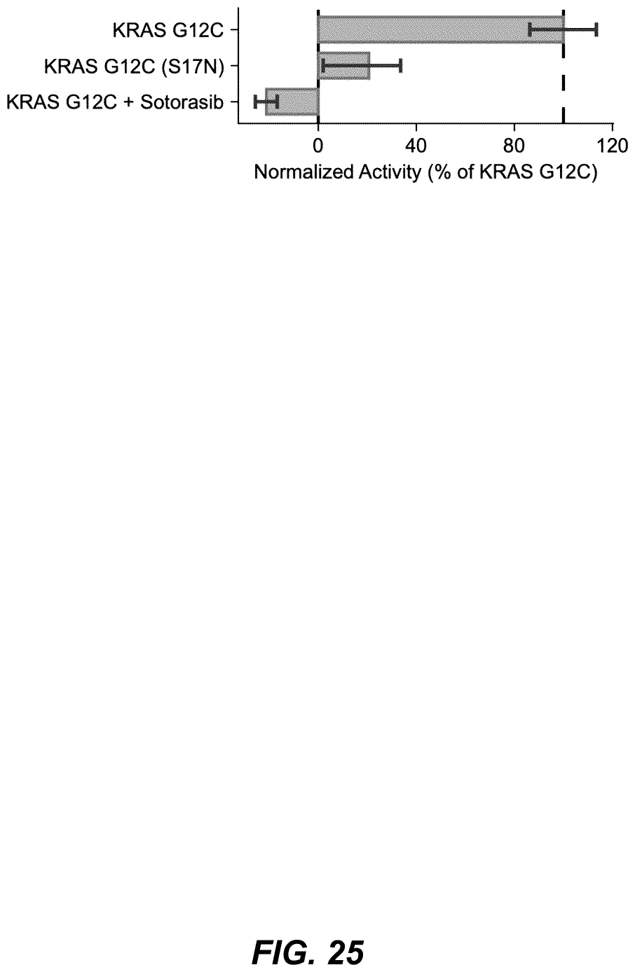

It was verified that circuit activation requires GTP-bound active KRAS. In HEK293 cells, sensor activity was tested against KRAS G12C in the presence of Sotorasib, and against the constitutively GDP-bound KRAS S17N mutant. Sotorasib abolished sensor activation, and KRAS S17N similarly failed to trigger the circuit (FIG. 25). These findings support the notion that activation requires the active (GTP-bound) form of KRAS, consistent with prior observations that KRAS's conformational state influences binder engagement.

### Expanded In Vitro Safety Panel

Because HEK293 is not an ideal model for translational safety assessment, circuits were tested across a panel of cell types relevant to common off-tumor, on-pathway toxicities associated with Ras/MAPK modulation. Specifically, a panel of cell lines were tested including: (i) Caco-2, an intestinal epithelium model to probe potential GI toxicity liabilities; (ii) HepaRG, a hepatocyte-like model to assess liver injury/hepatotoxicity risk; (iii) A-431, a keratinocyte/epidermoid epithelial model with EGFR amplification to evaluate potential skin toxicity toxicities; and (iv) MCF-10A, a non-tumorigenic epithelial model.

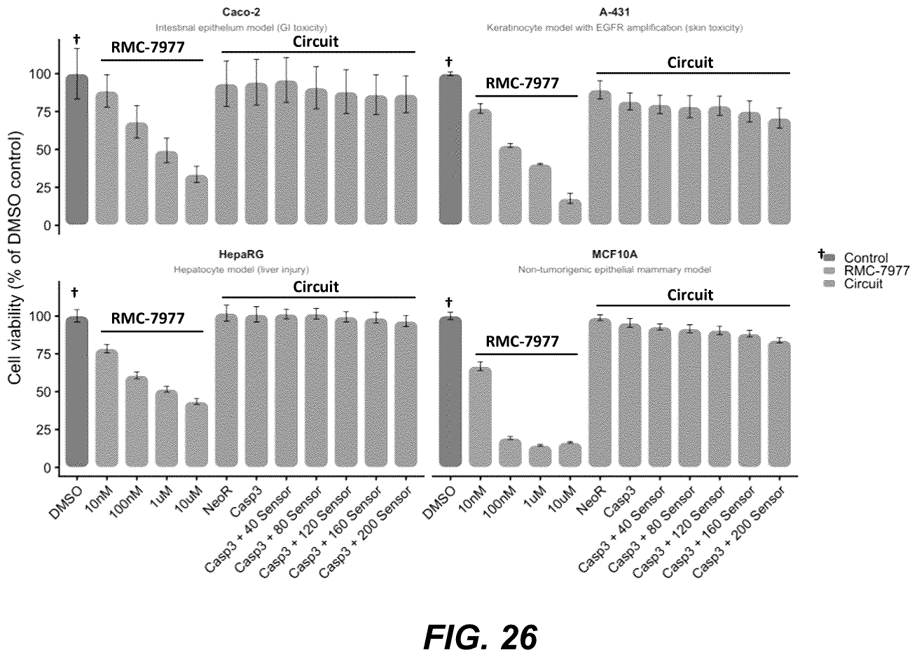

Due to the non-specific nature of RMC-7977 as a Pan-Ras inhibitor, widespread, dose-dependent reductions in cell viability were observed across the entire cell line panel (FIG. 26). In contrast, circuits had minimal effects on cell viability, even at high sensor concentrations (FIG. 26). These results are consistent with the molecular specificity of the 12VC1-based sensor, which shows no measurable affinity for wild-type Ras and thus remains unbound and inactive in wild-type cells.

### Caspase Inhibition Rescues Circuit-Induced Cell Death

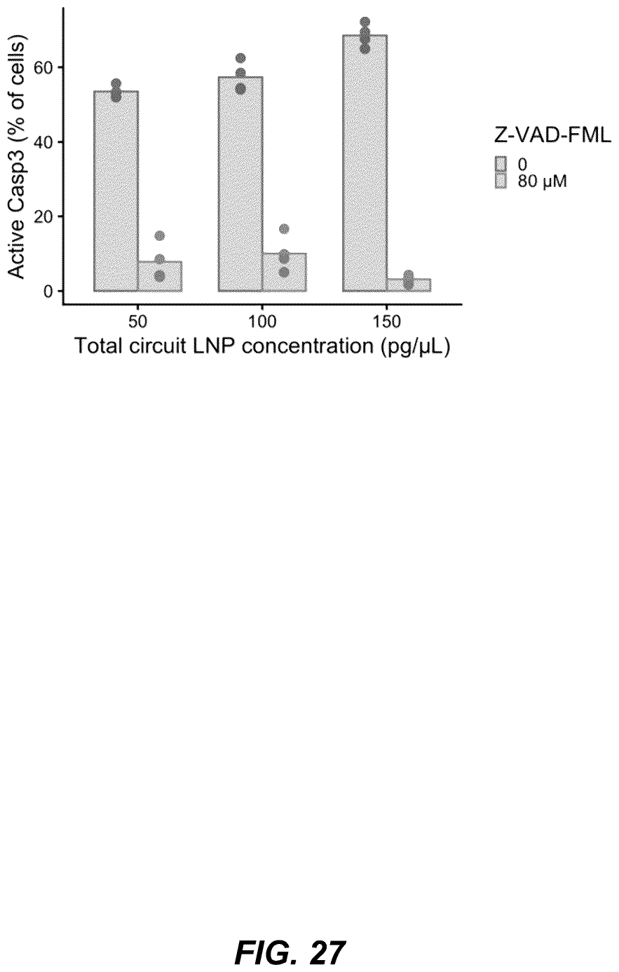

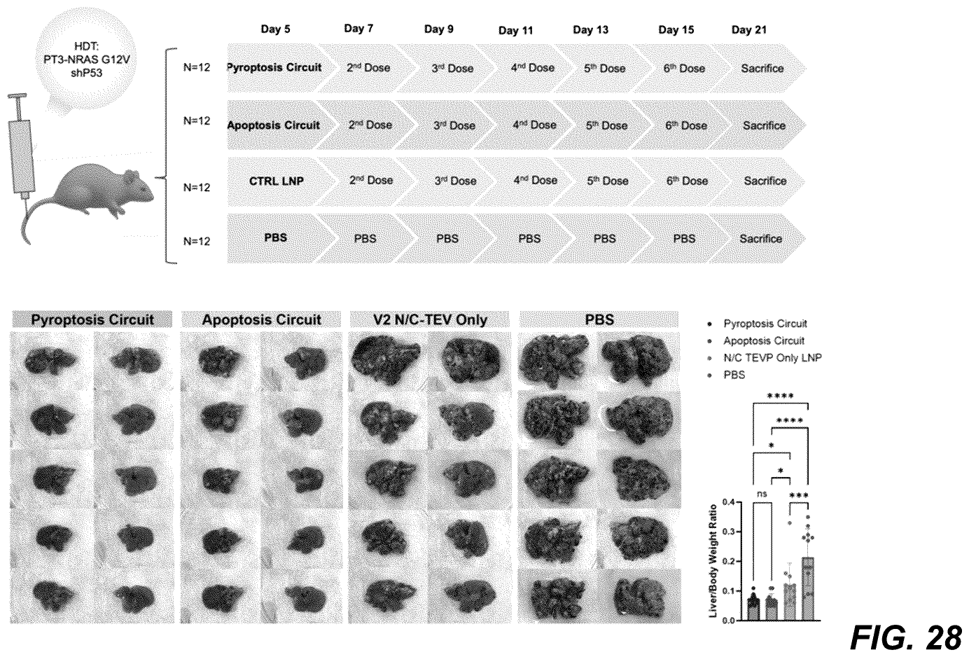

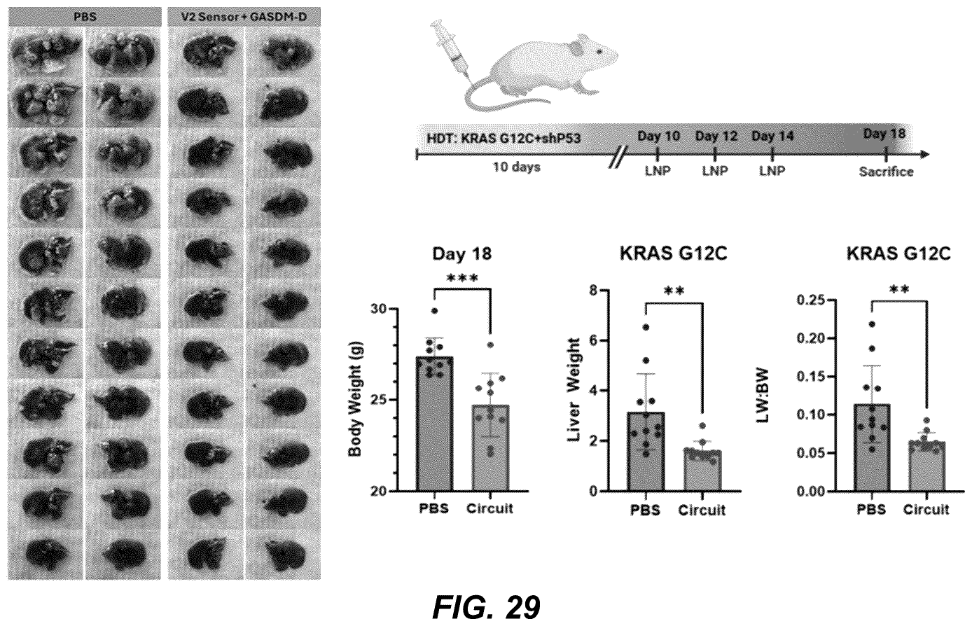

To confirm the apoptotic mechanism, it was further tested whether circuit-induced death can be blocked by a pan-caspase inhibitor. Co-treatment with Z-VAD-FMK ablated circuit-induced loss of viability and/or caspase activation, supporting that circuit killing proceeds through caspase-dependent apoptosis (FIG. 27).

### Circuits Impose Distinct Evolutionary Pressures Compared to Drugs

Pathway-modulating drugs often impose a continuous, intermediate fitness penalty. Cells can enter partially suppressed yet viable states that create time for transcriptional plasticity and reprogramming into stable resistance. Prior work has shown transient, rare-cell fluctuations can “burn in” stable resistance programs under drug exposure.

In contrast, the circuit imposes a more binary outcome: death or survival. When sufficiently activated, the circuit rapidly crosses apoptotic thresholds and commits cells to irreversible death on short timescales. If activation is insufficient (e.g., due to delivery variability or target abundance), circuit proteins degrade without sustained pathway suppression that can drive long-lived adaptive reprogramming. Additionally, circuits are envisioned as periodic administrations rather than continuous exposure, reducing sustained selective pressure.

Finally, circuits may create an “evolutionary trap”: reducing mutant Ras to evade sensing would also remove the oncogenic driver signal underlying the cancer phenotype, limiting the evolutionary space for escape without loss of tumor fitness.

### Apoptosis and Pyroptosis Circuits can Suppress Tumor Formation In Vivo

In this in vivo study, using an experimental setup analogous to that described above, surface tumor burden and liver-to-body weight ratio were evaluated across four treatment groups: a pyroptosis circuit (v2-s sensor+TEVP-activatable GSDMD), an apoptosis circuit (v2-s sensor+TEVP-activatable caspase-3), a TEVP-only LNP control, and PBS (negative control). Both circuit-treated groups exhibited low surface tumor burden and low liver/body weight ratios, with no statistically significant difference between the pyroptosis and apoptosis circuits (ns), suggesting comparable anti-tumor activity in the NRAS-HTVi model under these conditions. Notably, tumor suppression was observed using the v2-s sensor coupled directly to the TEV-activatable effector without an amplifier, supporting that an amplifier component is not required for the therapeutic effect in this model. In contrast, the TEVP-only LNP control showed an intermediate phenotype, with liver/body weight ratios generally higher than those of the circuit-treated groups but lower than PBS, suggesting partial anti-tumor activity, potentially resulting from LNP toxicity and/or TEVP immunogenicity. This control shows that the full circuit is required for successful tumor suppression and treatment.

### Circuits can Suppress KRAS-Driven Tumors In Vivo

The in vivo function of therapeutic circuits against KRAS G12C-driven tumors was further evaluated, adjusting the hydrodynamic tail vein injection (HTVi) model described in Methods above. Specifically, in this experiment, tumors were induced by HTVi of DNA encoding KRAS G12C together with a construct that reduces p53 expression and the SB100 transposase. Animals treated with pyroptosis circuits exhibited no or very few liver surface tumors and liver-to-body weight ratios comparable to those of healthy animals at endpoint. Collectively, these results demonstrate that the circuits can suppress KRAS-driven tumors in vivo.

Methods: Cell Staining with Intracellular Antibodies and Flow Cytometry

Cells were processed for intracellular staining using a two-step fixation/permeabilization procedure based on fixation buffer and methanol. Briefly, cells were maintained in culture wells containing approximately 3.3 mL of medium, and the culture supernatant is retained and transferred to a deep-well plate. Cells were detached by the addition of about 500 μL trypsin and incubation for approximately 15 minutes, followed by the addition of about 1 mL phosphate-buffered saline (PBS). The cell suspension was mixed by pipetting to wash away residual trypsin, then transferred to a deep-well plate. Cells are pelleted by centrifugation at about 700 g for about 5 minutes, and the supernatant is removed by decanting. The pellet was resuspended in approximately 250 μL DMEM and transferred to a U-bottom plate to yield a working volume of about 300 μL per well. In some embodiments, an aliquot corresponding to about 20% of the suspension (e.g., about 50 μL) was removed for Annexin staining, and the remaining portion (e.g., about 250 μL) was carried forward for intracellular fixation/permeabilization and staining. For fixation, an equal volume of 2×intracellular fixation buffer (e.g., 2×eBioscience™ IC Fixation Buffer) was added (e.g., about 250 μL), mixed thoroughly, and incubated at room temperature for about 20 minutes (optionally about 10-60 minutes), protected from light. Cells were then pelleted at about 700 g for about 5 minutes, and the supernatant was removed by decanting. For permeabilization, the pellet was resuspended in the residual volume and about 0.5 mL ice-cold 90-100% methanol (HPLC grade) was added, mixed (e.g., by vortexing), and incubated for at least about 30 minutes at about 2-8° C.; in this state, cells may optionally be stored at less than about-20° C. for up to about 4 weeks. Following methanol permeabilization, cells were washed by adding about 500 μL flow cytometry staining buffer (e.g., eBioscience™ Flow Cytometry Staining Buffer), pelleted at about 700 g for about 5 minutes, and decanted, and the wash was repeated. Cells were then resuspended in about 100 μL staining buffer containing one or more detectable labels and/or directly conjugated antibodies and incubated for about 30 minutes (optionally about 30-60 minutes) at room temperature, protected from light. Exemplary antibodies include, without limitation, an anti-active caspase-3 antibody (rabbit monoclonal; clone C92-605.rMAb; suitable for intracellular flow cytometry) and an anti-phospho-p44/42 MAPK (ERK1/2) (Thr202/Tyr204) antibody (mouse monoclonal; clone E10; Alexa Fluor® 647 conjugate). After staining, cells were washed by the addition of about 200 μL staining buffer, pelleted at about 700 g for about 5 minutes, decanted, and the wash was repeated. Finally, cells were resuspended in about 200 μL staining buffer, optionally dislodged from the well surface (e.g., by scraping) and transferred to a U-bottom flow plate, and analyzed by flow cytometry using an acquisition volume of about 200 μL per sample.

### Example 3

### Protease-Based Therapeutic Circuits that Selectively Kill RAS-Mutant Cancer Cells

Provided in this example are additional experimental data related to the circuits and methods described herein. Provided below are further findings that (a) define the mechanistic principles by which circuits differ from existing Ras-targeting drugs, (b) expand the circuit platform to additional Ras mutations, and (c) strengthen the safety and resistance profile.

### Circuit Variants and Engineered Controls

### G12D-Targeting Circuit Variant (12D4 Binder)

A new circuit variant was engineered by swapping the 12VC1 binder for the 12D4 monobody to target KRAS G12D, the most prevalent KRAS mutation (FIG. 19). This new circuit potently detects KRAS G12D while discriminating against both wild-type KRAS and KRAS G12C. This result demonstrates the modular architecture of the platform: the binder domain is interchangeable. It also allows for mixed circuits co-expressing G12C and G12D sensors for broader patient coverage.

### “Dead” Sensor Negative Control (F31A/V43A/F49A/Y74A)

A negative control sensor was engineered by introducing four point mutations (F31A/V43A/F49A/Y74A) into the 12VC1 binder to abrogate Ras binding (FIG. 24). This dead sensor exhibited no detectable protease activation even at high expression levels. This serves two purposes: (1) confirms circuit activity is strictly dependent on binder-target interactions, and (2) rules out spontaneous reconstitution of split-TEV protease components as a source of background signal.

Mechanistic Distinctions—how Circuits Differ from Drugs

Provided below are newly characterized mechanistic properties of the circuit, paired with therapeutic implications. These mechanistic distinctions collectively define a new therapeutic modality that is fundamentally different from existing Ras inhibitors.

Catalytic Execution vs. Stoichiometric Inhibition

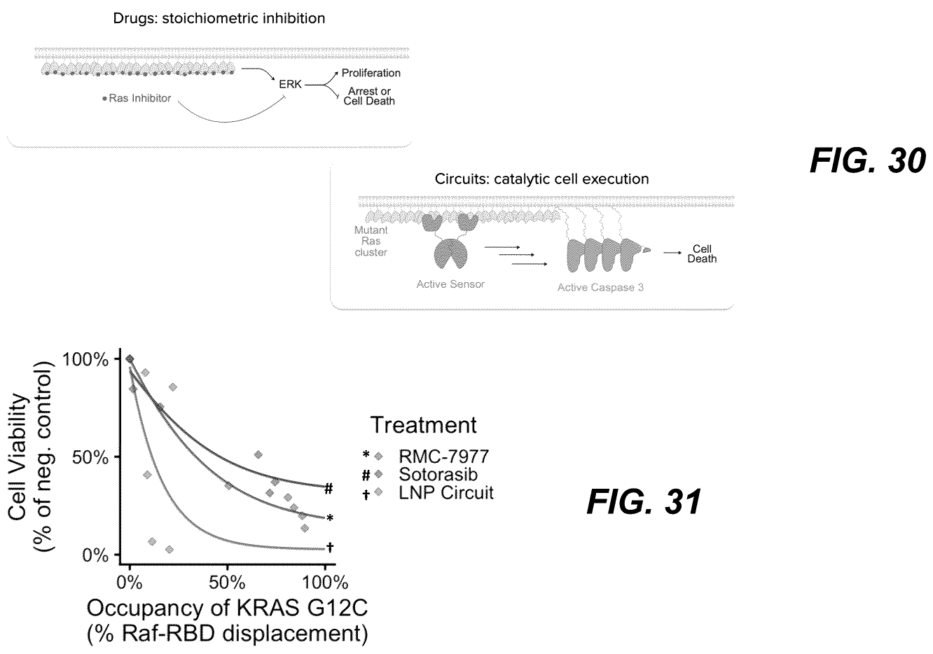

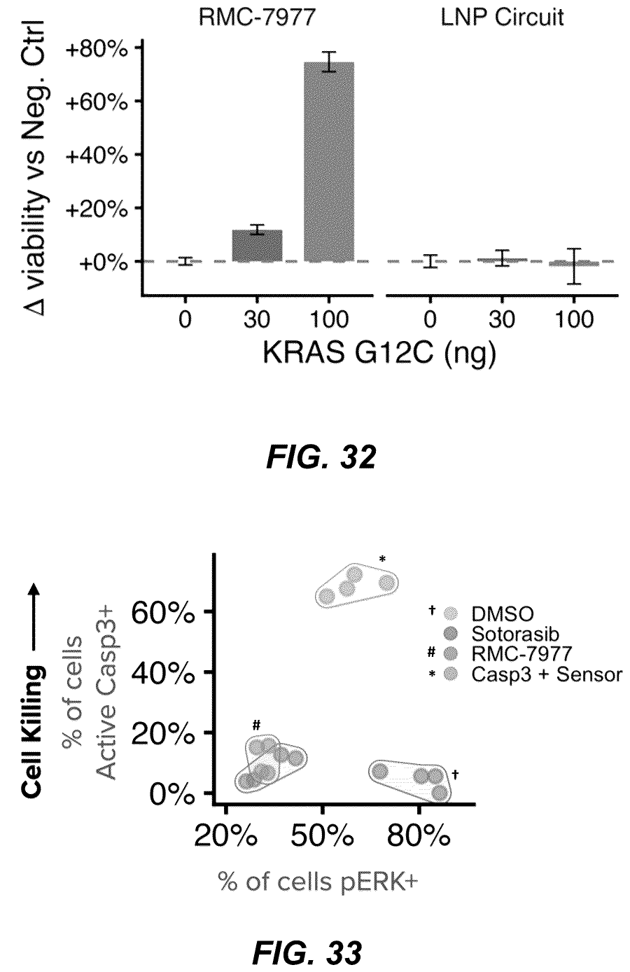

Mechanistic finding: Targeted therapies work stoichiometrically—each drug molecule blocks one target molecule, so effective killing requires near-saturating occupancy of the target. Using a split-luciferase KRAS occupancy reporter, it was found that Sotorasib and RMC-7977 require near-saturating KRAS occupancy before achieving meaningful cell killing, with a steep threshold below which cells remain viable. By contrast, at occupancy levels far below the drug-killing threshold, the circuit already eliminated nearly all Ras-mutant cells. This is because a single sensor reconstitution event triggers a catalytic cascade—one reconstituted protease cleaves multiple effector molecules, amplifying low-occupancy or transient binding into a decisive cell death output. See, e.g., FIG. 30-FIG. 32.

Therapeutic implication—robustness to Ras expression changes: The stoichiometric requirement of drugs means that increasing Ras expression (a common clinical resistance mechanism) dilutes drug occupancy below the killing threshold, rendering drugs less effective. For circuits, the opposite occurs: increased KRAS expression either has no effect on circuit efficacy (at saturating KRAS levels) or actually enhances it (at sub-saturating levels, where more target increases the frequency of sensor reconstitution events). The very mechanism that drives drug resistance, RAS amplification, may instead increase sensitivity to circuit therapy. This raises the possibility that patients who have progressed on Ras-targeting drugs can be preferentially responsive to circuit treatment.

Pathway Independence—Circuits Kill without Suppressing ERK Signaling

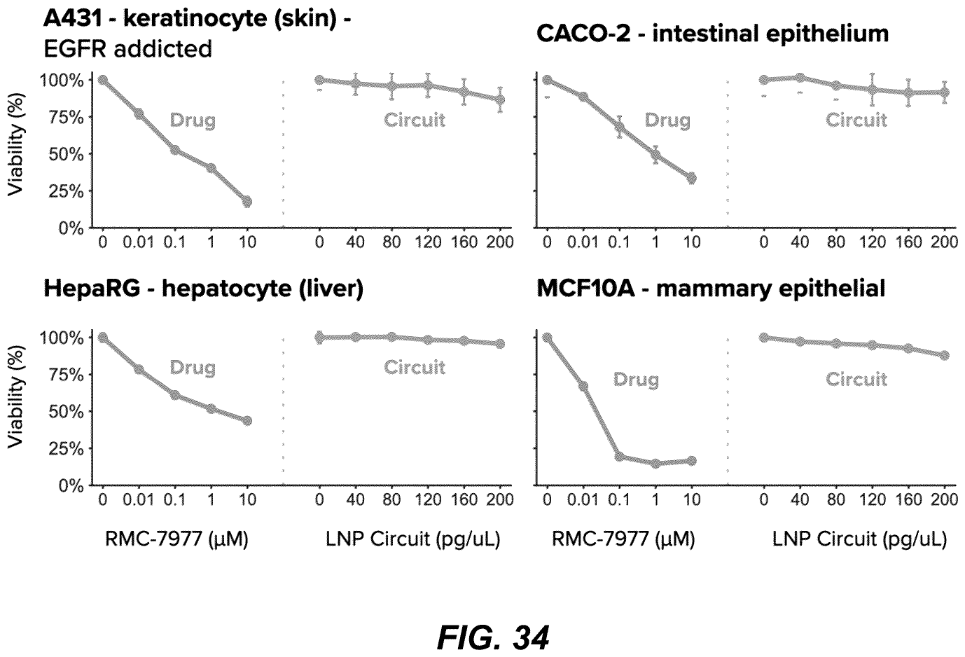

Mechanistic finding: Head-to-head measurement of pERK levels and cell viability showed that Sotorasib, RMC-7977, and the naked 12VC1 monobody all reduce pERK in proportion to their killing activity, consistent with a mechanism that requires sustained pathway suppression. The circuit drove near-complete cell death without any detectable reduction in pERK. The circuit uses mutant Ras as a molecular marker to trigger cell death, not as a signaling node to suppress. See, e.g., FIG. 33-FIG. 34 and FIG. 22B-FIG. 22C.

Therapeutic implication—safety in healthy tissue: Because the circuit does not suppress ERK signaling, it avoids the broad pathway perturbation that causes clinical toxicities of Ras inhibitors (skin rash, GI toxicity, etc.). In wild-type cells, the sensor has no affinity for wild-type Ras and remains unbound and inert. Consistent with this, circuit treatment had minimal effects on cell viability across a panel of wild-type cell lines representing tissues commonly implicated in Ras inhibitor toxicities (intestinal epithelial, hepatocyte-like, keratinocyte models), while RMC-7977 produced widespread dose-dependent viability reductions across the same panel. In Caco-2 intestinal epithelial cells, RMC-7977 strongly suppressed the canonical Ras target DUSP6 and broadly dysregulated ERK pathway gene expression; circuit treatment induced negligible transcriptional changes.

### Directions of Sense—Kill Mechanism of Action-Circuits do not Require Oncogene Addiction

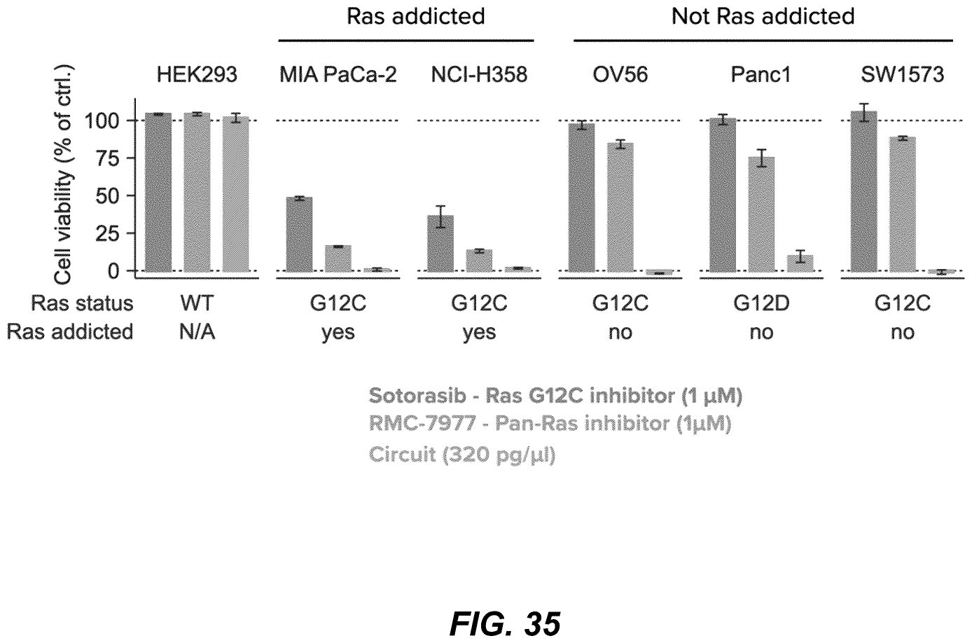

Mechanistic finding: Because circuits kill via marker detection (direct cell death induction) rather than signaling suppression (indirect effects), they are indifferent to whether a cell actually depends on Ras signaling for survival. Ras-mutant tumors vary considerably in oncogene addiction. Even among KRAS G12C lines, CRISPR dependency scores span a wide range. As shown in FIG. 35, Sotorasib and RMC-7977 were substantially less potent in non-addicted lines (SW1573, OV56, PANC-1), whereas the circuit killed all tested lines with comparable potency regardless of addiction status.

Therapeutic implication: Circuits expand the treatable patient population to include Ras-mutant tumors that do not respond to Ras inhibitors because they are not Ras-addicted. This represents a category of patients for whom no targeted Ras therapy currently works.

Cytotoxic Vs. Cytostatic Mechanism—Circuits have Reduced Susceptability to Resistance

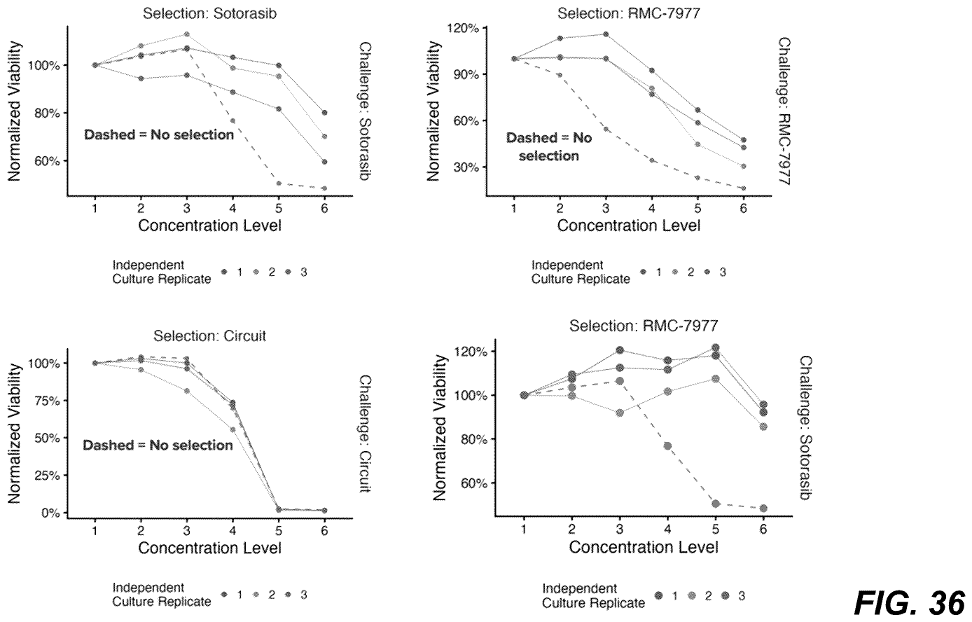

Mechanistic finding: Time-course viability measurements with an explicit Day 0 baseline showed that Sotorasib acts primarily as a cytostatic agent (slows proliferation, cell numbers remain at or above baseline), RMC-7977 induces a mixture of growth inhibition and partial toxicity, and the circuit is potently cytotoxic (cell populations drop rapidly below the Day 0 baseline to near-zero viability within days). Annexin V staining confirmed near-complete apoptotic program activation (>90% Annexin V positive) in circuit-treated Ras-mutant cells. Circuit-induced death was rescued by the pan-caspase inhibitor Z-VAD-FMK, confirming caspase-dependent apoptosis. See, e.g., FIG. 17A-FIG. 17B and FIG. 36.

Therapeutic implication—resistance prevention: The cytostatic-cytotoxic distinction directly impacts resistance. Cytostatic drugs leave viable cells under sustained stress, providing time for transcriptional reprogramming and resistance evolution. Cytotoxic circuits collapse this window—acting on the timescale of hours, they eliminate cells before resistance can emerge. Consistent with this, repeated treatment with Sotorasib and RMC-7977 in MIA PaCa-2 cells (7 weeks at EC70; 4 independent cultures) led to robust acquired resistance. Circuit selection (7 weeks at EC70; 4 independent cultures) had a minimal effect on the EC70 and maintained full killing efficiency. Furthermore, circuits remain effective in drug-resistant cells, whereas drugs exhibit cross-resistance.

### Activity Against Drug-Resistant Ras Variants

Circuit Targets Cells with Sotorasib- and RMC-7977-Resistant Cis-Mutations

The 12VC1-based sensor was tested against clinically relevant KRAS G12C variants known to confer resistance to approved Ras inhibitors through cis-mutations in the drug-binding pocket: (i) Sotorasib-resistant: H95D, Y96C, R68S; (ii) RMC-7977-resistant: Y71H, Y64C.

The sensor maintained robust activity against all tested resistant variants, comparable to its response to unmodified KRAS G12C. The KRAS residues required for drug binding are distinct from those required for 12VC1 recognition. See, e.g., FIG. 23.

### Sotorasib-Resistant Cells are Actually More Sensitive to the Circuit

Cross-resistance experiments showed that cells with acquired Sotorasib resistance (3-week selection; single culture) required 2.5-fold lower concentrations of circuit mRNA-LNP to achieve the same killing efficiency (EC30) as non-resistant cells. This is consistent with the principle described above: a common mechanism of Sotorasib resistance is increased Ras expression or amplification, which increases circuit sensitivity rather than undermining it. This effect differs across cultures, since Sotorasib resistance can also emerge through alternative mechanisms other than Ras amplification.

### Example 4

### Generalizability of the Sensing Paradigm

This Example provides experimental data related to generalizability of the disclosed circuits and methods.

### Proximity-Induced Split Protein Complementation for the Detection of Monomeric Proteins

### Beta-Catenin Concentration Sensing

In one illustrative embodiment, proximity-induced split protein complementation was used to detect beta-catenin (CTNNB1) abundance as a proxy for discriminating cancer cells harboring mutant beta-catenin from cells expressing wild-type beta-catenin.

### Background

Beta-catenin is an oncogenic protein frequently altered in cancer, including hepatocellular carcinoma. Mutations commonly occur at residues in the N-terminal unstructured region. In wild-type beta-catenin, these residues are phosphorylated, enabling recognition by degradation machinery and subsequent proteolysis. Mutation of these residues can impair phosphorylation and/or recognition, thereby reducing degradation and causing beta-catenin accumulation. In cells with oncogenic beta-catenin activation, non-degraded beta-catenin can accumulate in the nucleus. Beta-catenin does not generally self-assemble into dimers or higher-order oligomers of beta-catenin alone, although it can participate in larger protein complexes.

Beta-catenin also has a dual biological role, including regulation of cell-cell adhesion. Accordingly, a membrane-associated pool of beta-catenin is present in both healthy and malignant cells and is not necessarily indicative of oncogenic signaling. By contrast, a nuclear pool of beta-catenin is associated with mutant beta-catenin and/or active Wnt signaling.

### Sensor Design

Two distinct beta-catenin-binding moieties, derived, for example, from nanobodies and/or naturally occurring beta-catenin-interacting proteins, were fused to complementary portions of a split protease. Because the sensor components are relatively small, they can passively access the nucleus while remaining in the cytoplasm. One or more nuclear localization sequences can be additionally fused to the sensor components to enrich nuclear localization. Sensor output may be measured using an iProtease reporter or another split-effector-compatible reporter.

In some embodiments, the sensor is configured to detect the abundance of monomeric oncogenic beta-catenin while avoiding or reducing detection of the membrane-associated adhesion-related pool.

### Results

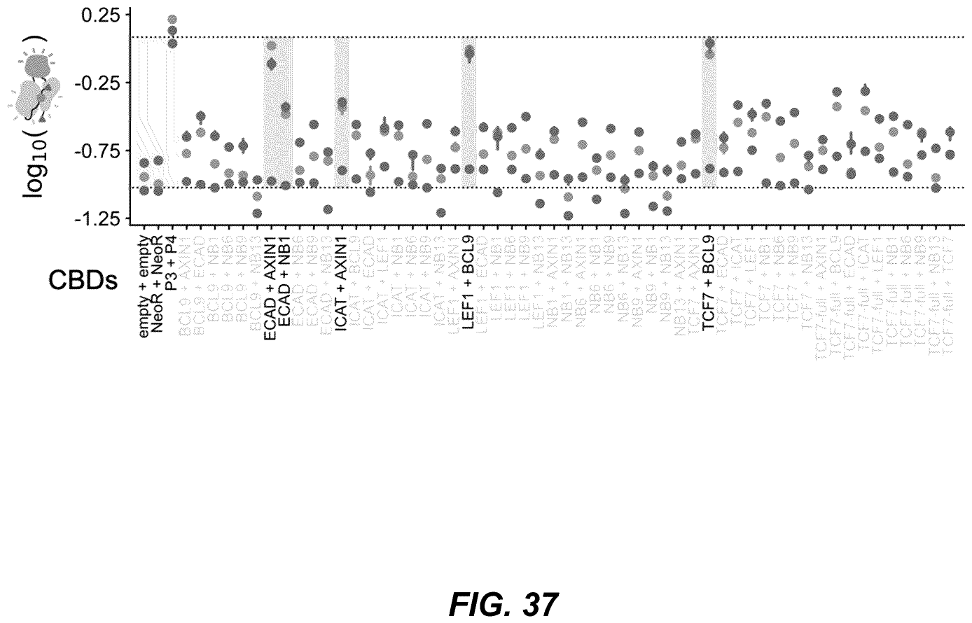

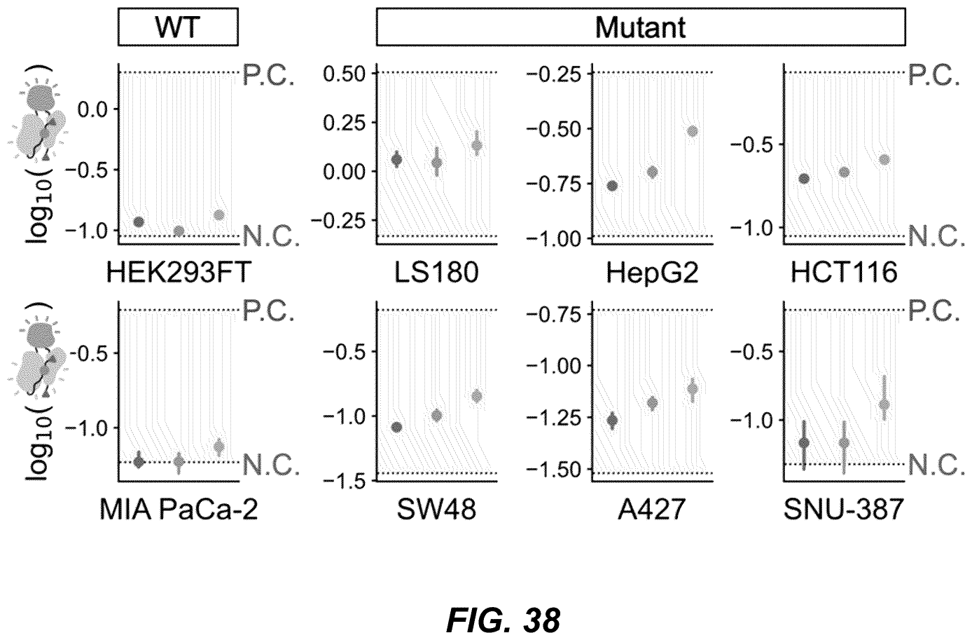

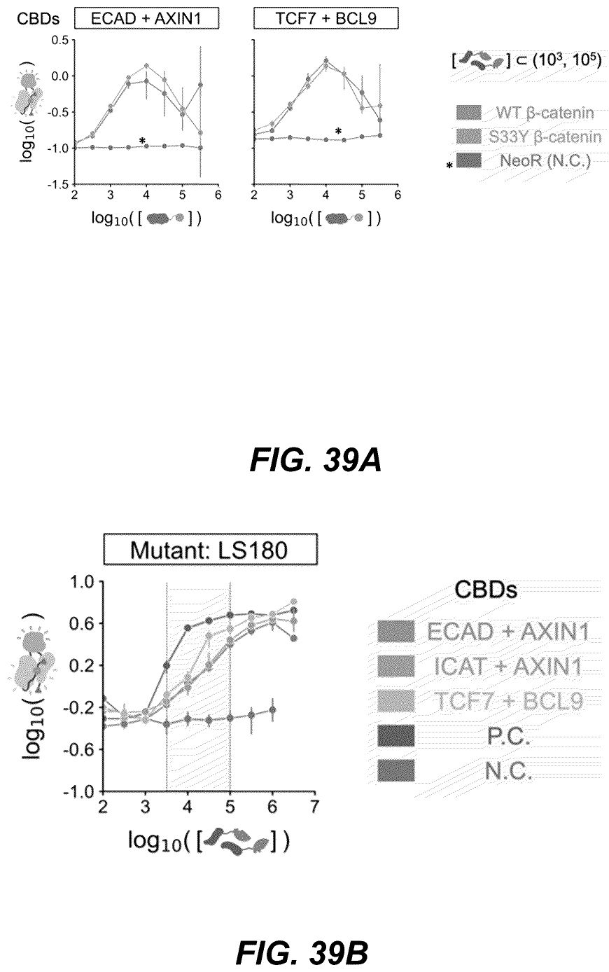

Representative designs enabled the detection of overexpressed beta-catenin (FIG. 37) and discrimination between cancer cells having wild-type versus mutant beta-catenin (FIG. 38). In overexpression experiments, both wild-type and mutant beta-catenin were detected when normalized to target expression, consistent with the sensor functioning as a concentration-responsive device (FIG. 37). Sensor output was dependent on target abundance (FIG. 39A-FIG. 39B). To reduce detection of the membrane-associated beta-catenin pool, at least one sensor binder was selected to compete with, but bind more weakly than, native membrane-associated interaction partners such as CTNNA1 and/or CDH1.

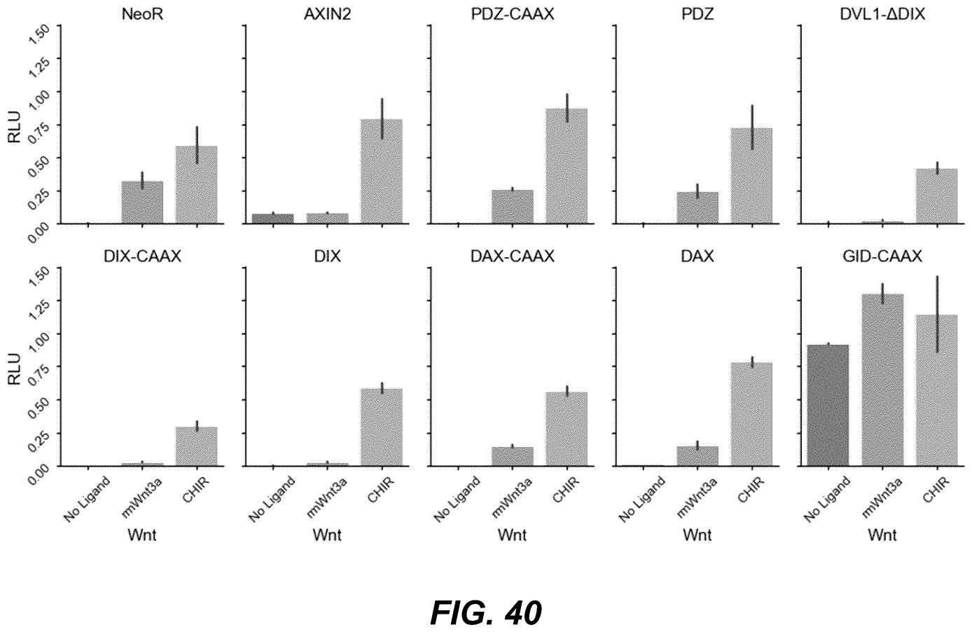

Additional binding agents may be co-administered with the sensor to inhibit native beta-catenin accumulation induced by Wnt ligands (FIG. 40), thereby biasing detection toward mutant beta-catenin rather than ligand-induced wild-type beta-catenin stabilization.

The above demonstrates that proximity-induced split protein complementation can be adapted to detect a non-oligomeric intracellular oncogenic protein based primarily on effective local target abundance and compartment-specific accessibility, rather than requiring stable self-oligomerization of the target.

### Proximity-Induced Split Protein Complementation for the Detection of Nuclear Oligomers

### p53 Concentration Sensing

In another illustrative embodiment, proximity-induced split protein complementation was used to detect p53 (TP53) abundance as a proxy for cancer status.

### Background

p53 is among the most frequently mutated genes in human cancer and ordinarily functions as a tumor suppressor. Certain mutations impair canonical tumor suppressor activity, while others confer gain-of-function properties, including altered DNA-binding and transcriptional programs that can promote oncogenesis. In some cancers, including certain non-small cell lung cancers, p53 protein levels are substantially elevated, and p53 staining is used as a diagnostic and/or prognostic marker. p53 forms tetramers in the nucleus.

### Sensor Design

In an exemplary design, a p53-binding moiety was fused to each of two complementary portions of a split protease. At low p53 concentrations, relatively few tetrameric assemblies are present, resulting in low levels of split-protease reconstitution. At higher p53 concentrations, the number of p53 tetramers increases, thereby increasing productive colocalization of the split protease halves and enhancing sensor activation. Because the sensor components are relatively small, they can passively access the nucleus. One or more nuclear localization sequences can be included to further enrich nuclear localization. Sensor output may be read out with an iProtease reporter or a comparable reporter system.

### Results

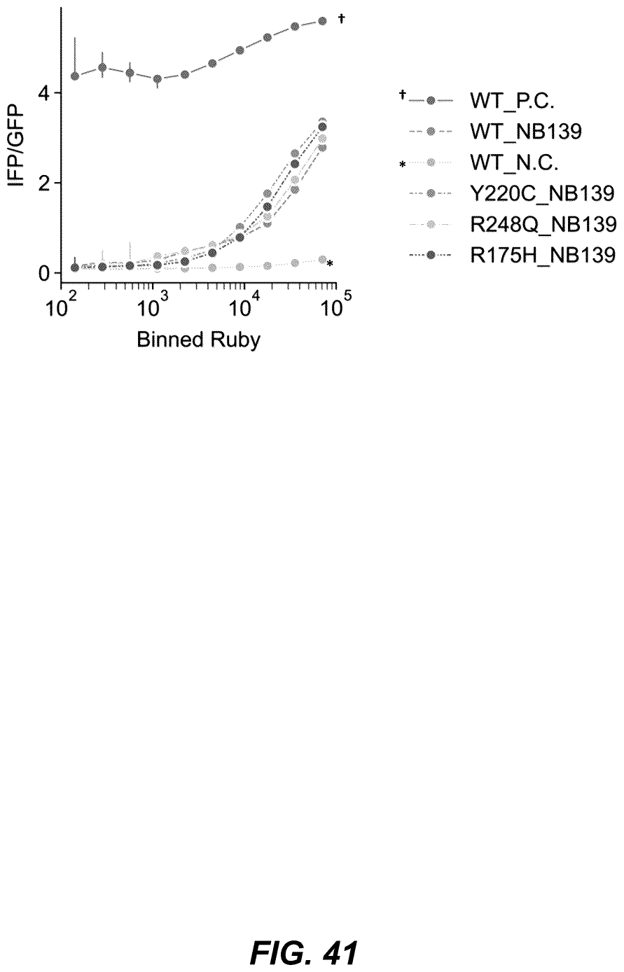

Representative sensors enabled robust, concentration-dependent detection of multiple p53 variants, including both mutant and wild-type p53, in overexpression settings. These results support the broader sensing paradigm for detection of oligomeric nuclear proteins, including tetrameric transcription factors. See, e.g., FIG. 41.

The above shows that the disclosed sensing approach can be extended beyond membrane-associated targets and can function in the nucleus to detect oligomeric proteins based on target abundance and multivalent proximity.

### Proximity-Induced Split Protein Complementation to Detect Mutant Nuclear Oligomers

### p53 Mutant-Conformation Sensing

In another illustrative embodiment, proximity-induced split protein complementation was used to detect the mutant conformational state of p53 (TP53), enabling discrimination of conformationally altered p53 from wild-type p53.

### Background

As described in above, p53 is among the most frequently mutated genes in human cancer, with missense mutations occurring in approximately 30-40% of all human tumors across diverse cancer types, including non-small cell lung cancer, ovarian cancer, colorectal cancer, and head and neck squamous cell carcinoma. p53 forms tetramers in the nucleus.

A substantial fraction of oncogenic p53 missense mutations, including frequently observed hotspot mutations such as R175H, Y220C, R248Q, R273H, G245S, and R249S, destabilize the DNA-binding domain of p53, causing partial or complete unfolding. This conformational destabilization exposes a cryptic epitope within the DNA-binding domain (FRHSV, SEQ ID NO: 361) that is buried and inaccessible in the correctly folded wild-type protein. This cryptic epitope is recognized by conformational antibodies such as pAb240 and is a hallmark of the mutant p53 conformational state. The degree to which this epitope is exposed varies among mutations, but a large class of clinically relevant mutations, often termed structural or conformational mutations, render this epitope accessible to binding agents.

The ability to detect the mutant conformational state of p53, rather than merely elevated p53 protein levels, is of clinical significance. Conformational detection enables discrimination of cells harboring oncogenic structural p53 mutations from cells with correctly folded wild-type p53, independent of expression levels. Such discrimination has potential applications in diagnostic classification, prognostic stratification, companion diagnostics for therapies targeting mutant p53 (including emerging conformational rescue agents), and genetically encoded cell-based therapeutic circuits that selectively activate in the presence of conformationally altered p53.

Existing methods for detecting the p53 mutant conformation, such as immunohistochemistry using pAb240, require fixed tissue samples and exogenously applied antibodies and are not genetically encodable. The sensing approach disclosed herein enables genetically encoded, cell-autonomous detection of p53 mutant conformation in living cells.

Experiments provided above showed the concentration-dependent sensing of p53 regardless of mutation status, the experiments described below demonstrates sensing that is selective for the mutant conformation.

### Sensor Design

de novo protein binders targeting the cryptic epitope of the p53 DNA-binding domain can be designed using computational protein design methods. In one implementation, approximately 90 candidate binders were generated using a diffusion-based de novo protein design tool (FreeBindCraft), specifying the cryptic epitope region as the target binding interface. The binders targeting the cryptic epitope can comprise nanobodies, antibody fragments, monobodies, peptides, engineered scaffold proteins, aptamers, or other affinity reagents, as long as they preferentially bind the mutant-exposed cryptic epitope over the folded wild-type conformation.

In the sensor configuration, one half of a split protease (e.g., the N-terminal half of TEV protease) is fused to a cryptic-epitope-targeting binder, and the other half (e.g., the C-terminal half of TEV protease) is fused to a second cryptic-epitope-targeting binder. The two binders may be identical or different. In other embodiments, one half is fused to a cryptic-epitope-targeting binder, and the other half is fused to a general p53-binding moiety. Because mutant p53 retains the ability to form tetramers, simultaneous binding of two binder-protease fusion proteins to exposed cryptic epitopes on adjacent subunits within a single p53 tetramer brings the split protease halves into proximity, enabling reconstitution and proteolytic activity. In wild-type p53 tetramers, the cryptic epitope is buried, the binders cannot productively engage the target, and the split protease halves are not brought into proximity. The sensor thus discriminates the mutant conformational state from wild-type on the basis of epitope accessibility rather than target abundance alone.

One or more nuclear localization sequences can be fused to the sensor components to enrich nuclear localization. In other embodiments, split effectors other than split proteases, such as split recombinases, split transcription factors, split Cas proteins, split cytokines, or other split reporter or actuator proteins, may be used in place of the split protease. Sensor output may be read out using an iProtease reporter or a comparable split-effector-compatible reporter system.

### Results

To evaluate whether candidate de novo binders recognize the cryptic epitope, each binder was fused to one half of a split protease, and a construct presenting the cryptic epitope or a control sequence was fused to the complementary half. Productive binding of the binder to its target brings the two protease halves into proximity, enabling reconstitution and reporter activation.

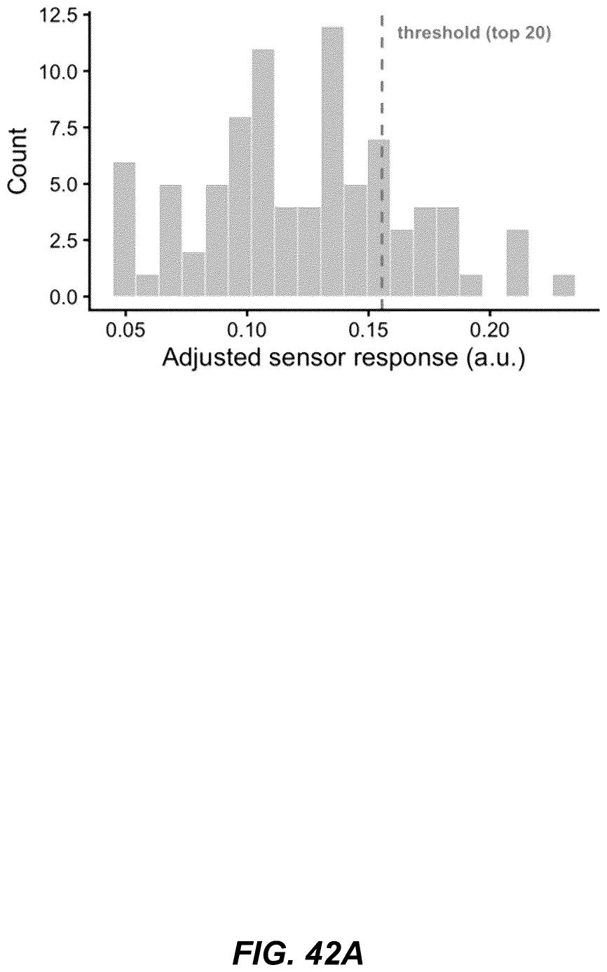

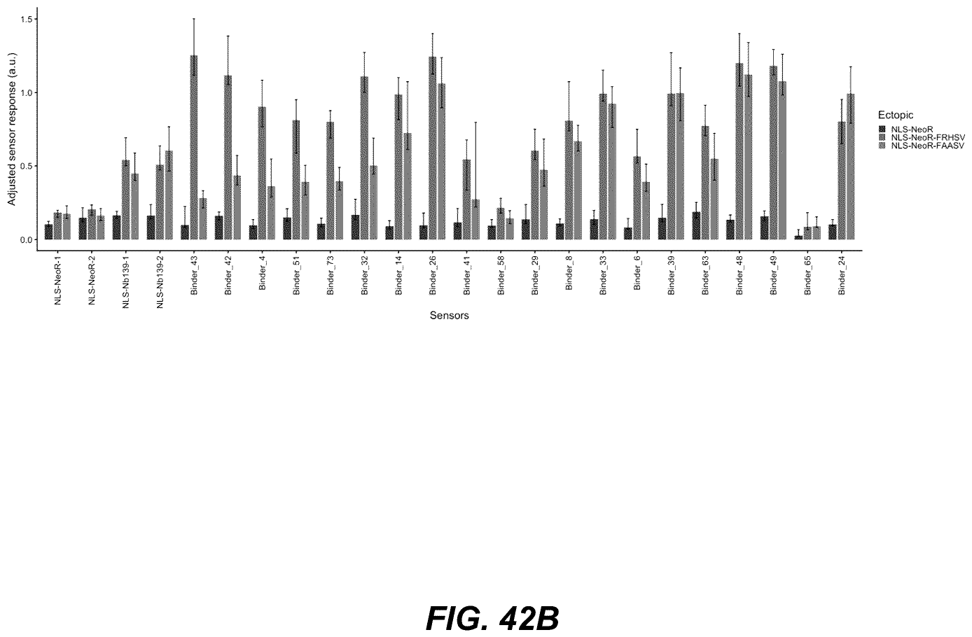

In a first screen, approximately 90 de novo binders were assessed for their ability to engage the cryptic epitope using this approach. Sensor activation was measured by tracking iProtease reporter activation with flow cytometry 48 hours after DNA transfection into HEK293FT cells. A response threshold was established, and approximately 20 binders exceeding this threshold were selected for further characterization (FIG. 42A).

In a second screen, the top approximately 20 binders were tested for epitope specificity. Each de novo binder, fused to one half of a split protease, was co-expressed with one of three target constructs fused to the complementary half of the split protease: (i) a negative control lacking any p53-derived sequence (NLS-NeoR), (ii) a construct presenting the wild-type cryptic epitope sequence (NLS-NeoR-FRHSV), or (iii) a construct presenting a mutated version of the cryptic epitope (NLS-NeoR-FAASV) that serves as a specificity control. Known p53-binding nanobodies (e.g., NB139 variants) and additional controls were included for reference. Among the tested de novo binders, several exhibited high sensor activation in the presence of the wild-type cryptic epitope sequence (FRHSV, SEQ ID NO: 361) with substantially lower activation against both the negative control and the mutated epitope variant (FAASV, SEQ ID NO: 362), thereby demonstrating specificity for the target conformational epitope (FIG. 42B).

The top specificity-validated de novo binders can be incorporated into the sensor configuration described above, in which both halves of the split protease are fused to cryptic-epitope-targeting binders, and evaluated against full-length wild-type and mutant p53 variants expressed in cells. Because the cryptic epitope is selectively exposed in conformationally destabilized mutant p53 and buried in correctly folded wild-type p53, such sensors are expected to exhibit preferential activation in the presence of full-length mutant p53 relative to full-length wild-type p53. This validation would confirm that the epitope specificity demonstrated with isolated epitope constructs translates to selective detection of the mutant conformational state in the context of the native, tetrameric, full-length protein.

### Conclusion.

The above demonstrates that de novo computationally designed protein binders can be generated to specifically recognize a conformation-dependent epitope of p53 and incorporated into the disclosed split-effector sensing architecture. The combination of conformational epitope specificity with oligomerization-dependent proximity sensing provides a sensor design capable of discriminating the mutant p53 conformational state from that of wild-type p53 based on epitope accessibility. This extends the sensing paradigm from abundance-based detection to conformation-selective detection and illustrates the broader applicability of the disclosed systems to targets in which disease-relevant states are characterized by conformational changes that expose or occlude specific binding epitopes.

### Example 5

### Binding Studies

Provided in this Example are data related to binding affinities of the oncoprotein binding domains of the disclosure. Binding affinities (Kd) of 12VC1 are as follows: KRAS G12C (GTP): 24.7 nM KRAS G12V (GTP): 100 nM KRAS WT (GTP): 9600 nM. For all GDP bound KRAS: affinity was outside detection limit (too weak).

Hence, 12VC1 (used in the v2 sensor) binds KRAS (G12C) and KRAS (G12V) in the active GTP-bound state with up to 400-fold greater affinity than wild-type KRAS. The structural basis for this selectivity was resolved by X-ray crystallography (PDB: 7L0G), which revealed a shallow pocket on 12VC1 that specifically accommodates the mutant side chain at residue 12.

Functional sensor discrimination (protease reporter assay) was performed. In our protease reporter assay, the v2 sensor showed ~2-5-fold activation in response to diverse oncogenic Ras mutants (G12C, G12V, G12S, G12A, and many others across a panel of 46 KRAS, HRAS, and NRAS mutations) relative to wild-type Ras, at matched Ras expression levels.

When transfected into cancer cell lines expressing Ras at physiological levels, the sensor showed strong protease activation in mutant Ras lines (MIA PaCa-2/KRAS\hat{ }G12C, NCI-H358/KRAS\hat{ }G12C, NCI-H441/KRAS\hat{ }G12V) while wild-type Ras lines (HEK293, PLC/PRF/5, SNU-423, SNU-449, SNU-475) showed activation near the negative control baseline.

It is emphasized that binding affinity is not the only factor contributing to the observed target-versus-non-target discrimination. Without being bound by any particular theory, the circuit architecture amplifies whatever binding selectivity exists through multiple layers of nonlinearity. First, the sensor is a split protease: binding recruits enzyme halves to membrane-localized Ras clusters, where reconstitution and catalytic turnover amplify the signal far beyond what stoichiometric binding would predict. Second, the optional protease amplifier (TEVP→TVMVP cascade) provides an additional gain stage. Third, and perhaps most importantly, cell death pathways are themselves threshold-dependent. A cell must accumulate sufficient active caspase-3 (for apoptosis) or gasdermin (for pyroptosis) to commit to death. Sub-threshold sensor activity in wild-type cells—resulting from the low residual binding of 12VC1 to wild-type Ras—simply never crosses this commitment point. The net effect is that a 400-fold difference in binding affinity is converted, through enzymatic amplification and death-pathway thresholding, into a near-binary kill/no-kill outcome.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. (canceled)

2. A synthetic protein circuit, comprising:
(a) a sensor module comprising:
a first polypeptide comprising a first oncoprotein binding domain and a first part of a first orthogonal protease domain, wherein the first oncoprotein binding domain is capable of binding a first oncoprotein to form a first oncoprotein-bound polypeptide;
a second polypeptide comprising a second oncoprotein binding domain and a second part of the first orthogonal protease domain, wherein the second oncoprotein binding domain is capable of binding a second oncoprotein to form a second oncoprotein-bound polypeptide, and
wherein the first part of the first orthogonal protease domain and the second part of the first orthogonal protease domain are capable of associating with each other to constitute a first orthogonal protease capable of being in a first orthogonal protease active state when the first oncoprotein and the second oncoprotein are in close proximity at a proximity site, wherein the first and/or second oncoprotein binding domain comprise a sequence at least 85% identical to the sequence of SEQ ID NO: 109 or SEQ ID NO: 111; and
(b) an effector module capable of inducing apoptosis or pyroptosis in a cell, wherein the first orthogonal protease in the first orthogonal protease active state is capable of activating the effector module, thereby inducing apoptosis or pyroptosis in the cell.

3. The synthetic protein circuit of claim 2, wherein the first oncoprotein and/or the second oncoprotein:
(i) comprise a monomer having at least two distinct binding sites;
(ii) comprise a dimer having at least two binding sites per dimer in sufficient proximity to promote association;
(iii) comprise an oligomer having at least two binding sites per oligomer in sufficient proximity to promote association;
(iv) comprise an aggregate having at least two binding sites per aggregate in sufficient proximity to promote association;
(v) comprise a multi-protein complex in which two or more constituent proteins collectively provide at least two binding sites in sufficient proximity for association;
(vi) comprise an onco-fusion protein; or
(vii) any combination of (i)-(vi).

4.-8. (canceled)

9. The synthetic protein circuit of claim 2, wherein the first part of the first orthogonal protease domain and the second part of the first orthogonal protease domain have weak association affinity.

10. The synthetic protein circuit of claim 2, wherein:
(i) the first oncoprotein binding domain and the second oncoprotein binding domain are identical or different; or
(ii) the first oncoprotein binding domain, the second oncoprotein binding domain, or both are specific to the first and/or second oncoprotein and do not bind or weakly bind a wild type version of the first and/or second oncoprotein; or
(iii) both (i) and (ii).

11.-14. (canceled)

15. The synthetic protein circuit of claim 2, wherein the first polypeptide and the second polypeptide are expressed in the cell on a single molecule as a proprotein, wherein the proprotein comprises one or more proprotein protease cleavage sites, and
wherein the first polypeptide and the second polypeptide are separated on the proprotein by a first proprotein protease cleavage site and a second proprotein protease cleavage site.

16. (canceled)

17. The synthetic protein circuit of claim 15, wherein the proprotein comprises,
from N-terminus to C-terminus:
the first polypeptide comprising the first oncoprotein binding domain and the first part of the first orthogonal protease domain;
a first proprotein protease cleavage site cleavable by the first orthogonal protease in the first orthogonal protease active state;
an optional linker;
a second proprotein protease cleavage site cleavable by the first orthogonal protease in the first orthogonal protease active state; and
the second polypeptide comprising the second oncoprotein binding domain and the second part of the first orthogonal protease domain.

18. The synthetic protein circuit of claim 15, wherein the first part of the first orthogonal protease domain and the second part of the first orthogonal protease domain of the proprotein are capable of associating with each other to form the first orthogonal protease in the first orthogonal protease active state, wherein the first orthogonal protease in the first orthogonal protease active state is capable of cleaving the proprotein at the first and/or second proprotein protease cleavage site, to release the first polypeptide and the second polypeptide as separate molecules in the cell.

19. (canceled)

20. (canceled)

21. The synthetic protein circuit of claim 2, wherein the first oncoprotein binding domain, the second oncoprotein binding domain, or both, comprise the sequence of SEQ ID NO: 109, SEQ ID NO: 110, or SEQ ID NO: 111.

22. (canceled)

23. The synthetic protein circuit of claim 15, wherein:
(i) the first proprotein protease cleavage site, the second proprotein protease cleavage site, or both, comprise the sequence of any one of SEQ ID NOs: 247, 253, and 255-258 or a sequence having one or two mismatches relative to any one of SEQ ID NOs: 247, 253, and 255-258; or
(ii) the proprotein comprises the sequence of any one of SEQ ID NOs: 112-113 or a sequence at least 85% identical to the sequence of any one of SEQ ID NOs: 112-113.

24. (canceled)

25. The synthetic protein circuit of claim 2, wherein:
(1) the first oncoprotein, the second oncoprotein, or both comprise at least one mutation associated with cancer;
(2) the first oncoprotein, the second oncoprotein, or both comprise a conformation, post-translational modification, interaction state, abundance, oligomerization state, multimerization state, and/or subcellular localization state associated with a disease or disorder;
(3) the first oncoprotein and the second oncoprotein are identical;
(4) the first oncoprotein, the second oncoprotein, or both are endogenous proteins; or
(5) any combination of (1)-(4).

26.-28. (canceled)

29. The synthetic protein circuit of claim 2, wherein the first oncoprotein, the second oncoprotein, or both comprise a RAS protein.

30. The synthetic protein circuit of claim 29, wherein the RAS protein comprises at least one mutation associated with cancer, wherein the at least one mutation comprises a mutation in KRAS, NRAS, and/or HRAS.

31. (canceled)

32. (canceled)

33. The synthetic protein circuit of claim 2, wherein the effector module comprises a synthetic apoptosis circuit comprising:
an apoptosis polypeptide comprising a large subunit of an apoptotic effector protein and a small subunit of an apoptotic effector protein separated by a first orthogonal protease cleavage site,
wherein two apoptosis polypeptides are capable of associating with each other to constitute an apoptotic protein complex in an apoptotic protein complex inactive state,
wherein the first orthogonal protease in a first orthogonal protease active state is capable of cleaving the first orthogonal protease cleavage site of the apoptosis polypeptide, and wherein the first orthogonal protease cleavage site of the apoptosis polypeptide being cleaved changes the apoptotic protein complex from the apoptotic protein complex inactive state to an apoptotic protein complex active state,
thereby activating the effector module, wherein the apoptotic protein complex in the apoptotic protein complex active state is capable of inducing apoptosis in the cell.

34. (canceled)

35. (canceled)

36. The synthetic protein circuit of claim 33, wherein:
the first orthogonal protease cleavage site comprises the sequence of any one of SEQ ID NOs 247, 253, and 255-258 or a sequence having one or two mismatches relative to any one of SEQ ID NOs 247, 253, and 255-258; and/or
the apoptosis polypeptide comprises the sequence of any one of SEQ ID NOs: 220-221 or a sequence at least 85% identical to any one of SEQ ID NOs: 220-221.

37. The synthetic protein circuit of claim 2, wherein the effector module comprises a synthetic pyroptosis circuit comprising:
a pyroptosis polypeptide comprising a pyroptosis effector domain and an inhibitory domain separated by a first orthogonal protease cleavage site, wherein the inhibitory domain is capable of inhibiting activity of the pyroptosis effector domain, thereby the pyroptosis polypeptide is in a pyroptosis polypeptide inactive state,
wherein the first orthogonal protease in a first orthogonal protease active state is capable of cleaving the first orthogonal protease cleavage site of the pyroptosis polypeptide, and wherein the first orthogonal protease cleavage site of the pyroptosis polypeptide being cleaved changes the pyroptosis polypeptide from the pyroptosis polypeptide inactive state to a pyroptosis polypeptide active state,
thereby activating the effector module, wherein the pyroptosis polypeptide in the pyroptosis polypeptide active state is capable of inducing pyroptosis in the cell.

38.-40. (canceled)

41. The synthetic protein circuit of claim 37, wherein:
the first orthogonal protease cleavage site comprises the sequence of any one of SEQ ID NOs 247, 253, and 255-258 or a sequence having one or two mismatches relative to any one of SEQ ID NOs 247, 253, and 255-258; and/or
the pyroptosis polypeptide comprises the sequence of any one of SEQ ID NOs: 237-241 or a sequence at least 85% identical to any one of SEQ ID NOs: 237-241.

42. The synthetic protein circuit of claim 2, wherein the first orthogonal protease comprises tobacco etch virus (TEV) protease, tobacco vein mottling virus (TVMV) protease, hepatitis C virus protease (HCVP), derivatives thereof, or any combination thereof.

43.-69. (canceled)

70. The synthetic protein circuit of claim 2, wherein the cell is a cancer cell.

71. A nucleic acid composition, comprising:
one or more polynucleotides encoding the synthetic protein circuit of claim 2, wherein the one or more polynucleotides comprise:
one or more first polynucleotides encoding the first polypeptide, the second polypeptide, the proprotein, or any combination thereof; and/or
one or more second polynucleotides encoding the apoptosis polypeptide and/or the pyroptosis polypeptide.

72.-87. (canceled)

88. A method of treating a disease or disorder characterized by aberrant signaling of one or more oncoproteins comprising: administering the nucleic acid composition of claim 74 to a subject in need thereof.

89.-99. (canceled)
